(19) United States
(12) Patent Application Publication
(10) Pub. No.: US 2024/0382651 A1
(43) Pub. Date: Nov. 21, 2024

(54) BUPIVACAINE-COLLAGEN DRUG DELIVERY IMPLANT AND METHODS OF MAKING AND USING THEREOF

(71) Applicant: INNOCOLL PHARMACEUTICALS LIMITED, MONKSLAND, ATHLONE (IE)

(72) Inventors: Gwendolyn Niebler, Chester Springs, PA (US); Alexandra Dietrich, Regensburg (DE); Louis Pascarella, Basking Ridge, NJ (US)

(21) Appl. No.: 18/293,185

(22) PCT Filed: Jul. 28, 2022
(86) PCT No.: PCT/EP2022/071280

§ 371 Date: Jan. 29, 2024

Related U.S. Application Data
(60) Provisional application No. 63/226,590, filed on Jul. 28, 2021.
(60) Provisional application No. 63/331,741, filed on Apr. 15, 2022.

(51) Int. Cl. A61L 27/24; A61L 27/54

(52) U.S. Cl. CPC A61L 27/24; A61L 27/54; A61L 2300/402

(57) ABSTRACT

In one aspect, the present disclosure provides a drug delivery device comprising a collagen matrix and an anesthetic drug substance. In another aspect, the present disclosure provides methods of isolating and maturing the collagen used to make the collagen matrix. In yet another aspect, the present disclosure provides methods of making the drug delivery device from the matured collagen.

### CROSS-REFERENCE TO RELATED APPLICATIONS

The present application is an International Application which claims priority to U.S. Provisional Application No. 63/226,590 filed Jul. 28, 2021 and U.S. Provisional Application No. 63/331,741 filed Apr. 15, 2022, each of which is incorporated by reference herein in its entirety.

### BACKGROUND

### Field

The present disclosure relates generally to the collagen implants for controlled drug release, an in particular, to implants for controlled release of an anesthetic.

### Background

Bupivacaine is indicated to provide local analgesia associated with surgical procedures and has been marketed in the United States for over 20 years as Marcaine™.

A surgical wound is a disruption of the normal structure and function of the skin and underlying soft tissue. Perioperative pain (including visceral pain, but not bony pain) associated with soft tissue surgery is mediated locally and through neural pathways, regardless of the type of soft tissue surgery. The pain is generally maximal during the first 1 to 3 days after surgery, with a gradual decrease over time. Bupivacaine provides analgesia by working at the local tissue level. It blocks the generation and conduction of nerve impulses, presumably by increasing the threshold for electrical excitation in the nerve, by slowing the propagation of the nerve impulse, and by reducing the rate of rise of the action potential in the nerves. It does not require systemic exposure to be effective. Given this, infiltration of surgical wounds with bupivacaine has become a widely accepted component of a multimodal approach to the management of acute postoperative pain after soft tissue surgery, with the goals of providing pain relief and limiting the need for opioids. Decreasing the exposure to opioids has important clinical implications. Opioids are associated with adverse events (AEs) that can impact recovery from surgery, and even short-term opioid use (e.g., 3 days) has been associated with a risk of long-term opioid use.

Bupivacaine, however, has a limited duration of effect (2 to 8 hours with infiltration) that does not align well with the temporal profile of maximum perioperative pain. Duration of effect with bupivacaine is primarily driven by the length of time that it is in contact with the nerves. Various attempts have been made to extend the delivery of bupivacaine to the tissues in the surgical wound. These approaches include administration of bupivacaine via continuous infusion pump, adding a vasoconstrictor such as epinephrine to bupivacaine to alter its removal from the site of action, or the use of drug delivery formulation technology. All of the available formulations of bupivacaine are liquid and have administration, duration of effect, and/or safety limitations. For example, pumps can become dislodged, epinephrine has its own side effect profile, and liquid formulations of bupivacaine need to be prepared for infiltration by being drawn into a syringe, which can result in dosing errors. In addition, human error during administration of liquid formulations of bupivacaine can result in unintended intravascular injection, which can lead to the drug's most serious safety issue, elevated systemic bupivacaine plasma concentrations and associated systemic bupivacaine toxicity, known as local anesthetic systemic toxicity (LAST). Local anesthetic systemic toxicity can result in serious central nervous system and cardiovascular system symptoms, including respiratory. There is no injection technique that is known to eliminate the risk of intravascular injection. High bupivacaine plasma levels may also result from overdose, rapid absorption from the injection site after a bolus of liquid bupivacaine, or diminished tolerance.

There is a need in the art for a drug delivery device comprising bupivacaine as well as methods of making and using such a device. The present disclosure addresses these unmet needs.

### SUMMARY

In one aspect, the disclosure provides a method of making a mature lyophilized milled collagen (LMC), the method comprising the steps of: (a) providing isolated collagen, optionally an isolated collagen dispersion; (b) freezing the isolated collagen; (c) dehydrating the frozen collagen; and (d) maturing the dehydrated collagen. In an embodiment, the dehydrated collagen is placed in a permeable pouch before step (d) and step (d) comprises heating the dehydrated collagen in an environment with controlled temperature and controlled humidity. In an embodiment, the dehydrated collagen is heated to about 40° C. in an environment of about 65% relative humidity. In an embodiment, the dehydrated collagen is maintained in the environment with controlled temperature and controlled humidity until the dehydrated collagen reaches a LOD (loss on drying) of about 18%. In an embodiment, an aqueous dispersion comprising 0.9 wt % of the dehydrated collagen matured to an LOD of about 18% has a viscosity of between about 110 cP and about 250 cP. In an embodiment, the method further comprises the step of (e) dehumidifying the mature LMC to form dehumidified mature LMC. In an embodiment, the mature LMC is dehumidified in a permeable pouch in an environment with controlled temperature and controlled humidity. In an embodiment, the mature LMC is dehumidified at a temperature of about 25° C. in an environment of about 15% relative humidity. In an embodiment, the matured collagen is dehumidified until a loss on drying of about 10% is reached.

In another aspect, the present disclosure provides a method of making a drug delivery device, the method comprising the steps of: (a) forming a dispersion of dehumidified mature lyophilized milled collagen (LMC); (b) adding a solution of bupivacaine, or a salt thereof, to the dispersion to form a bupivacaine-collagen mixture; (c) filling a container with the bupivacaine-collagen mixture; and (d) freeze drying the contents of the container to form a drug delivery device comprising a collagen matrix comprising bupivacaine, or a salt thereof. In an embodiment, the dehumidified mature LMC comprises dehydrated LMC that has a loss on drying (LOD) of about 18% which has been dehumidified to an LOD of about 10%. In an embodiment, the method further comprises the steps of: (e) placing the containers into a secondary packaging; (f) sterilizing the drug delivery device in the secondary packaging; and (g) aerating the drug delivery device in secondary packaging. In an embodiment, step (f) comprises sterilizing the drug delivery device with a mixture of about 6% ethylene oxide and about 94% CO₂. In an embodiment, step (g) comprises aerating the drug delivery device until it comprises less than about 175 ppm ethylene chlorohydrin. In an embodiment, step (g) comprises aerating the drug delivery device until it comprises less than about 0.9 ppm ethylene oxide, less than about 1,000 ppm ethylene glycol, or a combination thereof. In an embodiment, the drug delivery device comprises about 4 mg/cm³ to about 8 mg/cm³ collagen and about 6 mg/cm³ to about 10 mg/cm³ bupivacaine, or a salt thereof. In an embodiment, the drug delivery device comprises about 6 mg/cm³ collagen and about 8 mg/cm³ bupivacaine, or a salt thereof.

In yet another aspect, the present disclosure provides a drug delivery device made by the above method. In an embodiment, the device comprises a collagen matrix having dimensions of about 5 cm×5 cm×0.5 cm. In an embodiment, the collagen matrix comprises about 4 mg/cm³ to about 8 mg/cm³ collagen and about 6 mg/cm³ to about 10 mg/cm³ bupivacaine, or a salt thereof. In an embodiment, the collagen matrix comprises about 6 mg/cm³ collagen and about 8 mg/cm³ bupivacaine, or a salt thereof. In an embodiment, the device comprises at least one of (i)-(iii): (i) less than about 175 ppm ethylene chlorohydrin; (ii) less than about 0.9 ppm ethylene oxide; or (iii) less than about 1,000 ppm ethylene glycol. In an embodiment, bupivacaine, or a salt thereof, is released from the device with an in vitro release profile of 50%±10% in the first 30 minutes, 75% 10% within 2 hours, and greater than 80% within 6 hours. In an embodiment, the device has a dry tensile strength of about 1.6 N to about 2.4 N. In an embodiment, the device has a wet tensile strength of about 0.6 N to about 1.2 N.

In yet another aspect, the present disclosure provides a method of performing a soft tissue surgery procedure in a subject in need thereof, the method comprising placing a drug delivery device at a surgical site, wherein the device comprises a collagen matrix and bupivacaine, or a salt thereof. In an embodiment, the device comprises a collagen matrix having dimensions of about 5 cm×5 cm×0.5 cm. In an embodiment, the collagen matrix comprises about 4 mg/cm³ to about 8 mg/cm³ collagen and about 6 mg/cm³ to about 10 mg/cm³ bupivacaine, or a salt thereof. In an embodiment, the collagen matrix comprises about 6 mg/cm³ collagen and about 8 mg/cm³ bupivacaine, or a salt thereof. In an embodiment, the device comprises at least one of (i)-(iii): (i) less than about 175 ppm ethylene chlorohydrin; (ii) less than about 0.9 ppm ethylene oxide; or (iii) less than about 1,000 ppm ethylene glycol. In an embodiment, bupivacaine, or a salt thereof, is released from the device with an in vitro release profile of 50%±10% in the first 30 minutes, 75% 10% within 2 hours, and greater than 80% within 6 hours. In an embodiment, the device has a dry tensile strength of about 1.6 N to about 2.4 N. In an embodiment, the device has a wet tensile strength of about 0.6 N to about 1.2 N. In an embodiment, the surgery procedure is selected from: an abdominoplasty, an open ventral hernia repair, an open abdominal hysterectomy, a laparoscopic-assisted colectomy, a reduction mammoplasty, and combinations thereof. In an embodiment, bupivacaine, or a salt thereof, is present in the drug delivery device in an amount sufficient to provide a duration of local analgesia, local anesthesia, or nerve blockade which lasts for at least about one day after administration. In an embodiment, more than one drug delivery device is placed at the surgical site. In an embodiment, three drug delivery devices are placed at multiple layers in the soft tissue at the surgical site.

### BRIEF DESCRIPTION OF THE DRAWINGS

Various features of illustrative embodiments of the present disclosure are described below with reference to the drawings. The illustrated embodiments are intended to illustrate, but not to limit, the present disclosure. The drawings contain the following figures:

FIG. 1 depicts the process flow of milling lyophilized bovine tendons as well as the testing and specifications of lyophilized, milled bovine tendons.

FIG. 2 depicts the process flow of the extraction and purification of type I collagen.

FIG. 3 depicts a continuation of the process flow of the extraction and purification of type I collagen.

FIG. 4 depicts a continuation of the process flow of the extraction and purification of type I collagen.

FIG. 5 depicts a continuation of the process flow of the extraction and purification of type I collagen.

FIG. 6 depicts the process flow of the lyophilization of collagen.

FIG. 7 is a flowchart of the Xaracoll manufacturing process overview.

FIG. 8 is a Xaracoll process flow diagram depicting the bulk dispersion preparation.

FIG. 9 is a Xaracoll process flow diagram depicting the filling, sealing, and lyophilization.

FIG. 10 is the Xaracoll lyophilization program overview (method #1).

FIG. 11 is the Xaracoll lyophilization program overview (method #2)

FIG. 12 is the Xaracoll process flow diagram depicting inspection, secondary packaging, terminal sterilization, and aeration.

FIG. 13 is a table of the Xaracoll bulk manufacture data.

FIG. 14 is a table of the results of the amino acid analysis.

FIG. 15 is a table of the results of the free amine analysis.

FIGS. 16A-16F are tables of the LC/MS results. FIG. 16A: LC/MS results of sample T0. FIG. 16B: LC/MS results of sample A. FIG. 16C: LC/MS results of sample B. FIG. 16D: LC/MS results of sample B2. FIG. 16E: LC/MS results of sample C. FIG. 16F: LC/MS results of sample D.

FIG. 17 is a table of the results of the trypsin resistance analysis.

FIG. 18 depicts the SDS-PAGE gel. Lanes 1 and 8 are ASC-standard FILK 2019, lane 2 is lyophilized milled collagen (LMC) sample T0, lane 3 is LMC sample A, lane 4 is LMC sample B, lane 5 is LMC sample B2, lane 6 is LMC sample C, and lane 7 is LMC sample D.

FIG. 19 depicts the SEM images of LMC samples T0 (non matured), A, B, B2, C, and D.

FIG. 20 depicts the AFM images of LMC samples T0 (non matured), A, B, B2, C, and D.

FIG. 21 depicts the DSC peak denaturation temperature of LMC samples.

FIG. 22 depicts the DSC normalized denaturation enthalpy of LMC samples.

FIG. 23 contains tables of the reduced (upper part) and non-reduced (lower part) natural cross-link pattern of (m)LMC samples.

FIG. 24 is a table of the collagen glycosylation results.

FIG. 25 depicts the Xaracoll bulk filtration residue from sample A to sample PM (L to R).

FIG. 26 is a plot showing the particle size distribution of the Xaracoll bulk dispersions.

FIG. 27 provides images showing the Xaracoll dispersion stability after 24 h at room conditions, with strong phase separation for B2 and the onset of phase separation for B. The top images are samples A, B, and B2 (L to R) and the bottom images are C, D, and PM (L to R).

FIG. 28 provides images showing the Xaracoll dispersion stability after 1 week at room conditions with strong phase separation for B2 and B and the onset of phase separation for D. The top images are samples A, B, and B2 (L to R) and the bottom images are C, D, and PM (L to R).

FIG. 29 is a plot showing the particle size distribution of the Xaracoll bulk dispersions after 6 weeks in 0.1 M acetic acid.

FIG. 30 is a plot showing the particle size distribution of the Xaracoll bulk dispersions after 6 weeks in 0.01 M acetic acid.

FIG. 31 is a chart showing the change in particle size between measurements in 0.1 and 0.01 M acetic acid.

FIG. 32 is a chart of the free amine content of the Xaracoll matrices.

FIG. 33 is an SDS PAGE gel of the Xaracoll matrices. Lane 1 is sample A, lane 2 is sample B, lane 3 is sample B2, lane 4 is sample C, lane 5 is sample D, lane 6 is sample PM, and lane 7 is ASC-standard FILK 2019.

FIG. 34 is a chart of the results after trypsin digestion of the Xaracoll matrices.

FIG. 35 provides cross section SEM images of the Xaracoll matrices.

FIG. 36 provides AFM images of Xaracoll matrices PM, A, B, B2, C, and D.

FIG. 37 is a plot of the DSC peak denaturation temperature of the Xaracoll samples.

FIG. 38 is a plot of the DSC normalized denaturation enthalpy of the Xaracoll samples.

FIG. 39 is a plot and a table showing the average water uptake results.

FIG. 40 provides a visual of the water uptake for the Xaracoll matrices.

FIG. 41 is a plot of the tensile strength dry maximum of the Xaracoll matrices.

FIG. 42 is a plot of the tensile strength wet maximum of the Xaracoll matrices.

FIG. 43 depicts nonsterile Xaracoll matrices A, B, B2, C, D and PM during wet tensile strength testing. Top is sample A, B, and B2 (L to R) and bottom is sample C, D, and PM (L to R).

FIG. 44 depicts sterile Xaracoll matrices A, B, B2, C, D and PM during wet tensile strength testing. Top is sample A, B, and B2 (L to R) and bottom is sample C, D, and PM (L to R).

FIG. 45 is a plot of the resistance to pressure of the Xaracoll matrices.

FIG. 46 is an overview of the properties of the nonsterile Xaracoll matrices.

FIG. 47 is an overview of the properties of the sterile Xaracoll matrices.

FIG. 48 is a plot of the resistance to bending of the Xaracoll matrices.

FIG. 49 is a plot of the average dissolution profiles of Xaracoll from different mature LMC.

FIGS. 50A-50F are plots of the single and average dissolution profiles of each Xaracoll matrix sample. FIG. 50A is sample A. FIG. 50B is sample B. FIG. 50C is sample B2. FIG. 50D is sample C. FIG. 50E is sample D. FIG. 50F is sample PM.

FIG. 51 is an overview of the mature LMC samples in Example 5.

FIG. 52 is a chart of the mature LMC free amine analysis.

FIG. 53 is a chart of the mature LMC trypsin analysis.

FIG. 54 is an SDS PAGE gel of the mature LMC samples. Lanes 1 and 8 are ASC-standard FILK 2020, lanes 2 and 3 are sample A, lanes 4 and 5 are sample B and lanes 6 and 7 are sample C.

FIG. 55 provides SEM images of the mature LMC samples A, B, C, D, F, and G.

FIG. 56 provides AFM images of the mature LMC samples.

FIG. 57 is a plot of mLMC peak denaturation temperature measured by DSC.

FIG. 58 is a plot of mLMC normalized denaturation enthalpy measured by DSC.

FIG. 59 is a plot of the particle size distribution of the collagen bulk dispersions.

FIG. 60 is a plot of the particle size distribution bupivacaine HCl bulk dispersions.

FIG. 61 is an overview of the bulk dispersion properties.

FIG. 62 is an overview of the nonsterile Xaracoll matrices after 14 days equilibration.

FIG. 63 depicts the Xaracoll bulk filtration residue. Top is sample A, B, and C (L-R) and bottom is sample D, F, and G (L-R).

FIG. 64 is a plot of the Xaracoll matrix thickness data (n=6).

FIG. 65 is a plot of the Xaracoll matrix resistance to pressure data.

FIG. 66 depicts Xaracoll Matrices A, B, C (top L-R), D, F, and G (bottom L-R) during wet tensile strength testing.

FIG. 67 is a plot of the Xaracoll matrix wet tensile strength data.

FIG. 68 is an overview of the properties of the sterile Xaracoll matrices.

FIG. 69 is a plot of the Xaracoll matrix thickness data.

FIG. 70 is a plot of the Xaracoll matrix resistance to pressure data.

FIG. 71 depicts sterile Xaracoll matrices A, B, C (top L-R), D, F, and G (bottom L-R) during wet tensile strength testing.

FIG. 72 is a plot of the Xaracoll matrix wet tensile strength data.

FIG. 73 is a plot of the sterile Xaracoll matrices A, B, C, D, and F swelling time against mLMC viscosity.

FIG. 74 is a plot and a table of the average water uptake of the sterile Xaracoll matrices.

FIG. 75 is a plot of the dissolution profiles of Xaracoll batches A, B, C, D, F and G.

FIGS. 76A-76F are plots of the dissolution profile for each Xaracoll sample. FIG. 76A is sample A. FIG. 76B is sample B. FIG. 76C is sample C. FIG. 76D is sample D.

FIG. 76E is sample F. FIG. 76F is sample G.

FIG. 77 is a table showing Xaracoll release results at time points of specification.

FIG. 78 is a table of the gradients.

FIG. 79 is a plot of the Xaracoll peak denaturation temperature measured by DSC.

FIG. 80 is a plot of the Xaracoll normalized denaturation enthalpy measured by DSC.

FIG. 81 depicts cross section SEM images of the Xaracoll matrices (A-G).

FIG. 82 depicts “bowling” of Xaracoll matrices made from high viscosity dehydrated mature LMC. L-R: 191 cP, 336 cP, and 503 cP.

FIGS. 83A-83B illustrate the Mean Plasma Bupivacaine Concentration-Time Profiles; Mean (±SD) Plasma Bupivacaine Concentrations by Surgery Type on Linear (FIG. 83A) and Semi-Logarithmic Scale (FIG. 83B)—PK Analysis Set.

FIGS. 84A-84B illustrate the Mean Plasma Bupivacaine Concentration-Time Profiles; Mean (±SD) Plasma Bupivacaine Concentrations by Surgery Type on Linear (FIG. 84A) and Semi-Logarithmic Scale (FIG. 84B) (0-6 Hours)—PK Analysis Set.

FIGS. 85A-85B illustrate the Mean Plasma Bupivacaine Concentration-Time Profiles; Mean (±SE) Plasma Bupivacaine Concentrations by Surgery Type on Linear (FIG. 85A) and Semi-Logarithmic Scale (FIG. 85B)—PK Analysis Set.

FIGS. 86A-86B illustrate Box Plots of Plasma Bupivacaine Pharmacokinetic Parameters; FIG. 86A: Box Plots of Plasma Bupivacaine Cₘₐₓ by Surgery Type on Linear Scale—PK Analysis Set; the dashed line is the median; the solid line is the arithmetic mean. The ends of the “box” are the 25th and 75th percentiles. The whiskers show the lowest data value still within 1.5 IQR of the lower quartile, and the highest value still within 1.5 IQR of the upper quartile, where IQR is the interquartile range. Data values that do not fall between the whiskers are plotted as outliers; FIG. 86B: Box Plot of Plasma Bupivacaine AUC_{0-∞} by Surgery Type on Linear Scale—PK Analysis Set; The dashed line is the median; the solid line is the arithmetic mean. The ends of the “box” are the 25th and 75th percentiles. The whiskers show the lowest data value still within 1.5 IQR of the lower quartile, and the highest value still within 1.5 IQR of the upper quartile, where IQR is the interquartile range. Data values that do not fall between the whiskers are plotted as outliers.

FIG. 87 illustrates the comparison of time to first opioid use between treatment groups, and the difference in percentage of patients that stay opioid free on the drug delivery device described herein, compared to placebo implant.

### DETAILED DESCRIPTION

In one aspect, the present disclosure provides a drug delivery device comprising a fibrillar collagen matrix and at least one anesthetic drug substance. In an embodiment, the anesthetic drug substance is bupivacaine, or a salt thereof. In one embodiment, the anesthetic drug substance is bupivacaine HCl. In an embodiment, the drug delivery device can be implanted in a subject to provide pain management following a surgery. In one embodiment, the surgery is a soft tissue surgery.

In another aspect, the present disclosure provides methods of making the drug delivery device. In yet another aspect, the present disclosure provides methods of isolating the collagen that is used to make the collagen matrix of the drug delivery device as well as methods of making a mature LMC from the isolated collagen. In an embodiment, the mature LMC is dehumidified mature LMC.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

As used herein, the articles “a” and “an” are used to refer to one or to more than one (i.e., to at least one) of the grammatical object of the article. By way of example, “an element” means one element or more than one element.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale. For instance, when referring to a measurable value such as an amount, a temporal duration, and the like, the term “about” is meant to encompass variations of ±20% or +10%, more preferably ±5%, even more preferably ±1%, and still more preferably ±0.1% from the specified value, as such variations are appropriate to perform the disclosed methods.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The word “exemplary” is used herein to mean “serving as an example, instance, or illustration.” Any embodiment described herein as “exemplary” is not necessarily to be construed as preferred or advantageous over other embodiments.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

The terms “maturing” and “equilibration” are used interchangeably herein to refer to processing the dehydrated collagen under conditions suitable to allow ageing of the dehydrated collagen without substantial degradation or contamination.

The term “relative humidity” as used herein refers to a measure of the maximum amount of water in a mixture of gas and water vapor, optionally at a given gas temperature and atmospheric pressure, optionally at constant atmospheric pressure, optionally expressed as a percentage of the maximum amount of water vapor within the gas at the given gas temperature and atmospheric pressure. For the purposes of this specification, the term “relative humidity” is intended to mean a measure of the amount of water vapor in a mixture of environmental air and water vapor, in which the maturing step is conducted, at a constant atmospheric pressure, and expressed as a percentage. For the purposes of this specification, atmospheric pressure understood to be about 980 to about 1040 millibars.

The terms “Xaracoll” and “Xaracoll matrix” are used herein to refer to a drug delivery product comprising 100 mg of bupivacaine HCl homogeneously dispersed in 75 mg of collagen in a matrix measuring approximately 5 cm×5 cm×0.5 cm.

The term “dispersion” as used herein refers to a mixture in which collagen particles are dispersed in a fluid, optionally a liquid, further optionally an aqueous, medium. The collagen particles may comprise collagen molecules, or aggregates thereof; which are dispersed in a fluid, optionally a liquid, further optionally an aqueous, medium. Optionally, the collagen particles, which are dispersed in a fluid, optionally a liquid, further optionally an aqueous, medium; have a length (or maximum dimension) of at least about one micrometer.

A “subject” or “patient,” as used therein, may be a human or non-human mammal. Non-human mammals include, for example, livestock and pets, such as ovine, bovine, porcine, canine, feline and murine mammals. Preferably, the subject is a human.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention may be understood more readily by reference to the following detailed description taken in connection with the accompanying figures and examples, which form a part of this disclosure. It is to be understood that this invention is not limited to the specific methods, applications, conditions or parameters described and/or shown herein, and that the terminology used herein is for the purpose of describing particular embodiments by way of example only and is not intended to be limiting of the claimed invention.

In accordance with some embodiments of the present disclosure, a drug delivery device may include a collagen matrix and at least one drug substance. The at least one drug substance may be an anesthetic. In an embodiment, the drug substance comprises bupivacaine, or a salt thereof. In one embodiment, the drug substance comprises bupivacaine HCl. The drug substance may be substantially homogeneously dispersed in the collagen matrix.

In accordance with some embodiments, a method of administering a drug substance to a patient may include implanting a drug delivery device at a site within a patient where the drug is to be delivered, wherein the drug delivery device comprises a collagen matrix and the drug substance. The drug substance may be an anesthetic.

In an embodiment, the drug delivery device is implanted in a patient after a surgery in order to provide an anesthetic effect. In an embodiment, the surgery is a soft tissue surgery. In an embodiment, the soft tissue surgery is open ventral hernia repair, abdominoplasty, open abdominal hysterectomy, laparoscopic-assisted colectomy, or reduction mammoplasty.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of delivery of anticancer drugs, such embodiments can be used with various devices and implants. Furthermore, various applications of such embodiments and modifications thereto, which may occur to those who are skilled in the art, are also encompassed by the general concepts described herein.

The following disclosure describes the methods, systems, devices and kits associated with a drug delivery device for persistent, controlled delivery of a drug substance such as, for example, an anesthetic. However, those of skill in the art, upon understanding of the present disclosure, will be able to suitably modify the methods, systems, devices and kits disclosed herein for implanting other types of implants designed for controlled release of other drugs upon implantation.

### Drug Delivery Device

In one aspect, the present disclosure relates to a drug delivery device comprising a fibrillar collagen matrix and at least one anesthetic drug substance. In an embodiment, the at least one drug substance is substantially homogeneously dispersed in the collagen matrix. In an embodiment, the at least one drug substance is in an amount sufficient to provide a duration of local analgesia, local anesthesia or nerve blockade which lasts for at least about one day after administration. In an embodiment, the incorporation of the at least one drug substance in the fibrillar collagen matrix delays the release of the at least one drug substance from the fibrillar collagen matrix and, thereby, prolongs the duration of local analgesia, local anesthesia or nerve blockade to at least about one day after administration of the disclosed compositions. In an embodiment, the fibrillar collagen matrix prolongs the duration of local analgesia, local anesthesia or nerve blockade to at least about 72 hours after administration of the disclosed compositions.

Examples of drug delivery device including a collagen matrix and a drug substance include a bupivacaine-collagen implant which is described in U.S. Pat. No. 8,034,368, which is incorporated herein by reference in its entirety for all purposes. Formulations for and methods of obtaining collagen that can be used in a drug-release collagen implant are described in U.S. Pat. No. 10,487,134, which is incorporated herein by reference in its entirety for all purposes. Other examples of drug delivery implants for controlled, sustained drug delivery are described in International Patent Application Publication Nos. WO 2019/071243, WO 2019/071245, WO 2019071246, WO 2019/136490, WO 2019/221853, WO 2020/047013, and WO 2020/046973; US Patent Application Publication Nos. US 2020/0246255, US 2021/0069101, and US 2020/0368398; and Chinese Patent Application Publication Nos. CN 111432807, CN 111655303, and CN 112367980, each of which is incorporated herein by reference in its entirety for all purposes.

Suitable drug substances comprise amino amide anesthetics and amino ester anesthetics and their salts, hydrates and prodrugs. Such drug substances include, but are not limited to, amino amides such as Bupivacaine, Levobupivacaine, Lidocaine, Mepivacaine, Prilocaine, Ropivacaine, Articaine, Trimecaine and their salts and prodrugs; and amino esters such as Benzocaine, Chloroprocaine, Cocaine, Procaine, Tetracaine and their salts and prodrugs. Bupivacaine, and its salts and prodrugs is an optional drug substance. Mixtures of amino amides are contemplated, as are mixtures of amino esters. Mixtures of amino amides and amino esters are also contemplated.

The drug delivery device uses a collagen matrix to achieve extended delivery of the drug substance when placed in the surgical wound during soft tissue surgery. In an embodiment, the drug delivery device, while implanted in a patient, releases the drug substance at the treatment site over a period of time such as, for example, 12 hours, 24 hours, 2 days, 3 days, 4 days, 5 days, 6 days, 7 days, 8 days, 9 days, 10 days, 2 weeks, 3 weeks, 4 weeks, 5 weeks, 6 weeks, or any amount of time between any two of these periods. In an embodiment, the drug delivery device releases the drug substance over about 24 hours. In another embodiment, the drug delivery device releases the drug substance over about 72 hours. In another embodiment, the drug delivery device releases the drug substance over about 24 hours wherein the drug substance provides an anesthetic effect to the patient for more than 24 hours. In an embodiment, the drug delivery device releases the drug substance over about 24 hours wherein the drug substance provides an anesthetic effect to the patient for about 72 hours.

The release profile of the drug delivery device may be tailored depending on the drug and particular application of the drug. Thus, in some embodiments, the drug delivery device is configured to release the drug substance at a first rate for a first period of time and at a second rate for a second period of time. The first rate may be greater than the second rate. In an embodiment, 50%±10% of the drug substance is released in the first 30 minutes, 75%±10% of the drug substance is released within 2 hours, and greater than 80% of the drug substance is released within 6 hours.

In an embodiment, the extended delivery of the drug substance results in a reduction in pain intensity up to 72 hours and an analgesic temporal profile that matches that of maximal soft tissue postsurgical pain.

The collagen matrix in the drug delivery device is manufactured using proprietary technology, which results in a highly purified, porous, biocompatible, biodegradable, and bioresorbable collagen matrix that releases the locally acting drug substance over time into the surgical wound. In an embodiment, the drug substance comprises bupivacaine, or a salt thereof. In an embodiment, the drug substance comprises bupivacaine HCl.

In an embodiment, the collagen matrix comprises Type I collagen. Type I collagen is ubiquitous in humans and animals. Human and animal (e.g., bovine) Type I collagen have almost identical amino acid sequencing, including positioning of antigenic determinates outside of the triple helix. Bovine Type I collagen is used in numerous clinical applications. The lack of an adverse immune response to the use of xenogeneic collagen in implantable materials is attributed to the common nature between species of amino acid sequences and surface epitopes. When in its native ultrastructure, implanted xenogeneic material is generally recognized as “self” tissue, regardless of the species of origin, and is subjected to the fundamental biological process of degradation and integration into adjacent host tissues. Some collagen products, e.g., those used in the dermatologic field intended to remain intact in the body for an extended period of time, undergo structural modifications during manufacturing (e.g., cross-linking) designed to alter the rate of enzymatic degradation and remodeling. Collagen products without modifications degrade rapidly in vivo, generally over 1 to 3 months. In an embodiment, the collagen in the drug delivery device comprises collagen that has not undergone structural modifications and thus degrades over 1 to 3 months in vivo.

In an embodiment, the Type I collagen used in drug delivery device is purified from bovine Achilles tendons. In an embodiment, the Type I collagen is obtained exclusively from New Zealand closed herds that have been certified as transmissible spongiform encephalopathy-free and negligible for the risk of bovine spongiform encephalopathy. The tendon undergoes a collagen extraction and purification process that does not result in cross-linking (structural modification), which differs from collagen products designed to remain intact in the body for an extended period of time. Therefore, the finished collagen matrix of drug delivery device contains a significant content of tropocollagen microfibrils, which are more susceptible to enzymatic degradation and promote resorption in the body, in contrast to cross-linked collagen products.

The collagen matrix in drug delivery device is designed to act as a vehicle for delivery of bupivacaine into the wound and has no positive or detrimental effect on wound healing. After placement of the drug delivery device into the surgical site, it absorbs liquid from the site, resulting in dissolution and diffusion of bupivacaine from the porous lyophilized collagen matrix. In an embodiment, nearly 100% of bupivacaine is released from the matrix within 24 hours. The collagen matrix subsequently degrades via slow chemical and enzymatic hydrolysis to soluble peptides and amino acids, which are absorbed into the tissue. The quantity of matrix material decreases over time in the wound. In an embodiment, the collagen matrix based is no longer present by 56 days after placement in an animal.

In an embodiment, the drug delivery device has a length of about 50 mm, a width of about 50 mm, and a thickness of about 5 mm. In some embodiments, the drug delivery device may have any shape, in particular, cylindrical, semi-cylindrical, corrugated, cuboid, hexahedral, or any other shape.

In an embodiment, the drug delivery device comprises a collagen matrix having dimensions of about 5 cm×5 cm×0.5 cm. In an embodiment, the matrix comprises about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 50 mg to about 100 mg, about 65 mg to about 85 mg, or about 75 mg collagen. In an embodiment, the matrix comprises about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 50 mg to about 150 mg, about 75 mg to about 125 mg, about 90 mg to about 110 mg, or about 100 mg bupivacaine, or a salt thereof. In an embodiment, the matrix comprises about 0.5 mg/cm³ to about 20 mg/cm³, about 0.5 mg/cm³ to about 18 mg/cm³, about 0.5 mg/cm³ to about 16 mg/cm³, about 0.5 mg/cm³ to about 14 mg/cm³, about 0.5 mg/cm³ to about 12 mg/cm³, about 0.5 mg/cm³ to about 10 mg/cm³, about 2 mg/cm³ to about 10 mg/cm³, about 4 mg/cm³ to about 10 mg/cm³, about 4 mg/cm³ to about 8 mg/cm³, or about 6 mg/cm³ collagen. In an embodiment, the matrix comprises about 0.5 mg/cm³ to about 20 mg/cm³, about 0.5 mg/cm³ to about 18 mg/cm³, about 0.5 mg/cm³ to about 16 mg/cm³, about 0.5 mg/cm³ to about 14 mg/cm³, about 0.5 mg/cm³ to about 12 mg/cm³, about 2 mg/cm³ to about 12 mg/cm³, about 4 mg/cm³ to about 12 mg/cm³, about 4 mg/cm³ to about 10 mg/cm³, about 6 mg/cm³ to about 10 mg/cm³, or about 8 mg/cm³ bupivacaine.

In an embodiment, drug delivery device has a dry tensile strength of about 0.5 N to about 10 N, about 0.5 N to about 9 N, about 0.5 N to about 8 N, about 0.5 N to about 7 N, about 0.5 N to about 6 N, about 0.5 N to about 5 N, about 0.5 N to about 4 N, about 1 N to about 3.5 N, about 1.5 N to about 3 N, about 1.6 N to about 2.4, or about 1.8 N to about 2.2 N.

In an embodiment, the drug delivery device has a wet tensile strength of about 0.1 N to about 10 N, about 0.1 N to about 9 N, about 0.1 N to about 8 N, about 0.1 N to about 7 N, about 0.1 N to about 6 N, about 0.1 N to about 5 N, about 0.1 N to about 4 N, about 0.1 N to about 3 N, about 0.5 N to about 2.5 N, about 0.5 N to about 2 N, about 0.5 N to about 1.5 N, or about 0.6 N to about 1.2 N.

In an embodiment, the drug delivery device comprises 100 mg bupivacaine HCl (equivalent to 88.8 mg of bupivacaine) homogeneously dispersed in 75 mg of collagen in a matrix measuring approximately 5 cm×5 cm×0.5 cm. In an embodiment, the proposed dose of bupivacaine HCl is 300 mg (equivalent to a total of 266.4 mg of bupivacaine), achieved by implanting 3×100 mg collagen matrices during surgery. In an embodiment, the drug delivery device is implanted during a soft tissue surgery. In an embodiment, the drug delivery device is implanted at multiple layers in the soft tissue (e.g., between the fascia/muscle closure and in the layers below the skin closure).

In an embodiment, the drug delivery device is made using a method described elsewhere herein. In an embodiment, the drug delivery device is made using a method described herein from dehumidified mature LMC described elsewhere herein. In an embodiment, the dehumidified mature LMC is made using a method described elsewhere herein.

In an embodiment, using the dehumidified mature LMC prepared as described elsewhere herein to make a drug delivery device results in a drug delivery device with one or more different properties than a drug delivery device that is made from LMC that is not matured or is matured using a different process. In an embodiment, while an aqueous dispersion of 0.9 wt % dehydrated collagen matured to an LOD as described elsewhere herein has a viscosity within a desired range, a dispersion of unmatured dehydrated collagen or a dispersion of dehydrated collagen matured using different parameters and/or a different process has a viscosity below or above the desired range. In an embodiment, the desired viscosity range is between about 50 cP and about 500 cP, about 75 cP and about 500 cP, about 100 cP and about 500 cP, about 100 cP and about 450 cP, about 100 cP and about 400 cP, about 100 cP and about 350 cP, about 100 cP and about 300 cP, about 100 cP and about 250 cP, about 110 cP and about 250 cP, about 150 cP and about 250 cP, or about 160 cP and about 250 cP. In an embodiment, lower viscosities have a more adverse effect on the resulting drug delivery device. Therefore, in an embodiment, the dispersion of dehydrated collagen matured to an LOD as described elsewhere herein should have a viscosity of at least about 110 cP, at least about 120 cP, at least about 130 cP, at least about 140 cP, at least about 150 cP, at least about 160 cP, or at least about 170 cP, but may have a viscosity of greater than 250 cP.

In an embodiment, using unmatured LMC or matured LMC obtained from dehydrated collagen with a viscosity outside of the range about 110 cP-250 cP to make a drug delivery device results in a device with one or more different properties than a device made from the dehumidified mature LMC described elsewhere herein.

### Methods of Making Mature Lyophilized Milled Collagen (LMC)

In one aspect, the present disclosure relates to a method of making a mature LMC, the method comprising the steps of: (a) providing isolated collagen, optionally an isolated collagen dispersion; (b) freezing the isolated collagen; (c) dehydrating the frozen collagen; and (d) maturing the dehydrated collagen.

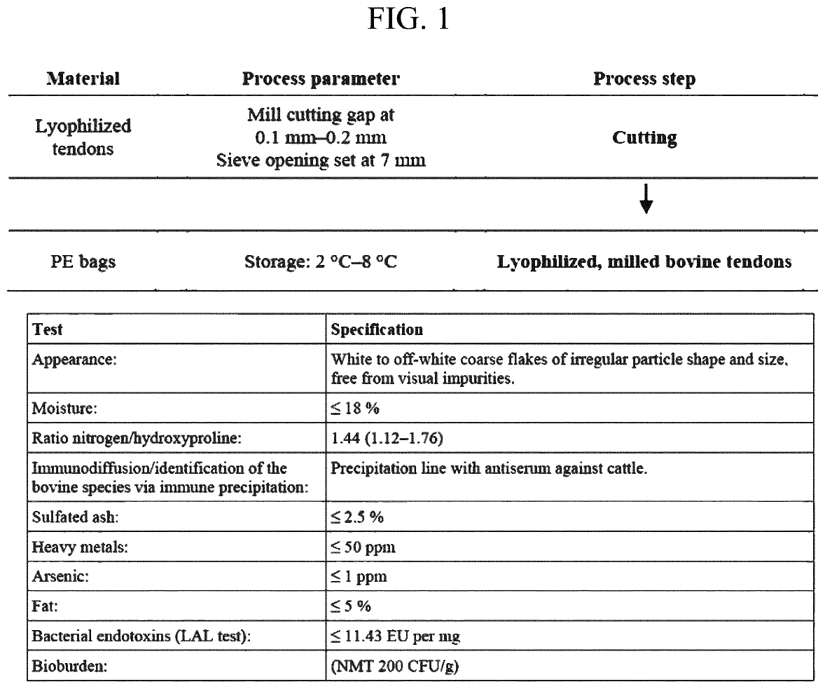

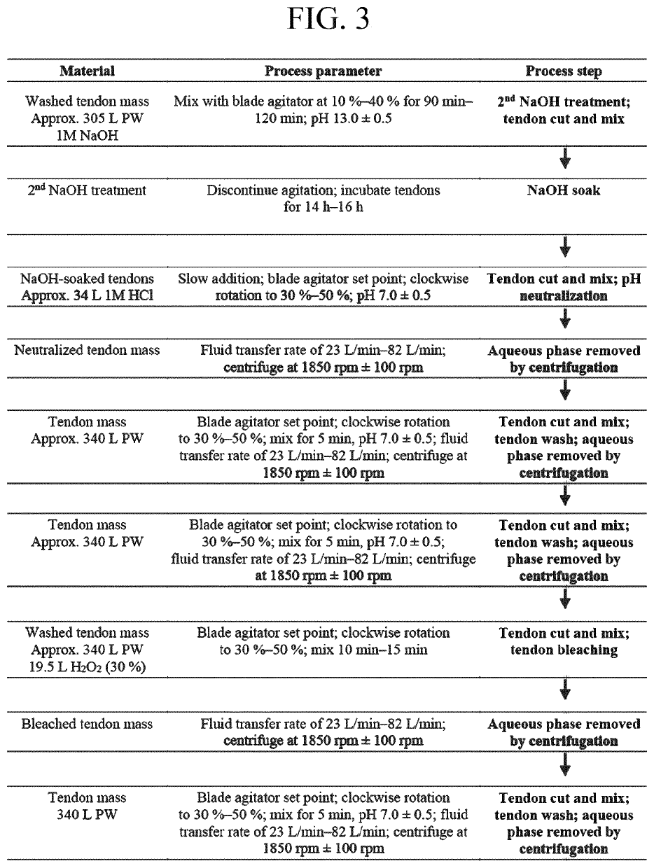

The collagen of step (a) can be isolated from equine or bovine tendons. In an embodiment, the collagen is isolated from bovine tendons. In an embodiment, the isolated collagen is obtained by milling the tendons to degrade the collagen source and treating the milled collagen to extract and purify Type I collagen. In an embodiment, the tendons are lyophilized. In an embodiment, the cutting gap of the mill is set to 0.1 mm-0.2 mm with a discharge sieve setting of 7 mm as detailed in FIG. 1. In an embodiment, the milled collagen is treated with 1 N sodium hydroxide (NaOH) to remove microbiological contamination such as prions, bacteria, and viruses. In one embodiment, the milled collagen is treated with TN NaOH by soaking the milled collagen for 60 min-90 min in 1 M NaOH at a pH>13.5. In an embodiment, the NaOH treated milled collagen is neutralized to a pH of about 7.0±0.5. In one embodiment, the NaOH treated milled collagen is transferred to a vessel containing a defined amount of purified water USP (PW) and neutralized with 1 M HCl to a pH of about 7.0±0.5 while mixing. Although not wishing to be limited by theory, it is believed that neutralization of the NaOH treated milled collagen results in the formation of NaCl, which aids in the removal of low molecular weight soluble intrinsic impurities of the collagen. After neutralization, the solid portion of the milled collagen is subsequently separated from the liquid portion through centrifugation. The solid portion is re-suspended in 0.9% NaCl, and mixed under slow agitation, which enhances the solubility of intrinsic impurities. Following centrifugation, the milled collagen is washed two additional times with PW to further remove low molecular weight soluble components from the collagen. In some embodiments, the milled collagen is treated a second time with NaOH. In one embodiment, the second NaOH treatment comprises treating the milled collagen with 0.1 M NaOH at a pH of about 13.0±0.5 for a period of 14 h-16 h incubation. Although not wishing to be limited by theory, it is believed that the second NaOH treatment contributes to the depletion of fat content and soluble impurities (FIG. 3). After the second NaOH treatment, the milled collagen is neutralized to a pH of about 7.0±0.5. In an embodiment, the second neutralization comprises treating the milled collagen with 1 M HCl to a pH of about 7.0±0.5 and washing the milled collagen twice with PW.

The milled collagen is treated with H₂O₂. In an embodiment, the milled collagen is treated for with about 1.7% H₂O₂ for about 10-15 mins. Although not wishing to be limited by theory, it is believed that the H₂O₂ bleaches the milled collagen and aids in further removal of fat content. The bleached milled collagen is centrifuged and washed with PW to remove residual peroxides. Although not wishing to be limited by theory, it is believed that the bleached milled collagen should be washed to remove residual peroxides that can cause inactivation of the pepsin used in a later process step.

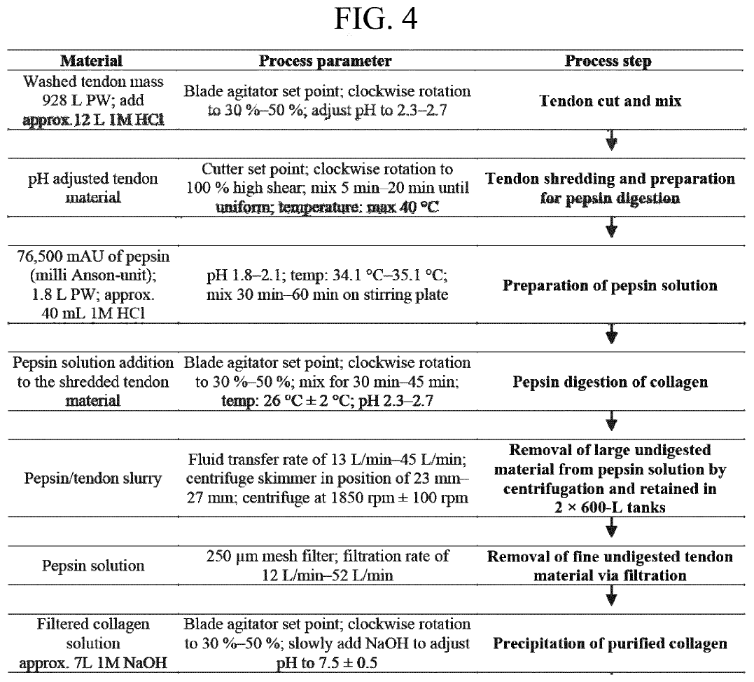

The bleached milled collagen is dispersed in acidified PW (pH of about 2.3-2.7) to affect swelling of the fibrous material (FIG. 4). The swollen milled collagen is then shredded. In an embodiment, the swollen milled collagen is shredded utilizing a tank bottom mounted rotor stator cutter. The shredded collagen is then combined with a prepared pepsin solution. In an embodiment, the pepsin solution is prepared by dissolving the pepsin in acidified water at pH of between about 1.8-2.1 and a temperature of between about 34.1° C.-35.1° C. for 30 min-60 min. Although not wishing to be limited by theory, it is believed that the pepsin reduces the potential for non-collagen protein contamination (eg, bovine serum protein), reduces the potential species related immunogenicity of the collagen through cleavage of telopeptides (i.e., the short non-helical domains at the end of the collagen chains), and/or removes viral contaminates.

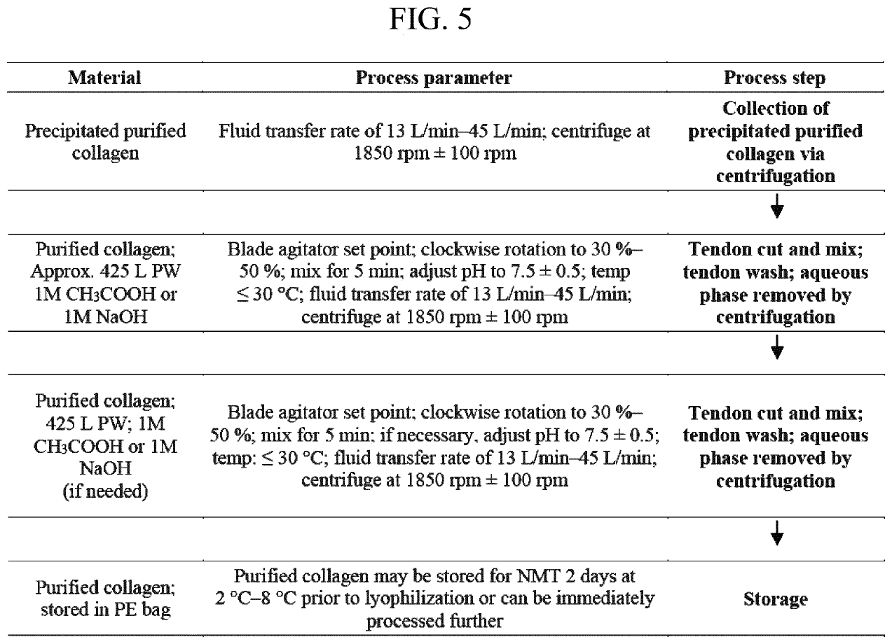

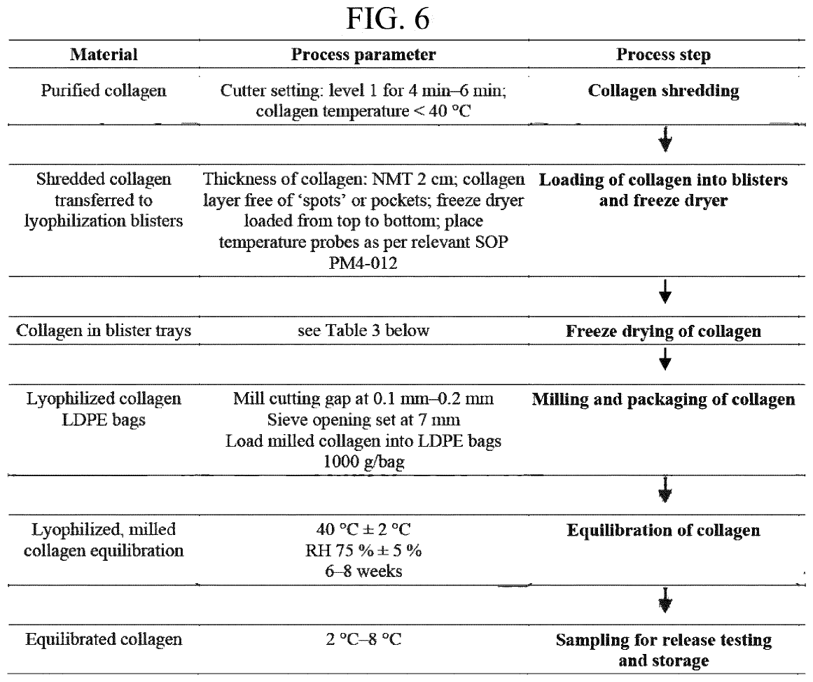

The pepsin treated milled collagen is then centrifuged, the solubilized collagen is retained, and the solid centrifugate is discarded. In an embodiment, the solubilized collagen is filtered. In one embodiment, the solubilized collagen is passed through a 250 μm filter. In an embodiment, the filtered collagen is precipitated through pH adjustment. In one embodiment, the pH adjustment comprises treating the filtered collagen with 1 M NaOH to a pH of about 7.5±0.5. The precipitated collagen is centrifuged, washed twice with PW, and pH adjusted to a pH of about 7.5±0.5 with acid and/or base as necessary to provide the isolated collagen (FIG. 5). In an embodiment, the acid is acetic acid and/or the base is NaOH. At this point in the manufacturing process, the isolated collagen may be placed into LDPE bags and stored before step (b). In an embodiment, the isolated collagen is stored for no more than 2 days at 2° C.-8° C. awaiting step (b) or immediately distributed in trays and lyophilized (FIG. 6).

In some embodiments, the providing step (step (a)) comprises the step of removing the fluid prior to the providing step. In some embodiments, the providing step comprises the step of removing at least some of the fluid prior to the providing step. In some embodiments, the providing step comprises the step of removing at least some of the fluid prior to the providing step; to provide an isolated collagen dispersion. In some embodiments, the fluid is a liquid. In some embodiments, the liquid is an aqueous medium. In some embodiments, the providing step comprises the step of removing the fluid prior to the providing step to provide a dispersion having a concentration of about 3-30%, optionally 3-4%, (w/w) collagen particles. In some embodiments, the fluid is a liquid. In some embodiments, the liquid is an aqueous medium.

In an embodiment, the freezing step (step (b)) comprises freezing to a temperature of about −33° C. to about −42° C. In one embodiment, the freezing step comprises freezing to a temperature of about −38° C. In some embodiments, the freezing step comprises freezing at a rate of about 0.3° C. to about 1.5° C. per minute, optionally a rate of about 0.5° C. per minute.

In an embodiment, the dehydrating step (step (c)) comprises removing the aqueous phase. In one embodiment, the dehydrating step comprises removing the aqueous phase by reducing the pressure. In one embodiment, the dehydrating step comprises removing the aqueous phase by reducing the pressure to about 0.05 to about 0.5 mbar. In one embodiment, the dehydrating step comprises removing the aqueous phase by applying a vacuum. In an embodiment, the dehydrating step comprises evacuating a chamber comprising the frozen collagen to a pressure of about 0.05 to about 0.5 mbar. In one embodiment, the dehydrating step comprises evacuating a chamber comprising the frozen collagen to a pressure of about 0.2 mbar.

Optionally or additionally, the dehydrating step comprises increasing the temperature of the frozen collagen. Further optionally or additionally, the dehydrating step comprises increasing the temperature of the frozen collagen under vacuum. Still further optionally or additionally, the dehydrating step comprises increasing the temperature of the collagen to about +30° C. Still further optionally or additionally, the dehydrating step comprises increasing the temperature of the collagen to about +30° C. under vacuum. In one embodiment, the dehydrating step comprises increasing the temperature of the collagen to about +30° C. under vacuum and holding the collagen at this temperature for about 15 to 36 hours. In one embodiment, the collagen is held at about +30° C. at a pressure of about 0.2 mbar for about 24-26 hours.

Optionally or additionally, the dehydrating step comprises increasing the temperature of the collagen to about +30° C. at a rate of about 0.3° C. to about 1.5° C. per minute, further optionally at a rate of about 0.5° C. per minute. Further optionally or additionally, the dehydrating step comprises increasing the temperature of the collagen to about +30° C. at a rate of about 0.3° C. to about 1.5° C. per minute, further optionally at a rate of about 0.5° C. per minute, under vacuum.

In an embodiment, the dehydrating step comprises decreasing the temperature to about +20° C. and holding the collagen at this temperature under vacuum. In an embodiment, the collagen is held at +20° C. at a pressure of about 0.05 to 0.5 mbar. In one embodiment, the collagen is held at +20° C. at a pressure of about 0.2 mbar. In one embodiment, the collagen is held at collagen is held at +20° C. under vacuum for about 1 hour.

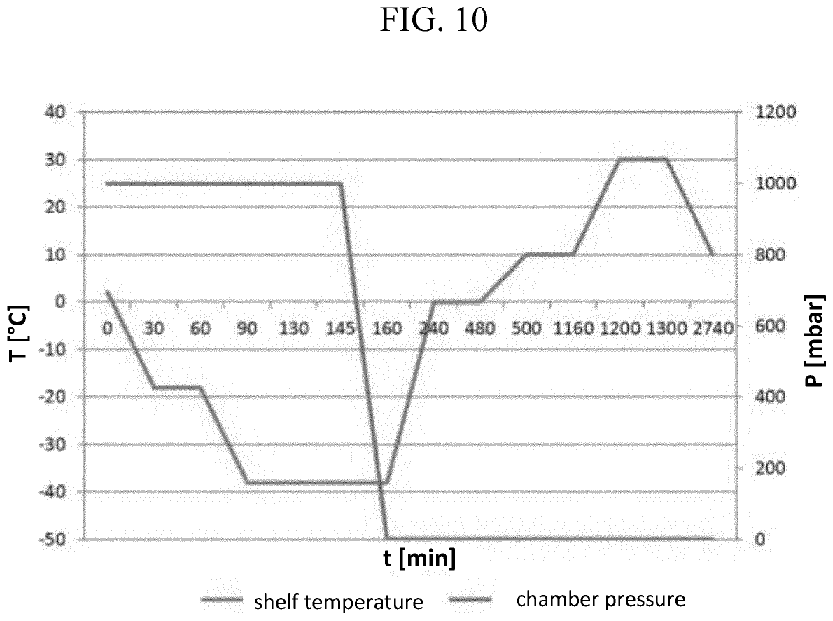

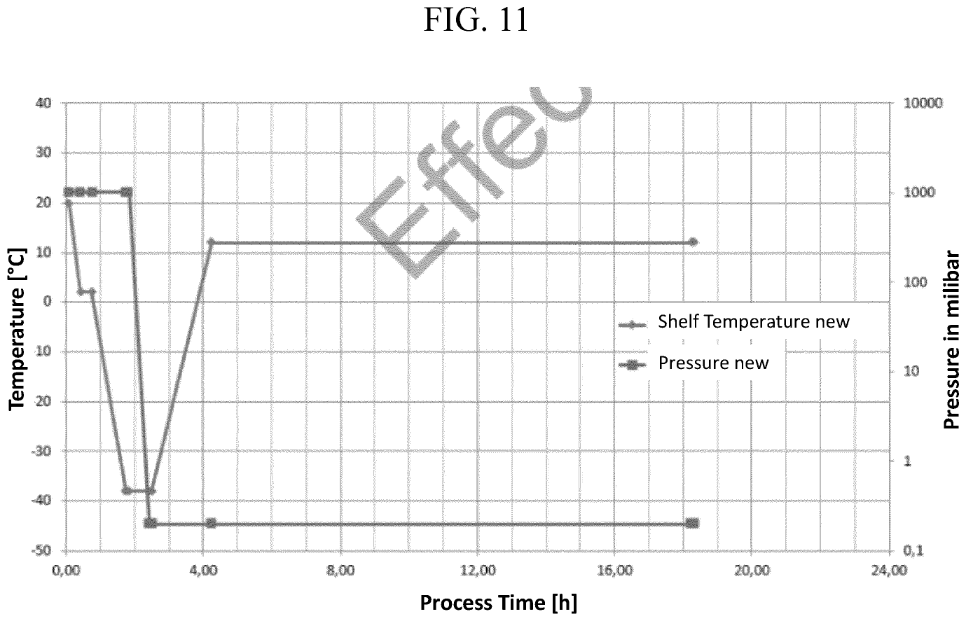

In an embodiment, steps (b) and (c) together comprise a step of lyophilizing the isolated collagen. In an embodiment, the lyophilization parameters are found in FIG. 10 and FIG. 11.

In an embodiment, the dehydrated collagen is placed in a permeable pouch before step (d). The permeable pouch can be any pouch, bag, container, box, etc. that is fully permeable, semi permeable, or partially permeable to the surrounding environment. In an embodiment, the permeable pouch is at least 50%, at least 60%, at least 70%, at least 80%, at least 90%, or at least 95% permeable to the surrounding environment. In one embodiment, the permeable pouch is a paper, Medipeel, or Stericlean pouch.

In an embodiment, step (d) comprises heating the dehydrated collagen in an environment with controlled humidity. In an embodiment, dehydrated collagen in a permeable pouch is heated in an environment with controlled humidity. In one embodiment, dehydrated collagen in a permeable pouch is heated to about 30° C. to about 50° C. in an environment of about 50% to about 75% relative humidity (RH). In one embodiment, dehydrated collagen in a permeable pouch is heated to about 40° C. in an environment of about 65% relative humidity (RH). In an embodiment, the dehydrated collagen is maintained in the heated environment with controlled humidity until the dehydrated collagen reaches an LOD (loss on drying) of between about 16% and about 20%. In an embodiment, the dehydrated collagen is maintained in the heated environment with controlled humidity until the dehydrated collagen reaches an LOD (loss on drying) of about 18%. In one embodiment, dehydrated collagen in a permeable pouch maintained at a temperature of about 40° C. and about 65% RH reaches an LOD of about 18% in about six to eight weeks.

In an embodiment, the dehydrated collagen is placed in a permeable pouch and heated to about 40° C. at about 60% RH for about four to seven days before the dehydrated collagen is heated to about 40° C. at about 65% RH.

In an embodiment, the dehydrated collagen is matured until an LOD of about 18% is reached. In an embodiment, an aqueous dispersion of 0.9 wt % dehydrated collagen matured to an LOD of about 18% has a viscosity of between about 100 cP and about 250 cP (measurement taken of the dispersion having a pH of about 4.5 and held at a temperature of about 40° C.). In an embodiment, an aqueous dispersion of 0.9 wt % dehydrated collagen matured to an LOD of about 18% has a viscosity of between about 150 cP and about 250 cP.

In an embodiment, the matured collagen is dehumidified to form dehumidified mature LMC. In one embodiment, the matured collagen is dehumidified in the permeable pouch. In an embodiment, the matured collagen is dehumidified in an environment with a controlled temperature and controlled RH. In an embodiment, the temperature is maintained at about 15° C. to about 35° C., about 18° C. to about 32° C., about 20° C. to about 30° C., or about 25° C. In an embodiment, the RH is maintained at about 5% to about 25%, about 8% to about 22%, about 10% to about 20%, about 12% to about 18%, or about 15%. In one embodiment, the matured collagen is dehumidified in a permeable pouch at a temperature of about 25° C. and a RH of about 15%. In an embodiment, the matured collagen is dehumidified until an LOD of about 8% to about 12% is reached. In one embodiment, the matured collagen is dehumidified until an LOD of about 10% is reached. In one embodiment, matured collagen in a permeable pouch is dehumidified for about one week at a temperature of about 25° C. and a RH of about 15% to reach an LOD of about 10%.

In an embodiment, the dehumidified mature LMC can be used directly in a dispersion which is mixed with one or more drug substances. In another embodiment, the dehumidified mature LMC is stored before further use. In one embodiment, the dehumidified mature LMC is sealed in an impermeable pouch before storage. In one embodiment, the dehumidified mature LMC is refrigerated for storage. In one embodiment, the dehumidified mature LMC is sealed in an impermeable pouch and stored in a refrigerator. In one embodiment, the impermeable pouch is an aluminum pouch.

It is understood that, in conducting the maturing step, the parameters of temperature, time, pressure, and relative humidity are not necessarily mutually exclusive, and the skilled person would recognize that as one parameter is varied, one or both of the other parameters may also be varied accordingly.

In some embodiments, the isolated collagen is fibrillar collagen. Fibrillar collagen from different sources may be used including commercially available fibrillar collagen, for example, biomedical collagen from Devro Biomedical Collagen, Australia. Currently there are five known types of fibrillar collagen; Type I, II, III, V and XI. Alternatively, collagen can be extracted from tendons or hides of different mammals, including human, horse, cattle, sheep and pigs. Collagen can also be extracted from a non-mammal such as fish. Details on the various types of collagen are described by Gelse et al., (Advanced Drug Delivery Reviews 55 (2003), 1531-1546), the whole contents of which are incorporated herein by reference. The present inventors have used a bovine-derived collagen Type I for the manufacture of bupivacaine-collagen sponges. Equine-derived collagen Type I is also suitable for use in the present invention, as are fibrillar collagen such as type I collagen from pigs and sheep. Type I collagen is a connective tissue extracted from animal tendons and other sources; in this case, the collagen is derived from bovine tendons. The Type I collagen consists of three approximately 1,050 amino-acid-long polypeptide chains, two alpha-1 chains, and one alpha-2 chain. These are coiled to form a right-hand helix (known as a triple helix) around a common axis. The rod-shaped molecule has a length of 2900 Angstrom, a diameter of 14 Angstrom and a molecular weight of approx. 300,000 Daltons. Type I collagen can be typified by its reaction with the protein core of another connective tissue component known as a proteoglycan. Type I collagen contains signaling regions that facilitate cell migration.

Optionally, the isolated collagen is selected from Type I collagen, Type II collagen, Type III collagen, and a mixture thereof. Still further optionally, the isolated collagen is Type I collagen.

### Methods of Making a Drug Delivery Device

In another aspect, the present disclosure relates to a method of making a drug delivery device, the method comprising the steps of: (a) forming a dispersion of dehumidified mature LMC; (b) adding a solution of bupivacaine, or a salt thereof, to the dispersion to form a bupivacaine-collagen mixture; (c) filling a container with the bupivacaine-collagen mixture; and (d) freeze drying the contents of the container to form a drug delivery device comprising a collagen matrix and bupivacaine, or a salt thereof.

In one embodiment, the collagen dispersion comprises an aqueous solution of dehumidified mature LMC. In an embodiment, the collagen dispersion comprises between about 0.1 wt % and about 10 wt %, about 0.1 wt % and about 8 wt %, about 0.1 wt % and about 6 wt %, about 0.1 wt % and about 4 wt %, about 0.1 wt % and about 2 wt %, about 0.1 wt % and about 1 wt %, about 0.4 wt % and about 0.8 wt %, or about 0.6 wt % dehumidified mature LMC. In one embodiment, the aqueous solution is acidic with a pH of about 3.0 to about 5.0, about 3.3 to about 4.7, about 3.6 to about 4.4, about 3.9 to about 4.2, or about 4.1. Although not wishing to be limited by theory, it is believed that the acidic conditions are required to ensure optimum swelling of the collagen during the homogenization step. In one embodiment, the collagen dispersion is maintained at a temperature of about 20° C. to about 50° C., about 24° C. to about 47° C., about 27° C. to about 44° C., about 30° C. to about 41° C., about 35° C. to about 41° C., or about 38° C. In one embodiment, acetic acid is used to acidify the aqueous solution. Although not wishing to be limited by theory, it is believed that using acetic acid rather than HCl leads to a reduction in the concentration of chloride in the drug delivery product and thus minimizes the formation of ECH during EO sterilization.

In an embodiment, the collagen dispersion is homogenized to ensure complete and uniform dispersion of the collagen. In one embodiment, the collagen dispersion is maintained at a temperature of below about 50° C., about 48° C., about 46° C., about 44° C., or about 42° C. during homogenization. The collagen dispersion should be maintained below the collagen denaturation temperature. In an embodiment, the collagen denaturation temperature is about 50° C. In an embodiment, the collagen dispersion is homogenized at about 3,000 rpm±100 rpm for about 5 min±1 min. In an embodiment, the homogenizer comprises a rotor head that can provide high shear forces to separate the fibrous mass of collagen that is present at the beginning of the dispersion preparation and thus to promote adequate swelling of the collagen.

A solution of bupivacaine is added to the collagen dispersion to form a bupivacaine-collagen mixture. In an embodiment, the solution of bupivacaine is an aqueous solution. In an embodiment, the solution of bupivacaine is added to the collagen dispersion such that the resulting bupivacaine-collagen mixture comprises about 0.1 wt % to about 10 wt %, about 0.1 wt % and about 8 wt %, about 0.1 wt % and about 6 wt %, about 0.1 wt % and about 4 wt %, about 0.1 wt % and about 2 wt %, about 0.1 wt % and about 1 wt %, about 0.4 wt % and about 1 wt %, about 0.6 wt % and about 1 wt %, or about 0.8 wt % bupivacaine. In an embodiment the bupivacaine is bupivacaine HCl. In an embodiment, the aqueous solution has an acidic pH. In one embodiment, the aqueous solution has a pH of about 3.0 to about 5.0, about 3.3 to about 4.7, about 3.6 to about 4.4, about 3.9 to about 4.2, or about 4.1. In one embodiment, the aqueous solution is acidified with acetic acid. In an embodiment, the solution of bupivacaine is maintained at a temperature of about 20° C. to about 50° C., about 24° C. to about 47° C., about 27° C. to about 44° C., about 30° C. to about 41° C., about 35° C. to about 41° C., or about 38° C. before it is added to the collagen dispersion. In one embodiment, the bupivacaine-collagen mixture is stirred. In one embodiment, the bupivacaine-collagen mixture is mixed using a homogenizer. In one embodiment, the bupivacaine-collagen mixture is mixed at about 3,000 rpm for about one minute. In an embodiment, the bupivacaine-collagen mixture is maintained at a temperature of about 20° C. to about 50° C., about 24° C. to about 47° C., about 27° C. to about 44° C., about 34° C. to about 44° C., about 38° C. to about 42° C., or about 40° C. In an embodiment, the bupivacaine-collagen mixture is maintained under low shear mixing to ensure temperature homogeneity.

In an embodiment, bupivacaine-collagen mixture is filtered. In one embodiment, bupivacaine-collagen mixture is filtered through a 250 μm filter. In an embodiment, after filtering, the bupivacaine-collagen mixture is maintained at a temperature of about 20° C. to about 50° C., about 24° C. to about 47° C., about 27° C. to about 44° C., about 34° C. to about 44° C., about 38° C. to about 42° C., or about 40° C. In an embodiment, the filtered bupivacaine-collagen mixture is maintained under low shear mixing to ensure temperature homogeneity.

A container is filled with the bupivacaine-collagen mixture or the filtered bupivacaine-collagen mixture. The container can be any type of container known to a person of skill in the art that does not react with the components of the mixture. In an embodiment, the container is a polyethylene terephthalate (PET). In one embodiment, the PET is chemically modified. In one embodiment, the chemically modified PET is glycol modified PET (PETG). In an embodiment, the container is a blister pack. In one embodiment, the container is a PETG blister pack. In an embodiment, the container has dimensions of about 5 cm×5 cm×1.5 cm. In an embodiment, the container is filled with about 10 g to about 15 g of the bupivacaine-collagen mixture or the filtered bupivacaine-collagen mixture.

In an embodiment, the filled container is covered with a lid. In another embodiment, the filled container is left uncovered. The lid can be any kind of gas-permeable lid known to a person of skill in the art. In one embodiment, the lid is a Tyvek lid. In one embodiment, the lid seals to the top of the container.

The filled container is freeze dried and the contents of the container form a collagen matrix comprising bupivacaine. In one embodiment, the filled container is freeze dried using a lyophilizer.

### Freeze Drying Method #1

In an embodiment, the filled container is placed on a shelf and the shelf is cooled to about 20° C., about 15° C., about 10° C., about 5° C., or about 2° C. In an embodiment, the shelf is then cooled further to initiate freezing of the contents of the filled container. In one embodiment, the shelf is cooled stepwise to about −5° C., about −10° C., about −15° C., about −20° C., about −25° C., about −30° C., about −35° C., or about −38° C. In one embodiment, in a first “step,” the shelf is cooled to about −18° C. In one embodiment, the frozen filled container is maintained at about −18° C. for a designated period of time. In one embodiment, the frozen filled container is maintained at about −18° C. for about 15 mins to about 5 hours, about 15 mins to about 4.5 hours, about 15 mins to about 4 hours, about 15 mins to about 3.5 hours, about 15 mins to about 3 hours, about 15 mins to about 2.5 hours, about 15 mins to about 2 hours, about 15 mins to about 1.5 hours, about 45 mins to about 1.5 hours, or about 1 hour. In an embodiment, in a second “step,” the shelf is cooled to a temperature of about −38° C. In an embodiment, the frozen filled container is maintained at this temperature for a designated time. In one embodiment, the frozen filled container is maintained at this temperature for about 30 mins to about 8 hours, about 30 mins to about 7 hours, about 30 mins to about 6 hours, about 30 mins to about 5 hours, about 30 mins to about 4 hours, about 30 mins to about 3 hours, about 30 mins to about 2 hours, about 1 hour to about 2 hours, or about 1.5 hours. In an embodiment after the temperature has been maintained for the designated time, the pressure is then reduced to about 0.05 to 0.5 mbar. In one embodiment, the pressure is reduced to about 0.2 mbar. In an embodiment, the temperature is increased under reduced pressure. In an embodiment, the temperature is increased to about 50° C., about 45° C., about 40° C., about 35° C., or about 30° C. In an embodiment, the temperature is increased stepwise. In one embodiment, the temperature is increased stepwise to about 30° C. In one embodiment, the temperature is increased stepwise for a first “step” of 0° C., a second “step” of 10° C., and a final “step” of 30° C. In one embodiment, the 0° C. temperature is held for about 4.5 to 5.5 hours at a pressure of about 0.2 mbar and/or the 10° C. temperature is held for about 10 to 12 hours at a pressure of about 0.2 mbar. In an embodiment, the contents of the container are dried at the increased temperature under reduced pressure. In one embodiment, the contents of the container are dried at about 30° C. and a pressure of 0.2 mbar. In one embodiment, the contents of the container are dried at about 30° C. and a pressure of 0.2 mbar in about 1.5 to 3 hours. In an embodiment, after the contents of the container are dried, the temperature is lowered to about 20° C., about 15° C., or about 10° C. under reduced pressure. In an embodiment, the pressure is returned to atmospheric pressure at the lowered temperature and the freeze drying process is complete.

### Freeze Drying Method #2

In an embodiment, the filled container is placed on a shelf and the shelf is cooled to about 20° C., about 15° C., about 10° C., about 5° C., or about 2° C. In one embodiment, the shelf is cooled from about 20° C. to about 2° C. in about 20 mins. In one embodiment, the filled container is then maintained at this temperature for a designated period of time. In one embodiment, the filled container is maintained at this temperature for about 5 mins to 1 hour, about 5 mins to about 50 mins, about 5 mins to about 40 mins, about 10 mins to about 35 mins, about 15 mins to about 30 mins or about 20 mins. In an embodiment, the shelf is then cooled further to initiate freezing of the contents of the filled container. In one embodiment, the shelf is cooled to about −5° C., about −10° C., about −15° C., about −20° C., about −25° C., about −30° C., about −35° C., or about −38° C. In one embodiment, the shelf is cooled to about −38° C. over about 1 hour. In an embodiment, the pressure is then reduced to about 0.05 mbar to about 0.5 mbar and the frozen filled container is maintained at the same temperature under reduced pressure. In one embodiment, the pressure is reduced to about 0.2 mbar. In one embodiment, the frozen filled container is maintained at the temperature for about 10 mins to about 3 hours, about 10 mins to about 2.5 hours, about 10 mins to about 2 hours, about 10 mins to about 1.5 hours, about 10 mins to about 1 hour, about 25 mins to about 50 mins, or about 40 mins at the reduced pressure. In one embodiment, the frozen filled container is maintained for about 40 mins at −38° C. at a pressure of about 0.2 mbar. In an embodiment, the temperature is increased under reduced pressure. In one embodiment, the temperature is increased to about 0° C., about 5° C., about 10° C., or about 12° C. In one embodiment, the temperature is increased over about 30 mins to about 8 hours, about 30 mins to about 7 hours, about 30 mins to about 6 hours, about 30 mins to about 5 hours, about 30 mins to about 4 hours, about 30 mins to about 3 hours, about 1 hour to about 3 hours, or about 1.5 hours to about 2 hours. In one embodiment, the temperature is increased to about 12° C. in about 1.5 hours to about 2 hours. In an embodiment, the increased temperature is maintained for about 12 to 16 hours. In an embodiment, the contents of the container are dried at the increased temperature under reduced pressure. In one embodiment, the contents of the container are dried at about 12° C. and a pressure of 0.2 mbar. In an embodiment, the pressure is returned to atmospheric pressure and the freeze drying process is complete.

In an embodiment, the resulting collagen matrix comprising bupivacaine is visually inspected for any physical defects (e.g., melt-backs, cosmetic defects, foreign particulates, presence of crystalline like structures, bubbles etc.). In one embodiment, any containers enclosing a collagen matrix comprising bupivacaine that has one or more physical defects is discarded. In an embodiment, the container is inspected for any physical and/or cosmetic defects. In one embodiment, a container with one or more physical and/or cosmetic defects is discarded (including the contents of the container, i.e., the collagen matrix comprising bupivacaine).

In embodiments wherein the filled container is freeze dried without a lid, a lid is placed on top of the container enclosing the collagen matrix comprising bupivacaine. In an embodiment, the lid is a gas-permeable lid. In one embodiment, the lid is a Tyvek lid. In one embodiment, the lid seals to the top of the container.

In an embodiment, the method further comprises step (e) placing the containers into a secondary packaging. In an embodiment, the method further comprises step (f) sterilizing the drug delivery device in the secondary packaging. In an embodiment, the method further comprises step (g) aerating the drug delivery device in secondary packaging. In an embodiment, the method further comprises each of steps (e)-(g).

In step (e), the container enclosing the drug delivery device is placed into a pouch. In one embodiment, three containers, each container enclosing a drug delivery device is placed into a frame for secure packaging within the pouch. The frame can be made of any material suitable for holding the containers without affecting the container itself or the contents. In one embodiment, the frame comprises PET. In one embodiment, the frame comprises chemically modified PET. In one embodiment, the frame comprises glycol modified PET (PETG). The pouch can be made of any material suitable for holding the containers without affecting the container itself, the contents of the container, and the optional frame holding the containers. In one embodiment, the pouch comprises PET and/or polyethylene glycol (PE). In one embodiment, the pouch comprises PET/PE. In an embodiment, at least a portion of the pouch is at least 50%, at least 60%, at least 70%, at least 80%, at least 90% or at least 95% permeable to a gas. In an embodiment, the pouch comprises a strip that is gas permeable. In one embodiment, the gas permeable strip is a Tyvek strip. In an embodiment, the pouch is sealed above the strip. In one embodiment, the pouch is heat sealed above the strip. In an embodiment, the pouch acts as secondary packaging for the containers enclosing the drug delivery device.

In step (f), the containers enclosing the drug delivery device are sterilized in the secondary packaging. In an embodiment, the sterilization step comprises exposing the secondary packaging to ethylene oxide (EO). In an embodiment, the ethylene oxide is mixed with a carrier. In one embodiment, the carrier is CO₂. In one embodiment, the mixture of carrier and EO comprises less than 15% EO. In one embodiment, the mixture comprises less than about 12%, about 10%, or about 8% EO. In one embodiment, the mixture comprises about 6% EO. While not wishing to be limited by theory, it is believed that the EO may react with available chloride ions from the bupivacaine HCl to form ethylene chlorohydrin (ECH) residues which need to be removed in a subsequent aeration step. Using less EO will therefore reduce the amount of ECH formed, decrease the aeration time needed, and/or decrease the amount of ECH remaining in the final product.

In an embodiment, the secondary packaging is placed into a chamber with a RH of greater than or equal to about 50%. In an embodiment, the pressure is increased in the chamber to a pressure of about 1.1 bar to about 4 bar, about 1.5 bar to about 3.5 bar, about 1.5 bar to about 3.0 bar, about 1.5 bar to about 2.5 bar, or about 2.0 bar. In an embodiment, the liquid carrier/EO mixture is added to the chamber via two gasifiers. In an embodiment, the chamber is at a temperature of about 25° C. to about 50° C., about 28° C. to about 47° C., about 31° C. to about 44° C., about 34° C. to about 44° C., about 36° C. to about 44° C., or about 44° C. In an embodiment, the secondary packaging and its contents are sterilized in the chamber for about 1 hour to about 10 hours, about 1 hour to about 9 hours, about 1 hour to about 8 hours, about 2 hours to about 8 hours, about 2 hours to about 7 hours, about 2 hours to about 6 hours, about 3 hours to about 6 hours, about 3 hours to about 5 hours, or about 4 hours.

In an embodiment, after sterilization, the secondary packaging and its contents undergo desorption of the EO gas in the chamber. In an embodiment, the chamber is maintained at about 22° C. to about 44° C. for the desorption process. In an embodiment, the desorption process takes at least 8 hours, 10 hours, 12 hours, 14 hours, 16 hours, or 18 hours. In an embodiment, the chamber is flushed with air during the desorption process. In one embodiment, the air is compressed air. In an embodiment, the pressure in the chamber is maintained between about −1.0 bar to 0.8 bar during the desorption process.

In step (g), the sterilized drug delivery device in the secondary packaging is aerated. In an embodiment, the aeration removes low level residue impurities in the drug delivery device. In one embodiment, the impurities comprise EO, ECH, and/or ethylene glycol (EGly). In an embodiment, the sterilized drug delivery device is aerated for about 1 week to about 20 weeks, about 1 week to about 18 weeks, about 1 week to about 16 weeks, about 1 week to about 14 weeks about 1 week to about 12 weeks, about 1 week to about 10 weeks, about 2 weeks to about 10 weeks, about 3 weeks to about 9 weeks, about 4 weeks to about 8 weeks, about 4 weeks to about 7 weeks, or about 5 weeks to about 6 weeks. In an embodiment, the temperature and/or RH during aeration is controlled. In an embodiment, the temperature is maintained between about 5° C. to about 50° C., about 5° C. to about 45° C., about 5° C. to about 40° C., about 5° C. to about 35° C., about 10° C. to about 35° C., about 12° C. to about 32° C., or about 15° C. to about 28° C. In an embodiment, the RH is maintained between about 18% and about 70%.

In an embodiment, the sterilized drug delivery device is aerated until it meets the specifications for EO, ECH, and/or EGly. In an embodiment, the device is aerated until it the concentration of EO in the device is less than about 50 ppm, less than about 45 ppm, less than about 40 ppm, less than about 35 ppm, less than about 30 ppm, less than about 25 ppm, less than about 20 ppm, less than about 15 ppm, less than about 10 ppm, less than about 5 ppm, or less than about 2.5 ppm. In one embodiment, the device is aerated until the concentration of EO is less than about 0.9 ppm. In an embodiment, the device is aerated until the concentration of ECH in the device is less than about 1,000 ppm, less than about 900 ppm, less than about 800 ppm, less than about 700 ppm, less than about 600 ppm, less than about 500 ppm, less than about 400 ppm, less than about 300 ppm, or less than about 200 ppm. In one embodiment, the device is aerated until the concentration of ECH in the device is less than about 175 ppm. In an embodiment, the device is aerated until it the concentration of EGly in the device is less than about 10,000 ppm, less than about 9,000 ppm, less than about 8,000 ppm, less than about 7,000 ppm, less than about 6,000 ppm, less than about 5,000 ppm, less than about 4,000 ppm, less than about 3,000 ppm, less than about 2,000 ppm, or less than about 1,000 ppm.

In an embodiment, the secondary packaging is terminally sealed after aeration of the drug delivery device. In one embodiment, the secondary packaging is sealed below the gas permeable portion of the pouch. In one embodiment, the secondary packaging is sealed below the gas permeable strip of the pouch. In an embodiment, the aerated drug delivery device in the terminally sealed secondary packaging is stored at about 20° C. to about 25° C. before use.

The following clauses describe certain embodiments.

Clause 1. A method of making a mature lyophilized milled collagen (LMC), the method comprising the steps of:
(a) providing isolated collagen, optionally an isolated collagen dispersion;
(b) freezing the isolated collagen;
(c) dehydrating the frozen collagen; and
(d) maturing the dehydrated collagen.

Clause 2. The method of clause 1, wherein the dehydrated collagen is placed in a permeable pouch before step (d) and step (d) comprises heating the dehydrated collagen in an environment with controlled temperature and controlled humidity.

Clause 3. The method of clause 2, wherein the dehydrated collagen is heated to about 40° C. in an environment of about 65% relative humidity.

Clause 4. The method of clause 2 or 3, wherein the dehydrated collagen is maintained in the environment with controlled temperature and controlled humidity until the dehydrated collagen reaches a LOD (loss on drying) of about 18%.

Clause 5. The method of clause 4, wherein an aqueous dispersion comprising 0.9 wt % of the dehydrated collagen matured to an LOD of about 18% has a viscosity of between about 110 cP and about 250 cP.

Clause 6. The method of any of clauses 1-5, wherein the method further comprises the step of
(e) dehumidifying the mature LMC to form dehumidified mature LMC.

Clause 7. The method of clause 6, wherein the mature LMC is dehumidified in a permeable pouch in an environment with controlled temperature and controlled humidity.

Clause 8. The method of clause 7, wherein the mature LMC is dehumidified at a temperature of about 25° C. in an environment of about 15% relative humidity.

Clause 9. The method of clause 7 or 8, wherein the matured collagen is dehumidified until a loss on drying of about 10% is reached.

Clause 10. A method of making a drug delivery device, the method comprising the steps of:
(a) forming a dispersion of dehumidified mature lyophilized milled collagen (LMC);
(b) adding a solution of bupivacaine, or a salt thereof, to the dispersion to form a bupivacaine-collagen mixture;
(c) filling a container with the bupivacaine-collagen mixture; and
(d) freeze drying the contents of the container to form a drug delivery device comprising a collagen matrix comprising bupivacaine, or a salt thereof.

Clause 11. The method of clause 10, wherein the dehumidified mature LMC comprises dehydrated LMC that has a loss on drying (LOD) of about 18% which has been dehumidified to an LOD of about 10%.

Clause 12. The method of clause 10 or 11, wherein the method further comprises the steps of:
(e) placing the containers into a secondary packaging;
(f) sterilizing the drug delivery device in the secondary packaging; and
(g) aerating the drug delivery device in secondary packaging.

Clause 13. The method of clause 12, wherein step (f) comprises sterilizing the drug delivery device with a mixture of about 6% ethylene oxide and about 94% CO₂.

Clause 14. The method of clause 12 or 13, wherein step (g) comprises aerating the drug delivery device until it comprises less than about 175 ppm ethylene chlorohydrin.

Clause 15. The method of any one of clauses 12-14, wherein step (g) comprises aerating the drug delivery device until it comprises less than about 0.9 ppm ethylene oxide, less than about 1,000 ppm ethylene glycol, or a combination thereof.

Clause 16. The method of any one of clauses 10-15, wherein the drug delivery device comprises about 4 mg/cm³ to about 8 mg/cm³ collagen and about 6 mg/cm³ to about 10 mg/cm³ bupivacaine, or a salt thereof.

Clause 17. The method of any one of clauses 10-16, wherein the drug delivery device comprises about 6 mg/cm³ collagen and about 8 mg/cm³ bupivacaine, or a salt thereof.

Clause 18. A drug delivery device made by the method of any one of clauses 10-17.

Clause 19. The drug delivery device of clause 18, wherein the device comprises a collagen matrix having dimensions of about 5 cm×5 cm×0.5 cm.

Clause 20. The drug delivery device of clause 18 or 19, wherein the collagen matrix comprises about 4 mg/cm³ to about 8 mg/cm³ collagen and about 6 mg/cm³ to about 10 mg/cm³ bupivacaine, or a salt thereof.

Clause 21. The drug delivery device of any one of clauses 18-20, wherein the collagen matrix comprises about 6 mg/cm³ collagen and about 8 mg/cm³ bupivacaine, or a salt thereof.

Clause 22. The drug delivery device of any one of clauses 18-21, wherein the device comprises at least one of (i)-(iii):
(i) less than about 175 ppm ethylene chlorohydrin;
(ii) less than about 0.9 ppm ethylene oxide; or
(iii) less than about 1,000 ppm ethylene glycol.

Clause 23. The drug delivery device of any one of clauses 18-22, wherein bupivacaine, or a salt thereof, is released from the device with an in vitro release profile of 50%+10% in the first 30 minutes, 75%+10% within 2 hours, and greater than 80% within 6 hours.

Clause 24. The drug delivery device of any one of clauses 18-23, wherein the device has a dry tensile strength of about 1.6 N to about 2.4 N.

Clause 25. The drug delivery device of any one of clauses 18-24, wherein the device has a wet tensile strength of about 0.6 N to about 1.2 N.

Clause 26. A method of performing a soft tissue surgery procedure in a subject in need thereof, the method comprising placing a drug delivery device at a surgical site, wherein the device comprises a collagen matrix and bupivacaine, or a salt thereof.

Clause 27. The method of clause 26, wherein the collagen matrix has dimensions of about 5 cm×5 cm×0.5 cm.

Clause 28. The method of clause 26 or 27, wherein the collagen matrix comprises about 4 mg/cm³ to about 8 mg/cm³ collagen and about 6 mg/cm³ to about 10 mg/cm³ bupivacaine, or a salt thereof.

Clause 29. The method of any one of clauses 26-28, wherein the collagen matrix comprises about 6 mg/cm³ collagen and about 8 mg/cm³ bupivacaine, or a salt thereof.

Clause 30. The method of any one of clauses 26-29, wherein the device comprises at least one of (i)-(iii):
(i) less than about 175 ppm ethylene chlorohydrin;
(ii) less than about 0.9 ppm ethylene oxide; or
(iii) less than about 1,000 ppm ethylene glycol.

Clause 31. The method of any one of clauses 26-30, wherein bupivacaine, or a salt thereof, is released from the device with an in vitro release profile of 50%±10% in the first 30 minutes, 75%±10% within 2 hours, and greater than 80% within 6 hours.

Clause 32. The method of any one of clauses 26-31, wherein the device has a dry tensile strength of about 1.6 N to about 2.4 N.

Clause 33. The method of any one of clauses 26-32, wherein the device has a wet tensile strength of about 0.6 N to about 1.2 N.

Clause 34. The method of any one of clauses 26-33, wherein the surgery procedure is selected from: an abdominoplasty, an open ventral hernia repair, an open abdominal hysterectomy, a laparoscopic-assisted colectomy, a reduction mammoplasty, and combinations thereof.

Clause 35. The method of any one of clauses 26-34, wherein bupivacaine, or a salt thereof, is present in the drug delivery device in an amount sufficient to provide a duration of local analgesia, local anesthesia, or nerve blockade which lasts for at least about one day after administration.

Clause 36. The method of clause 35, wherein bupivacaine, or a salt thereof, is present in the drug delivery device in an amount sufficient to provide a duration of local analgesia, local anesthesia, or nerve blockade which lasts for at least about 72 hours after administration.

Clause 37. The method of any one of clauses 26-36, wherein more than one drug delivery device is placed at the surgical site.

Clause 38. The method of any one of clauses 26-37, wherein three drug delivery devices are placed at multiple layers in the soft tissue at the surgical site.

Clause 101. A method of performing a soft tissue surgery procedure in a subject in need thereof, comprising placing the drug delivery device of any one of clauses 18-25 at a surgical site for controlled and/or sustained release of bupivacaine hydrochloride,
wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 102. The method of clause 101, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 103. The method of clause 101, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 104. The method of clause 101, wherein the surgery procedure is open ventral hernia repair comprising mesh placement at different subcutaneous layers, including intraperitoneal placement.

Clause 105. The method of clause 101, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 106. The method of clause 101, wherein the surgery procedure is open ventral hernia repair comprising onlay mesh repair, wherein the drug delivery device is placed in part in the preperitoneal space between the closed peritoneum and posterior rectus sheath, and in part laid in direct contact with the closed anterior rectus sheath.

Clause 107. The method of clause 101, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 108. The method of clause 101, wherein the surgery procedure is laparoscopic-assisted colectomy comprising placing the drug delivery device in part at the site of fascial repair, and in part superficially above the abdominal wall musculature and beneath extraction site incision.

Clause 109. The method of clause 101, wherein the surgery procedure is reduction mammoplasty comprising placing the drug delivery device in part in one or both breast pockets.

Clause 110. The method of any one of clauses 101 to 109, further comprising partitioning the drug delivery device into segments with a predetermined size, wherein each segment is placed at the surgical site independently.

Clause 111. The method of clause 110, wherein the release dissolution profile of the sum of drug delivery device segments is substantially similar to the release dissolution profile of the unpartitioned drug delivery device.

Clause 112. The method of clause 110, wherein the release dissolution profile of the sum of drug delivery device segments is within about 1% and about 15% at any point in time substantially similar to the release dissolution profile of the unpartitioned drug delivery device.

Clause 113. A kit for performing a soft tissue surgery procedure in a subject in need thereof, the kit comprising the drug delivery device of any one of clauses 18-25, and instructions for placement of the drug delivery device at a surgical site for controlled and/or sustained release of bupivacaine hydrochloride,
wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 114. The kit of clause 113, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 115 The kit of clause 113, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 116. The kit of clause 113, wherein the surgery procedure is open ventral hernia repair comprising mesh placement at different subcutaneous layers, including intraperitoneal placement.

Clause 117. The kit of clause 113, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 118. The kit of clause 113, wherein the surgery procedure is open ventral hernia repair comprising mesh repair, wherein the drug delivery device is placed in part in the preperitoneal space between the closed peritoneum and posterior rectus sheath, and in part laid in direct contact with the closed anterior rectus sheath.

Clause 119. The kit of clause 113, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 120. The kit of clause 113, wherein the surgery procedure is laparoscopic-assisted colectomy comprising placing the drug delivery device in part at the site of fascial repair, and in part superficially above the abdominal wall musculature and beneath extraction site incision.

Clause 121. The kit of clause 113, wherein the surgery procedure is reduction mammoplasty comprising placing the drug delivery device in part in one or both breast pockets.

Clause 122. The kit of any one of clauses 113 to 121, further comprising instructions for partitioning the drug delivery device into segments with a predetermined size, wherein each segment is placed at the surgical site independently.

Clause 123. The kit of clause 122, wherein the release dissolution profile of the sum of drug delivery device segments is substantially similar to the release dissolution profile of the unpartitioned drug delivery device.

Clause 124. The kit of clause 122, wherein the release dissolution profile of the sum of drug delivery device segments is within about 1% and about 15% at any point in time substantially similar to the release dissolution profile of the unpartitioned drug delivery device.

Clause 125. The drug delivery device of any one of clauses 118-125, wherein the drug delivery device comprises less than or equal to about 12.5 EU bacterial endotoxins.

Clause 201. A drug delivery device comprising a collagen matrix having dimensions of about 5 cm×5 cm×0.5 cm, wherein the collagen matrix comprises about 4 mg/cm³ to about 8 mg/cm³ collagen and about 6 mg/cm³ to about 10 mg/cm³ bupivacaine, or a salt thereof and wherein the device comprises less than about 175 ppm ethylene chlorohydrin.

Clause 202. The drug delivery device of clause 201, wherein the collagen matrix comprises about 6 mg/cm³ collagen and about 8 mg/cm³ bupivacaine, or a salt thereof.

Clause 203. The drug delivery device of clause 201 or 202, wherein the device comprises:
less than about 0.9 ppm ethylene oxide; and/or
less than about 1,000 ppm ethylene glycol.

Clause 204. The drug delivery device of any one of clauses 201-203, wherein the device comprises less than about 5 ppm elemental impurities.

Clause 205. The drug delivery device of any one of clauses 201-204, wherein the device has a dry tensile strength of about 1.6 N to about 2.4 N

Clause 206. The drug delivery device of any one of clauses 201-205, wherein the device has a wet tensile strength of about 0.6 N to about 1.2 N.

Clause 207. The drug delivery device of any one of clauses 201-206, wherein bupivacaine, or a salt thereof, is released from the device with an in vitro release profile of 50%±10% in the first 30 minutes, 75%±10% within 2 hours, and greater than 80% within 6 hours.

Clause 208. A method of making a mature lyophilized milled collagen (LMC), the method comprising the steps of:
(a) providing isolated collagen, optionally an isolated collagen dispersion;
(b) freezing the isolated collagen;
(c) dehydrating the frozen collagen; and
(d) maturing the dehydrated collagen.

Clause 209. The method of clause 208, wherein step (c) further comprises placing the dehydrated collagen in a permeable pouch before step (d).

Clause 210. The method of clause 209, wherein the maturing of step (d) comprises heating the dehydrated collagen in an environment with controlled temperature and controlled humidity.

Clause 211. The method of clause 210, comprising heating the dehydrated collagen to between about 35° C. and about 45° C. in an environment of between about 60% to about 70% relative humidity.

Clause 212. The method of clause 211, comprising heating the dehydrated collagen to about 40° C. in an environment of about 65% relative humidity.

Clause 213. The method of any one of clauses 210-212, further comprising maintaining the dehydrated collagen in the environment with controlled temperature and controlled humidity until the dehydrated collagen reaches a LOD (loss on drying) of between about 17% and about 22%.

Clause 214. The method of clause 213, wherein the dehydrated collagen reaches a LOD of about 18%.

Clause 215. The method of clause 214, wherein an aqueous dispersion comprising 0.9 wt % of the dehydrated collagen matured to an LOD of about 18% has a viscosity of between about 110 cP and about 250 cP.

Clause 216. The method of any of clauses 208-215, further comprising
(e) dehumidifying the mature LMC to form dehumidified mature LMC.

Clause 217. The method of clause 216, further comprising placing the mature LMC in a permeable pouch before step (e).

Clause 218. The method of clause 217, wherein the dehumidifying of step (e) comprises heating the mature LMC in an environment with controlled temperature and controlled humidity.

Clause 219. The method of clause 218, comprising dehumidifying the mature LMC at a temperature of between about 20° C. and about 30° C. in an environment of between about 10% and about 20% relative humidity.

Clause 220. The method of clause 219, comprising dehumidifying the mature LMC at a temperature of about 25° C. in an environment of about 15% relative humidity.

Clause 221. The method of any one of clauses 218-220, wherein the mature LMC is dehumidified until a loss on drying of between about 8% and about 12% is reached.

Clause 222. The method of clause 221, wherein the mature LMC is dehumidified until a loss on drying of about 10% is reached.

Clause 223. A method of making a drug delivery device, the method comprising the steps of
(a) forming a dispersion of dehumidified mature lyophilized milled collagen (LMC);
(b) adding a solution of bupivacaine, or a salt thereof, to the dispersion to form a bupivacaine-collagen mixture;
(c) filling a container with the bupivacaine-collagen mixture; and
(d) freeze drying the contents of the container to form a drug delivery device comprising a collagen matrix comprising bupivacaine, or a salt thereof.

Clause 224. The method of clause 223, wherein the dehumidified mature LMC comprises dehydrated LMC that has a loss on drying (LOD) of between about 17% and about 22% and which has been dehumidified to an LOD of between about 8% and about 12%.

Clause 225. The method of clause 224, wherein the dehydrated LMC has an LOD of about 18% and has been dehumidified to an LOD of about 10%.

Clause 226. The method of any one of clauses 223-225, wherein the method further comprises:
(e) placing the containers into a secondary packaging;
(f) sterilizing the drug delivery device in the secondary packaging; and
(g) aerating the drug delivery device in secondary packaging.

Clause 227. The method of clause 226, wherein step (f) comprises sterilizing the drug delivery device with a mixture of about 6% ethylene oxide and about 94% CO₂.

Clause 228. The method of clause 226 or 227, wherein step (g) comprises aerating the drug delivery device until it comprises less than about 175 ppm ethylene chlorohydrin.

Clause 229. The method of any one of clauses 226-228, wherein step (g) comprises aerating the drug delivery device until it comprises less than about 0.9 ppm ethylene oxide, less than about 1,000 ppm ethylene glycol, or a combination thereof.

Clause 230. The method of any one of clauses 223-229, wherein the drug delivery device comprises about 4 mg/cm³ to about 8 mg/cm³ collagen and about 6 mg/cm³ to about 10 mg/cm³ bupivacaine, or a salt thereof.

Clause 231. The method of any one of clauses 223-230, wherein the drug delivery device comprises about 6 mg/cm³ collagen and about 8 mg/cm³ bupivacaine, or a salt thereof.

Clause 232. A drug delivery device made by the method of any one of clauses 223-231.

Clause 233. A method of performing a soft tissue surgery procedure in a subject in need thereof, comprising placing the drug delivery device of any one of clauses 201-207 or 232 at a surgical site for controlled and/or sustained release of bupivacaine hydrochloride,
wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 234. The method of clause 233, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 235. The method of clause 233, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 236. The method of clause 233, wherein the surgery procedure is open ventral hernia repair comprising mesh placement at different subcutaneous layers, including intraperitoneal placement.

Clause 237. The method of clause 233, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 238. The method of clause 233, wherein the surgery procedure is open ventral hernia repair comprising onlay mesh repair, wherein the drug delivery device is placed in part in the preperitoneal space between the closed peritoneum and posterior rectus sheath, and in part laid in direct contact with the closed anterior rectus sheath.

Clause 239. The method of clause 233, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 240. The method of clause 233, wherein the surgery procedure is laparoscopic-assisted colectomy comprising placing the drug delivery device in part at the site of fascial repair, and in part superficially above the abdominal wall musculature and beneath extraction site incision.

Clause 241. The method of clause 233, wherein the surgery procedure is reduction mammoplasty comprising placing the drug delivery device in part in one or both breast pockets.

Clause 242. The method of any one of clauses 233-241, wherein more than one drug delivery device is placed at the surgical site.

Clause 243. The method of clause 242, wherein three drug delivery devices are placed at multiple layers in the soft tissue at the surgical site.

Clause 244. The method of any one of clauses 233-243, wherein bupivacaine, or a salt thereof, is present in the drug delivery device in an amount sufficient to provide a duration of local analgesia, local anesthesia, or nerve blockade which lasts for at least about one day after administration.

Clause 245. The method of clause 244, wherein bupivacaine, or a salt thereof, is present in the drug delivery device in an amount sufficient to provide a duration of local analgesia, local anesthesia, or nerve blockade which lasts for at least about 72 hours after administration.

Clause 246. The method of any one of clauses 233-241, further comprising partitioning the drug delivery device into segments with a predetermined size, wherein each segment is placed at the surgical site independently.

Clause 247. The method of clause 246, wherein the release dissolution profile of the sum of drug delivery device segments is substantially similar to the release dissolution profile of the unpartitioned drug delivery device.

Clause 248. The method of clause 247, wherein the release dissolution profile of the sum of drug delivery device segments is within about 1% and about 15% at any point in time substantially similar to the release dissolution profile of the unpartitioned drug delivery device.

Clause 249. A kit for performing a soft tissue surgery procedure in a subject in need thereof, the kit comprising the drug delivery device of any one of clauses 201-207 or 232, and instructions for placement of the drug delivery device at a surgical site for controlled and/or sustained release of bupivacaine hydrochloride,
wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 250. The kit of clause 249, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 251. The kit of clause 249, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 252. The kit of clause 249, wherein the surgery procedure is open ventral hernia repair comprising mesh placement at different subcutaneous layers, including intraperitoneal placement.

Clause 253. The kit of clause 249, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 254. The kit of clause 249, wherein the surgery procedure is open ventral hernia repair comprising mesh repair, wherein the drug delivery device is placed in part in the preperitoneal space between the closed peritoneum and posterior rectus sheath, and in part laid in direct contact with the closed anterior rectus sheath.

Clause 255. The kit of clause 249, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 256. The kit of clause 249, wherein the surgery procedure is laparoscopic-assisted colectomy comprising placing the drug delivery device in part at the site of fascial repair, and in part superficially above the abdominal wall musculature and beneath extraction site incision.

Clause 257. The kit of clause 249, wherein the surgery procedure is reduction mammoplasty comprising placing the drug delivery device in part in one or both breast pockets.

Clause 258. The kit of any one of clauses 249-257, further comprising instructions for partitioning the drug delivery device into segments with a predetermined size, wherein each segment is placed at the surgical site independently.

Clause 259. The kit of clause 258, wherein the release dissolution profile of the sum of drug delivery device segments is substantially similar to the release dissolution profile of the unpartitioned drug delivery device

Clause 260. The kit of clause 259, wherein the release dissolution profile of the sum of drug delivery device segments is within about 1% and about 15% at any point in time substantially similar to the release dissolution profile of the unpartitioned drug delivery device.

Clause 261. The drug delivery device of any one of clauses 201-206, wherein the drug delivery device comprises less than or equal to about 12.5 EU bacterial endotoxins.

Clause 301. A method of making a mature lyophilized milled collagen (LMC), the method comprising the steps of: (a) providing isolated collagen, optionally an isolated collagen dispersion; (b) freezing the isolated collagen; (c) dehydrating the frozen collagen; and (d) maturing the dehydrated collagen.

Clause 302. The method of clause 301, wherein the dehydrated collagen is placed in a permeable pouch before step (d) and step (d) comprises heating the dehydrated collagen in an environment with controlled temperature and controlled humidity.

Clause 303. The method of clause 302, wherein the dehydrated collagen is heated to about 40° C. in an environment of about 65% relative humidity.

Clause 304. The method of clause 302 or 303, wherein the dehydrated collagen is maintained in the environment with controlled temperature and controlled humidity until the dehydrated collagen reaches a LOD (loss on drying) of about 18%.

Clause 305. The method of clause 304, wherein an aqueous dispersion comprising 0.9 wt % of the dehydrated collagen matured to an LOD of about 18% has a viscosity of between about 110 cP and about 250 cP.

Clause 306. The method of any of clauses 301-305, wherein the method further comprises the step of (e) dehumidifying the mature LMC to form dehumidified mature LMC.

Clause 307. The method of clause 306, wherein the mature LMC is dehumidified in a permeable pouch in an environment with controlled temperature and controlled humidity.

Clause 308. The method of clause 307, wherein the mature LMC is dehumidified at a temperature of about 25° C. in an environment of about 15% relative humidity.

Clause 309. The method of clause 307 or 308, wherein the matured collagen is dehumidified until a loss on drying of about 10% is reached.

Clause 310. A method of making a drug delivery device, the method comprising the steps of: (a) forming a dispersion of dehumidified mature lyophilized milled collagen (LMC); (b) adding a solution of bupivacaine, or a salt thereof, to the dispersion to form a bupivacaine-collagen mixture; (c) filling a container with the bupivacaine-collagen mixture; and (d) freeze drying the contents of the container to form a drug delivery device comprising a collagen matrix comprising bupivacaine, or a salt thereof.

Clause 311. The method of clause 310, wherein the dehumidified mature LMC comprises dehydrated LMC that has a loss on drying (LOD) of about 18% which has been dehumidified to an LOD of about 10%.

Clause 312. The method of clause 310 or 311, wherein the method further comprises the steps of: (e) placing the containers into a secondary packaging; (f) sterilizing the drug delivery device in the secondary packaging; and (g) aerating the drug delivery device in secondary packaging.

Clause 313. The method of clause 312, wherein step (f) comprises sterilizing the drug delivery device with a mixture of about 6% ethylene oxide and about 94% CO₂.

Clause 314. The method of clause 312 or 313, wherein step (g) comprises aerating the drug delivery device until it comprises less than about 175 ppm ethylene chlorohydrin.

Clause 315. The method of any one of clauses 312-314, wherein step (g) comprises aerating the drug delivery device until it comprises less than about 0.9 ppm ethylene oxide, less than about 1,000 ppm ethylene glycol, or a combination thereof.

Clause 316. The method of any one of clauses 310-315, wherein the drug delivery device comprises about 4 mg/cm³ to about 8 mg/cm³ collagen and about 6 mg/cm³ to about 10 mg/cm³ bupivacaine, or a salt thereof.

Clause 317. The method of any one of clauses 310-316, wherein the drug delivery device comprises about 6 mg/cm³ collagen and about 8 mg/cm³ bupivacaine, or a salt thereof.

Clause 318. A drug delivery device made by the method of any one of clauses 310-317.

Clause 319. The drug delivery device of clause 318, wherein the device comprises a collagen matrix having dimensions of about 5 cm×5 cm×0.5 cm.

Clause 320. The drug delivery device of clause 318 or 319, wherein the collagen matrix comprises about 4 mg/cm³ to about 8 mg/cm³ collagen and about 6 mg/cm³ to about 10 mg/cm³ bupivacaine, or a salt thereof.

Clause 321. The drug delivery device of any one of clauses 318-320, wherein the collagen matrix comprises about 6 mg/cm³ collagen and about 8 mg/cm³ bupivacaine, or a salt thereof.

Clause 322. The drug delivery device of any one of clauses 318-321, wherein the device comprises at least one of (i)-(iii): (i) less than about 175 ppm ethylene chlorohydrin; (ii) less than about 0.9 ppm ethylene oxide; or (iii) less than about 1,000 ppm ethylene glycol.

Clause 323. The drug delivery device of any one of clauses 318-322, wherein bupivacaine, or a salt thereof, is released from the device with an in vitro release profile of 50%±10% in the first 30 minutes, 75%±10% within 2 hours, and greater than 80% within 6 hours.

Clause 324. The drug delivery device of any one of clauses 318-323, wherein the device has a dry tensile strength of about 1.6 N to about 2.4 N.

Clause 325. The drug delivery device of any one of clauses 318-324, wherein the device has a wet tensile strength of about 0.6 N to about 1.2 N.

Clause 326. A method of performing a soft tissue surgery procedure in a subject in need thereof, the method comprising placing a drug delivery device at a surgical site, wherein the device comprises a collagen matrix and bupivacaine, or a salt thereof.

Clause 327. The method of clause 326, wherein the collagen matrix has dimensions of about 5 cm×5 cm×0.5 cm.

Clause 328. The method of clause 326 or 327, wherein the collagen matrix comprises about 4 mg/cm³ to about 8 mg/cm³ collagen and about 6 mg/cm³ to about 10 mg/cm³ bupivacaine, or a salt thereof.

Clause 329. The method of any one of clauses 326-328, wherein the collagen matrix comprises about 6 mg/cm³ collagen and about 8 mg/cm³ bupivacaine, or a salt thereof.

Clause 330. The method of any one of clauses 326-329, wherein the device comprises at least one of (i)-(iii): (i) less than about 175 ppm ethylene chlorohydrin; (ii) less than about 0.9 ppm ethylene oxide; or (iii) less than about 1,000 ppm ethylene glycol.

Clause 331. The method of any one of clauses 326-330, wherein bupivacaine, or a salt thereof, is released from the device with an in vitro release profile of 50%±10% in the first 30 minutes, 75%±10% within 2 hours, and greater than 80% within 6 hours.

Clause 332. The method of any one of clauses 326-331, wherein the device has a dry tensile strength of about 1.6 N to about 2.4 N.

Clause 333. The method of any one of clauses 326-332, wherein the device has a wet tensile strength of about 0.6 N to about 1.2 N.

Clause 334. The method of any one of clauses 326-333, wherein the surgery procedure is selected from: an abdominoplasty, an open ventral hernia repair, an open abdominal hysterectomy, a laparoscopic-assisted colectomy, a reduction mammoplasty, and combinations thereof.

Clause 335. The method of any one of clauses 326-334, wherein bupivacaine, or a salt thereof, is present in the drug delivery device in an amount sufficient to provide a duration of local analgesia, local anesthesia, or nerve blockade which lasts for at least about one day after administration.

Clause 336. The method of clause 335, wherein bupivacaine, or a salt thereof, is present in the drug delivery device in an amount sufficient to provide a duration of local analgesia, local anesthesia, or nerve blockade which lasts for at least about 72 hours after administration.

Clause 337. The method of any one of clauses 326-336, wherein more than one drug delivery device is placed at the surgical site.

Clause 338. The method of any one of clauses 326-337, wherein three drug delivery devices are placed at multiple layers in the soft tissue at the surgical site.

Clause 339. A method of performing a soft tissue surgery procedure in a subject in need thereof, comprising placing the drug delivery device of any one of clauses 318-325 at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 340. The method of clause 339, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 341. The method of clause 339, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 342. The method of clause 339, wherein the surgery procedure is open ventral hernia repair comprising mesh placement at different subcutaneous layers, including intraperitoneal placement.

Clause 343. The method of clause 339, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 344. The method of clause 339, wherein the surgery procedure is open ventral hernia repair comprising onlay mesh repair, wherein the drug delivery device is placed in part in the preperitoneal space between the closed peritoneum and posterior rectus sheath, and in part laid in direct contact with the closed anterior rectus sheath.

Clause 345. The method of clause 339, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 346. The method of clause 339, wherein the surgery procedure is laparoscopic-assisted colectomy comprising placing the drug delivery device in part at the site of fascial repair, and in part superficially above the abdominal wall musculature and beneath extraction site incision.

Clause 347. The method of clause 339, wherein the surgery procedure is reduction mammoplasty comprising placing the drug delivery device in part in one or both breast pockets.

Clause 348. The method of any one of clauses 339 to 347, further comprising partitioning the drug delivery device into segments with a predetermined size, wherein each segment is placed at the surgical site independently.

Clause 349. The method of clause 348, wherein the release dissolution profile of the sum of drug delivery device segments is substantially similar to the release dissolution profile of the unpartitioned drug delivery device.

Clause 350. The method of clause 348, wherein the release dissolution profile of the sum of drug delivery device segments is within about 1% and about 15% at any point in time substantially similar to the release dissolution profile of the unpartitioned drug delivery device.

Clause 351. A kit for performing a soft tissue surgery procedure in a subject in need thereof, the kit comprising the drug delivery device of any one of clauses 318-325, and instructions for placement of the drug delivery device at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 352. The kit of clause 351, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 353. The kit of clause 351, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 354. The kit of clause 351, wherein the surgery procedure is open ventral hernia repair comprising mesh placement at different subcutaneous layers, including intraperitoneal placement.

Clause 355. The kit of clause 351, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 356. The kit of clause 351, wherein the surgery procedure is open ventral hernia repair comprising mesh repair, wherein the drug delivery device is placed in part in the preperitoneal space between the closed peritoneum and posterior rectus sheath, and in part laid in direct contact with the closed anterior rectus sheath.

Clause 357. The kit of clause 351, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 358. The kit of clause 351, wherein the surgery procedure is laparoscopic-assisted colectomy comprising placing the drug delivery device in part at the site of fascial repair, and in part superficially above the abdominal wall musculature and beneath extraction site incision.

Clause 359. The kit of clause 351, wherein the surgery procedure is reduction mammoplasty comprising placing the drug delivery device in part in one or both breast pockets.

Clause 360. The kit of any one of clauses 351 to 359, further comprising instructions for partitioning the drug delivery device into segments with a predetermined size, wherein each segment is placed at the surgical site independently.

Clause 361. The kit of clause 360, wherein the release dissolution profile of the sum of drug delivery device segments is substantially similar to the release dissolution profile of the unpartitioned drug delivery device.

Clause 362. The kit of clause 360, wherein the release dissolution profile of the sum of drug delivery device segments is within about 1% and about 15% at any point in time substantially similar to the release dissolution profile of the unpartitioned drug delivery device.

Clause 401. A drug delivery device comprising a collagen matrix and bupivacaine or a salt thereof, the drug delivery device comprising a level of impurities selected from less than about 175 ppm ethylene chlorohydrin, less than about 0.9 ppm ethylene oxide, less than about 1,000 ppm ethylene glycol, and less than about 5 ppm elemental impurities.

Clause 402a. The drug delivery device of clause 401, wherein the drug delivery device has a tensile strength selected from a dry tensile strength of about 1.6 N to about 2.4 N, and a wet tensile strength of about 0.6 N to about 1.2 N. Clause 402b. The drug delivery device of clause 401, wherein the drug delivery device has a dry tensile strength of about 1.5 N to about 2.5 N, about 1.4 N to about 2.4 N, or about 1.6 N to about 2.6 N. Clause 402c. The drug delivery device of clause 401, wherein the drug delivery device has a wet tensile strength of about 0.5 N to about 1.5 N, about 0.5 N to about 1.1 N, or about 0.6 N to about 1.3 N.

Clause 403a. The drug delivery device of clause 401 or 402, wherein the device comprises about 4 mg/cm³ to about 8 mg/cm³ collagen and about 6 mg/cm³ to about 10 mg/cm³ bupivacaine or a salt thereof. Clause 403b. The drug delivery device of clause 401 or 402, wherein the device comprises about 3 mg/cm³, about 4 mg/cm³, about 5 mg/cm³, about 6 mg/cm³, about 7 mg/cm³, about 8 mg/cm³, about 9 mg/cm³, or about 10 mg/cm³ collagen. Clause 403c. The drug delivery device of clause 401 or 402, wherein the device comprises about 4 mg/cm³, about 5 mg/cm³, about 6 mg/cm³, about 7 mg/cm³, about 8 mg/cm³, about 9 mg/cm³, about 10 mg/cm³, about 11 mg/cm³, or about 12 mg/cm³ bupivacaine or a salt thereof.

Clause 404. The drug delivery device of any one of clauses 401 to 403, wherein the collagen matrix comprises about 6 mg/cm³ collagen and about 8 mg/cm³ bupivacaine, or a salt thereof.

Clause 405. The drug delivery device of any one of clauses 401 to 404, comprising bupivacaine hydrochloride.

Clause 406a. The drug delivery device of any one of clauses 401 to 405, the device having dimensions of about 5 cm×about 5 cm×about 0.5 cm. Clause 406b. The drug delivery device of any one of clauses 401 to 405, the device having a length of about 4.5 cm, about 4.6 cm, about 4.7 cm, about 4.8 cm, about 4.9 cm, about 5 cm, about 5.1 cm, about 5.2 cm, about 5.3 cm, about 5.4 cm, or about 5.5 cm. Clause 406c. The drug delivery device of any one of clauses 401 to 405, the device having a width of about 4.5 cm, about 4.6 cm, about 4.7 cm, about 4.8 cm, about 4.9 cm, about 5 cm, about 5.1 cm, about 5.2 cm, about 5.3 cm, about 5.4 cm, or about 5.5 cm. Clause 406d. The drug delivery device of any one of clauses 401 to 405, the device having a thickness of about 0.45 cm, about 0.46 cm, about 0.47 cm, about 0.48 cm, about 0.49 cm, about 0.5 cm, about 0.51 cm, about 0.52 cm, about 0.53 cm, about 0.54 cm, or about 0.55 cm.

Clause 407. The drug delivery device of any one of clauses 401 to 406, wherein the bupivacaine, or a salt thereof, is released from the device with an in vitro release profile of 50%±10% in the first 30 minutes, 75%±10% within 2 hours, and greater than 80% within 6 hours.

Clause 408. A method of making a mature lyophilized milled collagen (mLMC), the method comprising: providing isolated collagen, optionally an isolated collagen dispersion; freezing the isolated collagen; dehydrating the frozen collagen; and maturing the dehydrated collagen.

Clause 409. The method of clause 408, further comprising placing the dehydrated collagen in a permeable pouch before the maturing.

Clause 410. The method of clause 408 or 409, wherein the maturing comprises heating the dehydrated collagen in an environment with controlled temperature and controlled humidity.

Clause 411. The method of clause 410, wherein the heating is between about 35° C. and about 45° C., at between about 60% to about 70% relative humidity.

Clause 412. The method of clause 411, wherein the heating is at about 40° C. in an environment of about 65% relative humidity.

Clause 413. The method of any one of clauses 410 to 412, further comprising maintaining the dehydrated collagen in the environment with controlled temperature and controlled humidity until the dehydrated collagen reaches a LOD (loss on drying) of between about 17% and about 22%.

Clause 414. The method of clause 413, wherein the dehydrated collagen reaches a LOD of about 18%.

Clause 415. The method of clause 414, wherein an aqueous dispersion comprising 0.9 wt % of the dehydrated collagen matured to an LOD of about 18% has a viscosity of between about 110 cP and about 250 cP.

Clause 416. The method of any of clauses 408 to 415, further comprising dehumidifying the mature LMC to form dehumidified mature LMC.

Clause 417. The method of clause 416, further comprising placing the mature LMC in a permeable pouch before dehumidifying.

Clause 418. The method of clause 417, wherein the dehumidifying comprises heating the mature LMC in an environment with controlled temperature and controlled humidity.

Clause 419. The method of clause 418, comprising dehumidifying the mature LMC at a temperature of between about 20° C. and about 30° C. in an environment of between about 10% and about 20% relative humidity.

Clause 420. The method of clause 419, comprising dehumidifying the mature LMC at a temperature of about 25° C. in an environment of about 15% relative humidity.

Clause 421. The method of any one of clauses 418 to 420, wherein the mature LMC is dehumidified until a loss on drying of between about 8% and about 12% is reached.

Clause 422. The method of clause 421, wherein the mature LMC is dehumidified until a loss on drying of about 10% is reached.

Clause 423. A method of making a drug delivery device, the method comprising: forming a dispersion of dehumidified mature lyophilized milled collagen (mLMC); adding a solution of bupivacaine, or a salt thereof, to the dispersion to form a bupivacaine-collagen mixture; filling a container with the bupivacaine-collagen mixture; and freeze drying the contents of the container to form a drug delivery device comprising a collagen matrix comprising bupivacaine, or a salt thereof.

Clause 424. The method of clause 423, wherein the dehumidified mature LMC comprises dehydrated LMC that has a loss on drying (LOD) of between about 17% and about 22% and which has been dehumidified to an LOD of between about 8% and about 12%.

Clause 425. The method of clause 424, wherein the dehydrated LMC has an LOD of about 18% and has been dehumidified to an LOD of about 10%.

Clause 426. The method of any one of clauses 423 to 425, wherein the method further comprises: placing the containers into a secondary packaging; sterilizing the drug delivery device in the secondary packaging; and aerating the drug delivery device in secondary packaging.

Clause 427a. The method of clause 426, wherein the sterilizing comprises sterilizing the drug delivery device with a mixture of about 6% ethylene oxide and about 94% CO₂. Clause 427b. The method of clause 426, wherein the sterilizing comprises sterilizing the drug delivery device with a gas mixture comprising about 4% to about 8% ethylene oxide. Clause 427c. The method of clause 426, wherein the sterilizing comprises sterilizing the drug delivery device with a gas mixture comprising about 4% ethylene oxide, about 5% ethylene oxide, about 6% ethylene oxide, about 7% ethylene oxide, or about 8% ethylene oxide. Clause 427d. The method of clause 426, wherein the sterilizing comprises sterilizing the drug delivery device with a gas mixture comprising about 96% CO₂, about 95% CO₂, about 94% CO₂, about 93% CO₂, or about 92% CO₂.

Clause 428a. The method of clause 426 or 427, wherein the aerating comprises aerating the drug delivery device until it comprises less than about 175 ppm ethylene chlorohydrin. Clause 428b. The method of clause 426 or 427, wherein the aerating comprises aerating the drug delivery device until it comprises less than about 200 ppm ethylene chlorohydrin. Clause 428c. The method of clause 426 or 427, wherein the aerating comprises aerating the drug delivery device until it comprises less than about 250 ppm ethylene chlorohydrin. Clause 428d. The method of clause 426 or 427, wherein the aerating comprises aerating the drug delivery device until it comprises less than about 300 ppm ethylene chlorohydrin. Clause 428e. The method of clause 426 or 427, wherein the aerating comprises aerating the drug delivery device until it comprises less than about 350 ppm ethylene chlorohydrin. Clause 428f. The method of clause 426 or 427, wherein the aerating comprises aerating the drug delivery device until it comprises less than about 400 ppm ethylene chlorohydrin. Clause 428g. The method of clause 426 or 427, wherein the aerating comprises aerating the drug delivery device until it comprises less than about 450 ppm ethylene chlorohydrin. Clause 428h. The method of clause 426 or 427, wherein the aerating comprises aerating the drug delivery device until it comprises less than about 500 ppm ethylene chlorohydrin.

Clause 429a. The method of any one of clauses 426 to 428, wherein the aerating comprises aerating the drug delivery device until it comprises less than about 0.9 ppm ethylene oxide, less than about 1,000 ppm ethylene glycol, or both. Clause 429b. The method of any one of clauses 426 to 428, wherein the aerating comprises aerating the drug delivery device until it comprises less than about 0.7 ppm ethylene oxide, about 0.8 ppm ethylene oxide, about ppm ethylene oxide, about 0.9 ppm ethylene oxide, about 1 ppm ethylene oxide, about 1.1 ppm ethylene oxide, about 1.2 ppm ethylene oxide, about 1.3 ppm ethylene oxide, about 1.4 ppm ethylene oxide, or about 1.5 ppm ethylene oxide. Clause 429c. The method of any one of clauses 426 to 428, wherein the aerating comprises aerating the drug delivery device until it comprises less than about 700 ppm ethylene glycol, about 800 ppm ethylene glycol, about 900 ppm ethylene glycol, about 1000 ppm ethylene glycol, about 1100 ppm ethylene glycol, about 1200 ppm ethylene glycol, about 1300 ppm ethylene glycol, about 1400 ppm ethylene glycol, or about 1500 ppm ethylene glycol.

Clause 430. The method of any one of clauses 423 to 429, wherein the drug delivery device comprises about 4 mg/cm³ to about 8 mg/cm³ collagen and about 6 mg/cm³ to about 10 mg/cm³ bupivacaine, or a salt thereof.

Clause 431. The method of any one of clauses 423 to 430, wherein the drug delivery device comprises about 6 mg/cm³ collagen and about 8 mg/cm³ bupivacaine, or a salt thereof.

Clause 432. A drug delivery device made by the method of any one of clauses 423 to 431.

Clause 433. A method of performing a soft tissue surgery procedure in a subject in need thereof, comprising placing the drug delivery device of any one of clauses 401 to 407, or 432, at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia, or nerve blockade which lasts for at least about one day.

Clause 434. The method of clause 433, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 435. The method of clause 433, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 436. The method of clause 433, wherein the surgery procedure is open ventral hernia repair comprising mesh placement at different subcutaneous layers, including intraperitoneal placement.

Clause 437. The method of clause 433, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 438. The method of clause 433, wherein the surgery procedure is open ventral hernia repair comprising onlay mesh repair, wherein the drug delivery device is placed in part in the preperitoneal space between the closed peritoneum and posterior rectus sheath, and in part laid in direct contact with the closed anterior rectus sheath.

Clause 439. The method of clause 433, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 440. The method of clause 433, wherein the surgery procedure is laparoscopic-assisted colectomy comprising placing the drug delivery device in part at the site of fascial repair, and in part superficially above the abdominal wall musculature and beneath extraction site incision.

Clause 441. The method of clause 433, wherein the surgery procedure is reduction mammoplasty comprising placing the drug delivery device in part in one or both breast pockets.

Clause 442. The method of any one of clauses 433 to 441, wherein more than one drug delivery device is placed at the surgical site.

Clause 443. The method of clause 442, wherein three drug delivery devices are placed at multiple layers in the soft tissue at the surgical site.

Clause 444. The method of any one of clauses 433 to 443, wherein bupivacaine, or a salt thereof, is present in the drug delivery device in an amount sufficient to provide a duration of local analgesia, local anesthesia, or nerve blockade which lasts for at least about one day after administration.

Clause 445. The method of clause 444, wherein bupivacaine, or a salt thereof, is present in the drug delivery device in an amount sufficient to provide a duration of local analgesia, local anesthesia, or nerve blockade which lasts for at least about 72 hours after administration.

Clause 446. The method of any one of clauses 433 to 445, further comprising partitioning the drug delivery device into segments with a predetermined size, wherein each segment is placed at the surgical site independently.

Clause 447. The method of clause 446, wherein the release dissolution profile of the sum of drug delivery device segments is substantially similar to the release dissolution profile of the unpartitioned drug delivery device.

Clause 448. The method of clause 447, wherein the release dissolution profile of the sum of drug delivery device segments is within about 1% and about 15% at any point in time substantially similar to the release dissolution profile of the unpartitioned drug delivery device.

Clause 449. A kit for performing a soft tissue surgery procedure in a subject in need thereof, the kit comprising the drug delivery device of any one of clauses 1-7, or 32, and instructions for placement of the drug delivery device at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

Clause 450. The kit of clause 449, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

Clause 451. The kit of clause 449, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

Clause 452. The kit of clause 449, wherein the surgery procedure is open ventral hernia repair comprising mesh placement at different subcutaneous layers, including intraperitoneal placement.

Clause 453. The kit of clause 449, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

Clause 454. The kit of clause 449, wherein the surgery procedure is open ventral hernia repair comprising mesh repair, wherein the drug delivery device is placed in part in the preperitoneal space between the closed peritoneum and posterior rectus sheath, and in part laid in direct contact with the closed anterior rectus sheath.

Clause 455. The kit of clause 449, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

Clause 456. The kit of clause 449, wherein the surgery procedure is laparoscopic-assisted colectomy comprising placing the drug delivery device in part at the site of fascial repair, and in part superficially above the abdominal wall musculature and beneath extraction site incision.

Clause 457. The kit of clause 449, wherein the surgery procedure is reduction mammoplasty comprising placing the drug delivery device in part in one or both breast pockets.

Clause 458. The kit of any one of clauses 449-457, further comprising instructions for partitioning the drug delivery device into segments with a predetermined size, wherein each segment is placed at the surgical site independently.

Clause 459. The kit of clause 458, wherein the release dissolution profile of the sum of drug delivery device segments is substantially similar to the release dissolution profile of the unpartitioned drug delivery device.

Clause 460. The kit of clause 459, wherein the release dissolution profile of the sum of drug delivery device segments is within about 1% and about 15% at any point in time substantially similar to the release dissolution profile of the unpartitioned drug delivery device.

### EXAMPLES

### Example 1: Collagen Isolation

### Manufacturing Process Overview

The manufacturing process for bovine purified type I collagen begins with the milling of the lyophilized bovine tendons to achieve uniform bovine tendon flakes. The raw material, bovine tendon flakes are proportioned and stored at 2° C.-8° C. until released for further processing. Processing of the milled tendons begins with the soaking of the milled tendons in a high pH sodium hydroxide (NaOH) solution for viral inactivation. Collagen is extracted and purified from the viral inactivated tendon slurry using sodium chloride (NaCl), NaOH and hydrogen peroxide (H₂O₂) solutions. The processed tendon slurry is then dispersed in an acidified aqueous solution to induce swelling of the fibrous material and the swollen tendon fibers are cut. Pepsin is added to aid in removal of impurities and provide an additional level (low pH) of viral inactivation. The solution (containing the purified collagen) is subsequently precipitated, and the purified collagen cake is cut to a smaller size and lyophilized. The lyophilized collagen is removed from the freeze dryer, milled into a fine flake powder and stored at 40° C.±2° C./75% RH for a period of 6 to 8 weeks to allow for equilibration of the material. End of the equilibration is determined based on a viscosity of NMT 250 cP resulting in a highly purified type I collagen raw material.

### Milling of Lyophilized Bovine Tendons

Bovine tendons are delivered lyophilized by the supplier. After incoming inspection and release of the material, pre-treatment will start. The manufacturing of bovine purified type I collagen is initiated by milling the lyophilized raw tendon material. The cutting gap of the mill is set to 0.1 mm-0.2 mm with a discharge sieve setting of 7 mm as detailed in FIG. 1. In-process samples of the lyophilized milled bovine tendons are obtained and quality control testing is performed against established material specifications detailed in Table 2. Milled tendons are collected in a plastic tote box and are subsequently aliquoted into polyethylene (PE) bags and stored at 2° C.-8° C. prior to further processing. Milled tendons may be stored at this temperature for up to 2 years.

### Extraction and Purification of Type I Collagen

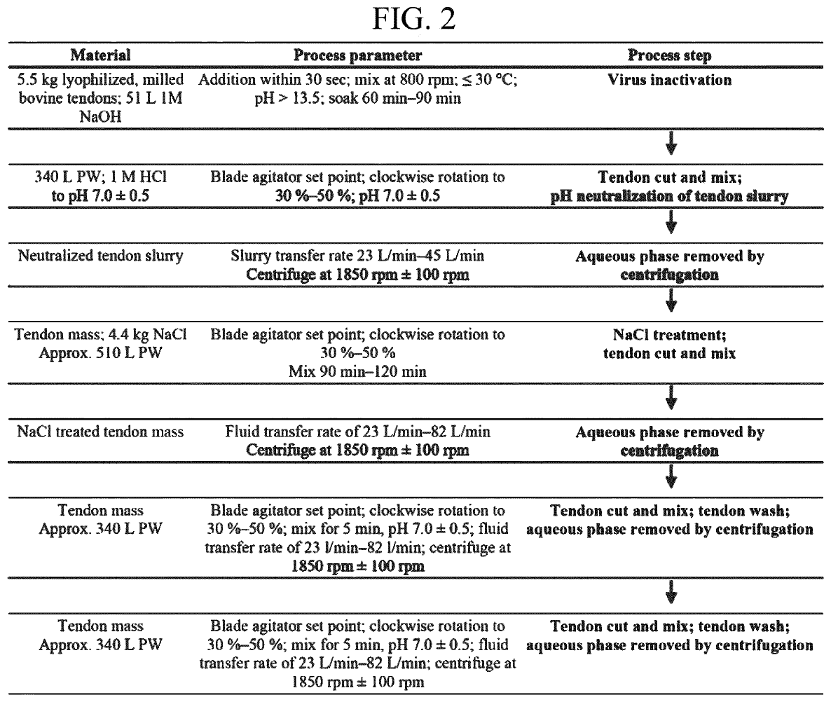

Lyophilized, milled bovine tendons are soaked for 60 min-90 min in IM NaOH at pH>13.5 for inactivation of potential adventitious contaminants like viruses and bacteria (FIG. 2). This manufacturing step (pH>13.5) is the first of two steps in the process that provides viral clearance. The NaOH treated bovine tendon material is transferred to a vessel containing a defined amount of purified water USP (PW) and is neutralized with 1 M HCl to pH 7.0±0.5, while mixing.

Neutralization of the slurry results in the formation of NaCl, which aids in the removal of low molecular weight soluble intrinsic impurities of the collagen. After neutralization, the solid portion of the tendon slurry is subsequently separated from the liquid portion through centrifugation. The solid tendon material is re-suspended in 0.9% NaCl, and mixed under slow agitation, which enhances the solubility of intrinsic impurities. Following centrifugation, the tendon material is washed two additional times with PW to further remove low molecular weight soluble components from the collagen.

A second NaOH treatment (0.1 M NaOH, pH 13.0±0.5 for a period of 14 h-16 h incubation) is performed, which contributes to the depletion of fat content and soluble impurities (FIG. 3). Following the second NaOH treatment, the tendon slurry is neutralized to a pH of 7.0±0.5 with 1 M HCl and washed twice with PW. The washed tendons are treated for 10 min-15 min with H₂O₂ solution (approx. 1.7%), which serves to bleach the tendon material and aids in further removal of fat content. The bleached tendon slurry is centrifuged and washed with PW to remove residual peroxides. This is important in that residual peroxide can cause inactivation of the pepsin used in a later process step.

The bleached tendon material is dispersed in acidified PW (pH 2.3-2.7) to affect swelling of the fibrous material (FIG. 4). The swollen tendon fibers are then shredded, utilizing a tank bottom mounted rotor stator cutter and combined with pre-prepared pepsin solution. Preparation of the pepsin solution consists of dissolving the pepsin in acidified water at pH 1.8-2.1 at 34.1° C.-35.1° C. for 30 min-60 min. The pepsin digestion step in the purification process reduces the potential for non-collagen protein contamination (eg, bovine serum protein), and reduces the potential species related immunogenicity of the collagen through cleavage of telopeptides (i.e., the short non-helical domains at the end of the collagen chains). In addition, the pepsin digestion step represents the second viral clearance step of the process. This step along with the initial NaOH soak comprise the collective process related mechanisms for viral clearance.

The tendon/pepsin slurry is centrifuged with the solubilized collagen retained and the solid centrifugate is discarded. The collagen solution is passed through a 250 μm filter and the purified collagen is precipitated through pH adjustment with 1 M NaOH to pH 7.5±0.5.

The purified collagen is centrifuged and washed twice with PW, pH adjusted to 7.5±0.5 with acetic acid and/or NaOH (FIG. 5). At this point in the manufacturing process, the purified collagen may be placed into LDPE bags and stored for no more than 2 days at 2° C.-8° C. awaiting lyophilization or immediately distributed in trays and lyophilized (FIG. 6).

In preparation for lyophilization, the wet collagen is placed into PETG trays and placed into the freeze dryer for further processing (FIG. 6). The lyophilization cycle for the purified collagen consists of:
freezing the wet collagen to −38° C.;
condenser temperature set to 75° C.;
evacuating the chamber to 0.2 mbar pressure;
ramping the shelf temperature to 30° C. and holding for 23.5 h;
cycle end by time after 23.5 h at 20° C.

The steps of the lyophilization cycle can be found in Table 1.

At the end of the lyophilization process, the collagen is removed from the freeze dryer, milled into a powder and stored at 40° C.±2° C., 75%±5% relative humidity for a period of 6 to 8 weeks. This final step acts to equilibrate physicochemical properties, such as viscosity during the manufacture of bulk dispersion of the collagen as part of the final manufacture of the Xaracoll combination drug product.

TABLE 1
Overview of the freeze dryer cycle for
the lyophilization of purified collagen

| | | Time | Temperature | Pressure |
|---|---|---|---|---|
| Step | Description | (hh:mm) | (° C.) | (mbar) |
| 1 | Freezing | 01:00 | −38 | Atmospheric |
| 2 | Freezing | 03:00 | −38 | Atmospheric |
| 3 | Preparation | 00:20 | −38 | Atmospheric |
| 4 | Preparation - vacuum | 00:15 | −38 | 0.2 |
| 5 | Drying | 02:10 | 30 | 0.2 |
| 6 | Drying | 23:30 | 30 | 0.2 |
| 7 | Final drying | 01:00 | 20 | 0.2 |

### Example 2: LMC Maturation

The LMC maturation process requires tight control of LMC loss on drying (LOD) during equilibration. Permeable pouches (e.g. Stericlean) are used, the equilibration temperature is maintained at 40° C., and the RH is maintained at 65%. Under these parameters, the LMC reaches a target equilibrium LOD of approximately 18% within short time of about four to eight weeks. At this LOD, a 0.9 wt % dispersion of LMC at a pH of about 4.5 and a temperature of about 40° C. viscosity has a viscosity of greater than about 150 cP and less than about 250 cP.

Afterwards, the LMC is partly dried to approximately 10% LOD at a temperature of 25° C. and 15% RH. The partly dried LMC is placed into a nonpermeable, tightly sealed, aluminum pouch and stored under refrigeration until used.

### Example 3: Preparation of Bupivacaine-Collagen Sponge

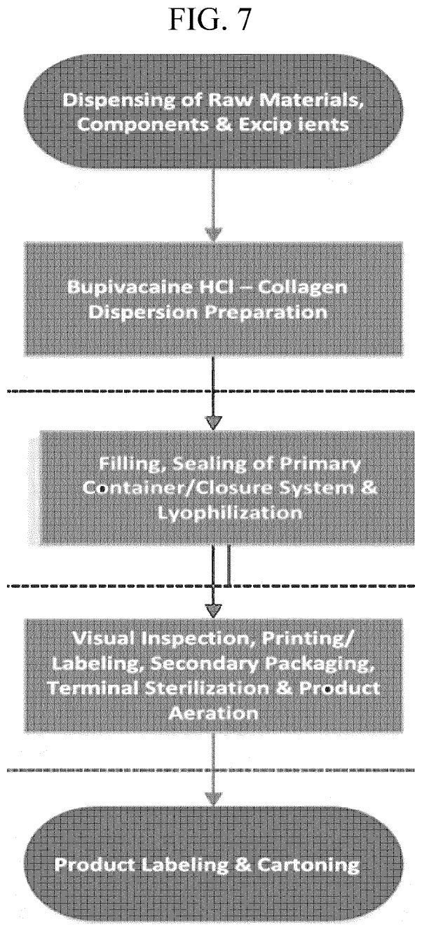

The target dispersion batch size in the process found below is 55 kg. The target dispersion batch size can be scaled up to about 70 kg with changes in the lyophilization process as detailed below. An overview of the preparation process is shown in FIG. 7.

### Bulk Dispersion Preparation

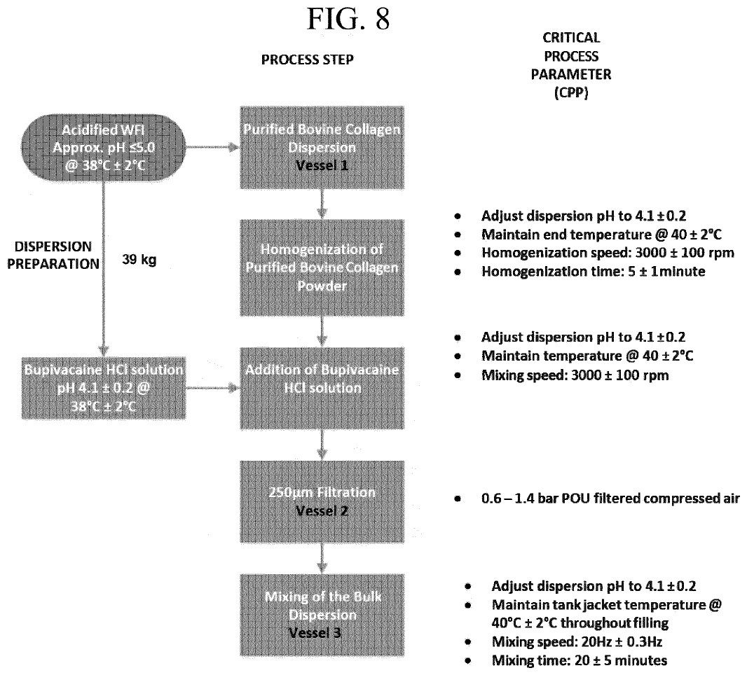

Acidified water (39 kg) is prepared for use in the process using acetic acid to adjust the pH of the WFI while controlling temperature to 38° C.±2° C. Highly purified type 1 collagen is dispersed in 39 kg of acidified WFI conditioned to 38° C.±2° C. (vessel 1) (FIG. 8). The collagen dispersion is homogenized at 3000 rpm±100 rpm for 5 min±1 min. During the homogenization process, the bulk dispersion temperature is controlled to remain below 42° C. Once homogenization of the dispersion is complete, the bulk temperature is maintained at 40° C.±2° C. The pH of the dispersion is adjusted, if necessary, to 4.1±0.2 with acetic acid. The high shear mixing required to effect collagen swelling and subsequent dispersion formation is accomplished by the use of a high-shear homogenizer. The homogenizer employed is an IKA Ultra Turrax TYP T115, equipped with a UTC 115 KT rotor-stator head, which is designed to create high shear forces by pulling the material through the rotating homogenizer head and forcing it against the proximal stationary stator head. It is this design that facilitates the high shear forces required to separate the fibrous collagen mass at the beginning of dispersion preparation.

The calculated quantity of bupivacaine HCl is dissolved in 14 kg of acidified WFI (pH 4.1±0.2) conditioned to 38° C.±2° C. An additional 1 kg of acidified WFI is used to rinse the container used to dispense the API after transfer to the collagen dispersion. This rinse is also added to the dispersion. Once dissolution of the bupivacaine solution is visually confirmed, it is added to the collagen dispersion and mixed using the homogenizer at 3000 rpms for 1 min. Once mixing of the bupivacaine-collagen is completed, the dispersion is transferred to vessel 2, which is able to be pressurized, and filtered through a 250 μm nylon bag filter and transferred to a closed heat-jacketed stainless-steel vessel. The jacket temperature is maintained at 40° C.±2° C. and the bupivacaine-collagen dispersion is maintained under low shear mixing (20 Hz±0.3 Hz) to ensure homogeneity of temperature in the drug-loaded collagen dispersion during filling.

### Product Filling, Sealing and Lyophilization

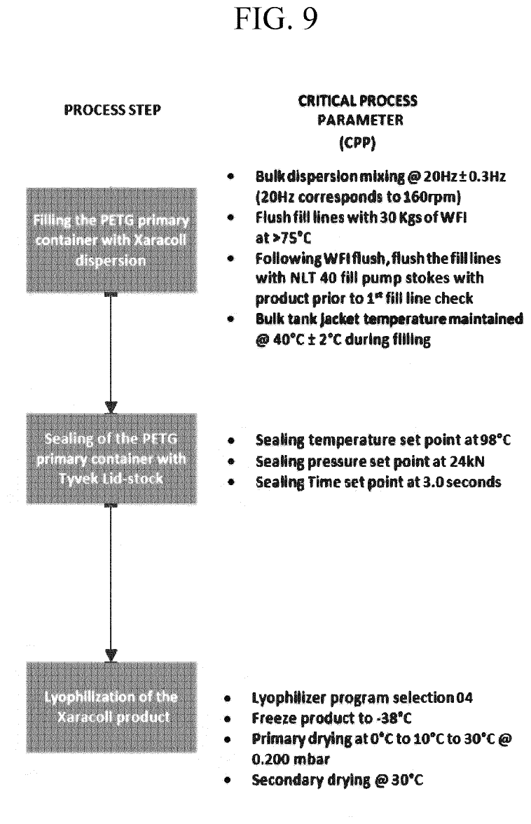

The filling process is performed within an ISO 5 classified zone using a 4-head Bausch & Strobel positive displacement filling system. Prior to the initiation of filling, the fill lines are flushed with WFI at a temperature of >75° C. After WFI flushing the tubing to the bulk XARACOLL dispersion line is connected and the fill lines are flushed with product dispersion. Each fill line assembly is flushed with 40 pump strokes of product, which is discarded prior to taking the initial fill weight control sample. The jacket temperature of the vessel containing the dispersion is maintained at 40° C.±2° C. throughout the filling process. Individual 5 cm×5 cm×1.5 cm PETG blisters are filled at a target fill weight of 12.5 g±0.5 g/blister (FIG. 9). Fill weight checks are performed at the beginning of the fill and at a minimum of 30-min intervals throughout the fill and finally at the end of the filling process.

In some aspects, the filled blisters are immediately sealed with a gas-permeable Tyvek lid-stock. In-process checks are performed to ensure that the Tyvek lid-stock seal is complete, no product splashes are observed and that the adhesive seam is confluent. In other aspects, the filled blisters are not sealed with a lid. Filled and sealed product (or filled and unsealed product) is transferred to the freeze dryer with shelf temperatures at ambient conditions. Once the freeze dryer has been loaded, product temperature load probes are placed in designated locations. In addition, probes are also employed providing data on shelf temperatures and chamber pressure. The door to the dryer chamber is closed and the lyophilization program is initiated.

Lyophilization method #1 (for 55 kg batches) (FIG. 10): The lyophilizer operating console permits process cycle programming and an automated program cycle has been established and validated for the product. The lyophilization program overview is shown in FIG. 4. The lyophilization program is started with the cooling of the shelves to 2° C. The shelf temperature is lowered in steps to initiate freezing. Following freezing to −38° C. for the designated time, the pressure within the freeze dryer chamber and condenser is reduced to 0.200 mbar and the shelf temperature is increased stepwise from 0° C. up to 10° C. and subsequently to 30° C. for the primary drying phase. Secondary drying of the product is performed at a freeze dryer shelf temperature to 30° C. while maintaining the chamber pressure at 0.200 mbar. Once the prescribed drying time at 30° C. is completed, the shelf temperature is lowered to 10° C. and kept there until the program cycle is stopped, the chamber pressure is brought to atmosphere with filtered air and the product is unloaded. Samples are pulled submitted for QC testing.

Lyophilization method #2 (for 70 kg batches) (FIG. 11): The program overview is shown in FIG. 10 and FIG. 11. The filled product is placed on a shelf at 20° C. and the shelf temperature is lowered to 2° C. The shelf is then cooled further to −38° C. to initiate freezing. Following freezing to −38° C. for the designated time, the pressure within the freeze dryer chamber and condenser is reduced to 0.200 mbar and the shelf temperature is increased to 12° C. for the drying phase. Once the prescribed drying time at 12° C. is completed, the chamber pressure is brought to atmosphere with filtered air and the product is unloaded. Samples are pulled submitted for QC testing.

### Visual Inspection and Secondary Packaging

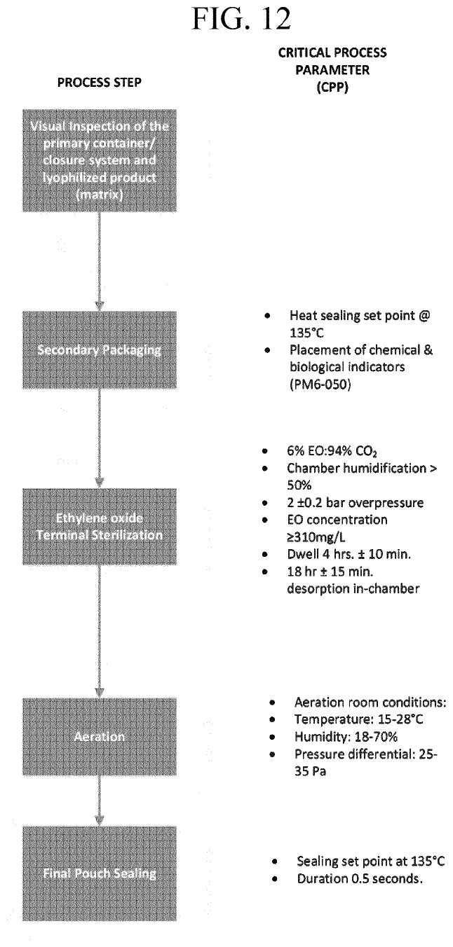

100% visual inspection of the product and primary container/closure performed (FIG. 12). Visual inspection is aided by the use of diffuse bottom lighting and is performed by qualified inspection operators. XARACOLL matrix implants are inspected in accordance with established inspection criteria for physical defects e.g., melt-backs, cosmetic defects, foreign particulates, presence of crystalline like structures, bubbles etc.). Similarly, the primary container/closure of each matrix implant blister is inspected for physical and cosmetic defects, sealing seam width etc. If the filled product was lyophilized without sealing with a lid, a Tyvek lid is placed on top of the primary container and sealed.

The bottom of each matrix implant blister is imprinted with the specific lot number, expiration date and “XARACOLL 100 mg.” The secondary packaging process proceeds with the placement of 3 individual PETG blisters, each containing a 100 mg XARACOLL bupivacaine-collagen matrix implant in a PETG frame. Product inspection and secondary packaging is conducted in ISO 8/GMP Class D cleanroom areas.

Each frame is placed into a PET/PE pouch containing a permeable Tyvek strip. The pouch is heat sealed above the Tyvek strip in preparation for EO terminal sterilization.

### EO Terminal Sterilization Process

EO biological indicators and EO chemical indicators are placed in designated locations determined during the EO sterilization validation studies. The product, packaged into the final sterile barrier system, is then terminally sterilized using EO gas. An automated/validated EO sterilization cycle is employed in the sterilization of XARACOLL. The sterilization process used in the production of the drug product is based on a 2-bar overpressure cycle, which is maintained for a sterilization period of 4 hours (gas exposure time for 6/94 EO/CO₂ gas mixture). Validation of the EO process is conducted in accordance with International Organization for Standardization [ISO] 11135 Medical Devices—Validation. Validation criteria employed an over-kill approach (i.e., confirmation of a SAL of 10-6 in a half cycle configuration).

### Post Ethylene Oxide Sterilization Aeration

After completion of post-sterilization flushing, the product is transferred to a holding area for longer-term aeration. This phase of the process serves to further scavenge low-level residual EO and ethylene chlorohydrin (ECH) and ethylene glycol (EGly) from the drug product and packaging. Samples are withdrawn for the lot beginning at week 5 in the aeration process and evaluated for the presence of EO, ECH & EGly. The drug product is held at room temperature until the limits for process residues meet established acceptance criteria.

Following completion of post-sterilization ventilation, the pouch containing the product is resealed below the gas permeable Tyvek window and this gas permeable (top) portion is then removed from the pouch. This results in a fully sealed pouch containing three terminally sterilized matrix products within the final container closure system. Samples are withdrawn and submitted to QC for final release testing.

### Product Labeling and Cartoning

Pre-printed pouch labels are imprinted with lot specific information (lot number and expiration date) are manually placed on the pouch. The labeled pouches are 2×100% inspected prior to insertion into a white chipboard carton, which is then imprinted the lot number, expiry dating, and 2D serialization code. The final packaged/cartoned XARACOLL product is inspected to confirm correct label information. Retention samples are obtained in accordance with established procedures and the final packaged XARACOLL product is stored at 20° C. to 25° C. (68° F. to 77° F.) awaiting final release by Qualified Person and Quality Assurance.

### Example 4: Effects of Low Viscosity on LMC and Resulting Xaracoll Matrices

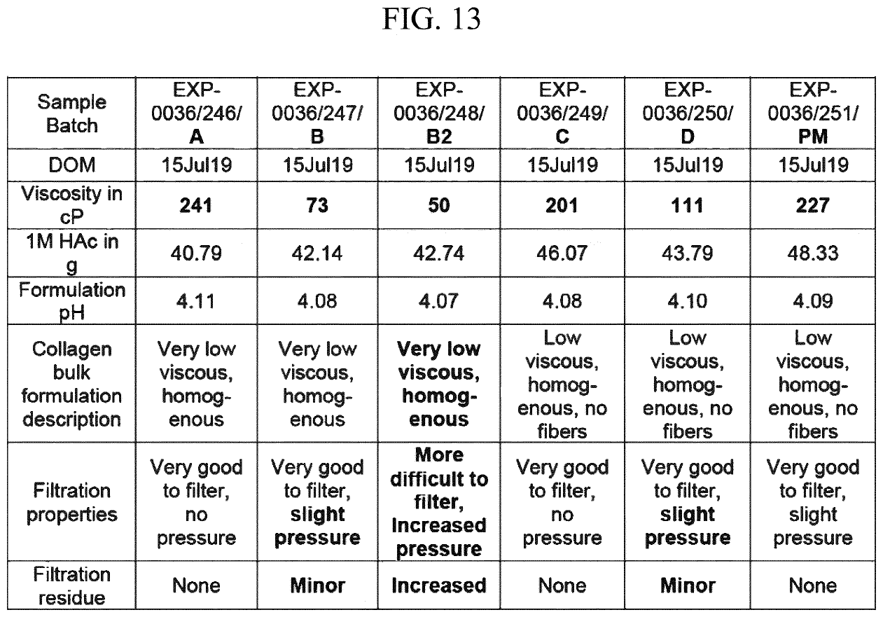

Xaracoll matrices were fabricated from dehumidified mature LMC within a desired viscosity range of about 110 cP to 250 cP. Matrices were also fabricated from mature LMC with a viscosity below the desired range. An overview of the Xaracoll matrix properties alongside the viscosity of the mature LMC used to fabricate the matrix can be found in FIG. 13.

### LMC Characterization

### Amino Acid Analysis

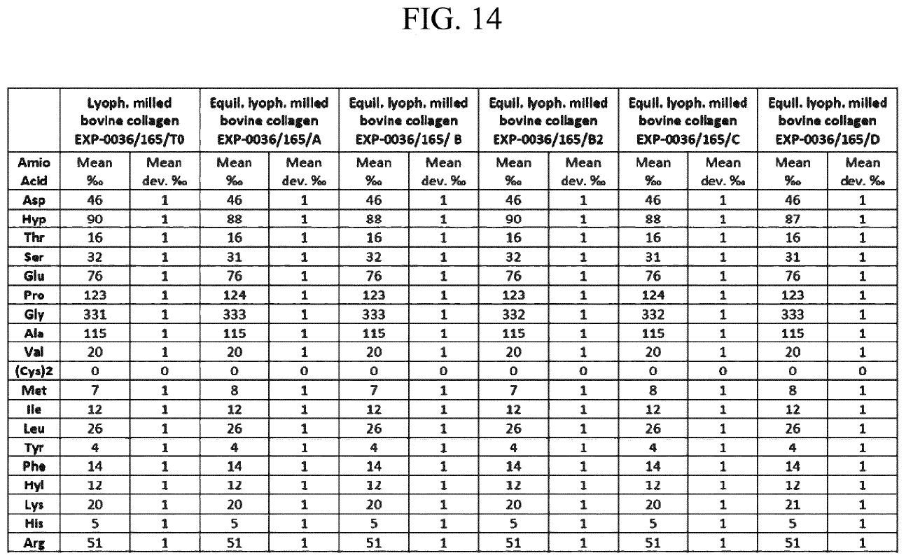

The amino acid composition was obtained for mature LMC samples (A, B, B2, C, and D) as well as a sample of non-matured, i.e., non-equilibrated LMC (T0) (FIG. 14). The amino acid composition can be used as fingerprint for type I collagen. Separation of the amino acids occurs by an ion exchange chromatography followed by a post column derivatization using ninhydrin which permits photometric detection of α-amino acids. The amino acids glycine, hydroxyproline and proline are determined at 440 nm, while the other amino acids are determined at 570 nm. For the quantitative determination, an external calibration is performed applying an amino acid standard solution. Prior to chromatography, the samples are hydrolyzed in 6 M hydrochloride acid containing β-mercaptoethanol at 110° C. for 20 h and excess acid is removed by evaporation.

### Determination of Free Amines by TNBS Derivatization

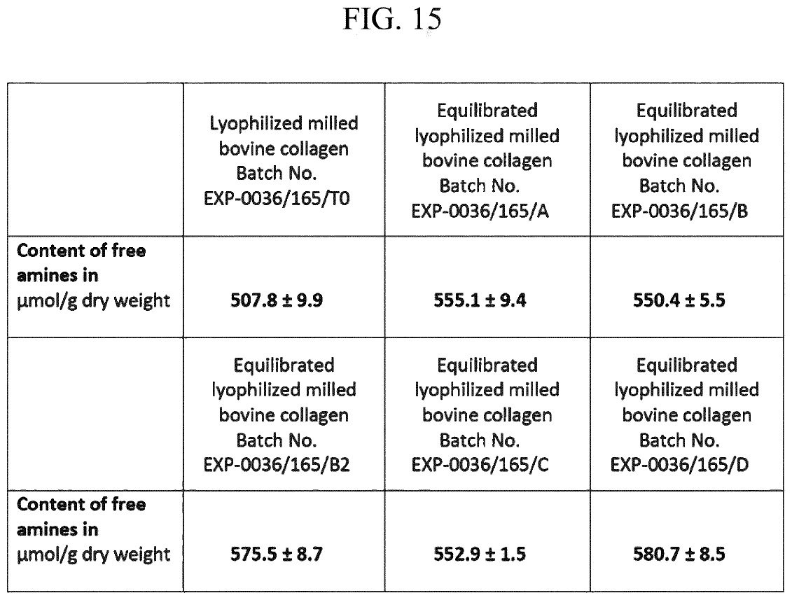

The content of free amines in collagen materials can be used to estimate their cross-linking degree, with a lower trinitrobenzene sulfonic acid (TNBS) read-out correlating with a higher consumption of ε-amino groups, i.e., higher level of cross-linking. Derivatization with trinitrobenzene sulfonic acid (TNBS) is used to detect the terminal α-amino groups as well as the free ε-amino groups in peptides and proteins, resulting in the formation of colored adducts measured by absorbance at 345 nm. The contents of free amines determined for the tested bovine collagen samples are shown in FIG. 15.

In this study, non-matured LMC showed an approximately 10% lower content of free amines compared to all equilibrated mLMC groups, indicating a higher degree of natural cross-links (FIG. 15). In the same line, over-matured (i.e., very low viscosity) mLMC (e.g., B2 and D) showed the tendency to even higher content of free amines.

### Protein Identification Via LC-MS

Collagen type-specific peptides can be detected by LC-MS providing information about the collagen type. Furthermore, non-collagen protein impurities can be detected by LC-MS. For LC-MS analysis, the samples were digested with trypsin and the resulting peptides were purified and separated on a reversed phase column using high performance liquid chromatography. The eluting peptides were analyzed on-line by tandem mass spectrometry using an ion trap instrument (QTRAP 4000, Sciex). Spectra were recorded by the Analyst program (Sciex) recording two product ion scans per full ms scan. The resulting peak lists were analyzed using the software ProteinPilot (ABSciex) and the database uniprot−bos+taurus+cont.fasta containing all entries for Bos taurus from the NCBI Database as of 23/08/17.

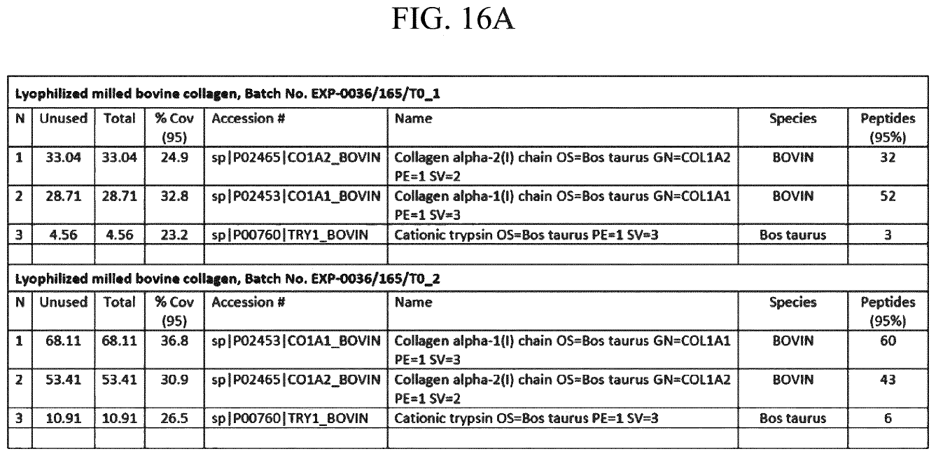

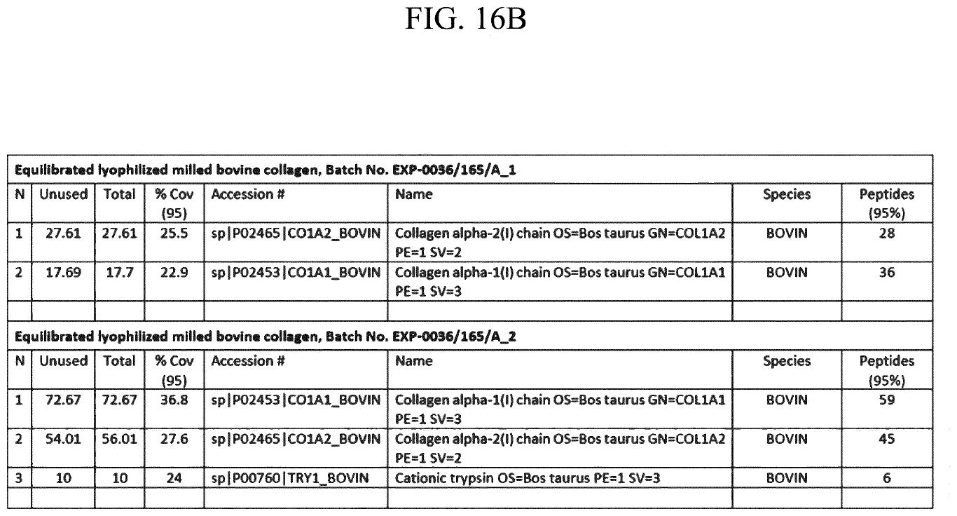

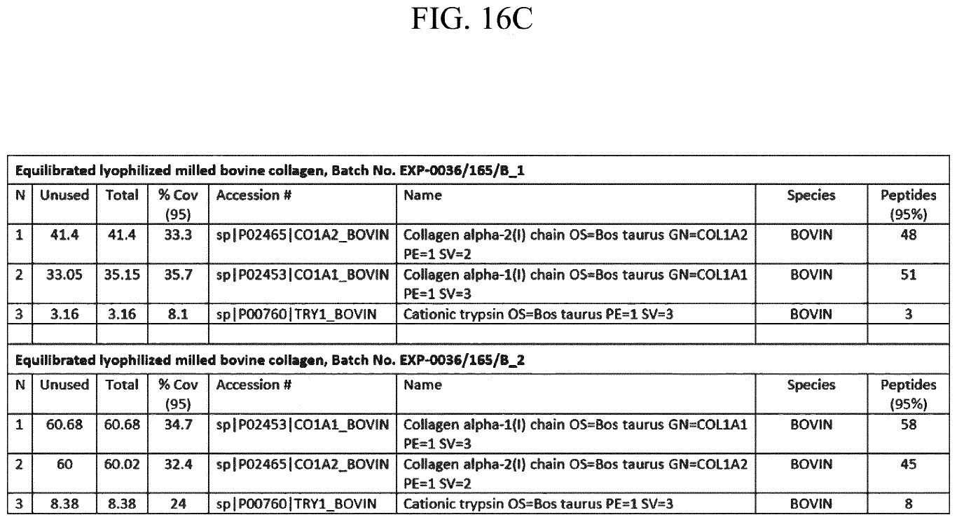

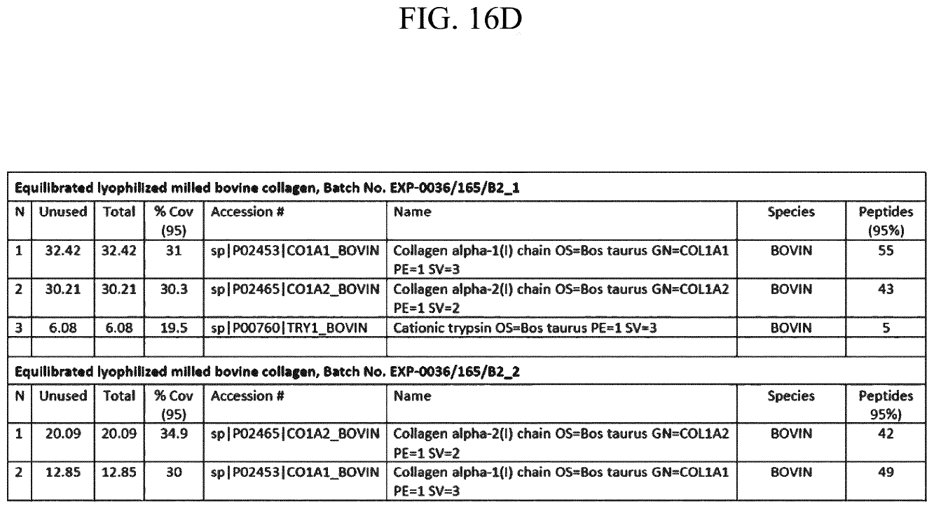

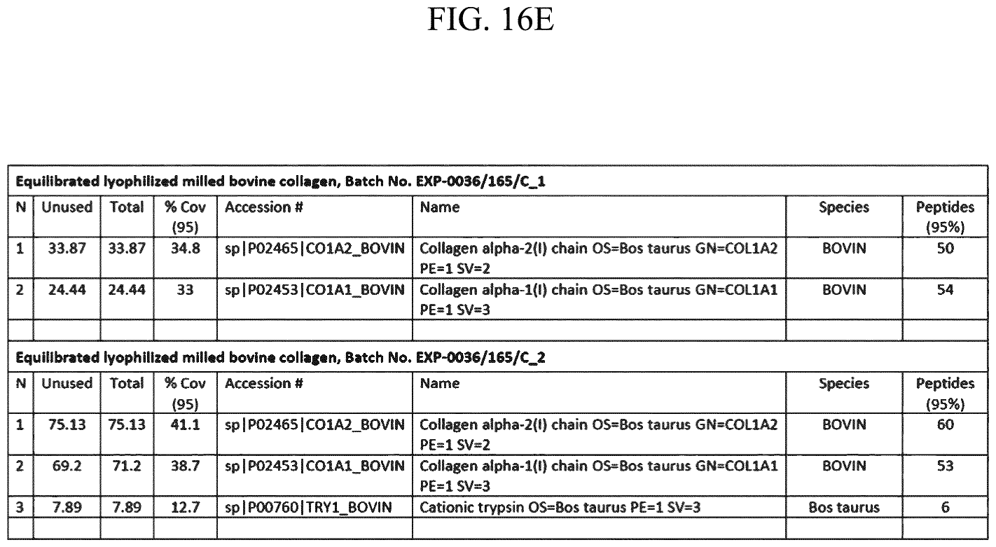

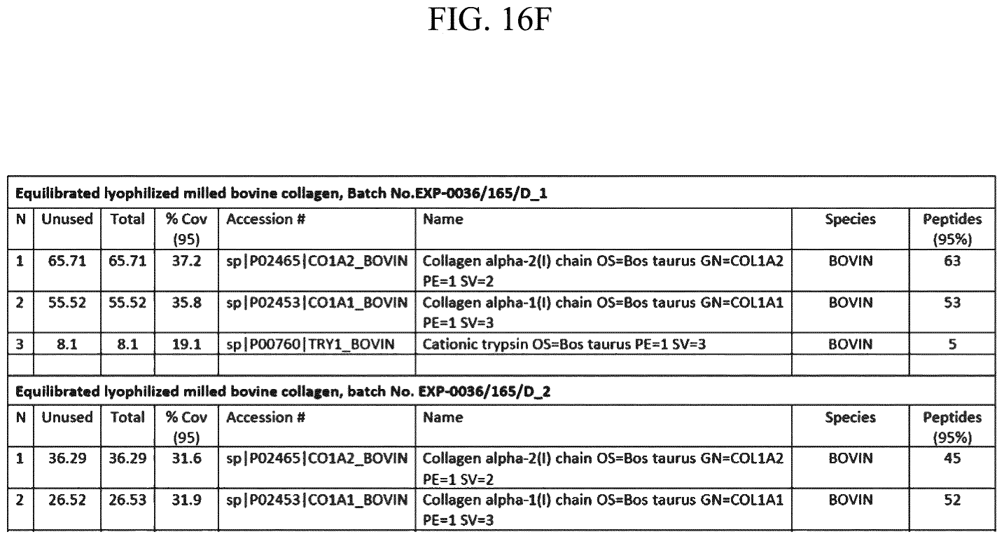

The results of the LC-MS analysis are summarized in FIGS. 16A-16E. Peptides corresponding to the α1- and α2-chain of collagen type I were detected in all samples, as expected. Additional peptides corresponding to trypsin are assumed to be derived from the tryptic digestion prior to LC-MS analysis. No other peptides—indicative for impurities—were found.

### Trypsin Resistance and SDS-PAGE

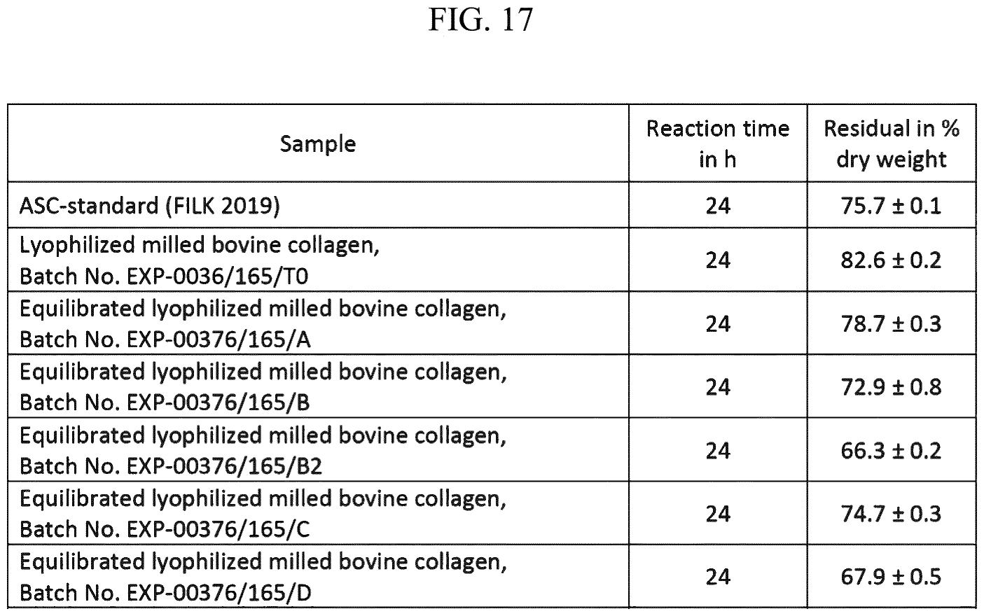

In the native collagen triple helix, the protein is protected from enzymatic degradation of most proteinases (with exception of e.g., collagenase) by steric hindrance. Resistance against enzymatic digestion by trypsin without an initial denaturation step is therefore used to determine the native portion of collagen materials. After incubation of the samples with the enzyme at 37° C. for a period of 24 h, the residues were washed, dried and determined gravimetrically. The supernatants of the incubations were collected, lyophilized and further analyzed by SDS-PAGE. For the different mLMC samples, an amount corresponding to approx. 20 μg protein was loaded onto the gel. The molecular weight cutoff of the gel, used for this analysis, was around 6 kDa. The weight portions remaining after incubating the different (m)LMC samples with trypsin for a period of 24 h are in FIG. 17.

### Impurities by SDS-PAGE

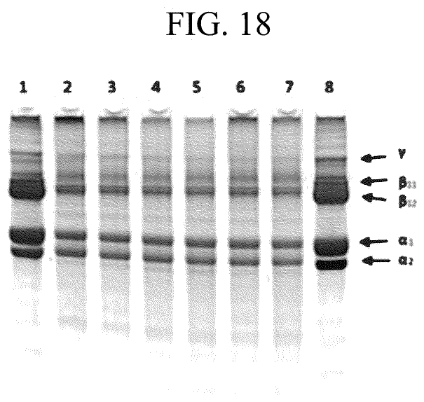

SDS-PAGE is used for identification of collagen related peptide chains as well as non-collagen protein impurities. To obtain the soluble part of the sample materials, the different bovine collagens were treated with 0.1 M acetic acid for 20 min at 60° C. After centrifugation, the supernatants were collected and lyophilized. Prior to SDS-PAGE, the lyophilized samples were re-dissolved in SDS-sample buffer and subsequently incubated at 95° C. for 2 min. 10 μL of this solution was loaded onto the gel. The solubility achieved during sample preparation ranged between 9 and 19% for the different bovine collagens. Taking the soluble fractions of the samples, SDS-PAGE analysis was carried out by applying a gel with a separation range from 200 to 6.5 kDa. FIG. 18 lanes 1 and 8 show an electrophoretic pattern that is characteristic for acid soluble collagen. The monomeric α1- and α2-chains, the dimeric β11- and β12-chains, the trimeric γ-chain as well as some high molecular weight components at the top of the gel were clearly visible. This band pattern of γ-, β- and α-chains was also found for all tested sample materials. Further peptide bands, indicating impurities by other proteins than collagen, were not detected.

### Scanning Electron Microscopy (SEM) Analysis

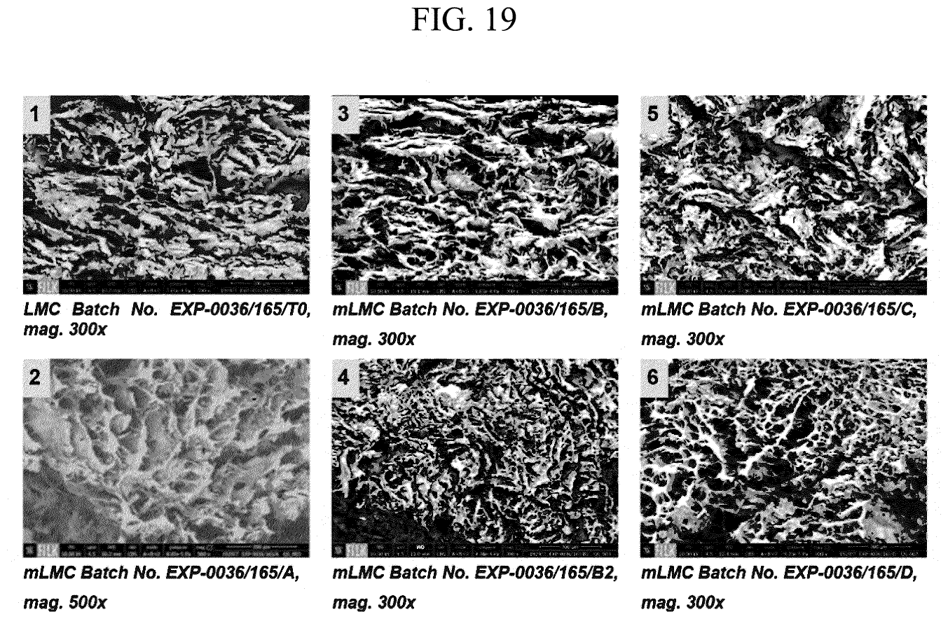

During SEM analysis, the surface and the cross section of a specimen are scanned with a focused beam of electrons. The electrons interact with atoms in the specimen producing various signals that are measured to generate an image of the surface topography and composition. SEM-images of the cross sections of the different mLMC samples are displayed in FIG. 19.

### Atomic Force Microscopy (AFM) Analysis

AFM combines the advantage of a very high magnification (near to that of Transmission Electron Microscopy) with the capability for three-dimensional resolution similar to Scanning Electron Microscopy. The underlying principle of AFM analysis is based on a laser beam pointed onto the backside of a cantilever to measure the cantilevers deflection. The reflected laser beam is then measured with a four-segmented photodiode to obtain the height information from the sample. For the analysis of the lyophilized bovine collagen, the AFM was used in the amplitude modulated contact mode (AC mode). In this mode, the tip oscillates near or at the cantilevers resonance frequency and the amplitude is used as the feedback mechanism. This is a soft imaging mode for soft or condensed matter, e. g. collagen. With AC mode, the height information (height image) and information of the viscoelastic properties (phase image) can be obtained. Sample preparation for AFM-analysis involved taking a small specimen from the sample and gently pressing it down onto a microscope slide.

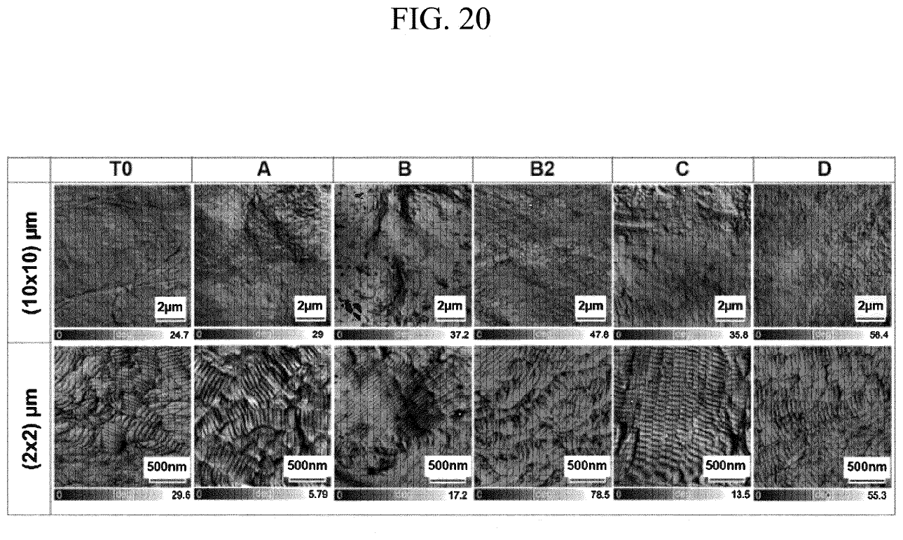

AFM images of the lyophilized milled bovine collagen samples are displayed in FIG. 20. The material is comprised of a collagen micro fibril assembly showing a disordered orientation. Fibrils with the typically collagen D-band pattern are visible. Possible image deficiencies might be explained by artefacts resulting from the pressing procedure during sample preparation or from the movement of the cantilevers tip.

### Differential Scanning Calorimetry (DSC)

The denaturation temperature and enthalpy of collagen materials is a measure of the structural stability correlating also with the level of crosslinking and/or denaturation of collagen structure. The denaturation temperature and enthalpy was determined using DSC on the Mettler Toledo DSC 3+ by placing the sample in a pressure-sealed aluminum crucible and ramping the temperature from 5 to 90° C. at a rate of 5° K/min. Prior to analysis, the sample is incubated in buffer solution at pH 7.4. An empty aluminum crucible is used as the reference. Collagen was measured using method QC6-058. The enthalpy results for collagen were normalized to the collagen dry weight.

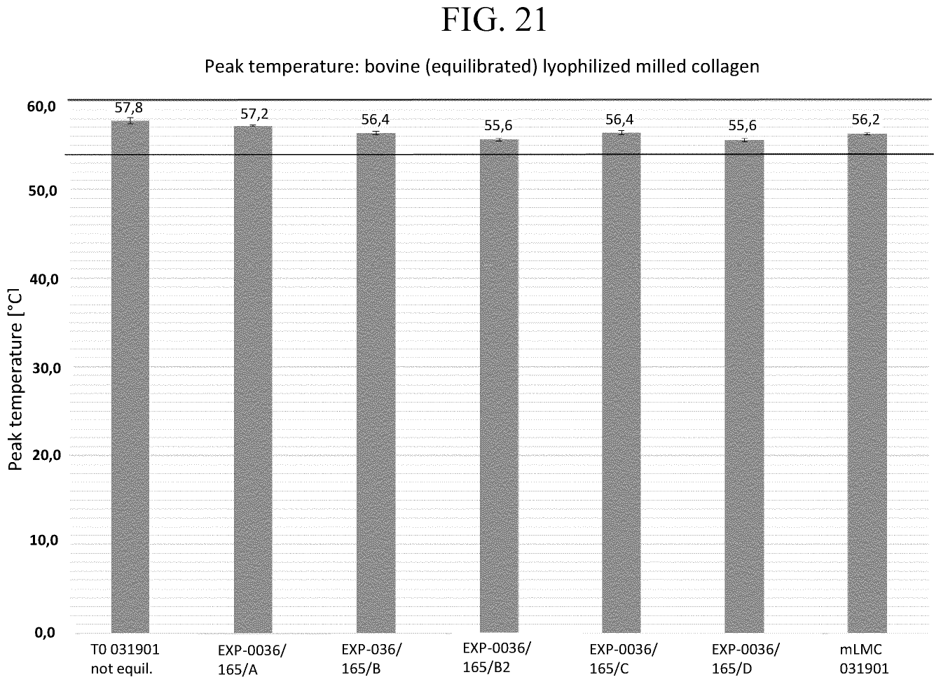

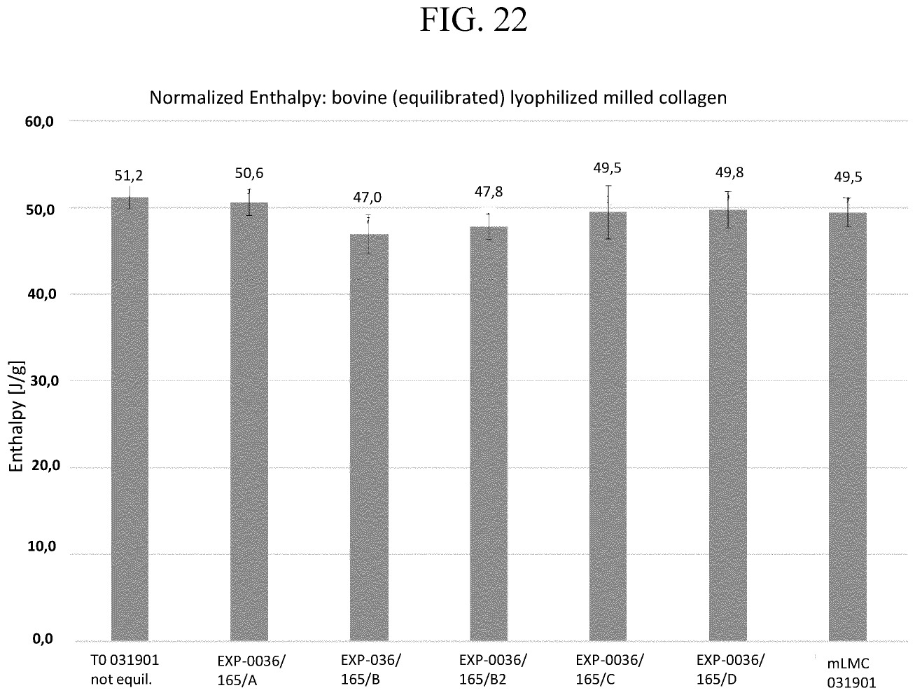

All samples are within the specification of the peak denaturation temperature (54 to 60° C.) (FIG. 21 and FIG. 22). There was low variability between the samples and a slight trend for lower peak denaturation temperature for B2 and D (FIG. 21). For the normalized enthalpy, there was high variability between the samples, however, somewhat lower enthalpy was found for B and B2 samples (FIG. 22).

### Analysis of Natural Cross Links

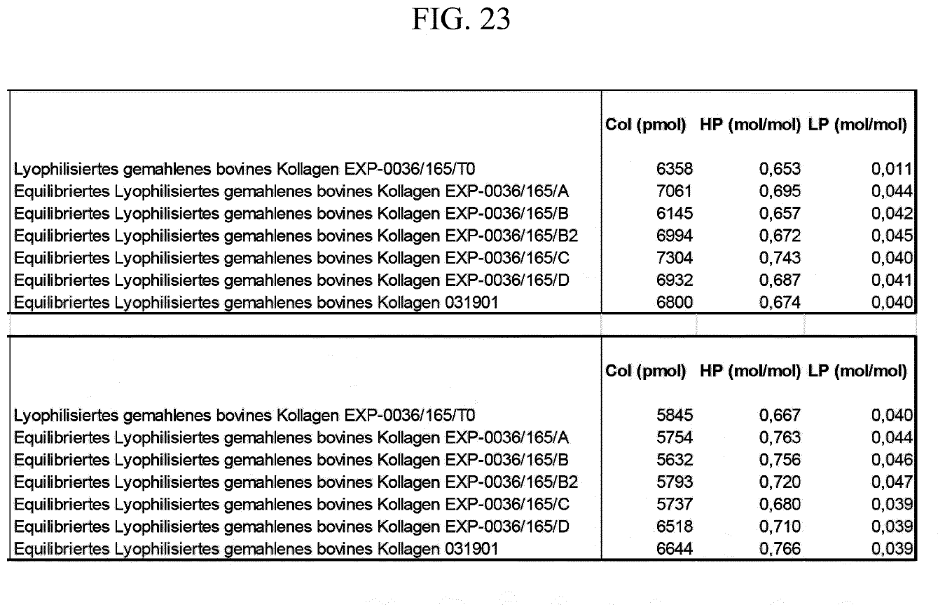

Collagen type I is characterized by a variety of intermolecular cross-links, including divalent and trivalent cross-links. The types of natural cross-link types in the mLMC samples were analyzed using the following procedure: Reduction of the samples (10-20 mg) with reduction buffer (25 mg NaBH₄/ml in 0.05 M NaH₂PO₄/0.15 M NaCl pH 7.4, cold room, overnight). The supernatant represents the water-soluble fraction of the samples. The reduction stabilizes reducible cross-links prior to acid hydrolysis (DHLNL, HLNL, HHMD). Thereafter, the samples were degraded by highly purified bacterial collagenase (Sigma, Type VII collagenase 50 CDU in Dulbecco's PBS with calcium chloride and magnesium chloride). The supernatant contained solubilized collagen. After hydrolysis of the samples (6 N HCl, 24 h, 110° C.), the amino acid analysis was performed by post column derivatization with ninhydrin. For cross-link analysis, an enrichment of collagen cross-links and a depletion of amino acids were performed using solid phase extraction. The analysis of the cross-links was performed by amino acid analysis (post column derivatization with ninhydrin) in the reduced and in the non-reduced state (FIG. 23).

All non-reduced samples showed a similar natural cross-link pattern (HP and LP), whereas a lower LP content was found in non-matured LMC compared to mLMC in reduced samples (FIG. 23).

### Analysis of Glycosylation

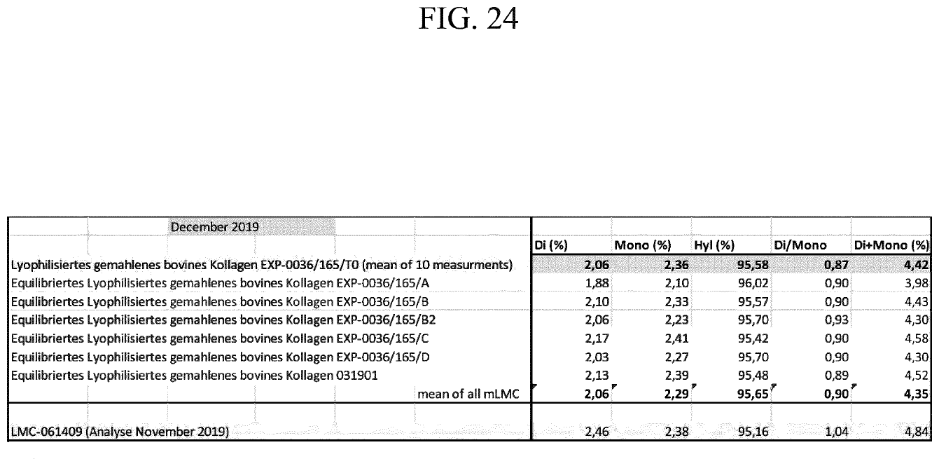

Glycoproteins, including glycosaminoglycans, are known as structural components covalently linked to collagen. Glycosylation was determined as follows: 10-20 mg of the sample were hydrolyzed (1000 μl 2 M KOH for 24 h, 110° C.). Precipitation of potassium perchlorate by acetic acid/perchloric acid (50 μl/150 μl 70%). Evaporation and solubilization of the supernatant in 500 μl water and purification by solid phase extraction. Analysis of the glycosylation by amino acid analysis (post column derivatization with ninhydrin). Analysis of the hydroxylysine content in a parallel sample after acidic hydrolysis (6 M HCl, 110° C., 24 h). The data indicate no significant differences between the different samples regarding glycosylation pattern, i.e., mono- and di-saccharides or Hydroxylysine (i. e., coupling point for saccharides) content (FIG. 24).

### Bulk Dispersion Characterization

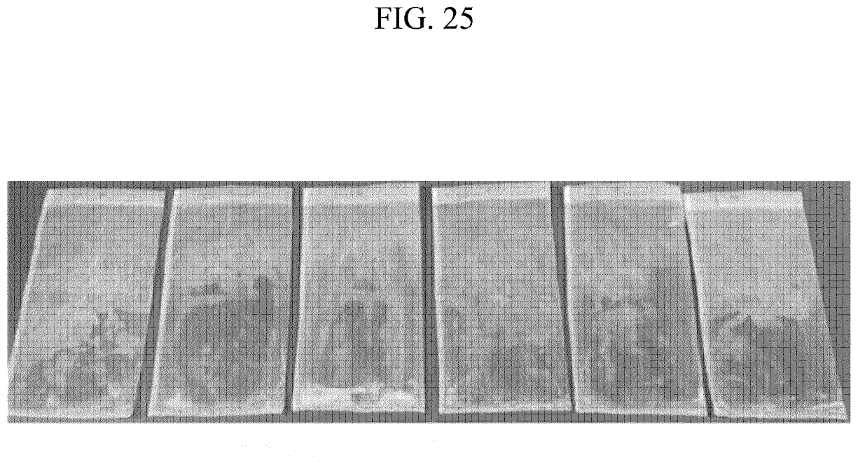

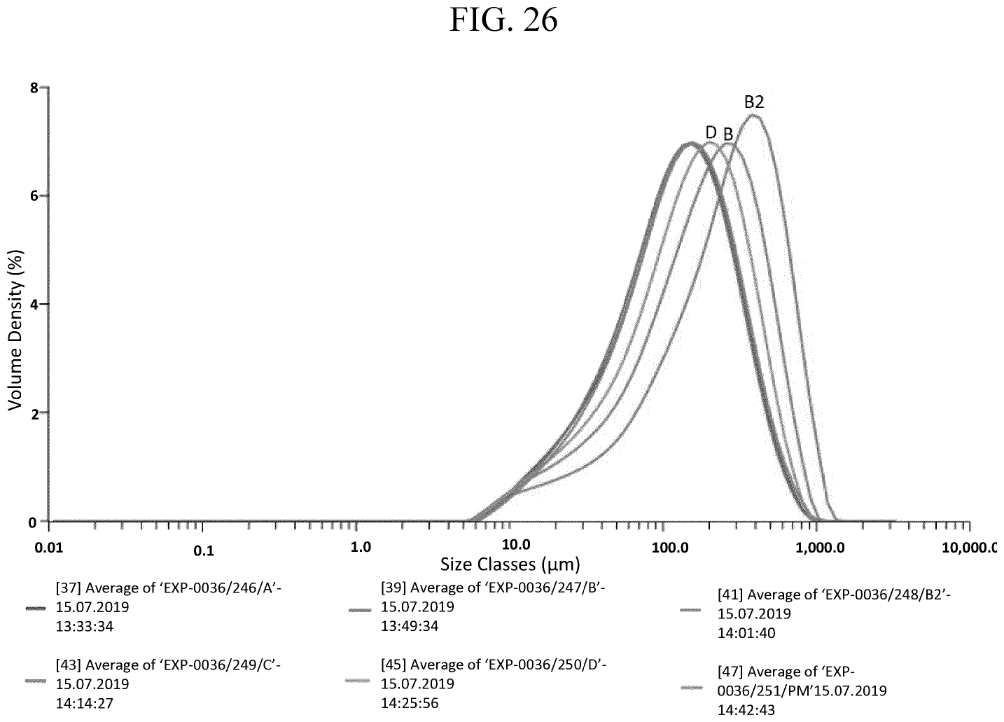

The filtration properties of the Xaracoll bulk formulations correlate with the mLMC viscosity, i.e., filtration more difficult at too low viscosity correlating with increased collagen fibers (B2>B/D). The amount of filter residue correlates with the mLMC viscosity, i. e., higher filtration residues at too low viscosity (B2>B/D) (FIG. 25 depicts the filter residue with mLMC viscosity. FIG. 26 depicts the particle size distribution in the Xaracoll bulk dispersion correlates with the mLMC viscosity, i.e., the lower viscosity the higher particle size distribution (B2>B>D>A/C/PM) (FIG. 26).

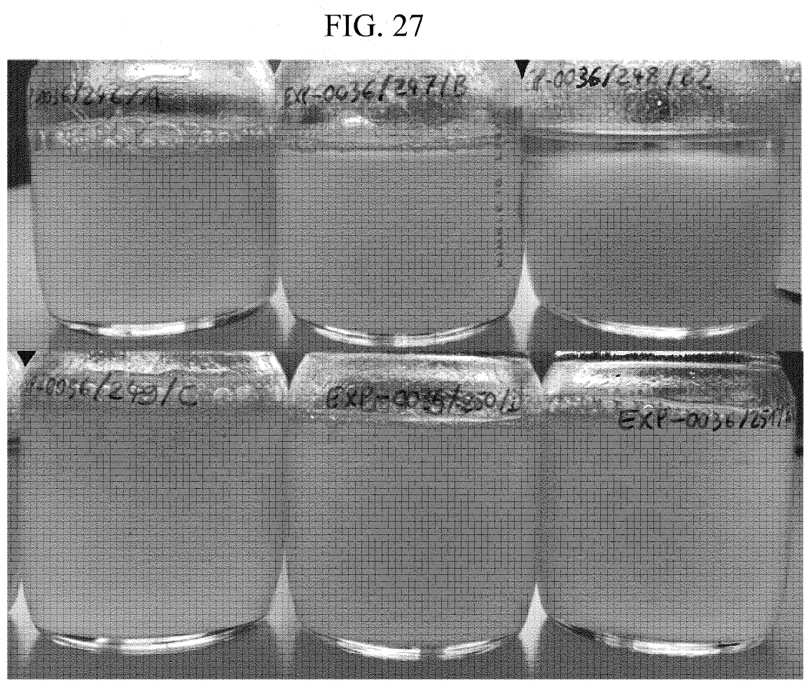

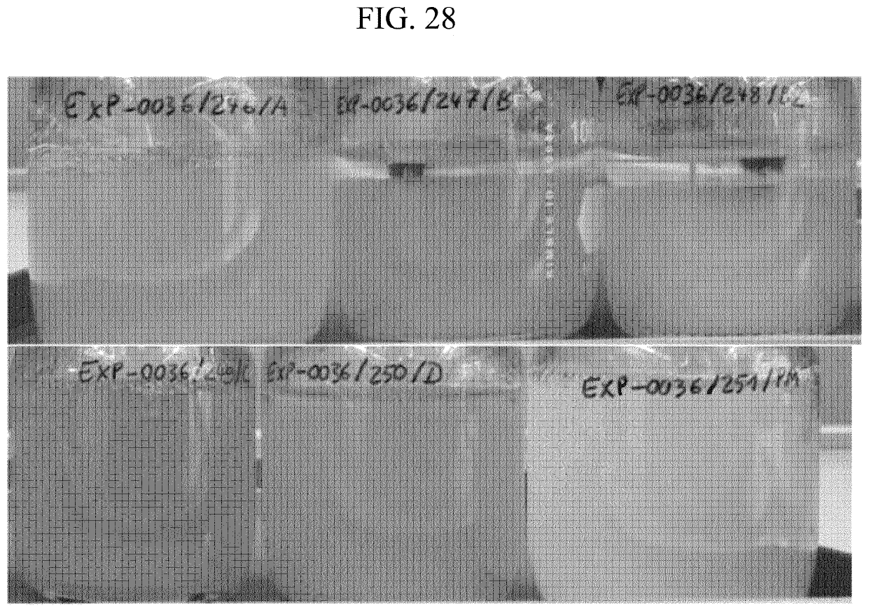

The Xaracoll bulk dispersion stability against phase separation correlates with the mLMC viscosity. Samples A, C and PM with LMC viscosity≥200 cP show no phase separation, sample B2 with LMC viscosity 50 cP shows strongest phase separation (B2>B>D) (FIG. 27 and FIG. 28 depict phase separation of the Xaracoll bulk dispersion at different mLMC viscosities.

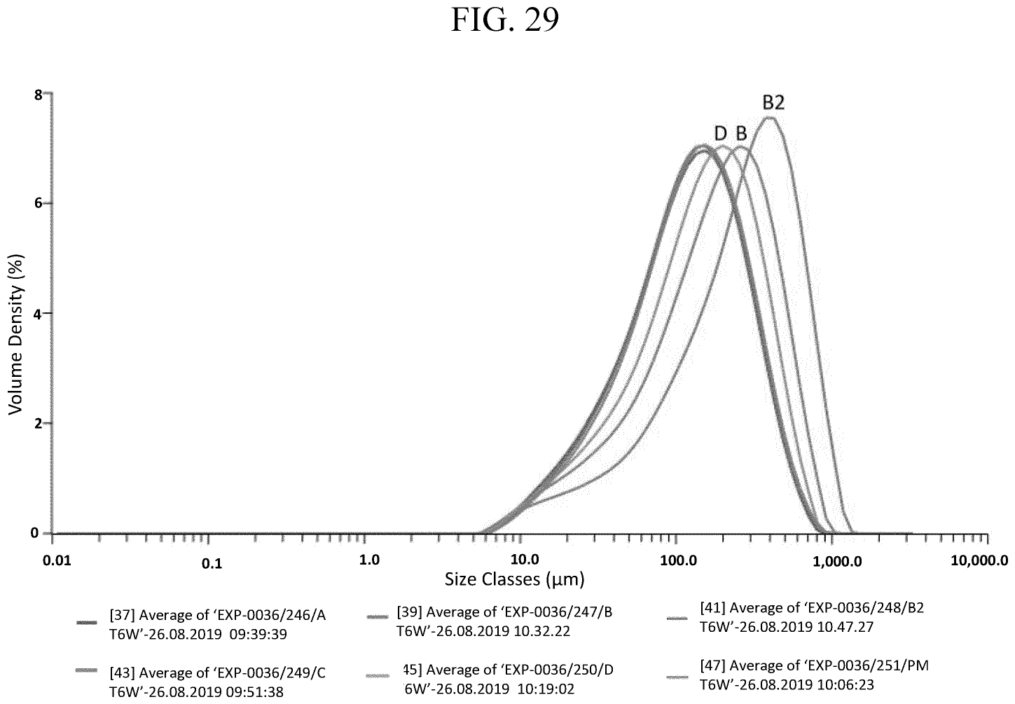

The Xaracoll bulk dispersion particle size distribution was measured after 6 weeks hold time in 0.1 M acetic acid at room conditions in order to investigate possible agglomeration (FIG. 29). The measurements for each sample show very low variability and no visible drift. There is no indication for de-agglomeration during the repeat measurements. The size distribution results are identical to the T0 results (FIG. 26) and the sample stability is excellent. Possible agglomeration is reversible in the default medium 0.1 M acetic acid.

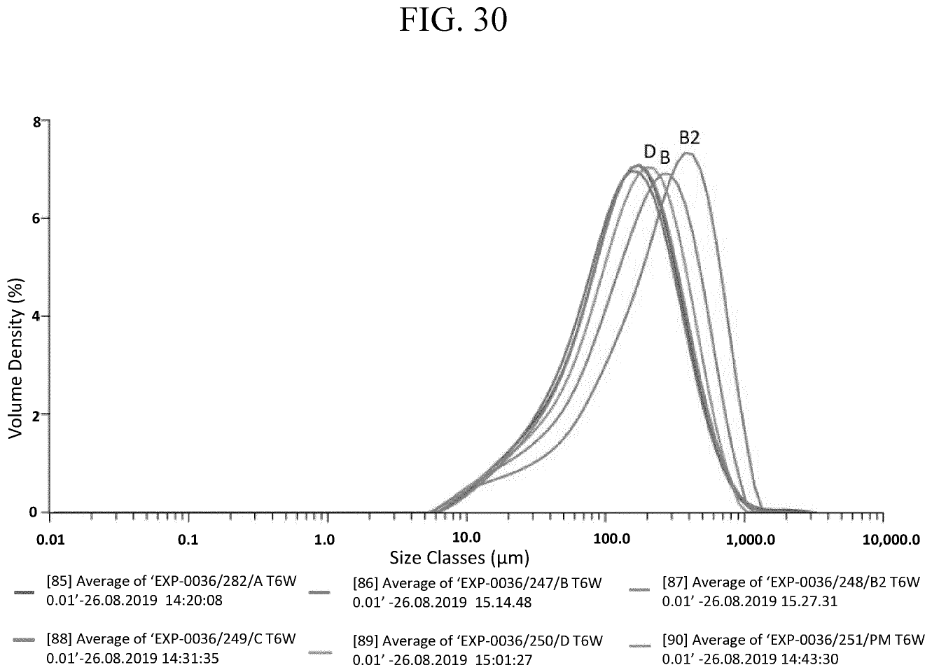

The measurements for each sample after 6 weeks hold time in 0.01 M acetic acid at room temperature show low variability and no visible drift. There is no indication for de-agglomeration during the repeat measurements. The results show a size shift compared to the results measured in 0.1 M acetic acid (FIG. 30).

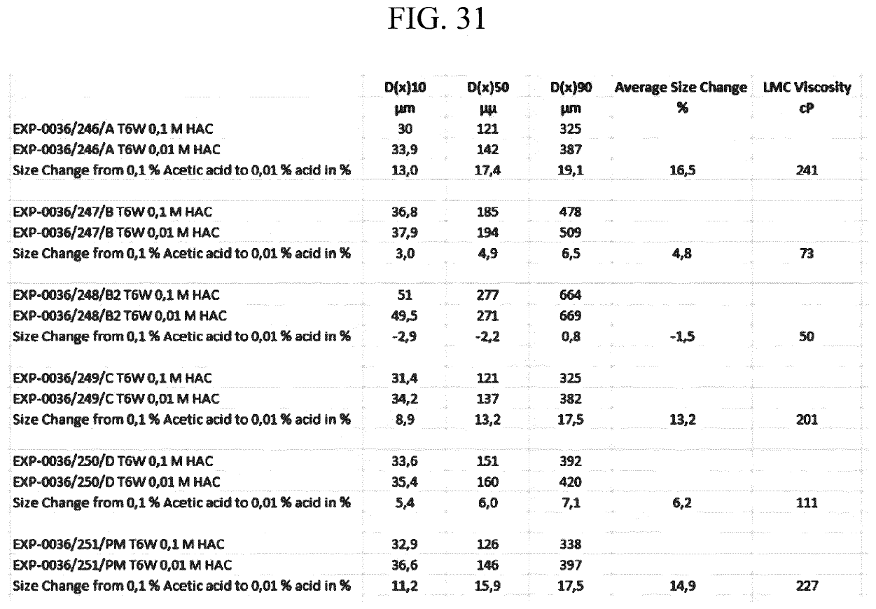

FIG. 31 depicts acid concentration-based size shift with the LMC viscosity.

### Bupivacaine-Collagen Sponge Characterization

### Determination of Free Amines by TNBS Derivatization

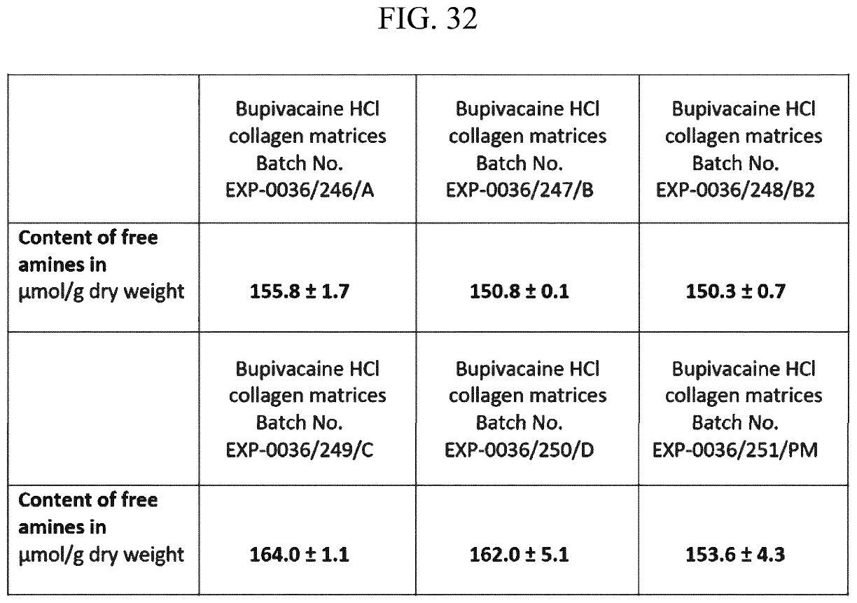

The determination of free amines is based on the reaction of TNBS with functional amino groups resulting in the formation of yellow colored adducts determined by measuring their absorbance at 345 nm. The content of free amines in collagen materials inversely correlates with their cross-linking degree. The contents of free amines determined for the tested bovine collagen samples are shown in FIG. 32.

### Testing of Impurities by SDS-PAGE

In this study, the identification of proteins as well as proteinogenic impurities was performed by SDS-PAGE. To obtain the soluble part of the sample materials, the different samples were treated with 0.1 M acetic acid for 20 min at 60° C. After centrifugation, the supernatants were collected and lyophilized. Prior to SDS-PAGE, the lyophilized samples were redissolved in SDS sample buffer and subsequently incubated at 95° C. for 2 min. 10 μl of this solution was loaded onto the gel.

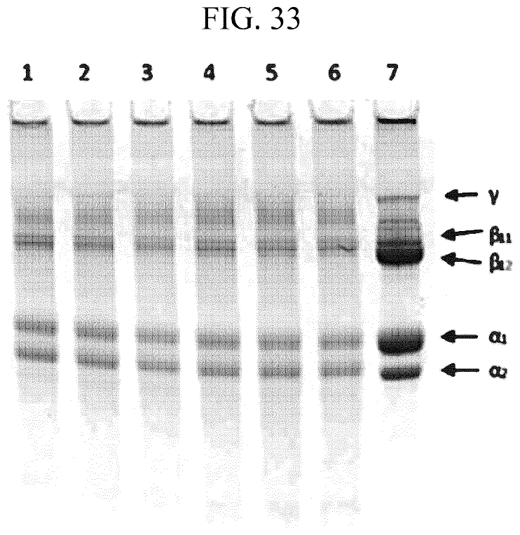

The solubility achieved during sample preparation ranged between 63 and 71%. Taking the soluble fractions of the samples, SDS-PAGE analysis was carried out by applying a gel with a separation range from 200 to 6.5 kDa. FIG. 33 lane 7 shows an electrophoretic pattern that is characteristic for acid soluble collagen. The monomeric α1- and α2-chains, the dimeric β11- and β12-chains, the trimeric γ-chain as well as some high molecular weight components at the top of the gel were clearly visible. This band pattern of γ-, β- and α-chains was also found for all tested sample materials. Further peptide bands, indicating impurities by other proteins than collagen, were not detected.

### Trypsin Resistance

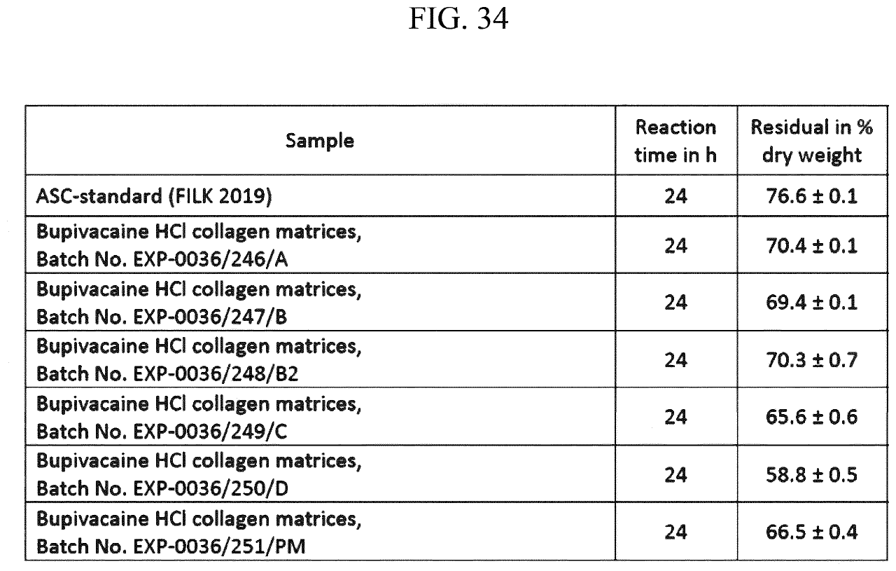

Resistance against enzymatic digestion by trypsin without an initial denaturation step is used to determine the native portion of collagen materials. After incubation of the samples with the enzyme at 37° C. for a period of 24 h, the residues are washed, dried and determined gravimetrically. The supernatants of the incubations were collected, lyophilized and further analyzed by SDS-PAGE. For the different matrix samples, an amount corresponding to approx. 40 μg protein was loaded onto the gel. The molecular weight cutoff of the gel, used for this analysis, was around 6 kDa. The weight portions remaining after incubating the different Xaracoll samples with trypsin for a period of 24 h are listed in FIG. 34.

### SEM Analysis

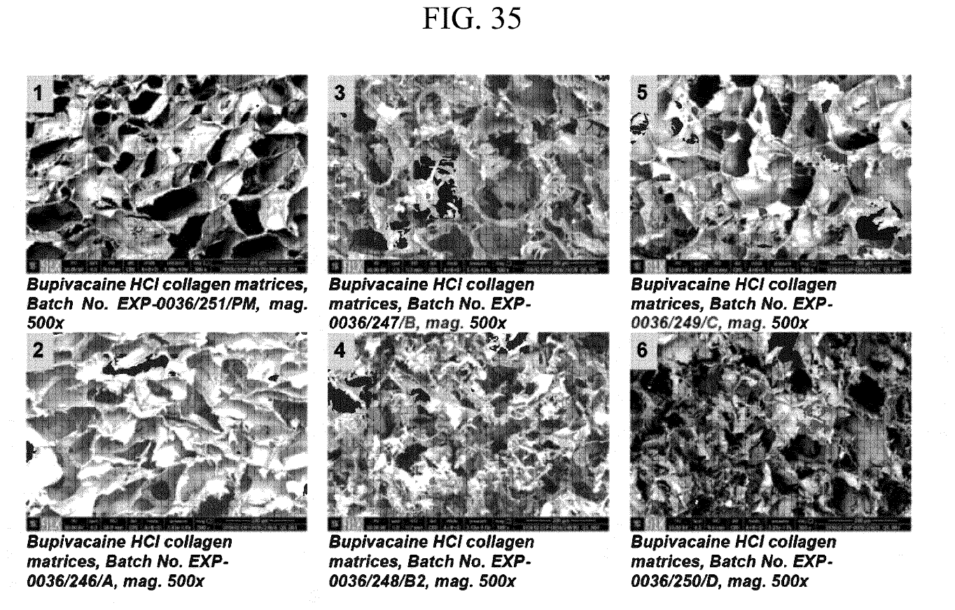

SEM images of the surfaces and cross sections of the different Xaracoll matrix samples are displayed in FIG. 35.

### AFM Analysis

AFM analysis follows a simple but effective measurement principle. A laser beam is pointed onto the backside of a cantilever to measure the cantilevers deflection. The reflected laser beam is than measured with a four-segmented photodiode to obtain the height information from the sample. For the analysis of the bovine collagen matrices, the AFM was used in the amplitude modulated contact mode (AC mode). In this mode, the tip oscillates near or at the cantilevers resonance frequency and the amplitude is used as the feedback mechanism. This is a soft imaging mode for soft or condensed matter, e. g. collagen. With AC mode, the height information (height image) and information of the viscoelastic properties (phase image) can be obtained. Sample preparation for AFM-analysis involved taking a small specimen from the sample and gently pressing it down onto a microscope slide.

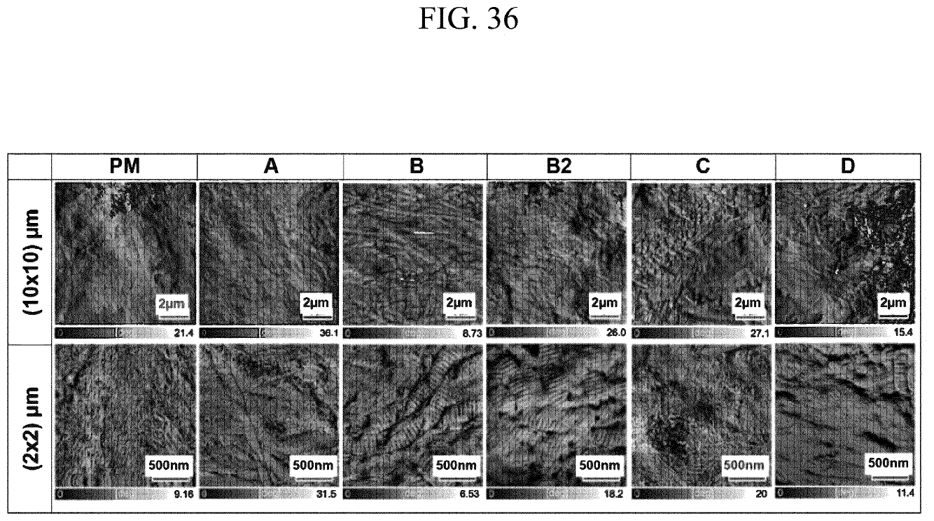

AFM images of the lyophilized bovine collagen matrix samples are displayed in FIG. 36. The material is comprised of a collagen microfibril assembly showing a disordered orientation.

### DSC

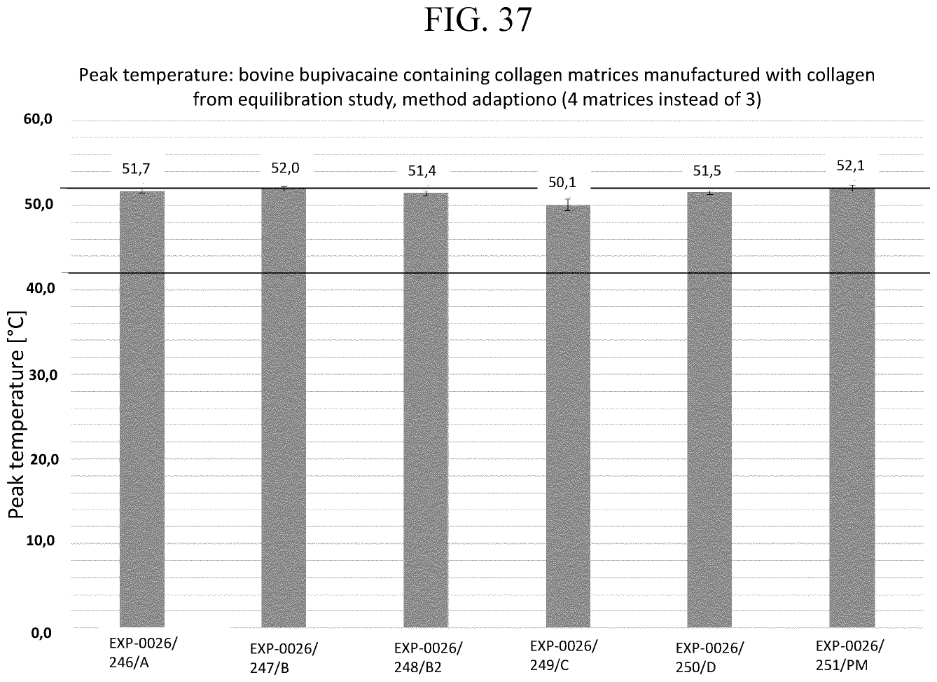

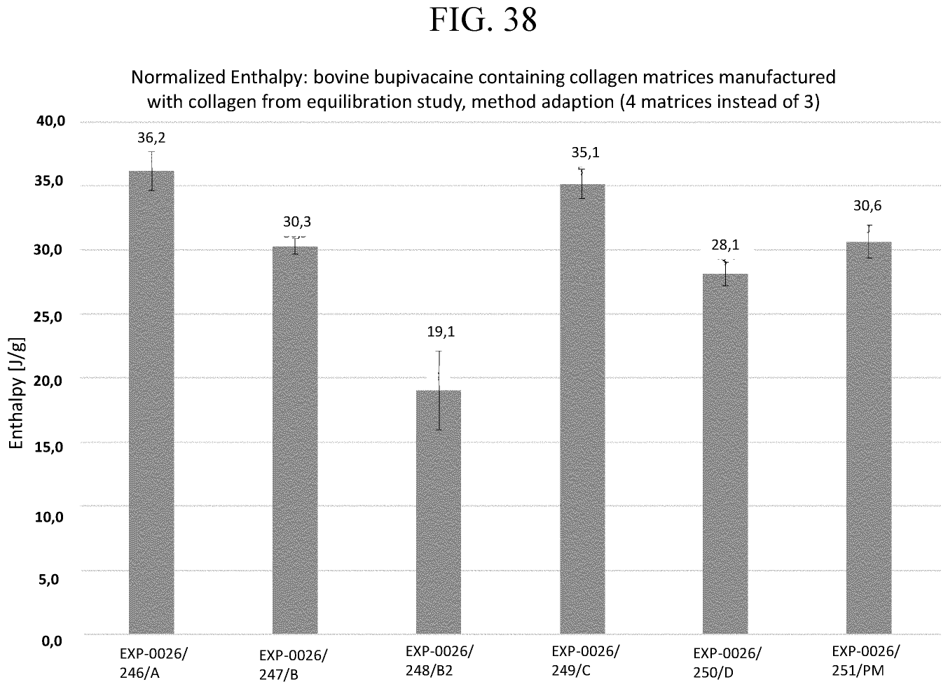

The thermal properties peak denaturation temperature (FIG. 37) and specific denaturation enthalpy (FIG. 38) of the sterile Xaracoll samples where measured on the Mettler Toledo DSC 3+. Xaracoll was measured using method QC6-059 but with 4 matrices in 10.5 g buffer instead of 3 matrices. The method was adapted because of visible inhomogeneity of the suspensions especially the suspensions from low viscous LMC (with strong variation of the normalized enthalpy results). Some single values of the peak denaturation temperature are above the specification limit (42 to 52° C.) with respect to the adapted method. There was low variability between the samples without a significant trend (FIG. 37). For the normalized enthalpy, there is a trend towards reduced enthalpy with low viscosity levels (most pronounced for B2) (FIG. 38).

### Water Uptake

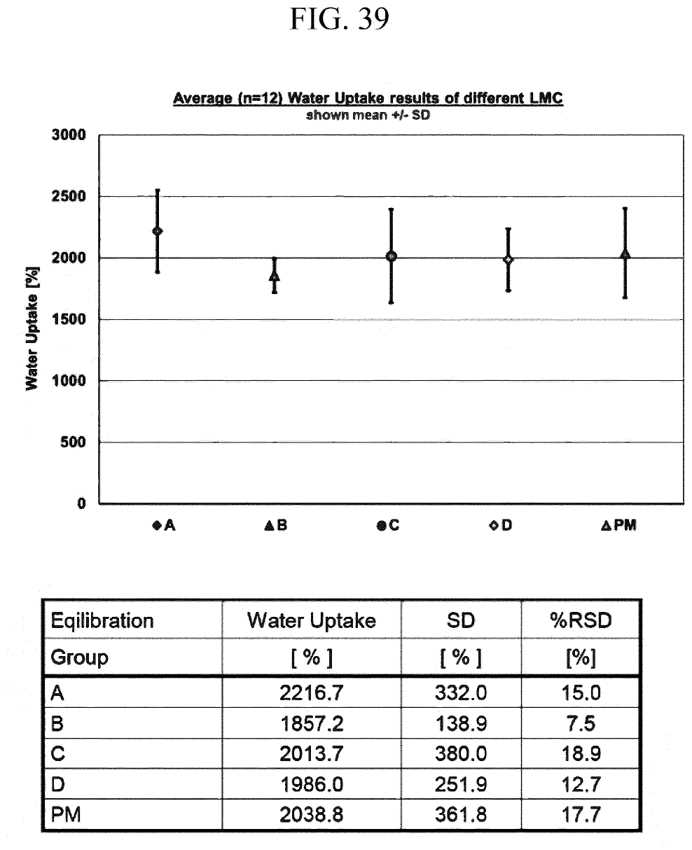

Water uptake was measured according to R&D SOP RD4-007-01. Twelve collagen matrices per each batch (EXP-0036/246/A, 247/B, 248/B2, 249/C, 250/D, 251/PM) were cut into 1.5 cm×4 cm pieces (cut at all 4 sides), weighed in the dry state on an analytical balance and soaked in WFI for 5 min. After that, the samples were drained for 3 min on an inclined plane. The weight of the wetted samples was measured again. The weight of the dry and wet pieces was used for calculating the water uptake (FIG. 39). It was not possible to measure the Water Uptake of the 248/B2 samples due to down sliding issues from the inclined plane during draining. ANOVA Analysis shows no significant different between the five groups (DF=57; P-Value 0.0988) (FIG. 39).

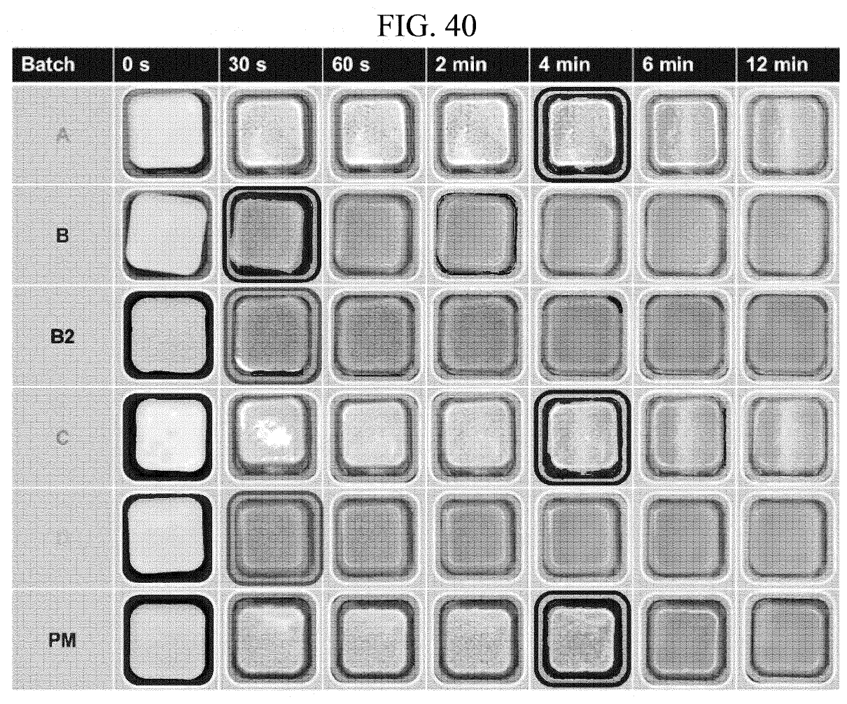

A visual water uptake kinetic was also evaluated (FIG. 40).

### Tensile Strength

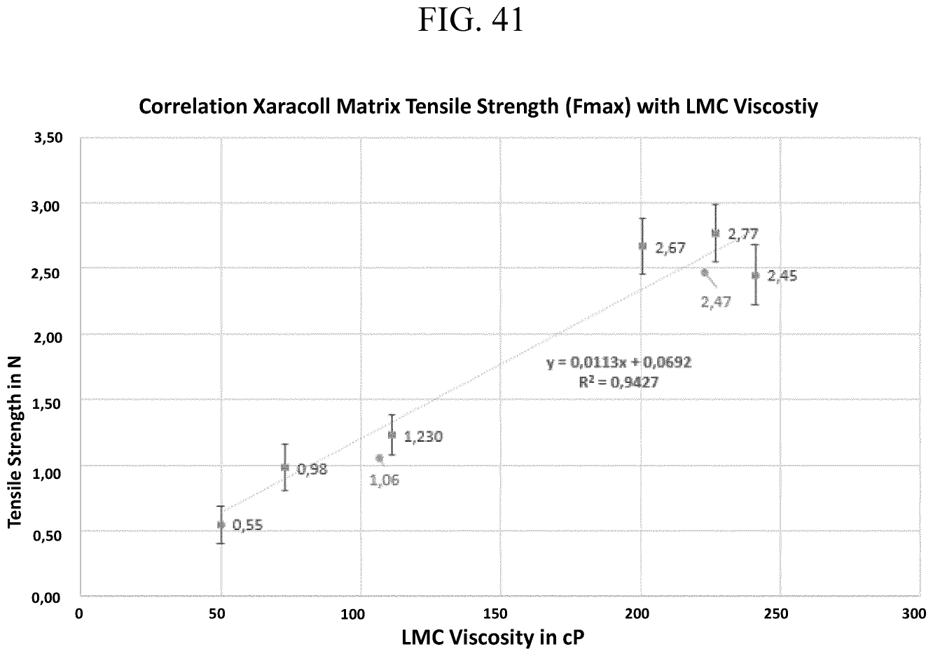

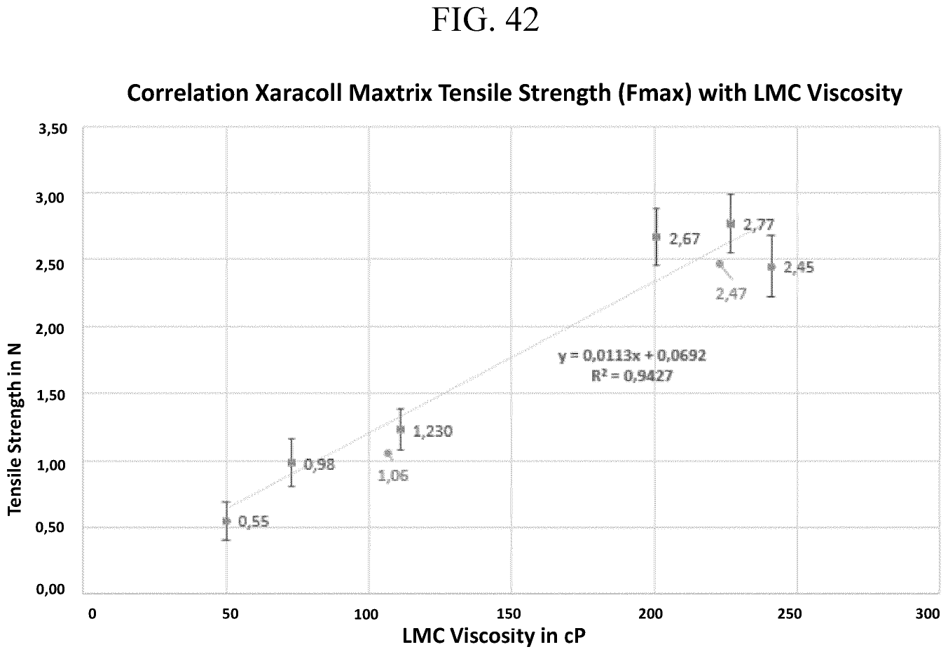

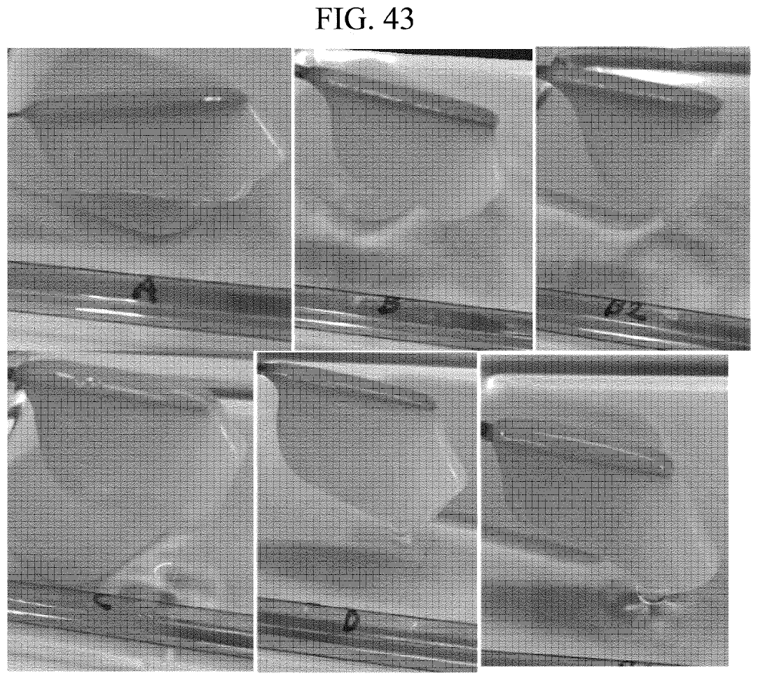

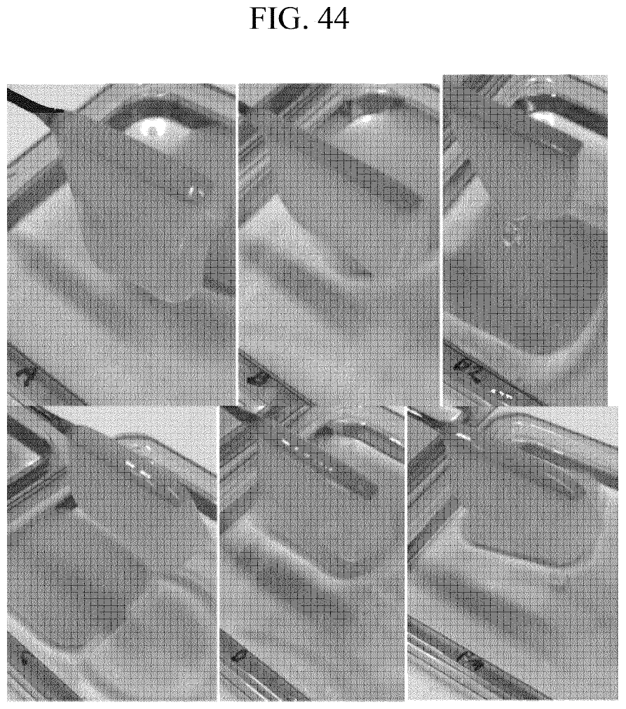

The tensile strength was measured on 12 samples in a dry state and in wet state for each batch. Test specimens with a width of 8 mm were prepared by cutting the matrices with a dumbbell-shaped cutting die (Lever Press Maeder APK T3-S-40). The seal strength was measured using the sealing strength tester Zwicki 500N. The samples were placed between the clamps with the wider areas at both ends and arrested by pneumatic force. The clamps were pulled apart until the sample ruptured and the maximum force was recorded (FIG. 41 and Table 2). The wet tensile strength was measured using the Finch apparatus with the same parameters as the dry tensile strength (FIG. 42, FIG. 43, FIG. 44 and Table 3).

The dry tensile strength of the Xaracoll matrices correlates with the mature LMC viscosity (FIG. 41 and Table 2). Matrices made from mature dehydrated collagen with a viscosity lower than about 110 cP have lower dry tensile strength than those made from mature dehydrated collagen with a viscosity between about 110 cP and 250 cP.

TABLE 2
Dry tensile strength of Xaracoll matrices (SD is standard deviation)

| | | | mLMC | | |
|---|---|---|---|---|---|
| | | | Viscosity | Deformation | |
| Sample | Fmax (N) | | (cP) | to Fmax (mm) | |
| A | 2.45 | (SD 0.228) | 241 | 2.60 | (SD 0.385) |
| B | 0.983 | (SD 0.173) | 73 | 2.03 | (SD 0.501) |
| B2 | 0.550 | (SD 0.144) | 50 | 1.44 | (SD 0.285) |
| C | 2.67 | (SD 0.214) | 201 | 2.60 | (SD 0.272) |
| D | 1.230 | (SD 0.155) | 111 | 1.99 | (SD 0.31) |
| PM | 2.77 | (SD 0.218) | 227 | 2.52 | (SD 0.268) |
| PM “standard” | 2.47 | (SD 0.31) | 223 | 1.40 | (SD 0.16) |
| PM “soft” | 1.06 | (SD 0.21) | 107 | 1.45 | (SD 0.36) |

The was also a clear correlation between mature LMC viscosity and wet tensile strength wherein Xaracoll matrices made from mature LMC with lower viscosities had lower wet tensile strength (FIG. 42, FIG. 43, FIG. 44 and Table 3). Additionally, sterile Xaracoll matrices were found to have increased wet tensile strength compared to non-sterile matrices.

TABLE 3
Wet tensile strength of Xaracoll matrices

| | | | mLMC | | |
|---|---|---|---|---|---|
| | | | Viscosity | Deformation | |
| Sample | Fmax (N) | | (cP) | to Fmax (mm) | |
| A | 0.620 | (SD 0.090) | 241 | 9.77 | (SD 0.618) |
| B | 0.091 | (SD 0.0089) | 73 | 6.11 | (0.419) |
| B2 | 0.030 | (SD 0.0045) | 50 | 5.10 | (SD 0.564) |
| C | 0.990 | (SD 0.102) | 201 | 10.31 | (SD 0.487) |
| D | 0.198 | (SD 0.013) | 111 | 7.78 | (SD 0.254) |
| PM | 0.869 | (SD 0.098) | 227 | 9.625 | (SD 0.356) |
| PM “standard” | 1.24 | (SD 0.148) | 223 | 15.20 | (SD 1.40) |
| PM “soft” | 0.447 | (SD 0.0615) | 107 | 11.03 | (SD 0.49) |

### Resistance to Pressure

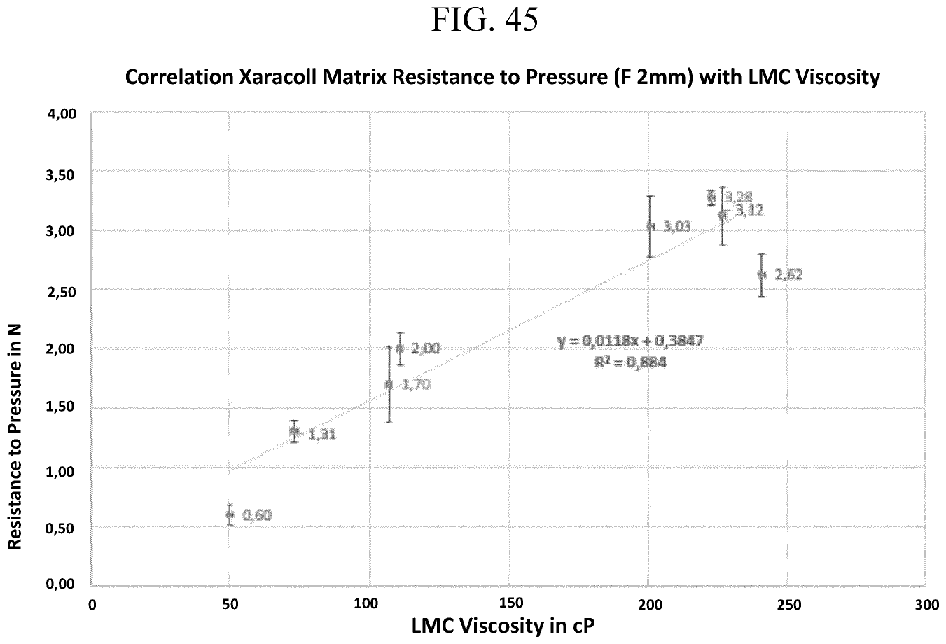

The resistance to pressure was measured on a total of 9 samples for each batch using the materials testing machine Zwicki 500N (FIG. 45 and Table 4). There is no sample preparation. Matrices were placed centered on a flat table, a spherical test body (diameter 1 inch) was lowered down and pushed into the matrix. Relative position of test body and force were recorded continuously. The difference in position from first contact (0.01N) to table is recognized as matrix thickness. The resulting force at 2 mm test body intrusion is the parameter for the resistance to pressure.

The resistance to pressure of Xaracoll matrices (sterile and nonsterile) correlates with the mature LMC viscosity. Matrices made from matured dehydrated collagen with a viscosity lower than 110 cP (viscosity measurement of a 0.9 wt % dispersion of matured dehydrated collagen at a pH of about 4.5 and a temperature of about 40° C.) have less resistance to pressure than those made from mature dehydrated collagen with a viscosity between about 110 cP and 250 cP

TABLE 4
Resistance to pressure and thickness values of Xaracoll matrices

| | | | mLMC | | |
|---|---|---|---|---|---|
| | | | Viscosity | Thickness | |
| Sample | F 2 mm (N) | | (cP) | (mm) | |
| A | 2.62 | (SD 0.18) | 241 | 5.02 | (SD 0.16) |
| B | 1.31 | (SD 0.093) | 73 | 5.17 | (SD 0.082) |
| B2 | 0.60 | (SD 0.083) | 50 | 5.19 | (SD 0.093) |
| C | 3.03 | (SD 0.26) | 201 | 4.98 | (SD 0.21) |
| D | 2.00 | (SD 0.13) | 111 | 5.09 | (SD 0.11) |
| PM | 3.12 | (SD 0.24) | 227 | 4.93 | (SD 0.19) |
| PM “standard” | 3.28 | (SD 0.32) | 223 | 4.58 | (SD 0.16) |
| PM “soft” | 1.70 | (SD 0.058) | 107 | 4.95 | (SD 0.054) |

### Swelling Time and Swelling Volume

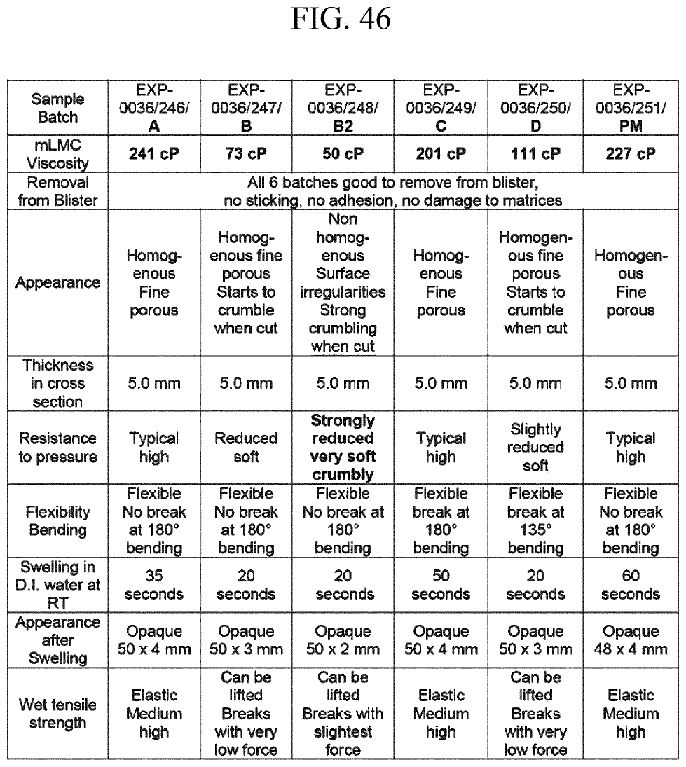

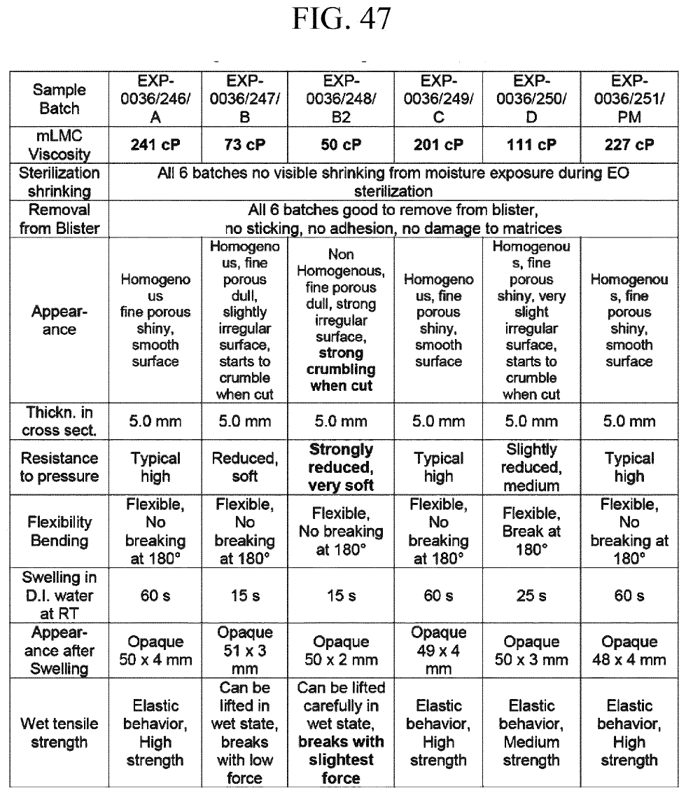

The swelling time in water of the Xaracoll matrices (sterile and nonsterile) also correlates with the mature LMC viscosity. Matrices made from mature dehydrated collagen with a viscosity lower than about 110 cP swell faster in water than those made from mature dehydrated collagen with a viscosity between about 110 cP and 250 cP. Additionally, sterile Xaracoll matrices were found to have increased resistance to pressure when compared to non-sterile Xaracoll matrices. FIG. 46 provides an overview of the swelling time in water of the nonsterile Xaracoll matrices while FIG. 47 provides an overview of the swelling time in water of the sterile Xaracoll matrices.

The swell volume (wet matrix thickness) of the Xaracoll matrices correlates with the mature LMC viscosity. Matrices made from mature dehydrated collagen with a viscosity lower than about 110 cP have lower wet matrix thickness than those made from mature dehydrated collagen with a viscosity between about 110 cP and 250 cP.

### Resistance to Bending

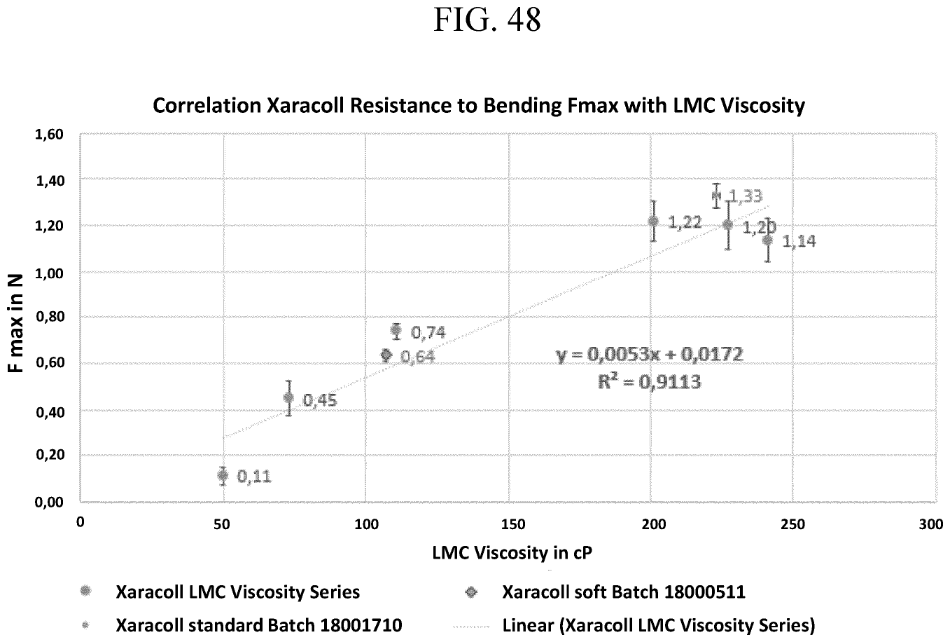

The resistance to bending was measured on a total of 9 samples for each batch using the materials testing machine Zwicki 500N (FIG. 48 and Table 5). There is no sample preparation. Matrices were placed centered on two supports in 35 mm distance, a blade like test body is lowered down in the middle and pushed into the matrix. Relative position of test body and force are recorded continuously. The matrix deforms and either breaks or slips through without break.

TABLE 5
Resistance to bending values of the Xaracoll matrices

| | mLMC Viscosity | Average | | RSD |
|---|---|---|---|---|
| Sample | (cP) | Fmax (N) | | (%) |
| A | 241 | 1.14 | (SD 0.10) | 8.6 |
| B | 73 | 0.45 | (SD 0.07) | 16.3 |
| B2 | 50 | 0.11 | (SD 0.04) | 32.6 |
| C | 201 | 1.22 | (SD 0.09) | 7.2 |
| D | 111 | 0.74 | (SD 0.03) | 4.6 |
| PM | 227 | 1.20 | (SD 0.11) | 8.8 |
| PM “standard” | 223 | 1.33 | (SD 0.0529) | 4.0 |
| PM “soft” | 107 | 0.64 | (SD 0.0244) | 3.9 |

### Dissolution

The Dissolution tests were performed based on standard operating procedure QC6-011. The collected samples were analyzed with UV HPLC based on SOP QC6-009. The 5×5 cm 100 mg Bupivacaine HCl collagen matrices were split over six separate Dissolution tests. In sum 8 samples of matrices per batch were measured. The keyword “Cross-Dissolution” means that the average curve was based on single results collected from different Dissolution test runs. The Dissolution tests were performed with an Agilent dissolution apparatus 708 DS and an Agilent dissolution sampling station 850 DS. A USP type II dissolution apparatus with 8 vessels was used. The samples were placed in stainless steel sinkers to prevent the samples from floating. The dissolution was performed in 500 ml PBS media (pH 6.8) at 37° C.; and with a rotation speed of 50 rpm. After 15, 30, 45, 60, 90, 120, 180, 240, 360, and 1440 min, samples of 4.5 ml were taken from each vessel. The taken media was replaced with the same amount of PBS pH 6.8. The collected sample solutions were analyzed with HPLC alliance Waters 2695 separation module on a Nucleodur C18 Gravity EC (125 mm length, 3 mm diameter, 3 μm particle) HPLC column via UV detection by detection wavelength of 230 nm. The results were given as average curve of n single sample results in one or more specific dissolution test runs.

The average dissolution curves of batch EXP-0036/246/A, 247/B, 248/B2, 249/C, 250/D, were compared with 251/PM with the f1/f2 factor method (Table 6).

TABLE 6
f1/f2 factor comparison of all batches with the PM batch.

| Comparison | f1 | f2 |
|---|---|---|
| PM vs A | 10.6 | 60.6 |
| PM vs B | 12.1 | 56.0 |
| PM vs B2 | 45.0 | 15.7 |
| PM vs C | 0.5 | 98.8 |
| PM vs D | 7.0 | 66.9 |

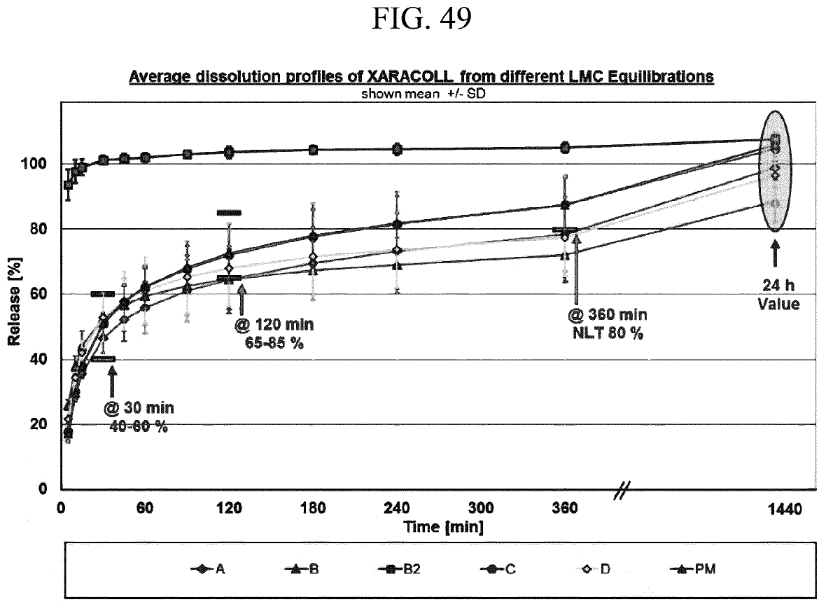

Table 7 and FIG. 49 depict the release results of different Xaracoll samples . . .

TABLE 7
Release results at time points of specification
(n = 12; n = 6 for sample B2)

| Sample | 30 mins | 120 mins | 360 mins |
|---|---|---|---|
| A | 46.4% | 64.4% | 78.4% |
| B | 52.6% | 64.6% | 71.1% |
| B2 | 101.2% | 103.6% | 105.1% |
| C | 50.9% | 72.0% | 87.5% |
| D | 52.9% | 68.0% | 77.5% |
| PM | 50.9% | 72.6% | 87.4% |

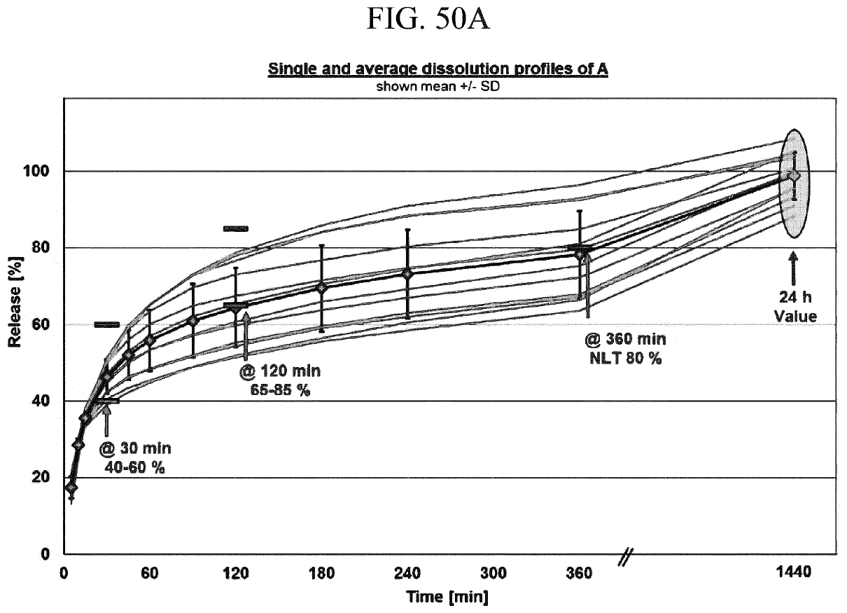

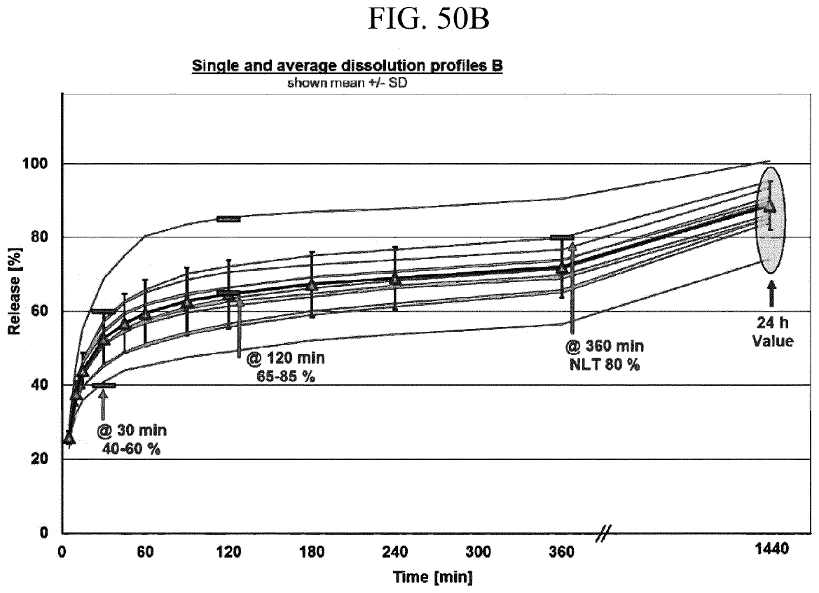

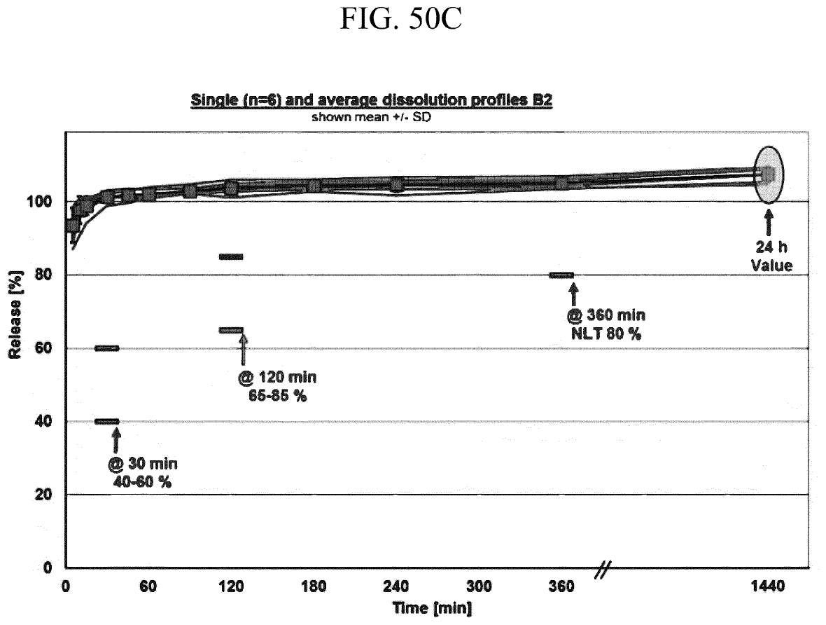

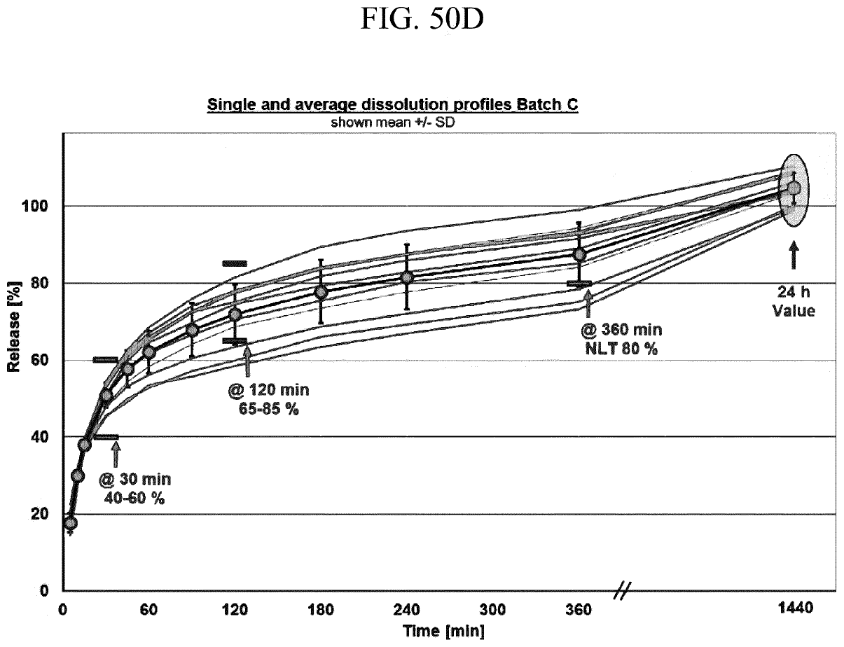

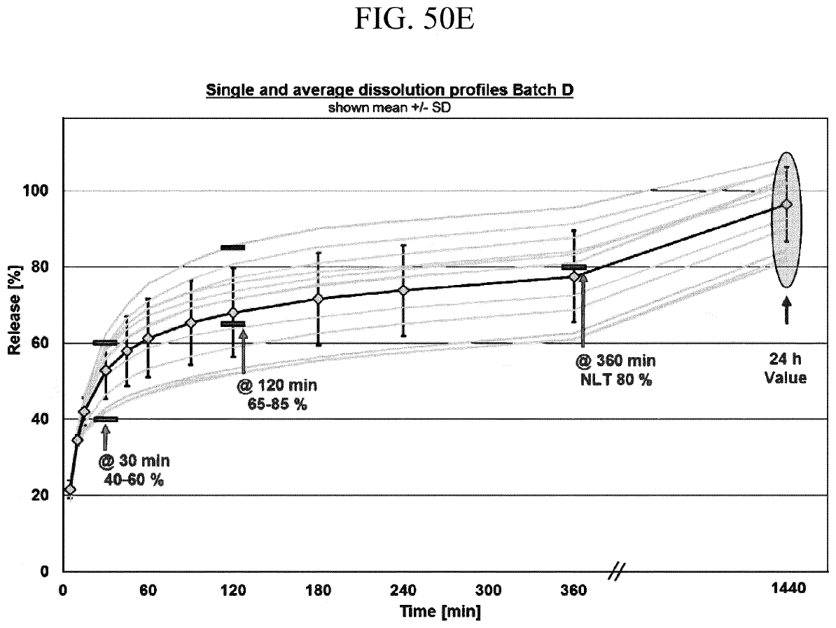

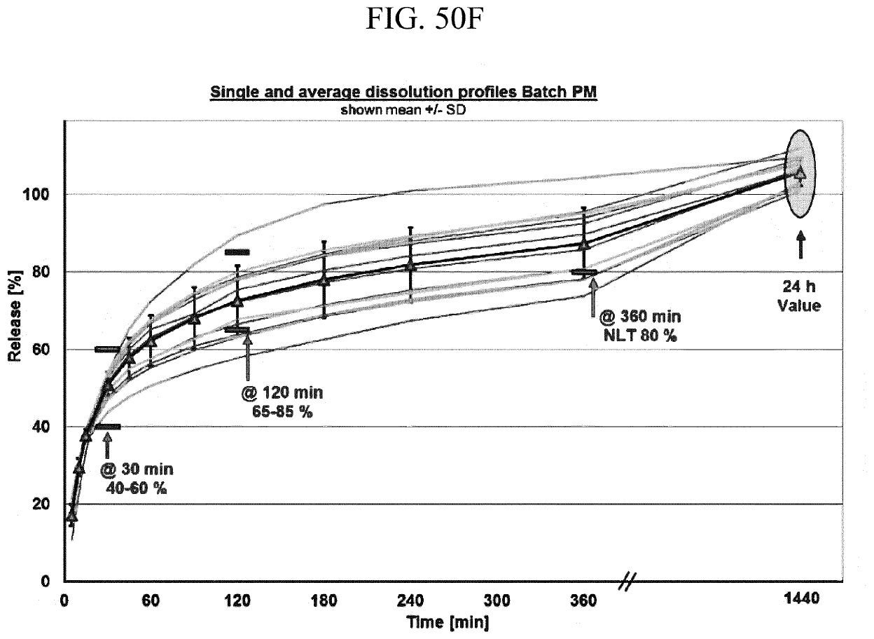

To categorize the different characteristics of the release profiles the slope at early to middle and middle to late dissolution is evaluated (Table 8 and FIGS. 50A-50F).

TABLE 8
List of slopes

| Batch | Slope 0-30 mins [%/min] | Slope 30-360 mins [%/min] |
|---|---|---|
| A | 1.55 | 0.10 |
| B | 1.75 | 0.06 |
| B2 | 3.37 | 0.01 |
| C | 1.70 | 0.11 |
| D | 1.76 | 0.07 |
| PM | 1.70 | 0.11 |

### Example 5: Effects of High Viscosity on LMC and Resulting Xaracoll Matrices

### Materials and Equipment

Bovine LMC CB0002. Batch 101805. as is with LOD of 3.46%
Bovine LMC CB0002. Batch 101805. pre humidified at 40° C. and 55% R.H. to 15.67% LOD
Medipeel pouch (PSYNGK88A. 300×580 mm). cut to 30 cm×37 cm
Twist Seal Pouch (Code: H0083. 5500 ml. 305×406 mm, Polyethylene (PE)), cut 30 cmx 37 cm
Climatic Chamber KBF 240 (400693) set to 40° C. and 75% R.H.

The samples in Medipeel pouches picked up moisture comparatively fast and were moisture equilibrated within 2 days, the equilibrium LOD at 40° C. and 75% R.H. is approximately 22-23%. The samples sealed into PE material picked up moisture much slower and did not equilibrate fully over the observation time span of 7 weeks. The equilibrium LOD at 40° C. and 75% with PE barrier is approximately 18%. The drop of the collagen viscosity is dependent on the LOD pick up:
After 34 days the closed (sealed) PE pouch packed mLMC sample C was at a viscosity level of 289 cP.
After 40 days the closed (sealed) PE pouch packed mLMC sample D was at a viscosity level of 238 cP.
In comparison, after 37 days the permeable Medipeel pouch packed mLMC sample F was already at 153 cP.

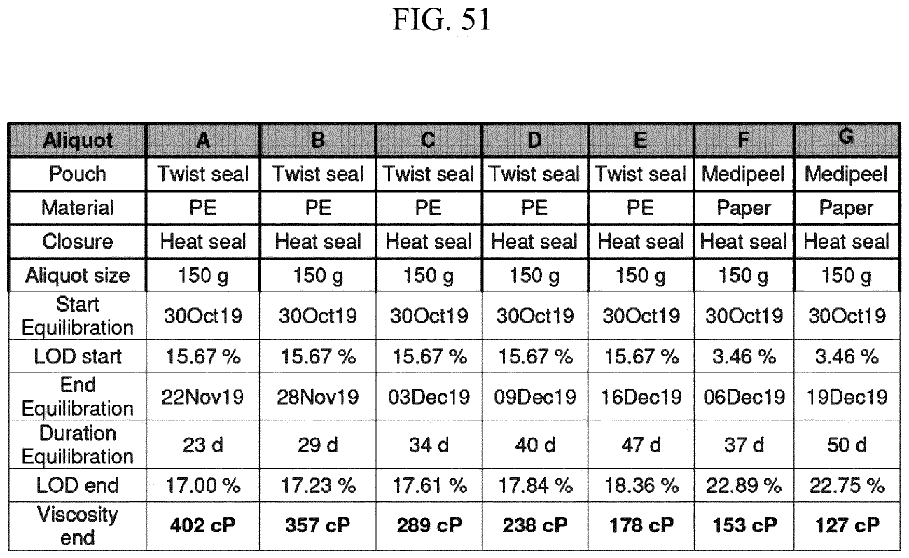

FIG. 51 provides an overview of the mature LMC samples.

mLMC Characterization

### Determination of Free Amines by TNBS Derivatization

The content of free amines in collagen materials can be used to estimate their cross-linking degree, with a lower TNBS read-out correlating with a higher consumption of ε-amino groups, i.e., higher level of cross-linking. Derivatization with TNBS is used to detect the terminal α-amino groups as well as the free ε-amino groups in peptides and proteins, resulting in the formation of colored adducts measured by absorbance at 345 nm.

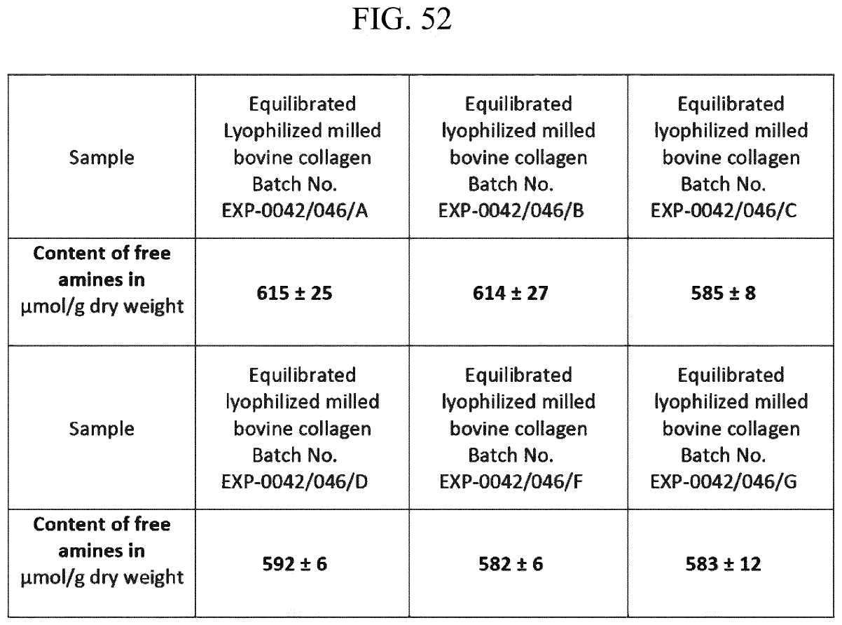

The contents of free amines determined for the tested bovine collagen samples are shown in FIG. 52. Taken together, the examples provided herein demonstrate that a composition comprising a modified collagen according to a first aspect of the present invention, or a modified collagen prepared according to a second aspect of the present invention—for example, membranes prepared from matured lyophilized milled collagen—exhibit significantly altered properties compared to membranes made from fresh collagen, frozen collagen, or non-matured lyophilized milled collagen. The maturing step providing the altered properties of aged collagen without the extended ageing period; and so can be particularly useful in the manufacture of compositions for use in preventing or treating surgical adhesions.

### Trypsin Resistance and SDS-PAGE

In the native collagen triple helix, the protein is protected from enzymatic degradation of most proteinases (with exception of e.g., collagenase) by steric hindrance. Therefore, resistance against enzymatic digestion by trypsin without an initial denaturation step is used to determine the native portion of collagen materials. In detail: after incubation of the samples with the enzyme at 37° C. for a period of 24 h, the residues were washed, dried and determined gravimetrically. The supernatants of the incubations were collected, lyophilized and further analyzed by SDS-PAGE. For the different mLMC samples, an amount corresponding to approx. 20 μg protein was loaded onto the gel. The molecular weight cutoff of the gel, used for this analysis, was around 6 kDa.

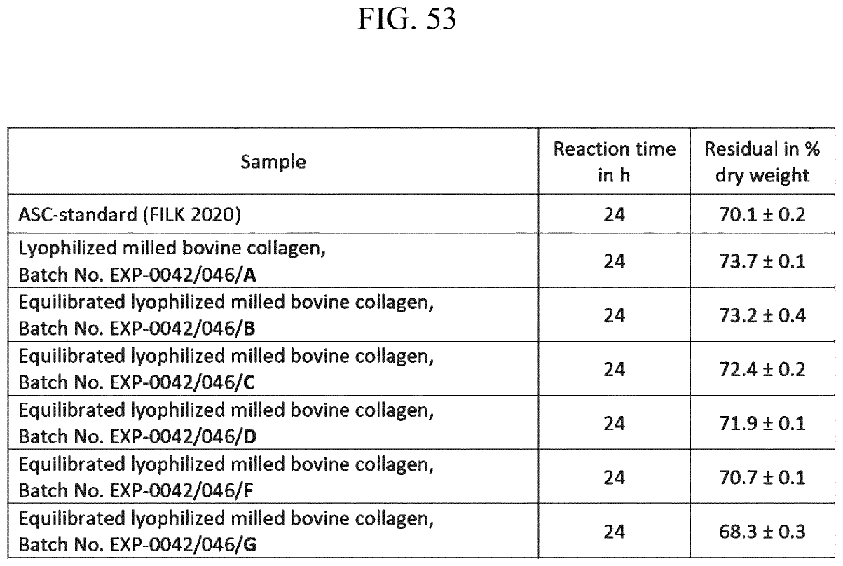

The weight portions remaining after incubating the different mLMC samples with trypsin for a period of 24 h are shown in FIG. 53. There was a slight tendency for lower post trypsin residuals (% dry weight), i.e., lower trypsin resistance with increased mLMC equilibration, i.e., lower viscosity of equilibrated mLMC.

### Testing of Impurities by SDS-PAGE

SDS-PAGE is used for identification of collagen related peptide chains as well as non-collagen protein impurities. To obtain the soluble part of the sample materials, the different Bovine Collagens were treated with 0.1 M acetic acid for 20 min at 60° C. After centrifugation, the supernatants were collected and lyophilized. Prior to SDS-PAGE, the lyophilized samples were dissolved in SDS-sample buffer and subsequently incubated at 95° C. for 2 min. 10 μl of this solution was loaded onto the gel.

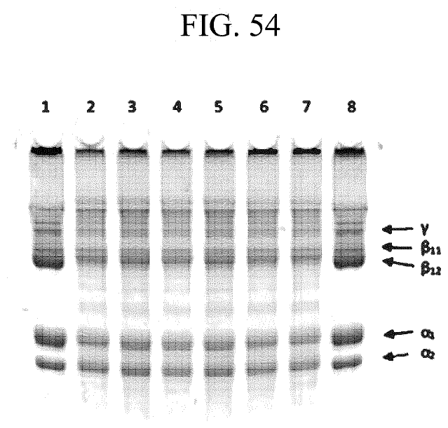

The solubility achieved during sample preparation ranged between 12 and 24% for the different bovine collagens. Taking the soluble fractions of the samples, SDS-PAGE analysis was carried out by applying a gel with a separation range from 200 to 6.5 kDa. FIG. 54 lanes 1 and 8 show an electrophoretic pattern that is characteristic for acid soluble collagen. The monomeric α1- and α2-chains, the dimeric β11- and β12-chains, the trimeric γ-chain as well as some high molecular weight components at the top of the gel were clearly visible. This band pattern of γ-, β- and α-chains was also found for all tested sample materials. Further peptide bands, indicating impurities by other proteins than collagen, were not detected.

### SEM Analysis

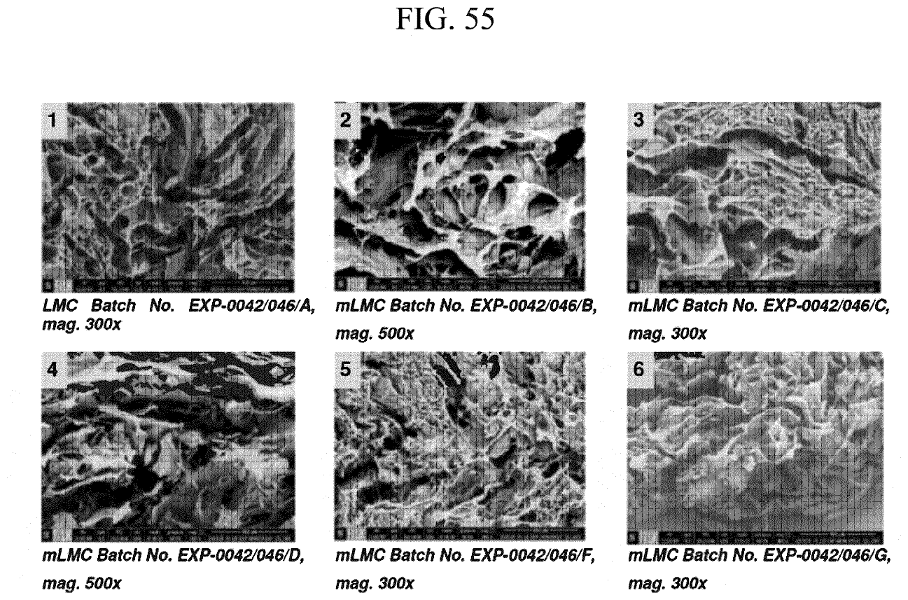

During SEM analysis, the surface and the cross section of a specimen are scanned with a focused beam of electrons. The electrons interact with atoms in the specimen producing various signals that are measured to generate an image of the surface topography and composition. SEM images of the cross sections of the different mLMC samples are displayed in FIG. 55.

### AFM Analysis

Atomic Force Microscopy combines the advantage of a very high magnification (near to that of Transmission Electron Microscopy) with the capability for three-dimensional resolution similar to Scanning Electron Microscopy. The underlying principle of AFM analysis is based on a laser beam pointed onto the backside of a cantilever to measure the cantilevers deflection. The reflected laser beam is then measured with a four-segmented photodiode to obtain the height information from the sample. For the analysis of the lyophilized bovine collagen, the AFM was used in the amplitude modulated contact mode (AC mode). In this mode, the tip oscillates near or at the cantilevers resonance frequency and the amplitude is used as the feedback mechanism. This is a soft imaging mode for soft or condensed matter, e.g., collagen. With AC mode, the height information (height image) and information of the viscoelastic properties (phase image) can be obtained. Sample preparation for AFM-analysis involved taking a small specimen from the sample and gently pressing it down onto a microscope slide.

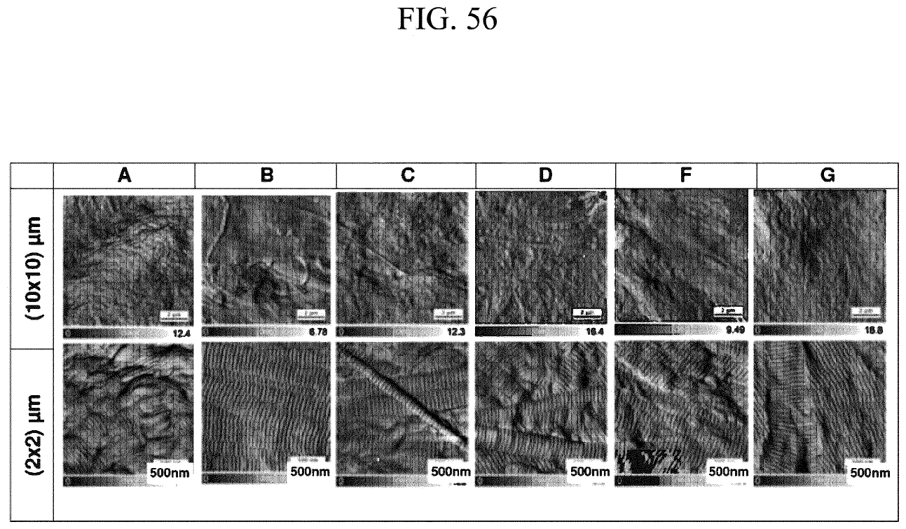

AFM images of the lyophilized milled bovine collagen samples are displayed in FIG. 56. The material is comprised of a collagen micro fibril assembly showing a disordered orientation. Fibrils with the typically collagen D-band pattern are visible. Possible image deficiencies might be explained by artefacts resulting from the pressing procedure during sample preparation or from the movement of the cantilevers tip.

### DSC

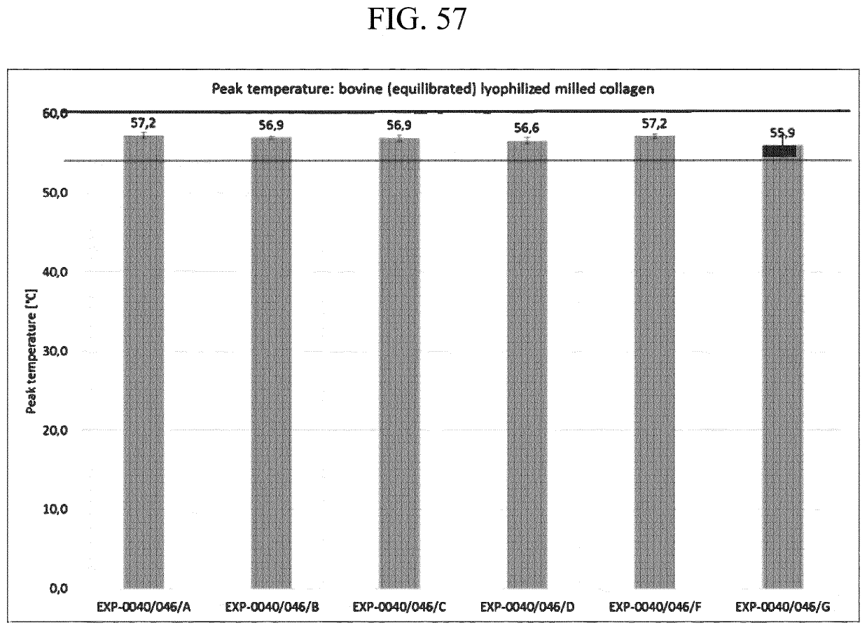

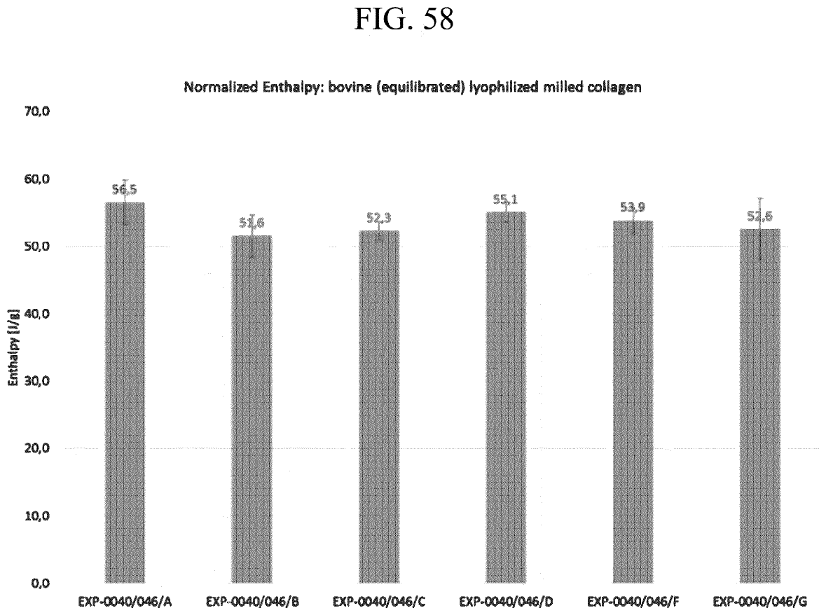

All peak denaturation temperature values are within specification (54-60° C.). The peak denaturation temperature is shown in FIG. 57 and the normalized enthalpy is shown in FIG. 58.

### Particle Size Distribution

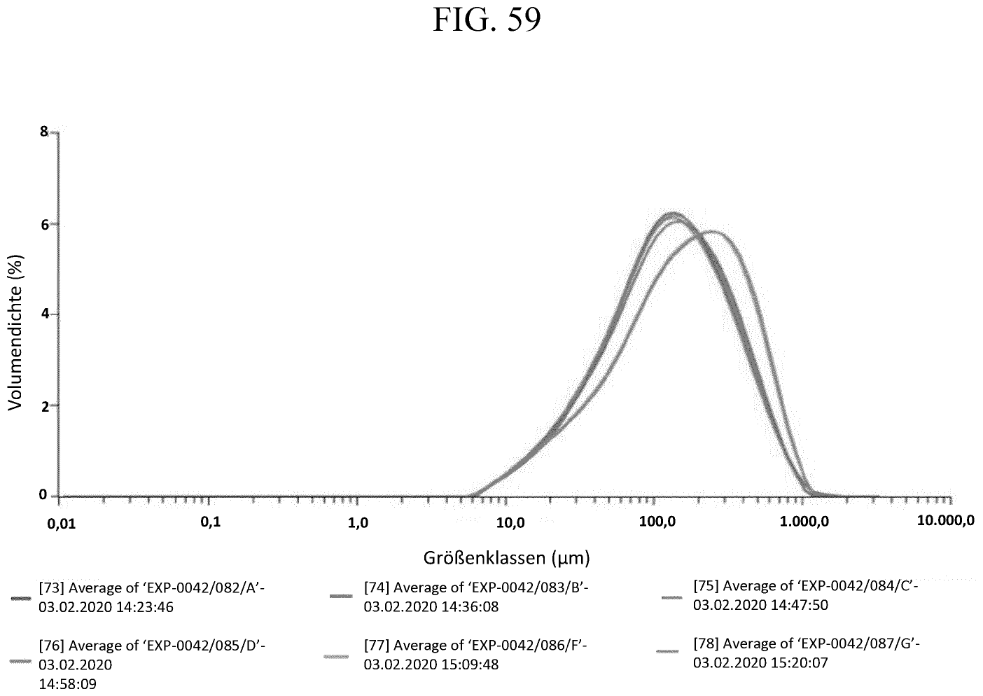

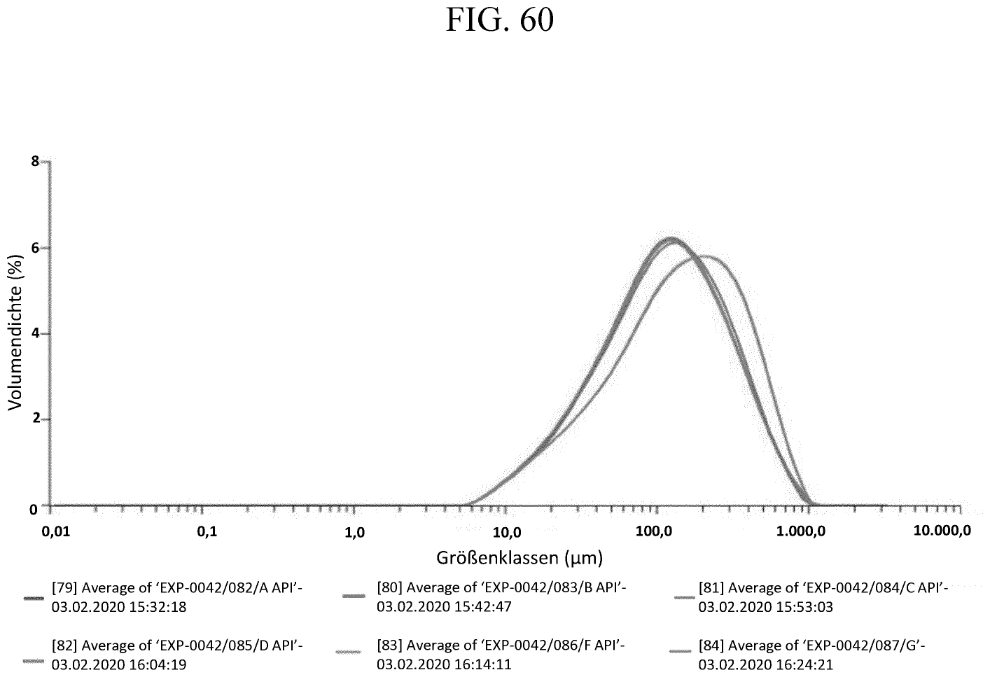

The particle size distribution for the collagen only Xaracoll bulk dispersion is shown in FIG. 59 and the API (bupivacaine) containing dispersion is shown in FIG. 60. Both groups show a similar shift in particle size distribution through addition of the API to the bulk dispersion.

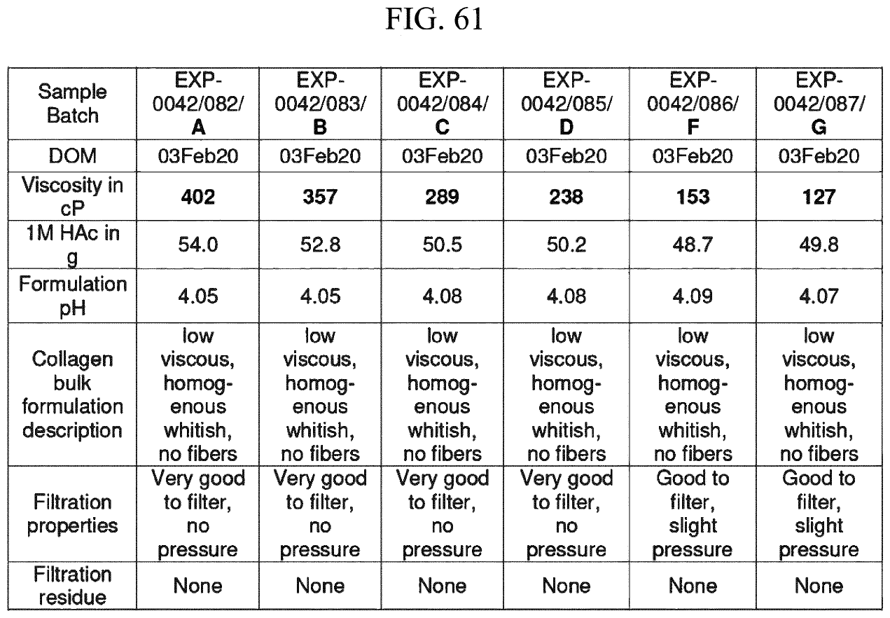

FIG. 61 provides an overview of the bulk dispersion properties discussed above.

### Xaracoll Matrix Manufacture & Analysis

### Matrix Manufacture

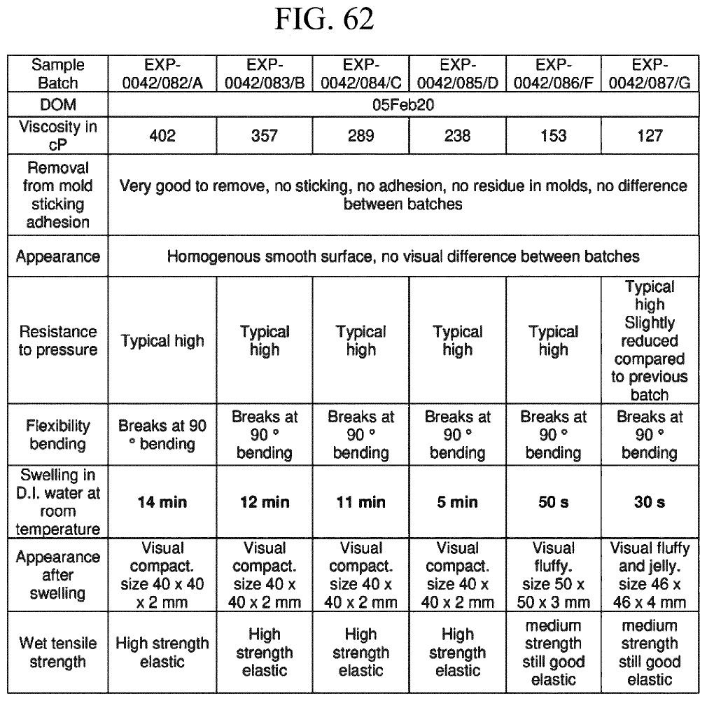

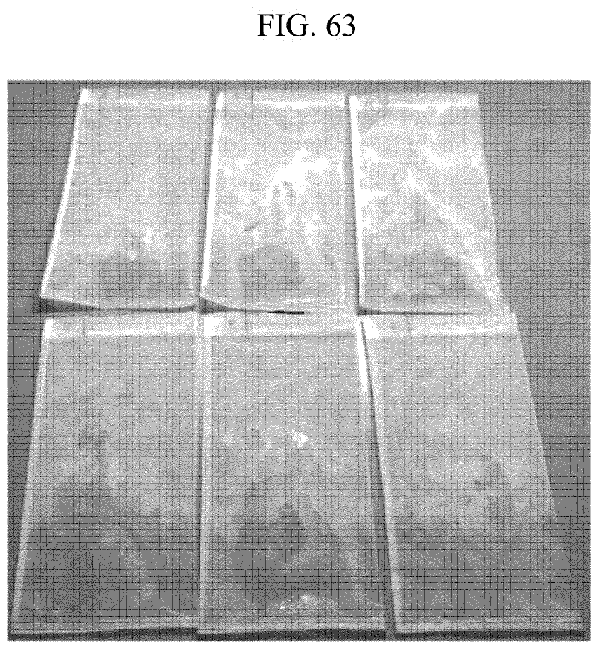

The Xaracoll matrices and their properties are summarized in FIG. 62. The filtration properties of the Xaracoll bulk formulations are shown in FIG. 63. There is a little force needed for the filtration.

### Nonsterile Matrix Property Evaluation

There was an mLMC viscosity-dependent swelling behavior of the manufactured Xaracoll matrices, with swelling time of matrices which used different mLMC viscosity for manufacturing are shown in FIG. 62.

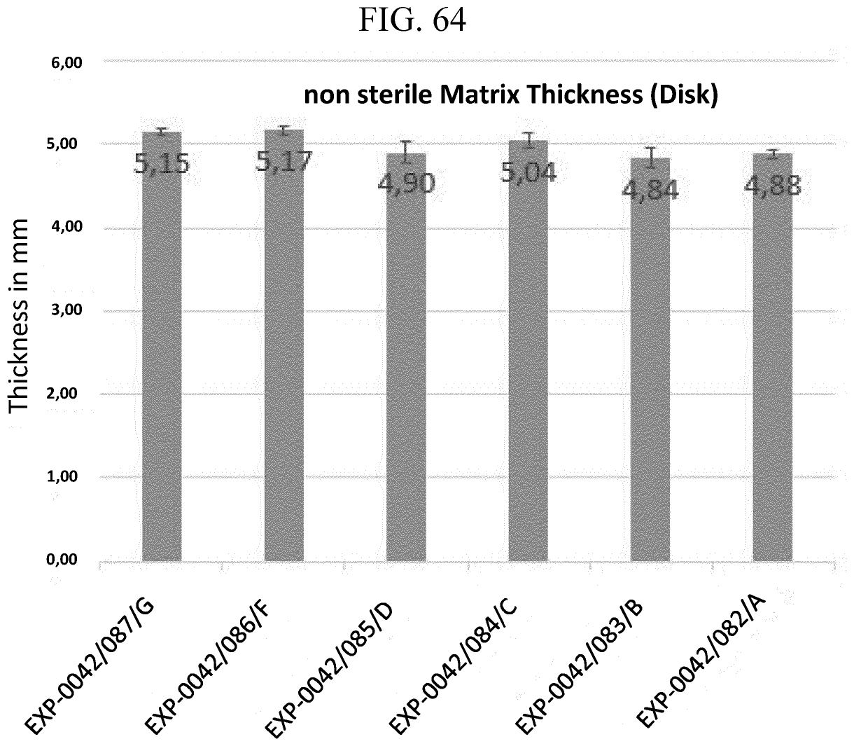

The middle thickness of the nonsterile Xaracoll matrix samples was measured in parallel to the resistance to pressure test using the Zwicki 500 N texture analyzer (FIG. 64).

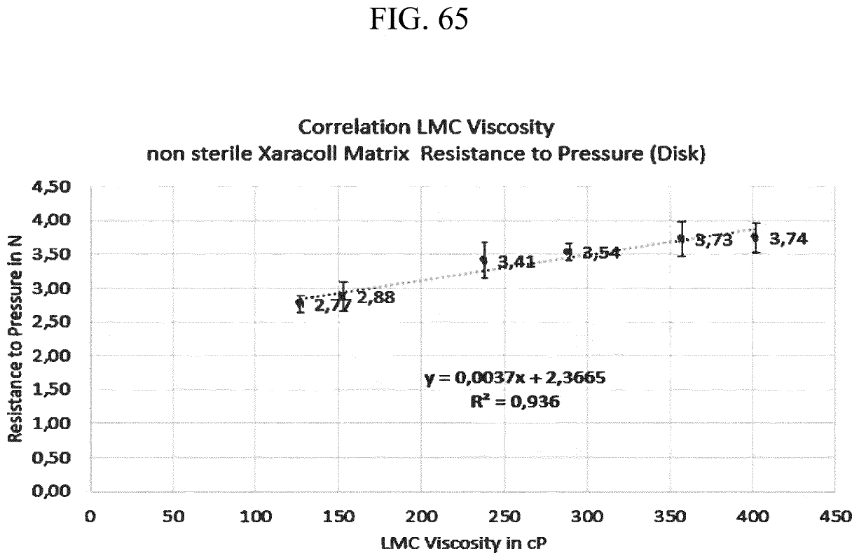

The resistance to pressure of the non-sterile Xaracoll matrix samples was measured using the Zwicki 500 N texture analyzer (FIG. 65).

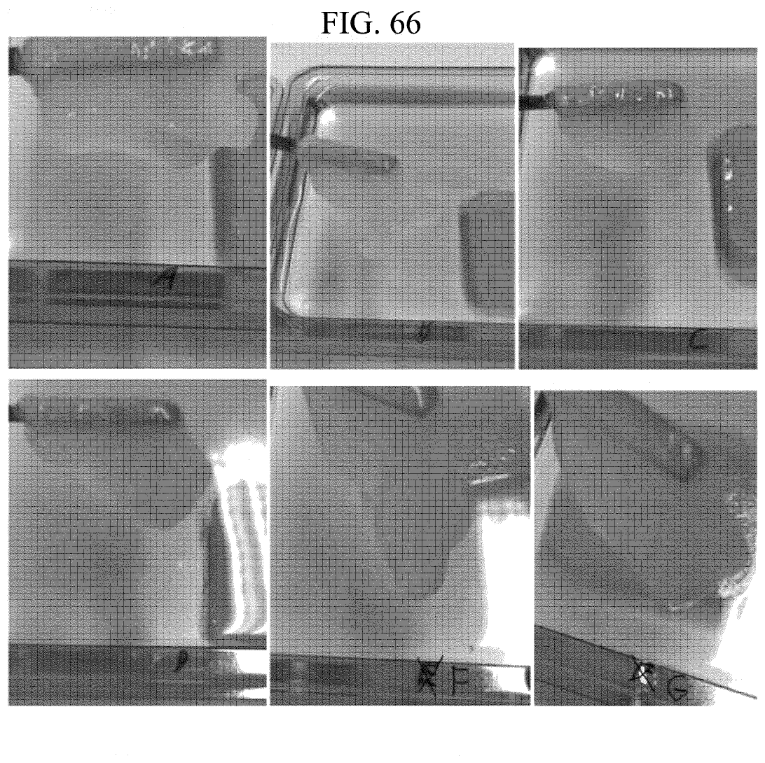

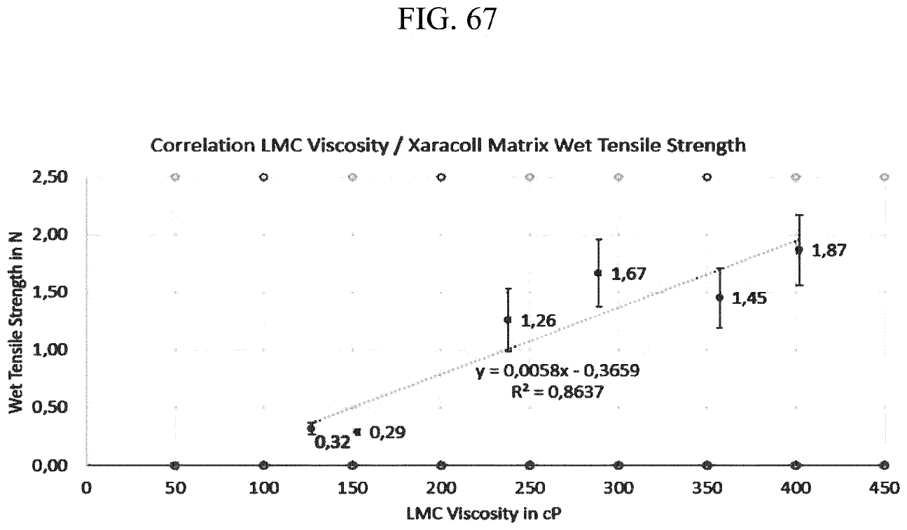

The wet tensile strength of the non-sterile Xaracoll matrix samples was measured using the Zwicki 500 N texture analyzer (FIG. 66 and FIG. 67).

### Sterile Matrix Property Evaluation

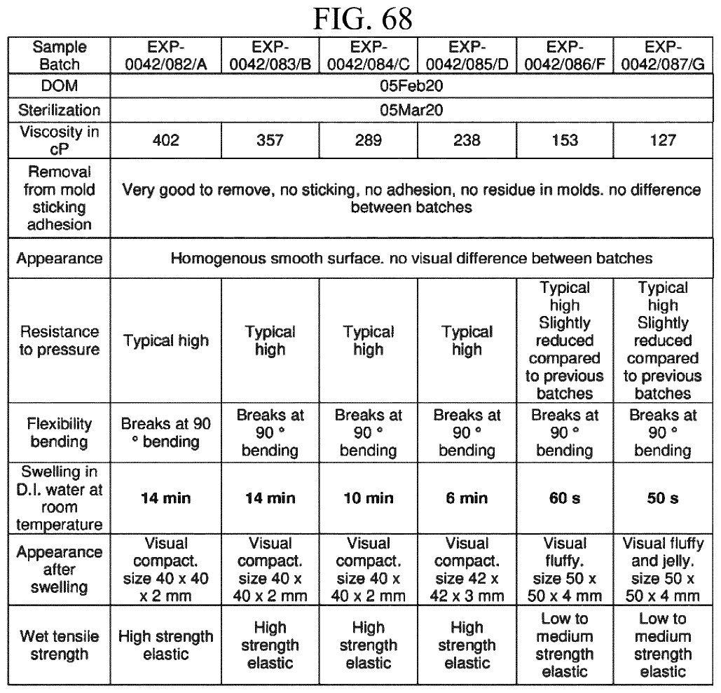

FIG. 68 provides an overview of the properties of the sterile matrices after 1 day of equilibration in the lab.

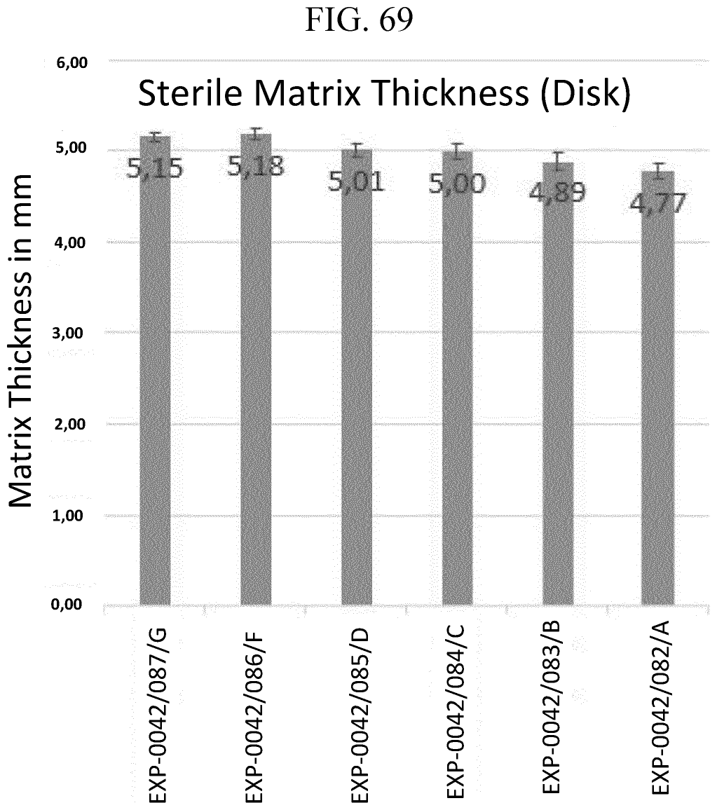

The middle thickness of the sterile Xaracoll matrix samples was measured in parallel to the resistance to pressure test using the Zwicki 500 N texture analyzer. There is no change in the matrix thickness through sterilization, indicating good moisture control in the sterilization process (FIG. 69).

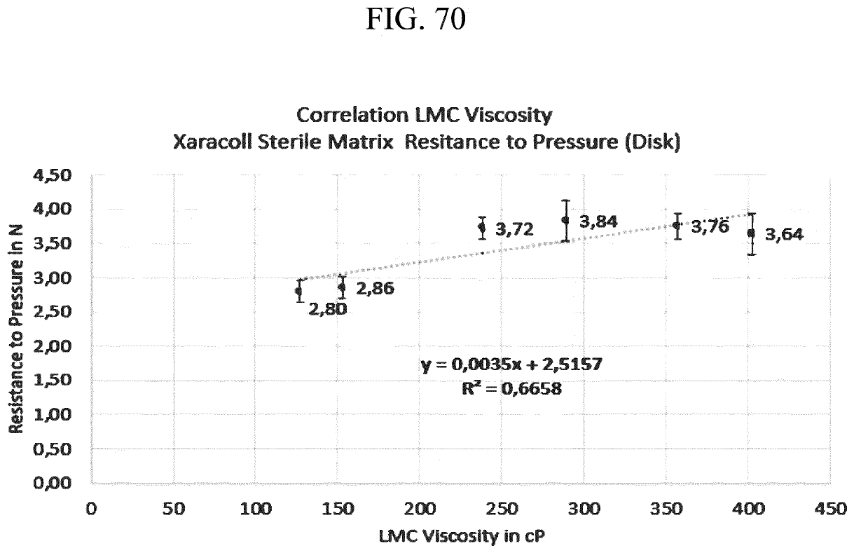

The resistance to pressure of the sterile Xaracoll matrix samples was measured, using the Zwicki 500 N texture analyzer (FIG. 70).

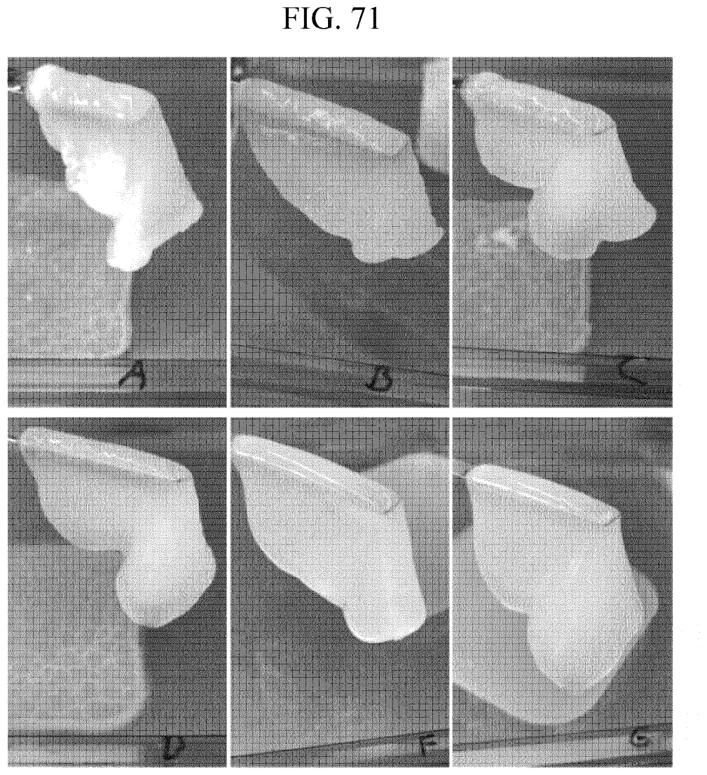

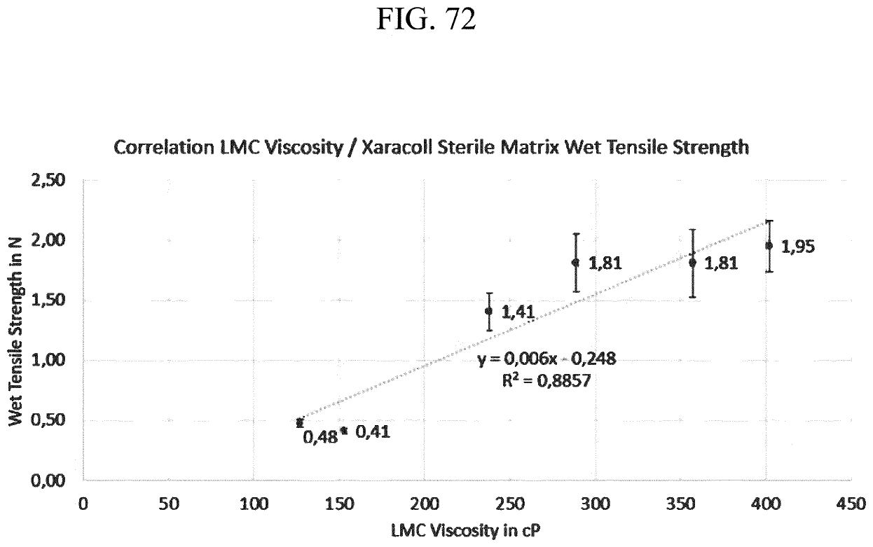

The wet tensile strength of the sterile Xaracoll matrix samples was measured, using the Zwicki 500 N texture analyzer. The tensile strength is increased by 0.17 N (in average) through sterilization (FIG. 71 and FIG. 72).

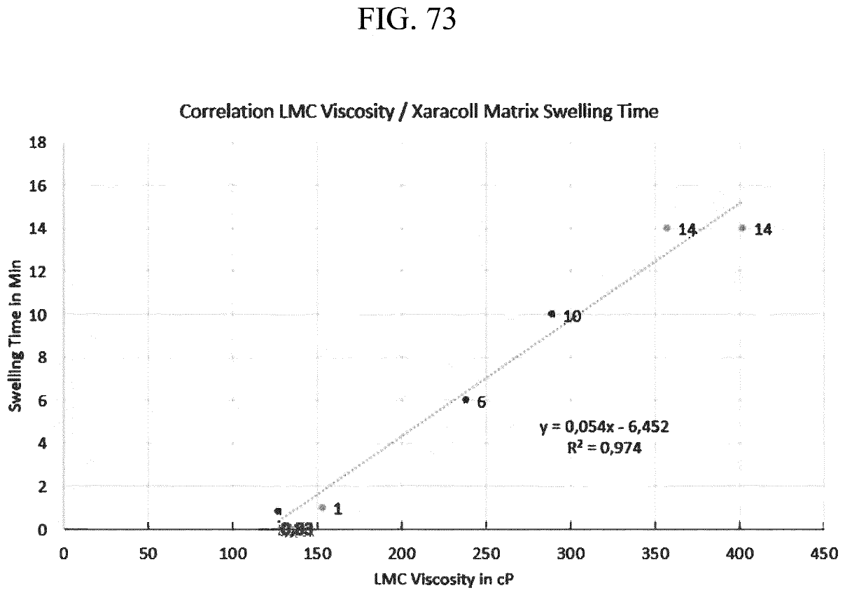

FIG. 73 depicts the swelling time of matrices with the viscosity of mLMC used for manufacturing.

### Overall Findings

The wet tensile strength increases by 0.17 N (in average) through sterilization. This seems to correlate with the observed swelling time. The resistance to pressure does not change through sterilization. The matrix thickness does not change through sterilization.

### Xaracoll Characterization

### Water Uptake

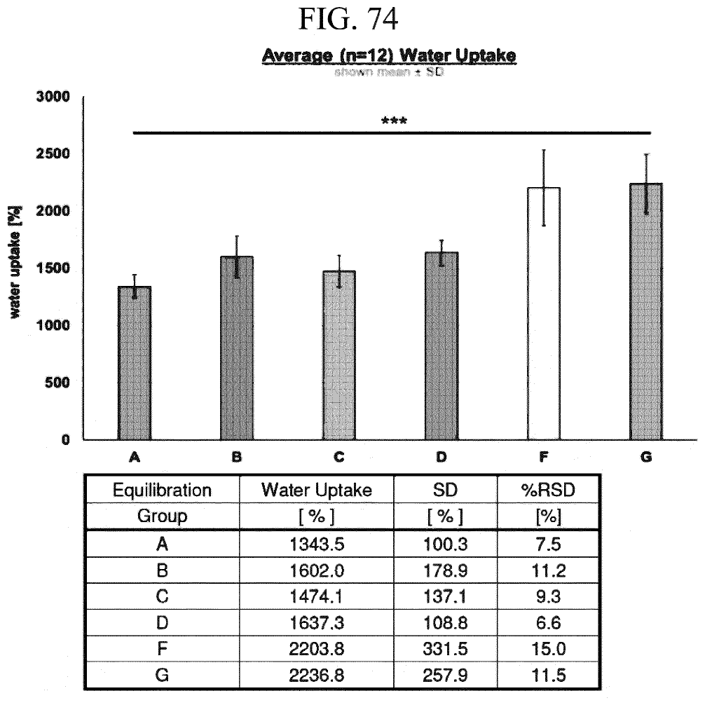

FIG. 74 depicts water uptake results.

### Dissolution

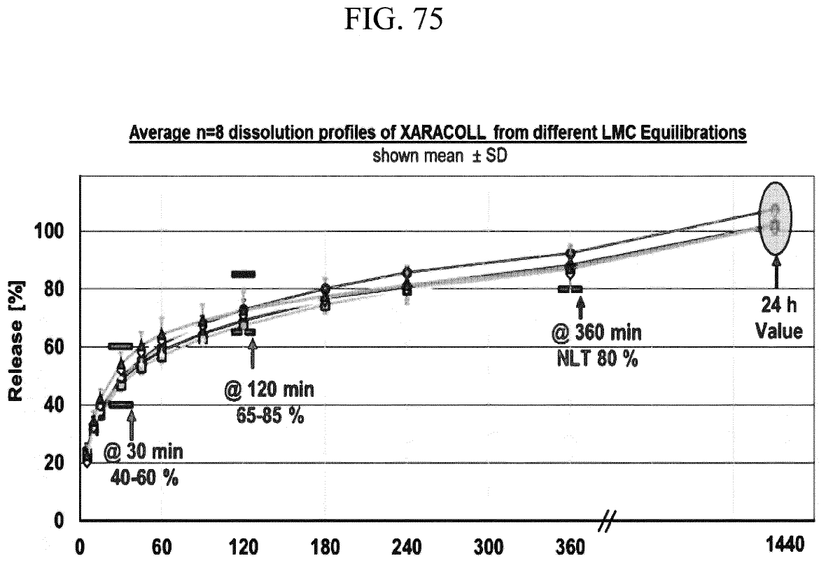

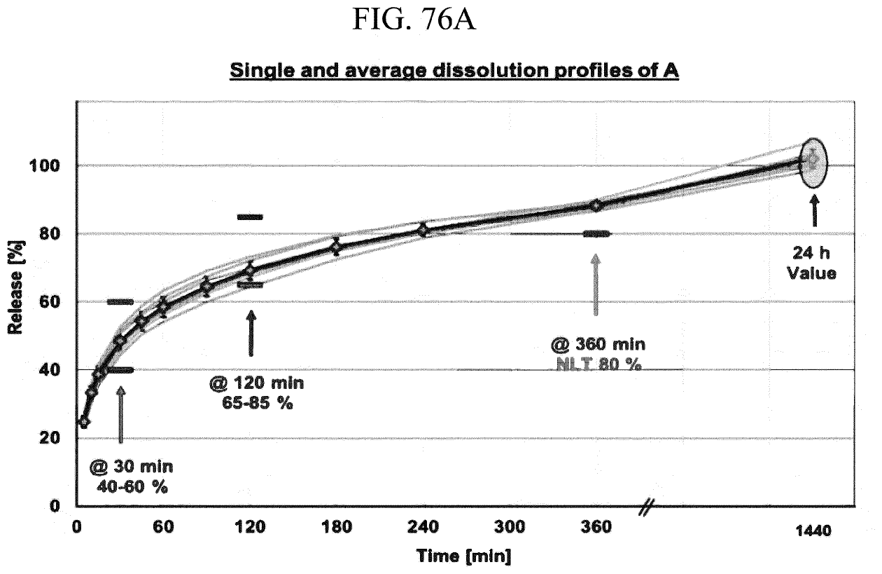

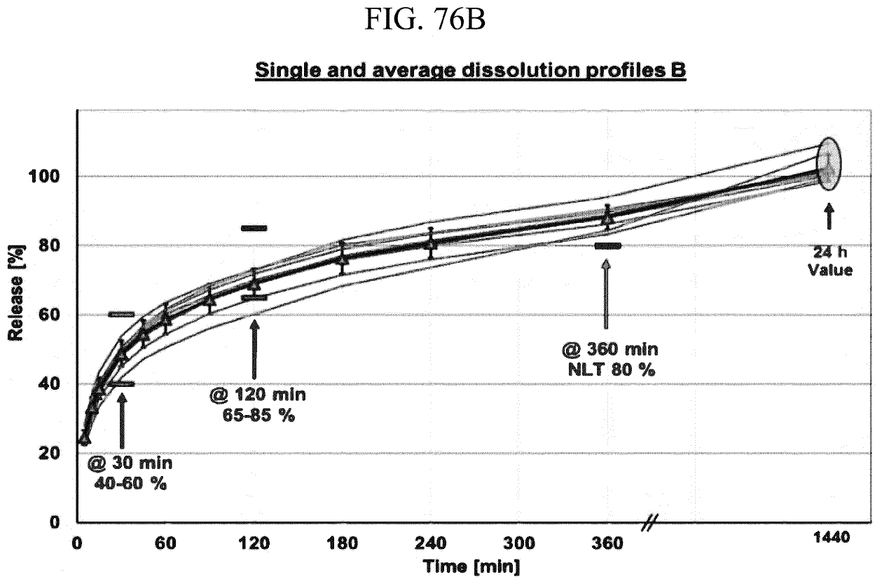

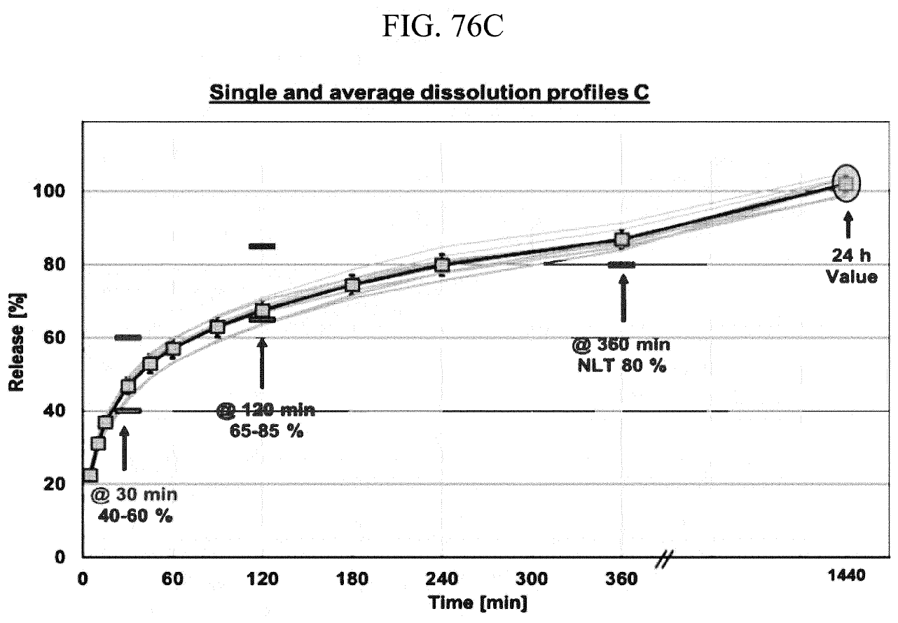

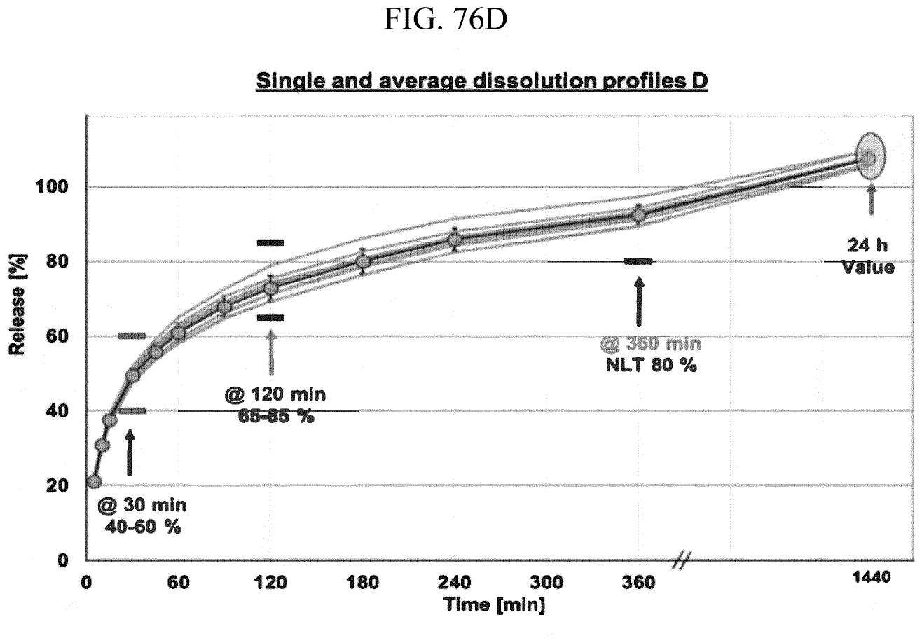

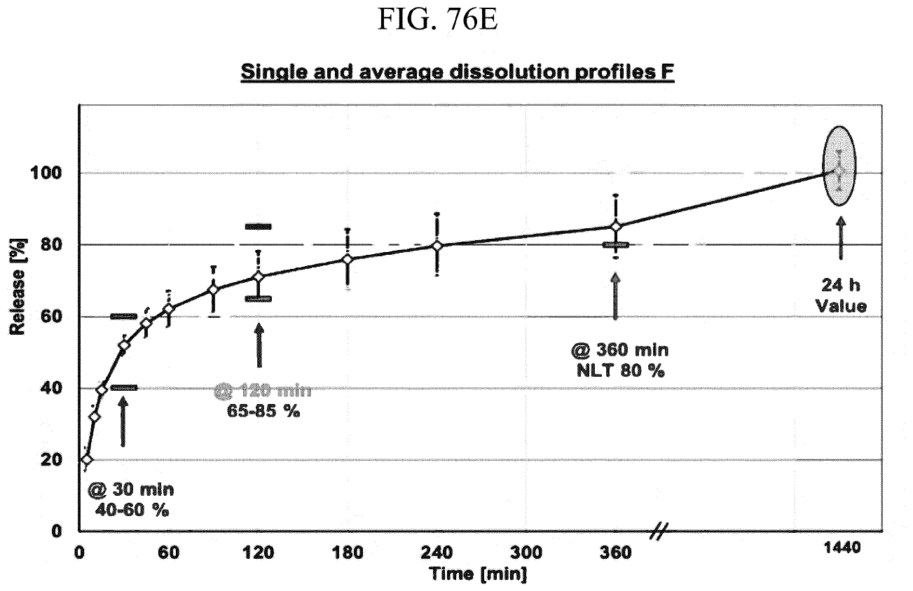

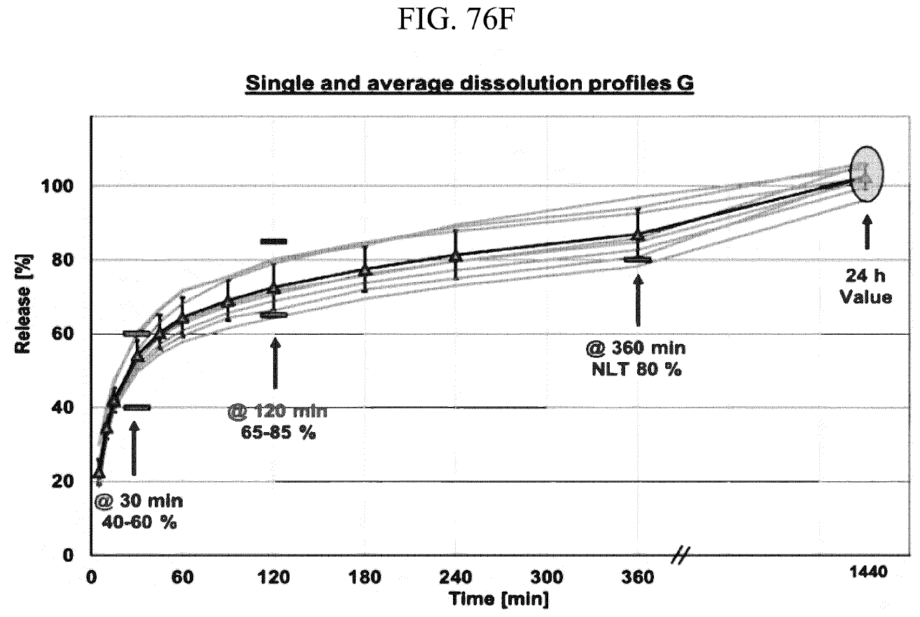

The average dissolution curves (FIG. 75) of all batches met the drug release acceptance criteria. At time point 30 min, all average curves are around the center point of 50% release, batch C with the lowest value of 46.9% release and batch G with highest value of 54.3% release. All dissolution single results met the 30 min acceptance criteria FIGS. (76A-76F). At time point 120 min, all average curves are below the center point of 75% release, batch C with the lowest value of 67.5% release and batch D with highest value of 72.9% release. At time point 360 min, all average curves show a release higher than 80%, batch F with the lowest value of 85.1% release and batch D with highest value of 92.4% release.

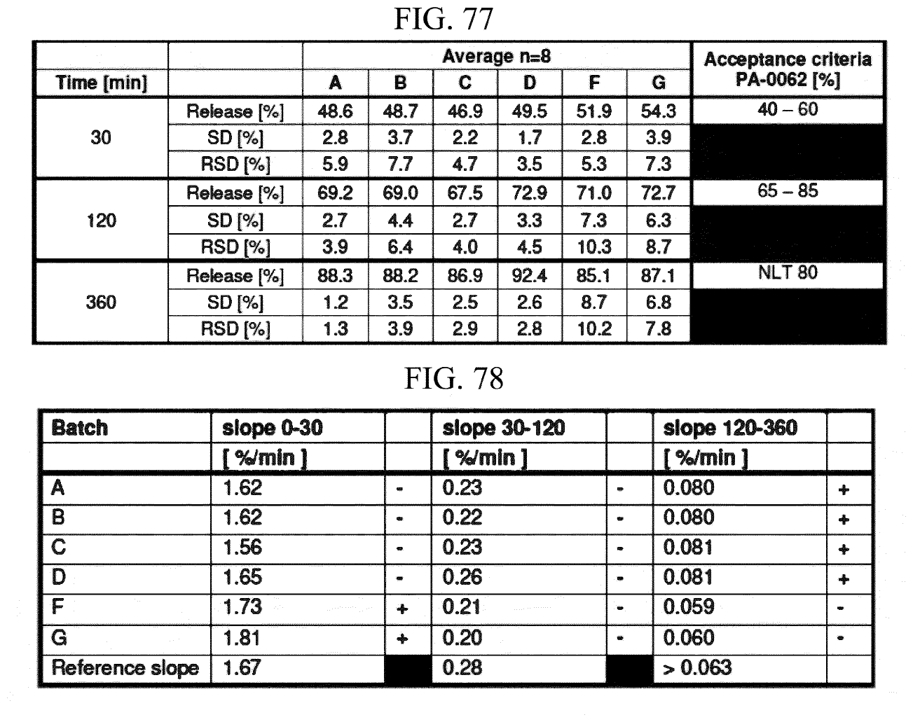

FIG. 77 shows the average results, SD and RSD at 30, 120 and 360 min.

To categorize the different characteristics of the release profiles, the gradients at 0-30, 30-120, and 120-360 were evaluated (FIG. 78). The ideal slope of a region is based on the acceptance criteria. The ideal drug release slope from start to 30 min is 50%/30 min=1.67%/min. The ideal drug release slope from 30 min to 120 min is 25%/90 min=0.28%/min. The slope to meet 80% at 360 minis 0.0625%/min (based on 65% at 120 min). The small column in the table indicates whether the slope is smaller (−), ideal (0) or greater (+).

The average dissolution curves of all batches were compared via f1 and f2 factor method (Table 9). The average curves of the batches EXP-0040/046/A-C, F-G were compared with the average curve of the for this purpose used reference batch EXP-0040/046/D.

TABLE 9
f1/f2-factor comparison of all batches with D batch

| Comparison | f1 | f2 |
|---|---|---|
| D vs A | 5.0 | 75.1 |
| D vs B | 5.0 | 74.8 |
| D vs C | 6.3 | 69.7 |
| D vs F | 4.0 | 76.6 |
| D vs G | 5.4 | 72.3 |

However, the f1 and f2 factor methods compare the whole dissolution profile, therefore this method cannot distinguish differences of the profiles if they compensate in sum.

### DSC

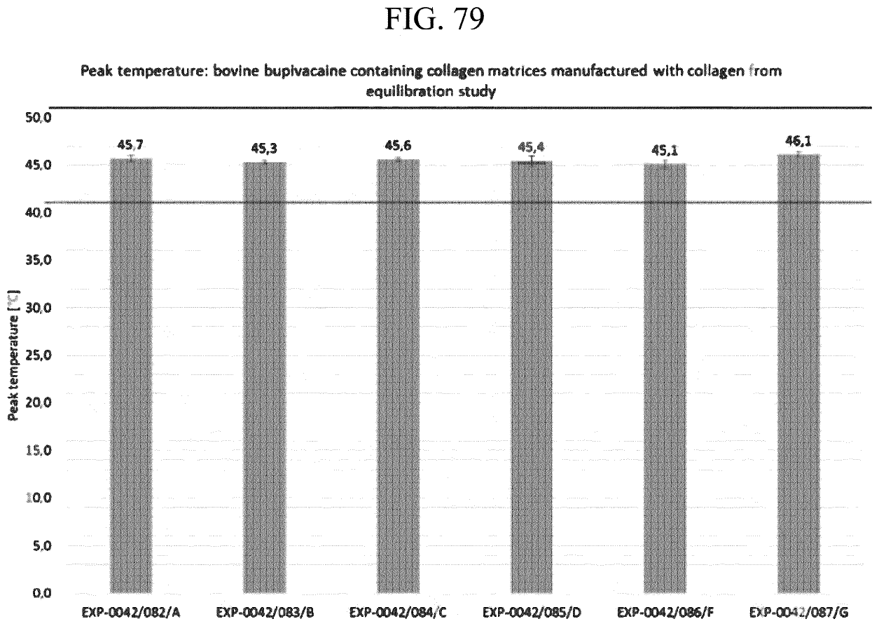

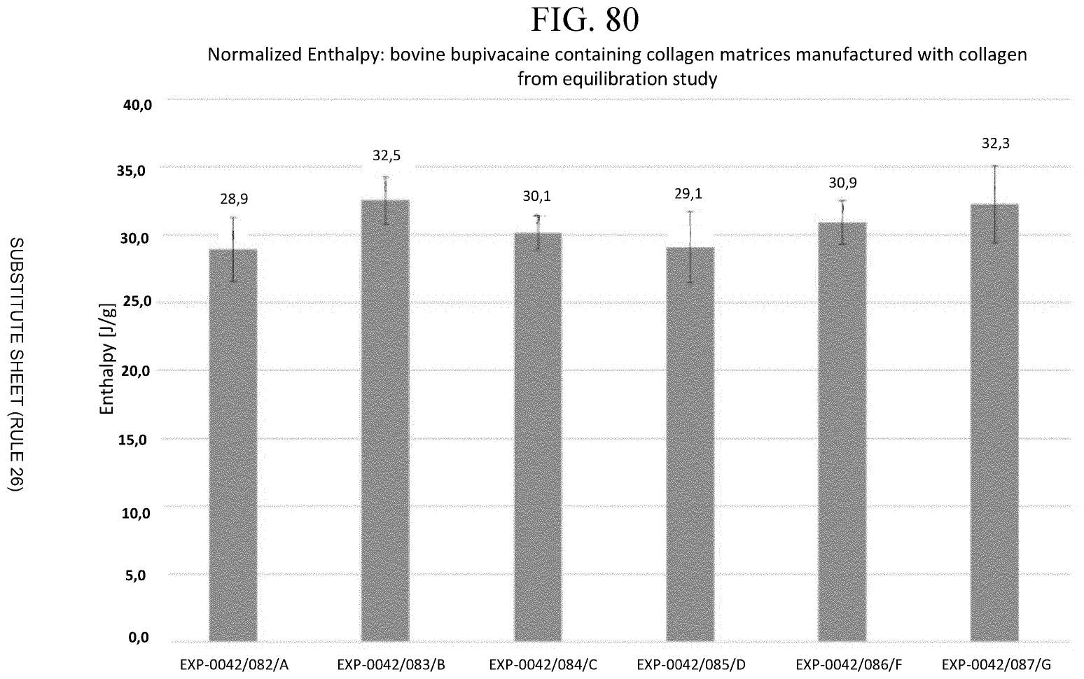

All peak denaturation temperature values are within specification (42-52° C.). (FIG. 79. FIG. 80 depicts the normalized enthalpy.

### SEM Analysis

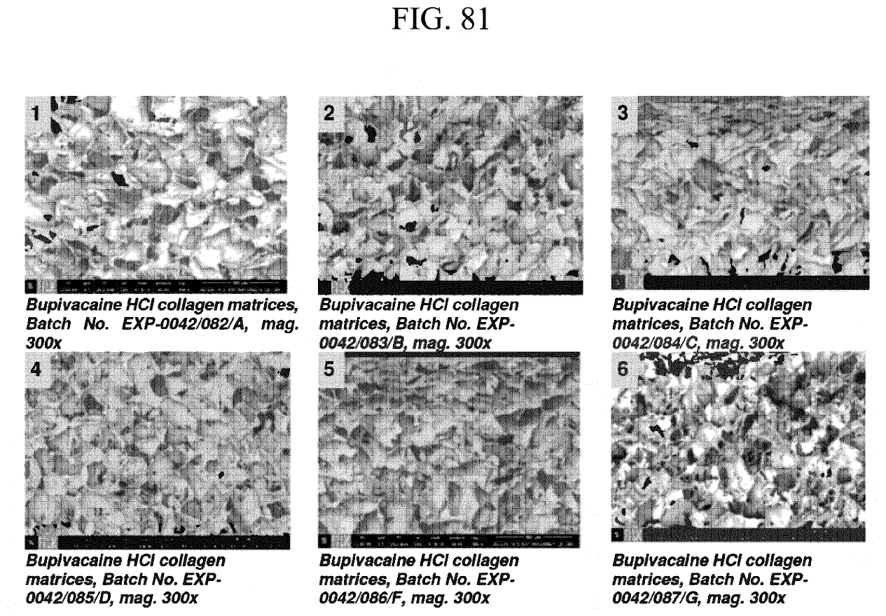

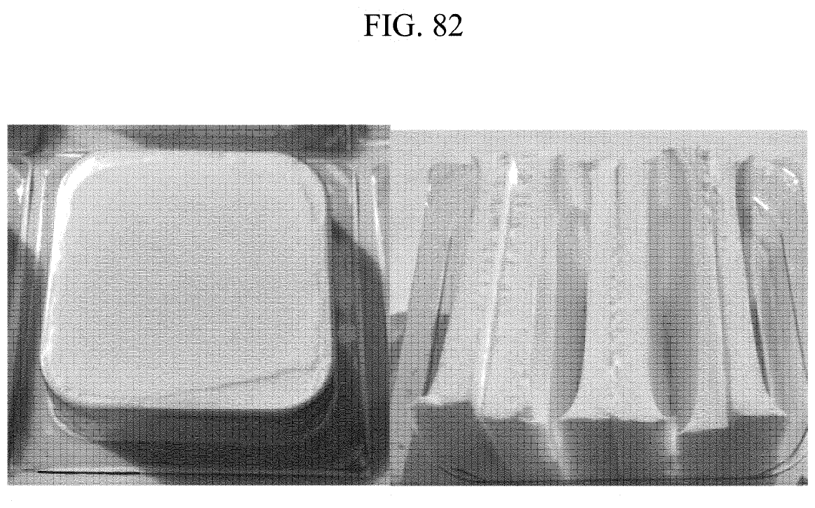

SEM images of the surfaces and cross sections of the different Xaracoll matrix samples are displayed in FIG. 81. FIG. 82 depicts “bowling” in some Xaracoll samples.

### CONCLUSIONS

Characterization of LMC equilibrated to different viscosities: There was a slight tendency for lower trypsin resistance with increased mLMC equilibration, i.e. lower viscosity of equilibrated mLMC. All other tested mLMC characterization parameters, including purity (SDS-PAGE), degree of natural cross-links (TNBS assay), and three-dimensional fine structure (AFM) were not affected by different degrees of equilibration (different viscosities).

Impact on Xaracoll Manufacturing: The full range of mLMC viscosity from 127 cP up to 402 cP can be processed without problems.

Impact of mLMC viscosity on Xaracoll characteristics: For both the non-sterile and the sterile matrix, a tendency for decreased swelling time with decreased mLMC viscosity was observed, however, all effects were considered uncritical in the tested range between 127 and 402 cP. There is a tendency for the accelerated matured samples for a nonlinear extra drop of swelling time. For both the non-sterile and the sterile matrix, a correlation of the wet tensile strength with the mLMC viscosity can be observed, the gradient is comparably steep. Furthermore, the wet tensile strength increases by 0.17 N (in average) after sterilization. Wet tensile strengths below 0.5 N were found for the two lowest mLMC viscosities, i.e., 127 cP and 153 cP. There is a tendency for the accelerated matured samples for a nonlinear extra drop of wet tensile strength. For both the non-sterile and the sterile matrix, a correlation of the resistance to pressure with the mLMC viscosity can be observed, the gradient is comparably shallow. The resistance to pressure does not change through sterilization, all effects were uncritical in the tested range between 127 and 402 cP.

SEM analyses show the typical honeycomb porous structure of collagen matrix over a broad range of mLMC viscosities, however, some disturbances of this structure at the lowest mLMC viscosity (i.e., 127 cP).

For several matrix parameters (swelling time, water uptake, wet tensile strength, dissolution) a non-linear correlation was observed for the accelerated matured samples. This indicates that moisture uptake to an LOD as high as 22% during maturation should be avoided. A target LOD of approximately 18% during the maturation process is suggested based on the available data.

### Example 6: Elemental Impurities in Xaracoll

Table 10 is a chart showing levels of elemental impurities detected in the Xaracoll product.

TABLE 10

| Batch no: | | | | 15021509 | 15031509 | 15041509 |
|---|---|---|---|---|---|---|
| Element | Mass | Class | Limit [ppm]¹ | [ppm] | [ppm] | [ppm] |
| Cd | 111 | 1 | 0.2 | 0.012 | 0.012 | 0.014 |
| Pb | 208 | 1 | 0.5 | <0.05 ppb | <0.05 ppb | <0.05 ppb |
| AS | 75 | 1 | 1.5 | 0.004 | 0.004 | 0.004 |
| Hg | 201 | 1 | 0.3 | <0.01 ppb | <0.01 ppb | <0.01 ppb |
| Co | 59 | 2A | 0.5 | 0.022 | 0.020 | 0.022 |
| V | 51 | 2A | 1 | 0.008 | 0.006 | 0.006 |
| Ni | 60 | 2A | 2 | 1.360 | 1.144 | 1.070 |
| Tl | 205 | 2B | 0.8 | 0.020 | 0.020 | 0.044 |
| Au | 197 | 2B | 10 | 0.012 | 0.010 | 0.010 |
| Pd | 105 | 2B | 1 | 0.016 | 0.014 | 0.012 |
| Ir | 193 | 2B | 1 | 0.089 | 0.078 | 0.058 |
| Os | 189 | 2B | 1 | <0.01 ppb | <0.01 ppb | <0.01 ppb |
| Rh | 103 | 2B | 1 | <0.02 ppb | <0.02 ppb | <0.02 ppb |
| Ru | 101 | 2B | 1 | <0.02 ppb | <0.02 ppb | <0.02 ppb |
| Se | 82 | 2B | 8 | 0.010 | <0.02 ppb | 0.102 |
| Ag | 107 | 2B | 1 | 0.069 | 0.061 | 0.080 |
| Pt | 195 | 2B | 1 | 0.012 | 0.012 | 0.010 |
| Li | 7 | 3 | 25 | 0.002 | 0.002 | 0.002 |
| Sb | 121 | 3 | 9 | 0.071 | 0.057 | 0.066 |
| Ba | 137 | 3 | 70 | 0.002 | 0.002 | <0.01 ppb |
| Mo | 95 | 3 | 150 | 0.053 | 0.027 | 0.030 |
| Cu | 63 | 3 | 30 | 0.678 | 0.735 | 0.819 |
| Sn | 118 | 3 | 60 | 0.006 | 0.006 | 0.004 |
| Cr | 52 | 3 | 110 | 1.110 | 0.748 | 0.839 |

¹Parenteral permitted daily exposure - ppm/day
ppm = parts per million;
ppb = parts per billion

### Example 7: Optimized EO Sterilization Process

Cycle development was initiated to optimize the critical sterilization parameters (primarily EO gas concentration, humidification, pressure, and cycle exposure time) for XARACOLL. Terminal EO sterilization cycle development activities resulted in the following critical cycle parameters:
EO:CO₂ gas composition/concentration ratio of 6% EO:94% CO2;
Pre-conditioning the chamber relative humidity to >50% for ≥18 min;
Sterilization temperature of 40° C.±4° C.;
2 bar±0.2 bar overpressure;
EO concentration≥310 mg/L;
Cycle dwell/exposure time: 4 h±10 min;
18-h desorption period in-chamber.

The major differences between the initial and optimized EO sterilization cycles are shown in Table 11.

TABLE 11

| | Process step | | | |
|---|---|---|---|---|
| | | | Optimized | |
| | Initial | | EO cycle | |
| parameter | EO cycle | | (current) | |
| Pre-conditioning | No | | Yes, RH >50% | |
| | | | for >18 min | |
| EO:CO₂ gas conc. ratio | 15%:85% | | 6%:94% | |
| Overpressure | 4 | bar | 2 bar (+/−0.2 bar) | |
| EO concentration | ~1300 | mg/L | 310 | mg/L |
| Cycle dwell/exposure time | 6 | hours | 4 | hours |
| Aeration times | >>5 | weeks | ≤5 | weeks |
| ECH residues | ≤1000 | ppm | ≤175 | ppm |

### Example 9: Non-Clinical Studies

Bupivacaine has been widely used as a local anesthetic and has demonstrated efficacy in animal models in multiple species (Li, et al., 2013, Hersh, et al., 1992). The pharmacology of bupivacaine is well understood.

The distribution, metabolism, and excretion of bupivacaine across species is characterized in the literature including data showing that, following absorption, bupivacaine is rapidly and readily distributed to tissues, with the highest concentrations in highly perfused tissues. In general, the metabolic profile of bupivacaine is similar across species although quantitatively different, with no novel metabolite identified in humans (Goehl, et al., 1973, Carson 2000). The excretion of bupivacaine in monkeys and humans is predominantly in the urine; in rats, excretion in urine and feces is generally similar; and in dogs, only small amounts of excretion of bupivacaine in the urine have been reported. The dog excretion data in the literature for bupivacaine is consistent with excretion data obtained in the dog following implantation of INL-001, which showed approximately 0.16% of the bupivacaine dose was collected in the urine over a 72-hour period. The genotoxicity of bupivacaine HCl was evaluated in 4 in vitro studies and 2 in vivo studies, with no safety concerns identified.

Clinically significant pharmacokinetic drug interactions with bupivacaine have been reported with verapamil, diazepam, and cimetidine in humans, animals, and/or in vitro test systems. These interactions occurred with bupivacaine administered epidurally or intravenously (iv).

The uniformity of bupivacaine in the INL-001 implant and the release of bupivacaine from the implant was evaluated in in vitro and/or in vivo assessments. The in vitro uniformity study demonstrated that bupivacaine content is homogeneously dispersed throughout INL 001. An in vitro dissolution study of a whole matrix found that bupivacaine was released as early as 5 minutes and complete release of bupivacaine occurred by 24 hours. An in vivo study showed the in vitro drug release profile was similar to that seen in vivo in dogs with INL-001 implanted into the abdomen and subcutaneous tissues through a surgical incision.

Two studies were conducted in which INL-001 was surgically implanted in rats followed by a 56 day postsurgical period to assess potential local and systemic effects, determine potential effects on wound healing, and characterize the resolution of any implant related findings. The initial study was conducted with early development drug product and the second study with the clinical Phase 3/commercial formulation using generally the same study design. INL-001 was well tolerated following implantation. By day 28 following INL-001 implantation, attrition of the implant was approximately 95% and the implant was not observed microscopically by day 56 after dosing. INL-001-associated findings were limited to observations at the injection site, including transient edema through day 10 and microscopic findings associated with the repair process.

Biocompatibility studies were conducted with extracts from INL-001 and the drug-free implant; these studies did not identify any safety concerns. There was no evidence of acute systemic toxicity or effects on body temperature (i.e., pyrogenicity) associated with administration of drug free implant extract or evidence of skin irritation, sensitization, or genotoxicity following exposure to bupivacaine implant extract.

### Example 10: Clinical Studies

The clinical pharmacology, efficacy, and safety of INL-001 (at single doses of 100, 150, 200, and 300 mg) have been evaluated in 11 completed clinical studies (Phases 1-3) in adults, including 2 well controlled pivotal Phase 3 studies of INL-001 in open inguinal hernia repair, at its recommended approved dose (three 100-mg bupivacaine HCl implants).

In a pharmacokinetic/relative bioavailability study in patients following inguinal hernioplasty (Study INN-CB-022), the commercial formulation of INL 001 at a dose of 300 mg was compared with Marcaine 0.25% injection at a dose of 175 mg (the maximum recommended single dose). Quantifiable bupivacaine concentrations were evident at the first posttreatment time point measured (30 minutes) for all patients treated with INL-001 or Marcaine. Bupivacaine concentrations were detectable through the 96-hour posttreatment time point (last time point) in both treatment groups but at higher concentrations with INL-001 than with Marcaine. Pharmacokinetic analysis led to the following additional conclusions:

For the INL-001 treatment group, the mean maximum observed plasma concentration (Cₘₐₓ) (minimum, maximum) was 663.412 ng/mL (274.00 ng/mL, 1230.00 ng/mL) compared with a mean Cₘₐₓ (minimum, maximum) for the Marcaine treatment group of 641.000 ng/mL (275.00 ng/mL, 1140.00 ng/mL).

For the INL-001 treatment group, the median time to maximum observed plasma concentration (Tₘₐₓ) was 3.03 hours with a mean terminal elimination half-life (t_{1/2}) of 18.95 hours compared with a Tₘₐₓ of 1.01 hours and a mean t_{1/2} of 9.08 hours for the Marcaine treatment group.

For INL-001, the geometric means for area under the concentration-time curve (AUC) from Time 0 to the last quantifiable concentration (AUC₀₋ₗₐₛₜ) was 18186.9 h·ng/mL and the AUC from Time 0 extrapolated through infinity (AUC_{0-∞}) was 19012.5 h·ng/mL. For Marcaine, the geometric means for AUC₀₋ₗₐₛₜ and AUC_{0-∞} were 8836.9 h·ng/mL and 8920.1 h·ng/mL, respectively.

In clinical studies of an earlier bupivacaine collagen implant formulation, following implantation of INL-001 at 100, 150, and 200 mg in various abdominopelvic surgeries, quantifiable bupivacaine plasma concentrations were observed from 30 minutes after placement (at the first posttreatment time point). Bupivacaine concentrations increased in a slightly higher than dose-proportional manner with increasing doses of INL-001.

The efficacy of INL-001 was evaluated in 2 multicenter, double-blind, placebo controlled Phase 3 studies in adults that independently demonstrated the effectiveness of locally placed INL 001 (300 mg implanted in layers at the surgical site) in reducing both pain intensity and the need for opioid rescue analgesia after surgery; together, these studies demonstrate the reproducibility of the INL-001 treatment effects (Studies INN-CB-014 and INN-CB-016). In each study, INL-001 achieved the primary endpoint, with patients treated with INL-001 experiencing statistically significantly less pain (p≤0.0004) as evaluated by the (time weighted) sum of pain intensity (SPI) through 24 hours (SPI24). These reductions in pain intensity were coupled with less total use of opioid rescue analgesic medication (TOpA) in the INL-001 treatment group compared with the placebo implant group. In data pooled from the 2 pivotal studies, patients used statistically significantly (p≤0.0004) less opioid rescue analgesia and had statistically significantly (p=0.0007) fewer opioid-related treatment emergent adverse events (i.e., nausea, vomiting, and constipation) over the post-implantation period compared with subjects in the combined placebo implant group.

The existing INL-001 safety database is derived from a clinical development program of 11 studies conducted in soft tissue surgeries in adults, including the 2 positive Phase 3 studies in inguinal hernia repair. A total of 892 adult patients have received collagen matrix implants in this program (612 INL-001 and 280 placebo implants). Of the 892 patients, 816 patients underwent inguinal hernia repair, 69 patients underwent hysterectomy, and 7 patients underwent other types of soft-tissue surgeries (i.e., nonlaparoscopic benign gynecological procedure other than hysterectomy or elective surgery requiring a vertical or transverse abdominal incision).

Across these 11 studies: adverse events occurring at an incidence of 2% or more patients following administration of INL-001 at 300 mg and at a higher incidence than placebo implants, respectively, were somnolence (19.2% vs 13.9%), dizziness (16.4% vs 13.9%), incision site swelling (13% vs 10.7%), incision site pain (11.7% vs 11.4%), restlessness (7.7% vs 6.8%), dysgeusia (7.5% vs 4.6%), vision blurred (4.9% vs 2.1%), headache (4.7% vs 2.1%), tremor (4.5% vs 2.1%), postprocedural discharge (4.3% vs 3.6%), scrotal swelling (2.8% vs 1.8%), seroma (2.6% vs 1.8%), oral hypoesthesia (2.6% vs 1.4%), pyrexia (2.3% vs 1.8%), and wound dehiscence (2.1% vs 1.8%).

In these studies, adverse events considered by the investigator to be treatment related following INL-001 placement in the surgical site occurred at a rate of 1.5% or less. The only treatment-related adverse event that occurred in 1% or more of all patients who received INL-001 at 300 mg was dysgeusia (1.3%), which also occurred in 0.7% of patients in the placebo group.

Across the INL-001 clinical development program, 16 patients experienced 1 or more serious adverse events: 11 patients in the INL-001 (including earlier formulation) treatment group and 5 patients in the placebo implant or other comparator group. Serious adverse events reported in the INL-001 treatment group included wound infection and seroma. One patient had the INL-001 implant removed after placement of an earlier formulation of bupivacaine collagen matrix implant during bladder sling surgery (see event described below).

There were no verbatim reports of systemic bupivacaine toxicity or LAST during any inguinal hernia repair study done as part of the development program for INL-001. The safety assessments conducted during the development program included monitoring adverse events, measurement of vital signs, and assessments with multiday cardiac Holter monitors. These assessments revealed no constellation of neurologic or cardiovascular (CV) signs or symptoms to suggest systemic bupivacaine toxicity in patients undergoing open inguinal hernia repair receiving INL 001.

One patient experienced signs and symptoms thought to be consistent with LAST approximately 4 hours after administration of an earlier formulation of the INL-001 (at 150 mg) following bladder sling surgery. Treatment included administration of lipid emulsion and surgical removal of the INL-001 implants.

Across the INL-001 clinical development program, incision-site adverse events occurring with an incidence of 2% or more in either the INL-001 (including earlier formulation) or placebo group compared with a non-implant comparator treatment group (n=52) included swelling, pain, other complication, postprocedural discharge, erythema, dehiscence, and inflammation.

### Example 11: Study for Postoperative Pain Management Following Soft-Tissue Surgery

The primary objective of the study is to evaluate the safety and tolerability of the INL-001 implant in patients following open ventral hernia repair, abdominoplasty, open abdominal hysterectomy, laparoscopic-assisted colectomy, and reduction mammoplasty.

The secondary objective of the study is to characterize the pharmacokinetic profile of bupivacaine from the INL 001 implant through 96 hours after implantation in patients following study surgery.

Exploratory objectives are: (i) to assess the impact of the INL-001 implant on pain intensity in patients over time through 72 hours after implantation following study surgery; (ii) to assess the impact of the INL-001 implant on postsurgical opioid use in patients following study surgery; (iii) to assess the impact of opioid medication use, with the Opioid Related Symptom Distress Scale (OR-SDS), after implantation of INL 001 in patients following study surgery; and (iv) to explore health-related quality of life in patients after implantation of INL 001 following study surgery.

### General Study Design

The study is designed to be a multicenter, Phase 3, open-label, safety, tolerability, and characterization of pharmacokinetics study of the INL-001 (bupivacaine HCl) implant, at 300 mg, in patients following various soft-tissue surgeries: open ventral hernia repair, abdominoplasty, open abdominal hysterectomy, laparoscopic-assisted colectomy, and reduction mammoplasty. After a screening period, on the day of surgery (study day 1), eligible patients undergo study surgery under general anesthesia and have INL-001 implanted intraoperatively. Efficacy is also an exploratory measure in this study.

The duration of study participation for each patient is a maximum of 75 (±4) days, consisting of a screening period (up to 45 days before surgery), an inpatient period (preoperative, intraoperative, postoperative) of approximately 5 days, and an outpatient follow-up period (up to 30 days [±3 days] after treatment) including an end-of-study visit. Posttreatment (time measured from Time 0 [placement of first INL-001 implant]) safety assessments are made throughout the study after the informed consent form (ICF) is signed, and as specifically scheduled through 96 hours posttreatment, on day 7 (±1 day) (telephone), on day 15 (±3 days) (clinic visit), and on day 30 (±3 days) (clinic visit). Unless the investigator determines further hospitalization is necessary, patients are discharged approximately 96 hours posttreatment (inpatient day 5). Efficacy assessments are made through 72 hours after treatment.

During the screening period, all patients provide informed consent and undergo eligibility and other screening and safety assessments (medical history including review of prior medications, physical examination, urine drug screen, serum pregnancy test for women of childbearing potential, clinical laboratory tests [hematology, chemistry, urinalysis], vital signs measurement, and 12-lead electrocardiography [ECG]). Vital signs include body temperature, pulse, systolic and diastolic blood pressure, respiration rate, and oxygen saturation measured by pulse oximetry. The reason(s) a patient does not meet screening criteria are recorded. In addition, the investigator administers the Pain Catastrophizing Scale (PCS) (screening only) as an exploratory measure and the 15 item Quality of Recovery (QoR-15) questionnaire (baseline). Recording of adverse events and concomitant medication use commences once a patient signs an ICF.

On the day of surgery (day 1), patient eligibility is reconfirmed before the start of surgery (including medical history, urine drug screen, urine pregnancy test for women of childbearing potential, vital signs), and adverse events and prior/concomitant medications are reviewed.

Patients undergo study surgery using standard surgical procedures conducted under general anesthesia with no other local anesthetic used at the surgical site. All packs/gauze should be removed and adequate hemostasis must be achieved prior to skin closure. Surgical drains should be placed at the discretion of the surgeon and their use recorded. Ancillary procedures (e.g., liposuction) are prohibited.

Placement of study drug is detailed elsewhere herein. The time of the first placement of study drug (placement of first implant) is considered Time 0 and is recorded. Use of analgesic and all medications during surgery is recorded. At the surgeon's discretion, if a significant surgical/medical complication is encountered during surgery, study drug is not implanted and the patient is considered enrolled but not treated.

After surgery, patients are transferred to a postanesthesia care unit (PACU) or other postoperative recovery area for monitored observation. The times patients enter and are discharged from the PACU are recorded to calculate time to discharge from the PACU. Patients are monitored with pulse oximetry starting in the PACU through 24 hours posttreatment. After leaving the PACU (time in PACU to be at the discretion of the investigator), patients are placed in the postoperative unit or clinical research unit for domiciled observation. Vital signs, including body temperature, pulse, systolic and diastolic blood pressure, respiration rate, and oxygen saturation measured by pulse oximetry, are assessed at multiple time points through discharge, and in the clinic on days 15 and 30; 12-lead ECG will be done on day 30.

Adverse event and concomitant medication information, including use of rescue medication, are collected throughout the study (inpatient and outpatient). Surgical wound healing assessments will be made at 24, 48, 72, and 96 hours after Time 0, and on days 7, 15, and 30 using the specified list and assessed for and recorded as adverse events as appropriate. The Southampton Wound Grading System will be completed 72 hours (±3 hours) posttreatment and on days 15 and 30. Assessment for signs and symptoms potentially indicative of systemic bupivacaine toxicity are made after Time 0 at the following time points: 0.5, 1, 2, 3, and 4 hours (each ±15 minutes), and 5, 7, 9, 12, 15, 18, 24, 48, 72, and 96 hours (each ±1 hour), and days 7 (±1 day) and 15 (±3 days) using the specified list and assessment made and recorded as adverse events as appropriate.

At any time that a patient is determined to be exhibiting signs and/or symptoms suggestive of systemic bupivacaine toxicity, at the discretion of the investigator, a bupivacaine blood sample will be collected and 12 lead ECG are performed. The patient may be treated at the discretion of the investigator, including obtaining repeat bupivacaine blood concentrations, 12 lead ECG, or removal of the implants.

Blood samples for pharmacokinetic assessments are collected from patients at the following posttreatment time points: 0.5, 1, 1.5, 2, 3, 4, 5 (all ±15 minutes); 6, 8, 10, 12, 18 (all ±1 hour); 24, 36, 48, 72, 96 (all ±3 hours) hours. Concentrations of bupivacaine in plasma are assayed using a validated bioanalytical method.

After surgery, patient reports of pain intensity using an 11 point numeric pain rating scale (NPRS) are recorded at multiple time points through 96 hours (discharge). Scheduled pain intensity scores are recorded after Time 0 at 0.5, 1, 2, 3, 4, 5, 6, 8, 10, 12, 18, 20, 24, 28, 32, 36, 48, 72, and 96 hours. Each assessment prior to hour 10 has a ±15-minute window; each assessment after and including hour 10 has a ±30-minute window. The 0.5 hour and 1 hour NPRS assessments may be omitted if, on the basis of clinical judgement, the patient is not yet awake and alert enough to appropriately answer the NPRS after surgery. Pain intensity assessments scheduled between 2400 (midnight) and 0600 (6 am) may be limited to collection every 4 hours if the patient is sleeping. However, consecutive pain assessments may not be missed, and the hour 12, 24, 48, and 72 posttreatment pain assessments is to be completed even if they fall between 2400 (midnight) and 0600 (6 am). A pain intensity score is also be collected before any rescue pain medication use.

The QoR-15 questionnaire (see Section 8.2.4) will be administered 24, 48, 72, and 96 hours posttreatment and on day 7. The Patient Global Assessment (PGA) is administered 24, 48, 72, and 96 hours posttreatment and on day 7 by study staff. Patients are asked to rate how well their pain has been controlled during the study on the basis of the following criteria: 0 poor, 1 fair, 2 good, 3 very good, or 4 excellent. The OR SDS is administered at 24, 48, 72, and 96 hours posttreatment.

Patients are permitted rescue medication to manage breakthrough pain when it occurs. Oral acetaminophen at 1000 mg every 4-6 hours as needed for pain (maximum daily dosage 3000 mg) and/or oxycodone 5-mg tablet(s) may be given (not to exceed 10 mg in a 4-hour period during the inpatient stay). Immediately prior to receiving any rescue medication, a pain intensity score is recorded. If the NPRS score is 4 or less, patients are discouraged from taking opioid rescue medication; however, rescue medication may be requested and provided at any time. If patients require opioid rescue medication, but are unable to take oral medications, they are permitted to receive intravenous (iv) morphine (2-3 mg) every 3 hours until they are able to take oral rescue medication. As assessed by the investigator, if a patient's pain is not relieved by oxycodone and/or acetaminophen, the patient is not yet eligible for further treatment with oxycodone and/or acetaminophen, and more than 3 hours have passed since the previous iv morphine dose, a patient may receive a dose of iv morphine (2 3 mg) for pain relief. If the pain remains unrelieved or increases in intensity before additional rescue medication is allowed, additional treatment options will be discussed with the medical monitor.

Following discharge, to report an adverse event, a patient contacts study staff by telephone and report adverse event information, including incidence, duration, and any associated treatment. Patients with pain intensity scores of 4 or more at discharge is given a written prescription for immediate release oxycodone at a dosage of 5-10 mg every 4-6 hours as needed as rescue medication for breakthrough pain. Patients prescribed opioid rescue medication will also be permitted to take oral acetaminophen at 1000 mg every 4-6 hours (maximum daily dosage 3000 mg) and/or ibuprofen at 400 mg every 4-6 hours as needed for pain, on an outpatient basis. Patients with pain intensity scores of less than 4 at discharge are instructed to take oral acetaminophen at 1000 mg every 4-6 hours as needed for pain (maximum daily dosage 3000 mg) and/or ibuprofen at 400 mg every 4-6 hours as needed for pain, on an outpatient basis. Patients who do not receive a written prescription for oxycodone upon discharge are permitted to request immediate-release oxycodone 5-10 mg if their pain is unrelieved by acetaminophen. Use of opioids, acetaminophen, nonsteroidal anti-inflammatory drugs (NSAIDs), or any other medications after discharge from the hospital will be recorded, with data reviewed by study staff at subsequent contacts.

A detailed table of procedures and assessments is provided in Table 4.

### Planned Number of Patients and Countries

Approximately 140 patients will be screened to achieve a planned number of approximately 100 enrolled patients (20 per each of the 5 surgery types). Once 20 evaluable patients for a study surgery type are completed, enrollment in that surgery type will be halted. Enrollment for open abdominal hysterectomy may not achieve 20 patients but all efforts will be made to achieve this goal.

The number of evaluable patients is planned to be approximately 100. Details about the definition of evaluable patients and sample size are given herein.

The study is planned to be conducted in the US at a number of investigational centers to be determined. The study is expected to start at a time to be determined. Screening to the end of the study for each patient will be approximately 75 (±4) days.

### Justification for Study Design and Selection of Population

This is an open-label study designed primarily to assess the safety and characterize the pharmacokinetics of INL-001 in adult patients scheduled to undergo soft tissue surgery, including open ventral hernia repair, abdominoplasty, open abdominal hysterectomy, laparoscopic-assisted colectomy, and reduction mammoplasty. The open-label nature of the study allows for bupivacaine plasma samples to be obtained.

The surgical procedures selected for evaluation in the study are representative of the diversity of soft-tissue surgical procedures currently conducted. Justifications for selection of each surgery type are:

Open ventral hernia repair is representative of nonvisceral abdominopelvic procedures that may utilize a variety of surgical incisions and involve abdominal wall penetration and superficial peritoneal involvement (e.g., laparotomy, surgical abscess removal). Repair of the hernia can involve mesh placement at different subcutaneous layers, including intraperitoneal placement, and incorporates dissection and manipulation of abdominal wall muscular sheaths.

Abdominoplasty is representative of procedures with large incision lengths, incisions that involve intrusion of the superficial dermal layers and vascular surgical sites.

Laparoscopic-assisted colectomy is conducted through the use of a 3- to 6-cm abdominal incision for placement of an extraction site for removal of viscera in addition to several (e.g., 3) trocar ports (Heili et al 1999, Vanderpool and Westmoreland 2000). The extraction site incision and subsequent linea alba (if dissected), musculature, and deep fascial layer dissection provides a sufficient surface area for placement of INL-001 at multiple soft-tissue layers. Laparoscopic-assisted colectomy represents an area of superficial vasculature; however, the extensive dissection through the abdominal wall provides an opportunity to evaluate the absorption profile of INL-001 following placement at deep abdominal levels which supply blood to highly vascular muscle.

Open abdominal hysterectomy is representative of surgical procedures in which the peritoneal cavity is entered to either extract or modify visceral organs (e.g., cholecystectomy, nephrectomy, and colectomy).

Reduction mammoplasty is unique from the other soft-tissue surgical models utilized in this study in that it involves thoracic placement of INL-001 and thus different vasculature as well. In addition, the dose of INL-001 is administered across 2 separate breast pockets. Reduction mammoplasty surgery allows for the evaluation of INL-001 with substantially different vasculature than the other surgeries under investigation.

### Stopping Rules for the Study

Innocoll reserves the right to discontinue the study for safety or administrative reasons at any time.

The study will be stopped, until further benefit-risk evaluation is made, if 2 patients require removal of the INL-001 implants due to suspected systemic bupivacaine toxicity as outlined below in Section 4.3.2.

During the conduct of the study, serious adverse events will be reviewed (see Section 6.1.5), as they are reported from the investigational centers, to identify safety concerns.

The study may also be terminated by the sponsor for any reason at any time. For example, the sponsor could terminate the study in the event of: (i) new toxicologic or pharmacologic findings or safety issues from any source (e.g., other clinical studies, postmarketing experience) that invalidate the earlier positive benefit-risk assessment; or discontinuation of the development of the investigational medical product.

If the entire study is stopped or if elements of the study are stopped, the patients whose participation is terminated early will be monitored according to withdrawal criteria and procedures.

If the study is terminated prematurely, investigator(s) will inform their patients and arrange their appropriate follow-up.

### Schedule of Study Procedures and Assessments

Study procedures and assessments by visit with their respective time points are presented in Table 4. Detailed descriptions of each method of procedures and assessments are provided herein (safety assessments), (pharmacokinetics assessments), and (exploratory efficacy assessments). The end of the study is defined as the last visit of the last patient.

TABLE 4
Schedule of Procedures/Assessments

| | | | | In-patient postoperative | | | | Out-patient postoperative | | |
|---|---|---|---|---|---|---|---|---|---|---|
| | Screening | | | period ^{b} (hours relative to | | | | period (relative to day | | |
| | period | Day of surgery | | Time 0 implantation and day | | | | of surgery) | | |
| | (up to | (day 1)• | | is relative to day of surgery) | | | | | Day | Day 30/ |
| | 45 days | Immediate | Intra- | Day 2 | Day 3 | Day 4 | Day 5 | Day | 15 ±3 d | EOS ±3 d |
| Procedure/ | before | preoperative | operative | (through | (through | (through | (through | 7 ±1 d | (clinic | (clinic |
| Assessment ^{a} | surgery) | period | period | 24 h) | 48 h) | 72 h) | 96 h) | (telephone) | visit) | visit) |
| Written informed | X | | | | | | | | | |
| consent | | | | | | | | | | |
| Study-drug kit | | X | | | | | | | | |
| number assignment | | (enrolled) | | | | | | | | |
| (after preoperative | | | | | | | | | | |
| assessments) | | | | | | | | | | |
| Demographics/Medical | X | X | | | | | | | | |
| history | | (update) | | | | | | | | |
| Inclusion/Exclusion | X | X | | | | | | | | |
| criteria | | | | | | | | | | |
| Urine drug screen | X | X | | | | | | | | |
| (results before study- | | | | | | | | | | |
| drug kit assignment) | | | | | | | | | | |
| Pregnancy test (for | X | X | | | | | | | | X |
| women of childbearing | (serum) | (urine) | | | | | | | | (urine) |
| potential) (results | | | | | | | | | | |
| before study-drug | | | | | | | | | | |
| kit assignment) | | | | | | | | | | |
| Prior/Concomitant | X | [symbol] | | | | | | | | |
| medications (including | | | | | | | | | | |
| opioids and other | | | | | | | | | | |
| analgesics) | | | | | | | | | | |
| Clinical laboratory | X | | | | | | | | | X |
| tests | (blood/ | | | | | | | | | (blood |
| | urine) | | | | | | | | | only) |
| Vital signs | X | X | | X | X | X | X | | X | X |
| (see details and time | | | | | | | | | | |
| points below) | | | | | | | | | | |
| Electrocardiography | X | | | | | | | | | X |
| (12 lead) | | | | | | | | | | |
| Physical examination | X | | | | | | | | | X |
| (screening body | | | | | | | | | | |
| weight and height/ | | | | | | | | | | |
| posttreatment weight | | | | | | | | | | |
| only) | | | | | | | | | | |
| Adverse events | X | [symbol] | | | | | | | | |
| Pain Catastrophizing | X | | | | | | | | | |
| Scale (PCS) | | | | | | | | | | |
| 15-Item Quality of | X | | | X | X | X | X | X | | |
| Recovery-(QoR-15) | | | | | | | | | | |
| Blood collection | | X | | X | X | X | X | | | |
| for pharmacokinetics | | | | | | | | | | |
| (see time points | | | | | | | | | | |
| below) | | | | | | | | | | |
| Surgery/Implantation | | | X | | | | | | | |
| | | | (Time 0 is | | | | | | | |
| | | | recorded | | | | | | | |
| | | | implantation | | | | | | | |
| | | | time of first | | | | | | | |
| | | | implant) | | | | | | | |
| Assessment of signs | | | | X | X | X | X | X | X | |
| and symptoms poten- | | | | | | | | | | |
| tially indicative of | | | | | | | | | | |
| systemic bupivacaine | | | | | | | | | | |
| toxicity (see time | | | | | | | | | | |
| points below) | | | | | | | | | | |
| Assessment of signs | | | | X | X | X | X | X | X | X |
| and symptoms related | | | | | | | | | | |
| to wound healing | | | | | | | | | | |
| Southampton Wound | | | | | | X | | | X | X |
| Grading System | | | | | | | | | | |
| Numeric pain rating | | | | X | X | X | X | | | |
| scale (NPRS) (see | | | | | | | | | | |
| time points below) | | | | | | | | | | |
| Opioid Related | | | | X | X | X | X | | | |
| Symptom Distress | | | | | | | | | | |
| Scale (OR-SDS) | | | | | | | | | | |
| Patient Global | | | | X | X | X | X | X | | |
| Assessment (PGA) | | | | | | | | | | |
| Discharge ^{c} | | | | | | | X | | | |

Footnotes to table and detailed time points on next page.
Footnotes to table and detailed time points:
^{a} Procedures and assessments have timing windows that may go beyond those specified.
^{b} The times patients enter and are discharged from the postanesthesia care unit (PACU) will be recorded.
^{c} Patients will be discharged after all procedures/assessments have been completed. Whether a patient is prescribed opioid pain medication at hospital discharge will be recorded.
d = day(s); EOS = end of study; h = hour(s); m = minute(s).
Time points for record of vital signs measurements after Time 0: 0.5 hour (±5 m); 1, 2, 4 hours (±15 m); 8, 12 hours (±2 h); 24, 48, 72 hours, (each ±3 h); 96 hours (±4 h) (prior to discharge); days 15 (±3 d) and 30 (±3 d). (Vital signs include body temperature, pulse, systolic and diastolic blood pressure, respiration rate, and oxygen saturation measured by pulse oximetry.)
NOTE:
Measurements from 0.5 through 12 hours will/may occur on day 1.
Time points for assessment of signs and symptoms potentially indicative of systemic bupivacaine toxicity after Time 0: 0.5, 1, 2, 3, and 4 hours (each ±15 m), and 5, 7, 9, 12, 15, 18, 24, 48, 72, and 96 hours (each ±1 h), and days 7 (±1 day) and 15 (±3 days).
NOTE:
Assessments from 0.5 through 18 hours will/may occur on day 1.
Time points for NPRS for pain intensity after Time 0: 0.5, 1, 2, 3, 4, 5, 6, 8 hours (each ±15 m); 10, 12, 18, 20, 24, 28, 32, 36, 48, 72, 96 hours (each ±30 m). (The 0.5-hour and 1-hour NPRS assessment may be omitted if, on the basis of clinical judgment, the patient is not yet awake and alert enough to appropriately answer the NPRS after surgery. In the case of use of a rescue pain medication, scores will also be obtained within 15 minutes before any rescue medication use.)
NOTE:
Assessments done 0.5 through 20 hours will/may occur on day 1. No NPRS scores will be recorded after discharge.
Time points (hours for blood collection for pharmacokinetic assessments relative to Time 0):
(NOTE: Blood collection from 0.5 h through 18 h will/may occur on day 1.)
immediately preoperative
0.5, 1, 1.5, 2, 3, 4, 5 (all ±15 m)
6, 8, 10, 12, 18 (all ±1 h)
24, 36, 48, 72, 96 (all ±3 h)

### Treatment

### Investigational Medicinal Product Used in the Study and Other Treatment Information

The study drug used in this study is: INL-001 [XARACOLL (bupivacaine hydrochloride) implant].

INL-001 is a drug-device combination product containing 100 mg of bupivacaine HCl per implant, equivalent to 88.8 mg of bupivacaine, for placement in the surgical site. The dose to be evaluated is three 100-mg implants (300 mg bupivacaine HCl), equivalent to 266.4 mg of bupivacaine. Each implant is 5 cm×5 cm×0.5 cm in size and is white to off-white in color. Implants are terminally sterilized.

Additional details may be found in the IB for INL-001.

### Anesthetic Protocol

The anesthetic regimen used follows standard anesthetic procedures for each respective surgical model. Other than the study drug (which contains bupivacaine), no administration of local anesthetic to the patient is allowed (via intrathecal administration, nerve block, infiltration, or any other means). Lidocaine HCl 1% injection at a dose of no more than 20 mg may be administered once through iv access to decrease venous irritation (e.g., as caused by propofol) at the time of surgical anesthesia.

### Placement of Study Drug

Surgery is performed using standard surgical procedures. INL-001 is administered as three 100-mg implants, for a total dose of 300 mg bupivacaine HCL. Implants may be divided to accommodate placement, but an individual implant may not be cut into more than 2 parts. An implant may be divided disproportionately (e.g., divided into 2 pieces with the implant divided by ¼, leaving ¾ remaining, and both divisions placed), if needed to accommodate placement, but no individual division should be less than approximately ¼ of an intact implant. Placement of 3 INL-001 implants for the 5 surgery types is:

For open ventral hernia with primary suture repair and mesh underlay, inlay, or onlay:

Underlay or inlay mesh repair: Following mesh placement and peritoneal closure, 1½ matrix implants should be placed in the preperitoneal space between the closed peritoneum and posterior rectus sheath. The remaining 1½ matrix implants should be placed subcutaneously lying in direct contact with the closed anterior rectus sheath with the subcutaneous tissue closed over the matrices. Repair of multiple hernias through a single incision is permitted provided only 1 mesh is used.

Onlay mesh repair: Prior to mesh placement, 1½ matrix implants should be placed in the preperitoneal space between the closed peritoneum and posterior rectus sheath, then the remaining 1½ matrix implants should be laid in direct contact with the closed anterior rectus sheath. The mesh should then be placed over the matrices. The mesh should then be fixed and the subcutaneous tissue closed according to standard protocol. Repair of multiple hernias through a single incision is permitted provided only 1 mesh is used.

### For Abdominoplasty:

Following tissue removal and repair of the abdominal musculature (if conducted), 3 matrix implants should be placed in the subcutaneous space between the scarpa fascia and deep dermis. The implants should, to the greatest extent possible, be placed so they span the fascia that is exposed prior to surgical closure.

For Open Abdominal Hysterectomy (with or without Adnexectomy)

One implant should be placed at the vaginal vault. One matrix implant should be divided and placed at the site of the peritoneal incision between the visceral peritoneum and muscle. One matrix implant should be placed on the fascia immediately below the subcutaneous fat under the site of the incision.

### For Laparoscopic-Assisted Colectomy

One and half implants should be placed at the site of fascial repair and one and a half implants should be placed superficially above the abdominal wall musculature and beneath extraction site incision

### For Reduction Mammoplasty

One and a half implants should be placed in each breast pocket.

### Clinical Laboratory Tests

### Hematology, Serum Chemistry, and Urinalysis

Blood and urine samples for clinical laboratory tests will be collected at the screening visit and at the day 30 follow-up visit (see Table 4).

The following clinical laboratory tests are performed:

TABLE 5
Clinical Laboratory Tests

| Hematology | | |
|---|---|---|
| (complete blood count | | |
| [CBC]) | Serum chemistry: | Urinalysis |
| Hematocrit | Blood urea nitrogen | Color and |
| Hemoglobin | Creatinine | appearance |
| Platelet count | Total bilirubin | Specific gravity |
| White blood cell (WBC) | Alanine aminotransferase | pH |
| count | (ALT) | Ketones |
| WBC differential | Aspartate aminotransferase | Protein |
| (percentage and absolute): | (AST) | Blood |
| basophils | Alkaline phosphatase | Glucose |
| eosinophils | Sodium | |
| lymphocytes | Potassium | |
| monocytes | Calcium | |
| neutrophils | Chloride | |
| | Phosphate | |
| | Serum bicarbonate | |
| | Total cholesterol | |
| | Glucose (nonfasting) | |
| | Albumin | |

The following additional laboratory tests are also performed:

For women of childbearing potential, a serum sample for pregnancy test is collected at screening, and a urine sample on the day of surgery (with results available before study drug kit assignment) and on day 30.

All clinical laboratory test results outside the reference range are assessed by the investigator as belonging to one of the following categories: abnormal and not clinically significant; and abnormal and clinically significant.

A laboratory test result that is judged by the investigator as clinically significant is recorded both on the source documentation and the CRF as an adverse event and will be monitored as described herein. An event may include a laboratory or diagnostic test abnormality that results in the withdrawal of the patient from the study, the temporary or permanent withdrawal of medical treatment, or further diagnostic work up. (NOTE: Abnormal laboratory or diagnostic test results at the screening visit that preclude a patient from entering the study or receiving study drug are not considered adverse events.)

Clinical laboratory tests (serum chemistry, hematology, and urinalysis) are performed at the time points detailed in Table 4. Blood samples (approximately 16 mL total per patient) will be collected. Clinical laboratory tests are performed using the central laboratory.

### Urine Drug Screen

A urine drug screen is performed at the screening visit and immediately before surgery (see Table 4). Urine screening is done for drugs of abuse/misuse, with testing during the screening period and on the day of surgery (with results available before study drug kit number assignment).

A positive result for any excluded drugs of misuse/abuse or their metabolites without medical explanation will preclude the patient from enrollment or continued participation in the study.

### Vital Signs

Vital signs, including body temperature, pulse, systolic and diastolic blood pressure, respiration rate, and oxygen saturation, are measured/recorded at screening, preoperatively, and at the following posttreatment time points: 0.5 hours (±5 minutes); 1, 2, 4 hours (±15 minutes); 8, 12 hours (±2 hours); 24, 48, 72 hours (±3 hours); 96 (±4 hours); day 15 (±3 days); and day 30 (±3 days) (see Table 4).

Oxygen saturation will be monitored by pulse oximetry during the inpatient study period. Oxygen saturation is recorded at the individual time points listed above. Pulse oximeter alarms should be set according to clinic standards, with oxygen desaturation that occurs in concordance with the delay period and the specified limits recorded as an adverse event. Patients should be evaluated to ensure proper pulse oximeter placement and to ensure desaturation is not due to patient movement or device-related issues.

All vital sign results outside the reference ranges are judged by the investigator as belonging to one of the following categories: abnormal and not clinically significant; and abnormal and clinically significant.

### Physical Examinations

A complete physical examination are performed at screening and on day 30 (±3 days) after treatment (including screening body weight and height and posttreatment weight only) (see Table 4). A complete physical examination includes at a minimum skin, lungs, CV, respiratory, gastrointestinal, musculoskeletal, and neurological assessments. Any physical examination finding that is judged by the investigator as clinically significant (except at the screening visit) are considered an adverse event, recorded in the CRF, and monitored. Investigators should pay special attention to clinical signs related to previous serious diseases.

### Electrocardiography

A standard 12 lead ECG is performed locally and recorded (after the patient has been supine for at least 5 minutes) at screening and on day 30 (±3 days) (see Table 4). All ECG recordings is identified with the patient number, date, and time of the recording.

All ECG results outside of the reference ranges should be evaluated and are judged as belonging to one of the following categories: abnormal and not clinically significant; and abnormal and clinically significant.

Any ECG finding that is judged as clinically significant (except at the screening visit) are considered an adverse event, recorded on the source documentation and on the CRF, and monitored.

### Assessment of Pharmacokinetics

Instructions for the collection and handling of pharmacokinetic samples are provided by the sponsor. The dates and times of INL-001 placement and the date and time (24-hour clock time) of each pharmacokinetic sample collected are recorded both on the source documentation and in the CRF.

Details on sample handling, storage, shipment, and analysis are given in the laboratory manual or in respective instruction given in a separate document.

Blood samples (approximately 75 mL total per patient) are collected via indwelling catheter at the time points shown below for measurement of plasma concentration of bupivacaine.

Patients will undergo blood collection for pharmacokinetic assessments immediately preoperative, and 0.5, 1, 1.5, 2, 3, 4, 5, 6, 8, 10, 12, 18, 24, 36, 48, 72, and 96 hours after Time 0 (see Table 4).

NOTE: Time points for blood collection for pharmacokinetic assessment (pretreatment and posttreatment relative to Time 0):
immediately preoperative
0.5, 1, 1.5, 2, 3, 4, 5 hours (all ±15 minutes)
6, 8, 10, 12, 18 hours (all ±1 hour)
24, 36, 48, 72 hours (all ±3 hours)
96 hours (±4 hours)

Samples are analyzed for concentration of bupivacaine in plasma using an appropriate validated method.

The pharmacokinetic parameters to be calculated for each patient include:
maximum observed plasma concentration (Cₘₐₓ)
time to maximum observed plasma concentration (Tₘₐₓ)
time of first measurable plasma concentration after Time 0 (T_{lag})
terminal elimination half-life (t_{1/2})
terminal phase rate constant (γ_{Z})
area under the plasma concentration-time curve (AUC) through last observed concentration (AUC₀₋ₗₐₛₜ)
AUC extrapolated through infinity (AUC_{0-∞})

A pharmacokinetics analysis plan (PAP) will be developed and approved before the final pharmacokinetic analysis, and will include the methods to calculate the pharmacokinetic parameters and detail on the summary of pharmacokinetic parameters.

### Example 12: Non-Clinical Study for Postoperative Analgesic Efficacy and Safety of INL-001 Versus Placebo Collagen Implant in Patients Undergoing Abdominoplasty

Bupivacaine has been widely used as a local anesthetic and has demonstrated efficacy in animal models in multiple species (Li et al 2013, Hersh et al 1992). The pharmacology of bupivacaine is well understood.

The distribution, metabolism, and excretion of bupivacaine across species is characterized in the literature including data showing that, following absorption, bupivacaine is rapidly and readily distributed to tissues, with the highest concentrations in highly perfused tissues. In general, the metabolic profile of bupivacaine is similar across species although quantitatively different, with no novel metabolite identified in humans (Goehl et al 1973, Carson 2000). The excretion of bupivacaine in monkeys and humans is predominantly in the urine; in rats, excretion in urine and feces is generally similar; and in dogs, only small amounts of excretion of bupivacaine in the urine have been reported. The dog excretion data in the literature for bupivacaine is consistent with excretion data obtained in the dog following implantation of INL-001, which showed approximately 0.16% of the bupivacaine dose was collected in the urine over a 72-hour period. The genotoxicity of bupivacaine HCl was evaluated in 4 in vitro studies and 2 in vivo studies, with no safety concerns identified.

Clinically significant pharmacokinetic drug interactions with bupivacaine have been reported with verapamil, diazepam, and cimetidine in humans, animals, and/or in vitro test systems. These interactions occurred with bupivacaine administered epidurally or intravenously (iv).

The uniformity of bupivacaine in the INL-001 implant and the release of bupivacaine from the implant was evaluated in in vitro and/or in vivo assessments. The in vitro uniformity study demonstrated that bupivacaine content is homogeneously dispersed throughout INL 001. An in vitro dissolution study of a whole matrix found that bupivacaine was released as early as 5 minutes and complete release of bupivacaine occurred by 24 hours. An in vivo study showed the in vitro drug release profile was similar to that seen in vivo in dogs with INL-001 implanted into the abdomen and subcutaneous tissues through a surgical incision.

Two studies were conducted in which INL-001 was surgically implanted in rats followed by a 56 day postsurgical period to assess potential local and systemic effects, determine potential effects on wound healing, and characterize the resolution of any implant related findings. The initial study was conducted with early development drug product and the second study with the clinical Phase 3/commercial formulation using generally the same study design. INL-001 was well tolerated following implantation. By day 28 following INL-001 implantation, attrition of the implant was approximately 95% and the implant was not observed microscopically by day 56 after dosing. INL-001-associated findings were limited to observations at the injection site, including transient edema through day 10 and microscopic findings associated with the repair process.

Biocompatibility studies were conducted with extracts from INL-001 and the drug-free implant; these studies did not identify any safety concerns. There was no evidence of acute systemic toxicity or effects on body temperature (i.e., pyrogenicity) associated with administration of drug free implant extract or evidence of skin irritation, sensitization, or genotoxicity following exposure to bupivacaine implant extract.

### Example 11: Clinical Study for Postoperative Analgesic Efficacy and Safety of INL-001 Versus Placebo Collagen Implant in Patients Undergoing Abdominoplasty

The clinical pharmacology, efficacy, and safety of INL-001 (at single doses of 100, 150, 200, and 300 mg) have been evaluated in 11 completed clinical studies (Phases 1-3) in adults, including 2 well controlled pivotal Phase 3 studies of INL-001 in open inguinal hernia repair, at its recommended approved dose (three 100-mg bupivacaine HCl implants).

In a pharmacokinetic/relative bioavailability study in patients following inguinal hernioplasty (Study INN-CB-022), the commercial formulation of INL 001 at a dose of 300 mg was compared with Marcaine 0.25% injection at a dose of 175 mg (the maximum recommended single dose). Quantifiable bupivacaine concentrations were evident at the first posttreatment time point measured (30 minutes) for all patients treated with INL-001 or Marcaine. Bupivacaine concentrations were detectable through the 96-hour posttreatment time point (last time point) in both treatment groups but at higher concentrations with INL-001 than with Marcaine. Pharmacokinetic analysis led to the following additional conclusions:

For the INL-001 treatment group, the mean maximum observed plasma concentration (Cₘₐₓ) (minimum, maximum) was 663.412 ng/mL (274.00 ng/mL, 1230.00 ng/mL) compared with a mean Cₘₐₓ (minimum, maximum) for the Marcaine treatment group of 641.000 ng/mL (275.00 ng/mL, 1140.00 ng/mL).

For the INL-001 treatment group, the median time to maximum observed plasma concentration (Tₘₐₓ) was 3.03 hours with a mean terminal elimination half-life (t_{1/2}) of 18.95 hours compared with a Tₘₐₓ of 1.01 hours and a mean t_{1/2} of 9.08 hours for the Marcaine treatment group.

For INL-001, the geometric means for area under the concentration-time curve (AUC) through last observed concentration (AUC₀₋ₗₐₛₜ) was 18186.9 h·ng/mL and the AUC extrapolated through infinity (AUC₀₋ₗₐₛₜ) was 19012.5 h·ng/mL. For Marcaine, the geometric means for AUC₀₋ₗₐₛₜ and AUC₀₋ₗₐₛₜ were 8836.9 h·ng/mL and 8920.1 h·ng/mL, respectively.

In clinical studies of an earlier bupivacaine collagen implant formulation, following implantation of INL-001 at 100, 150, and 200 mg in various abdominopelvic surgeries, quantifiable bupivacaine plasma concentrations were observed from 30 minutes after placement (at the first posttreatment time point). Bupivacaine concentrations increased in a slightly higher than dose-proportional manner with increasing doses of INL-001.

The efficacy of INL-001 was evaluated in 2 multicenter, double-blind, placebo controlled Phase 3 studies in adults that independently demonstrated the effectiveness of locally placed INL 001 (300 mg implanted in layers at the surgical site) in reducing both pain intensity and the need for opioid rescue analgesia after surgery; together, these studies demonstrate the reproducibility of the INL-001 treatment effects (Studies INN-CB-014 and INN-CB-016). In each study, INL-001 achieved the primary endpoint, with patients treated with INL-001 experiencing statistically significantly less pain (p≤0.0004) as evaluated by the (time weighted) sum of pain intensity through 24 hours (SPI24). These reductions in pain intensity were coupled with less total opioid rescue analgesic medication use in the INL-001 treatment group compared with the placebo implant group. In data pooled from the 2 pivotal studies, patients used statistically significantly (p≤0.0004) less opioid rescue analgesia and had statistically significantly (p=0.0007) fewer opioid-related treatment emergent adverse events (i.e., nausea, vomiting, and constipation) over the post-implantation period compared with subjects in the combined placebo implant group.

The existing INL-001 safety database is derived from a clinical development program of 11 studies conducted in soft tissue surgeries in adults, including the 2 positive Phase 3 studies in inguinal hernia repair. A total of 892 adult patients have received collagen matrix implants in this program (612 INL-001 and 280 placebo implants). Of the 892 patients, 816 patients underwent inguinal hernia repair, 69 patients underwent hysterectomy, and 7 patients underwent other types of soft-tissue surgeries (i.e., nonlaparoscopic benign gynecological procedure other than hysterectomy or elective surgery requiring a vertical or transverse abdominal incision).

Across these 11 studies:

Adverse events occurring at an incidence of 2% or more patients following administration of INL-001 at 300 mg and at a higher incidence than placebo implants, respectively, were somnolence (19.2% vs 13.9%), dizziness (16.4% vs 13.9%), incision site swelling (13% vs 10.7%), incision site pain (11.7% vs 11.4%), restlessness (7.7% vs 6.8%), dysgeusia (7.5% vs 4.6%), vision blurred (4.9% vs 2.1%), headache (4.7% vs 2.1%), tremor (4.5% vs 2.1%), postprocedural discharge (4.3% vs 3.6%), scrotal swelling (2.8% vs 1.8%), seroma (2.6% vs 1.8%), oral hypoesthesia (2.6% vs 1.4%), pyrexia (2.3% vs 1.8%), and wound dehiscence (2.1% vs 1.8%).

In these studies, adverse events considered by the investigator to be treatment related following INL-001 placement in the surgical site occurred at a rate of 1.5% or less. The only treatment-related adverse event that occurred in 1% or more of all patients who received INL-001 at 300 mg was dysgeusia (1.3%), which also occurred in 0.7% of patients in the placebo group.

Across the INL-001 clinical development program, 16 patients experienced 1 or more serious adverse events: 11 patients in the INL-001 (including earlier formulation) treatment group and 5 patients in the placebo implant or other comparator group. Serious adverse events reported in the INL-001 treatment group included wound infection and seroma. One patient had the INL-001 implant removed after placement of an earlier formulation of bupivacaine collagen matrix implant during bladder sling surgery (see event described below).

There were no verbatim reports of systemic bupivacaine toxicity or LAST during any inguinal hernia repair study done as part of the development program for INL-001. The safety assessments conducted during the development program included monitoring adverse events, measurement of vital signs, and assessments with multiday cardiac Holter monitors. These assessments revealed no constellation of neurologic or cardiovascular (CV) signs or symptoms to suggest systemic bupivacaine toxicity in patients undergoing open inguinal hernia repair receiving INL 001.

One patient experienced signs and symptoms thought to be consistent with LAST approximately 4 hours after administration of an earlier formulation of the INL-001 (at 150 mg) following bladder sling surgery. Treatment included administration of lipid emulsion and surgical removal of the INL-001 implants.

Across the INL-001 clinical development program, incision-site adverse events occurring with an incidence of 2% or more in either the INL-001 (including earlier formulation) or placebo group compared with a non-implant comparator treatment group (n=52) included swelling, pain, other complication, postprocedural discharge, erythema, dehiscence, and inflammation.

### Example 13: Study for Postoperative Analgesic Efficacy and Safety of INL-001 Versus Placebo Collagen Implant in Patients Undergoing Abdominoplasty

This is a multicenter, randomized, double-blind, placebo-controlled efficacy and safety study of the INL 001 (bupivacaine HCl) implant, at 300 mg, in patients following abdominoplasty. On the day of surgery (study day 1), eligible patients are randomly assigned to treatment in a 1:1 ratio to receive either INL-001 (three 100-mg implants containing a total dose of 300 mg of bupivacaine HCl) or 3 placebo collagen implants. Patients then undergo abdominoplasty under general anesthesia and have INL-001 or placebo implanted intraoperatively.

The duration of study participation for each patient is a maximum of 75 (±4) days, consisting of a screening period (up to 45 days before surgery), an inpatient period (preoperative, intraoperative, immediately postoperative) of approximately 4 days, and an outpatient follow-up period (up to 30 days [±3 days] after treatment) including an end-of-study visit. Efficacy assessments are made through 72 hours after treatment (after implant placement). Posttreatment (time measured from Time 0 [placement of first implant]) safety assessments are made throughout the study after the informed consent form (ICF) is signed, and as specifically scheduled through 72 hours posttreatment, on day 7 (±1 day) (telephone), on day 15 (±3 days) (clinic visit), and on day 30 (±3 days) (clinic visit). Unless the investigator determines further hospitalization is necessary, the patient is discharged on the day occurring 72 hours (day 4) after surgery.

During the screening period, all patients provide informed consent and undergo eligibility and other screening and safety assessments (medical history including review of prior medications, physical examination, urine drug screen, serum pregnancy test for women of childbearing potential, clinical laboratory tests [hematology, chemistry, urinalysis], vital signs measurement, and 12-lead electrocardiography [ECG]). Vital signs include body temperature, pulse, systolic and diastolic blood pressure, respiration rate, and oxygen saturation measured by pulse oximetry. The reason(s) a patient does not meet screening criteria is recorded, if applicable. Recording of adverse events and concomitant medication use commences once a patient signs the ICF.

On the day of surgery (day 1), patient eligibility is reconfirmed before the start of surgery (including medical history, urine drug screen, urine pregnancy test for women of childbearing potential, vital signs), patients are randomly assigned to treatment with study drug (INL-001 or placebo collagen implant), and adverse events and prior/concomitant medications are reviewed.

Patients undergo an abdominoplasty with rectus sheath plication using standard surgical procedures conducted under general anesthesia (see anesthesia protocol below in Section 5.1.1), with no other local anesthetic used at the surgical site. All patients should undergo an abdominoplasty procedure with an incision that does not extend above the umbilicus. The approach should be anterior. The incision should in general be from one anterior superior iliac spine (ASIS) to the other. The exact incision length may vary depending on the patient's anatomy and the desired cosmetic outcome. All packs/gauze should be removed and adequate hemostasis must be achieved prior to skin closure. Surgical drains should be placed at the discretion of the surgeon and their use recorded. Ancillary procedures (e.g., liposuction, breast augmentation/reduction) are prohibited.

Placement of study drug is detailed elsewhere herein. The time of the first placement of study drug (placement of first implant) is considered Time 0 and is recorded. Use of analgesic and all medications during surgery is recorded. At the surgeon's discretion, if a significant surgical/medical complication is encountered during surgery, study drug will not be implanted and the patient is considered enrolled but not treated.

After surgery, patients are transferred to a postanesthesia care unit (PACU) or other postoperative recovery area for monitored observation. The times patients enter and are discharged from the PACU are recorded to calculate time to discharge from the PACU. Patients are monitored with pulse oximetry starting in the PACU through 24 hours posttreatment. After leaving the PACU (time in PACU to be at the discretion of the investigator), patients are placed in the postoperative unit or clinical research unit for domiciled observation. Vital signs, including body temperature, pulse, systolic and diastolic blood pressure, respiration rate, and oxygen saturation measured by pulse oximetry, are assessed at multiple time points through discharge, and in the clinic on days 15 and 30; 12-lead ECG will be done on day 30.

Adverse event and concomitant medication information, including use of rescue medication, are collected throughout the study (inpatient and outpatient). Surgical wound healing assessments are made at 24, 48, and 72 hours after Time 0 and on days 7, 15, and 30 using the specified list and assessed for and recorded as adverse events as appropriate. The Southampton Wound Grading System is also completed 72 hours posttreatment/prior to discharge (±4 hours) and on days 15 and 30. Assessment for signs and symptoms potentially indicative of systemic bupivacaine toxicity is made after Time 0 at the following time points: 0.5, 1, 2, 3, and 4 hours (each ±15 minutes), and 5, 7, 9, 12, 15, 18, 24, 48, and 72 hours (each ±1 hour), and days 7 (±1 day) and 15 (±3 days) using the specified list and assessment made and recorded as adverse events as appropriate.

At any time that a patient is determined to be exhibiting signs and/or symptoms suggestive of systemic bupivacaine toxicity, at the discretion of the investigator, a bupivacaine blood sample will be collected and 12 lead ECG will be performed. The patient may be treated at the discretion of the investigator, including obtaining repeat bupivacaine blood levels, 12-lead ECG, or removal of the implants.

After surgery, patient reports of pain intensity using an 11 point numeric pain rating scale (NPRS) are recorded at multiple time points through 72 hours posttreatment. Scheduled pain intensity scores are recorded after Time 0 at 0.5, 1, 2, 3, 4, 5, 6, 8, 10, 12, 18, 20, 24, 28, 32, 36, 48, and 72 hours. Each assessment prior to hour 10 has a ±15-minute window; each assessment after and including hour 10 has a ±30-minute window. The 0.5 hour and 1 hour NPRS assessments may be omitted if, on the basis of clinical judgement, the patient is not yet awake and alert enough to appropriately answer the NPRS after surgery. Pain intensity assessments scheduled between 2400 (midnight) and 0600 (6 am) may be limited to collection every 4 hours if the patient is sleeping. However, consecutive pain assessments may not be missed, and the hour 12, 24, 48, and 72 posttreatment pain assessments must be completed even if they fall between 2400 (midnight) and 0600 (6 am). A pain intensity score is also collected before any rescue pain medication use.

Patients are permitted rescue medication to manage breakthrough pain when it occurs. Oral acetaminophen at 1000 mg every 4-6 hours as needed for pain (maximum daily dosage 3000 mg) and/or oxycodone 5-mg tablet(s) may be given (not to exceed 10 mg in a 4-hour period during the inpatient stay). Immediately prior to receiving any rescue medication, a pain intensity score must be recorded. If the NPRS score is 4 or less, patients are discouraged from taking opioid rescue medication; however, rescue medication may be requested and provided at any time. If patients require opioid rescue medication, but are unable to take oral medications, they are permitted to receive intravenous (iv) morphine (2-3 mg) every 3 hours until they are able to take oral rescue medication. As assessed by the investigator, if a patient's pain is not relieved by oxycodone and/or acetaminophen, the patient is not yet eligible for further treatment with oxycodone and/or acetaminophen, and more than 3 hours have passed since the previous iv morphine dose, a patient may receive a dose of iv morphine (2-3 mg) for pain relief. If the pain remains unrelieved or increases in intensity before additional rescue medication is allowed, additional treatment options will be discussed with the medical monitor.

Following discharge, to report an adverse event, a patient contacts study staff by telephone and report adverse event information, including incidence, duration, and any associated treatment. Patients with pain intensity scores of 4 or more at discharge are given a written prescription for immediate release oxycodone at a dosage of 5-10 mg every 4-6 hours as needed as rescue medication for breakthrough pain. Patients prescribed opioid rescue medication are also permitted to take oral acetaminophen at 1000 mg every 4-6 hours (maximum daily dosage 3000 mg) and/or ibuprofen at 400 mg every 4-6 hours as needed for pain, on an outpatient basis. Patients with pain intensity scores of less than 4 at discharge are instructed to take oral acetaminophen at 1000 mg every 4-6 hours as needed for pain (maximum daily dosage 3000 mg) and/or ibuprofen at 400 mg every 4-6 hours as needed for pain, on an outpatient basis. Patients who do not receive a written prescription for oxycodone upon discharge are permitted to request immediate-release oxycodone 5-10 mg if their pain is unrelieved by acetaminophen. Use of opioids, acetaminophen, nonsteroidal anti-inflammatory drugs (NSAIDs), or any other medications after discharge from the hospital is recorded, with data reviewed by study staff at subsequent contacts.

A detailed table of procedures and assessments is provided in Table 6.

### Planned Number of Patients and Countries

Up to approximately 432 patients will be screened in order to randomize 372 patients to achieve a minimum of 360 patients randomized and treated [180 evaluable patients per treatment group]).

The study is expected to start at a time to be determined, with an estimated enrollment period of approximately 6 months, including an interim analysis. Screening to the end of the study for each patient will be approximately 75 (±4) days.

### Justification for Study Design and Selection of Population

This is a randomized, double-blind, placebo-controlled study designed primarily to assess the efficacy and safety of INL-001 in adult patients scheduled for abdominoplasty. The study design employed in this study has previously been used successfully in 2 clinical Phase 3 studies conducted to demonstrate the efficacy and safety of INL 001 to manage acute postoperative pain in patients undergoing open inguinal hernia repair.

### Stopping Rules for the Study

Innocoll reserves the right to discontinue the study for safety or administrative reasons at any time.

The study will be stopped, until further benefit-risk evaluation is made, if 2 patients require removal of the INL-001 implants due to suspected systemic bupivacaine toxicity.

During the conduct of the study, serious adverse events are reviewed, as they are reported from the investigational centers, to identify safety concerns.

The study may also be terminated by the sponsor for any reason at any time. For example, the sponsor could terminate the study in the event of (i) new toxicologic or pharmacologic findings or safety issues from any source (e.g., other clinical studies, postmarketing experience) that invalidate the earlier positive benefit-risk assessment; or (ii) discontinuation of the development of the investigational medical product.

If the entire study is stopped or if elements of the study are stopped, the patients whose participation is terminated early will be monitored according to withdrawal criteria and procedures.

If the study is terminated prematurely, investigator(s) will inform their patients and arrange their appropriate follow-up.

### Schedule of Study Procedures and Assessments

Study procedures and assessments by visit with their respective time points are presented in Table 6. The end of study is defined as the last visit of the last patient.

TABLE 6
Schedule of Procedures/Assessments

| | | | | In-patient postoperative period ^{b} | | | | | |
|---|---|---|---|---|---|---|---|---|---|
| | Screening | | | (hours relative to Time 0 | | | Out-patient postoperative period | | |
| | period | Day of surgery | | implantation and day is relative to | | | (relative to day of surgery) | | |
| | (up to | (day 1)• | | day of surgery) | | | | Day | Day 30/ |
| | 45 days | Immediate | Intra- | Day 2 | Day 3 | Day 4 | Day | 15 ±3 d | EOS ±3 d |
| Procedure/ | before | preoperative | operative | (through | (through | (through | 7 ±1 d | (clinic | (clinic |
| Assessment ^{a} | surgery) | period | period | 24 h) | 48 h) | 72 h) | (telephone) | visit) | visit) |
| Written informed | X | | | | | | | | |
| consent | | | | | | | | | |
| Study-drug kit | | X | | | | | | | |
| number assignment | | (enrolled) | | | | | | | |
| (after preoperative | | | | | | | | | |
| assessments) | | | | | | | | | |
| Demographics/Medical | X | X | | | | | | | |
| history | | (update) | | | | | | | |
| Inclusion/Exclusion | X | X | | | | | | | |
| criteria | | | | | | | | | |
| Urine drug screen | X | X | | | | | | | |
| (results before study- | | | | | | | | | |
| drug kit assignment) | | | | | | | | | |
| Pregnancy test (for | X | X | | | | | | | X |
| women of childbearing | (serum) | (urine) | | | | | | | (urine) |
| potential) (results | | | | | | | | | |
| before study-drug | | | | | | | | | |
| kit assignment) | | | | | | | | | |
| Prior/Concomitant | X | [symbol] | | | | | | | |
| medications (including | | | | | | | | | |
| opioids and other | | | | | | | | | |
| analgesics) | | | | | | | | | |
| Clinical laboratory | X | | | | | | | | X |
| tests | (blood/ | | | | | | | | (blood |
| | urine) | | | | | | | | only) |
| Vital signs | X | X | | X | X | X | | X | X |
| (see details and time | | | | | | | | | |
| points below) | | | | | | | | | |
| Electrocardiography | X | | | | | | | | X |
| (12 lead) | | | | | | | | | |
| Physical examination | X | | | | | | | | X |
| (screening body | | | | | | | | | |
| weight and height/ | | | | | | | | | |
| posttreatment weight | | | | | | | | | |
| only) | | | | | | | | | |
| Adverse events | X | [symbol] | | | | | | | |
| Surgery/Implantation | | | X | | | | | | |
| | | | (Time 0 is | | | | | | |
| | | | recorded | | | | | | |
| | | | implantation | | | | | | |
| | | | time of first | | | | | | |
| | | | implant) | | | | | | |
| Assessment of signs | | | | X | X | X | X | X | |
| and symptoms poten- | | | | | | | | | |
| tially indicative of | | | | | | | | | |
| systemic bupivacaine | | | | | | | | | |
| toxicity (see time | | | | | | | | | |
| points below) | | | | | | | | | |
| Assessment of signs | | | | X | X | X | X | X | X |
| and symptoms related | | | | | | (prior to | | | |
| to wound healing | | | | | | discharge) | | | |
| Southampton Wound | | | | | | X | | X | X |
| Grading System | | | | | | (prior to | | | |
| | | | | | | discharge) | | | |
| Numeric pain rating | | | | X | X | X | | | |
| scale (NPRS) (see | | | | | | | | | |
| time points below) | | | | | | | | | |
| Discharge ^{c} | | | | | | X | | | |

Footnotes to table and detailed time points on next page.
Footnotes to table and detailed time points:
^{a} Procedures and assessments have timing windows that may go beyond those specified.
^{b} The times patients enter and are discharged from the postanesthesia care unit (PACU) will be recorded.
^{c} Patients will be discharged after all procedures/assessments have been completed. Whether a patient is prescribed opioid pain medication at hospital discharge will be recorded.
d = day(s); EOS = end of study; h = hour(s); m = minute(s).
Time points for record of vital signs measurements after Time 0: 0.5 hour (±5 m); 1, 2, 4 hours (±15 m); 8, 12 hours (±2 h); 24, 48 hours (±3 h); 72 hours (±4 h) (prior to discharge); days 15 (±3 d) and 30 (±3 d). (Vital signs include body temperature, pulse, systolic and diastolic blood pressure, respiration rate, and oxygen saturation measured by pulse oximetry.)
NOTE:
Measurements from 0.5 through 12 hours will/may occur on day 1.
Time points for assessment of signs and symptoms potentially indicative of systemic bupivacaine toxicity after Time 0: 0.5, 1, 2, 3, and 4 hours (each ±15 m); 5, 7, 9, 12, 15, 18, 24, 48, and 72 hours (each ±1 h); and days 7 (±1 day) and 15 (±3 days).
NOTE:
Assessments from 0.5 through 18 hours will/may occur on day 1.
Time points for NPRS for pain intensity after Time 0: 0.5, 1, 2, 3, 4, 5, 6, 8 hours (each ±15 m); 10, 12, 18, 20, 24, 28, 32, 36, 48, 72 hours (each ±30 m). (The 0.5-hour and 1-hour NPRS assessment may be omitted if, on the basis of clinical judgment, the patient is not yet awake and alert enough to appropriately answer the NPRS after surgery. In the case of use of a rescue pain medication, scores will also be obtained within 15 minutes before any rescue medication use.)
NOTE:
Assessments done 0.5 through 20 hours will/may occur on day 1. No NPRS scores will be collected after discharge.

### Treatment

### Investigational Medicinal Product Used in the Study and Other Treatment Information

The study drugs used in this study are: INL-001 [XARACOLL (bupivacaine hydrochloride) implant] and placebo collagen implant.

INL-001 is a drug-device combination product containing 100 mg of bupivacaine HCl per implant, equivalent to 88.8 mg of bupivacaine, for placement in the surgical site. The dose to be evaluated is three 100-mg implants (300 mg bupivacaine HCl), equivalent to 266.4 mg of bupivacaine. Each implant is 5 cm×5 cm×0.5 cm in size and is white to off-white in color. Placebo implants contain collagen but no bupivacaine. Implants are terminally sterilized.

Additional details may be found in the IB for INL-001.

### Anesthetic Protocol

The standardized anesthetic regimen includes general anesthesia with fentanyl (maximum dose of 4 mcg/kg) and propofol (dose at discretion of the anesthesia provider), with or without volatile anesthetics or muscle relaxants. The standardized anesthetic regimen is a guide that should be followed to minimize interpatient variability to the greatest extent possible. However, it is understood that hemodynamic fluctuations and other intraoperative events may necessitate some deviation from this standard regimen. Neuraxial techniques, such as epidural and spinal anesthesia, are not allowed. No epinephrine is permitted during the procedure. No local anesthetic other than INL-001 (study drug) in the surgical field or regional anesthesia is permitted. Lidocaine HCl 1% injection at a dose of no more than 20 mg may be administered once through iv access to decrease venous irritation (e.g., as caused by propofol) at the time of surgical anesthesia. Intraoperatively, fentanyl (maximum dose of 4 mcg/kg) is permitted for analgesia. No other analgesic agents may be used during the procedure including, but not limited to, opioids (other than fentanyl), acetaminophen (oral or iv), NSAIDs (e.g., ketorolac or COX-2 inhibitors), ketamine, pregabalin, and others. A preoperative dose of an antiemetic, ondansetron iv 4 mg, for nausea prophylaxis is allowed; however, postoperative antiemetic medications should be given to treat only patients who report nausea and/or vomiting. Administration of fentanyl should be avoided 30 minutes prior to the anticipated conclusion of the procedure if medically acceptable in the judgement of the anesthesiologist.

### Placement of Study Drug

Following tissue removal and repair of the abdominal musculature, 2 implants should be placed on the rectus diastasis at the site of rectus sheath plication and 1 implant should be placed below the abdominal incision between Scarpa's fascia and the subcutaneous fat. The implants should, to the greatest extent possible, be placed so they span the fascia that is exposed prior to surgical closure. Implants may be divided to accommodate placement, but an individual implant may not be cut into more than 2 halves.

### Clinical Laboratory Tests

### Hematology, Serum Chemistry, and Urinalysis

Blood and urine samples for clinical laboratory tests are collected at the screening visit and at the day 30 follow-up visit (see Table 6).

The following clinical laboratory tests are performed:

TABLE 7
Clinical Laboratory Tests

| Hematology | | |
|---|---|---|
| (complete blood | Serum | |
| count [CBC]) | chemistry: | Urinalysis |
| Hematocrit | Blood urea nitrogen | Color and |
| Hemoglobin | Creatinine | appearance |
| Platelet count | Total bilirubin | Specific gravity |
| White blood cell | Alanine | pH |
| (WBC) count | aminotransferase (ALT) | Ketones |
| WBC differential | Aspartate | Protein |
| (percentage and absolute): | aminotransferase (AST) | Blood |
| basophils | Alkaline phosphatase | Glucose |
| eosinophils | Sodium | |
| lymphocytes | Potassium | |
| monocytes | Calcium | |
| neutrophils | Chloride | |
| | Phosphate | |
| | Serum bicarbonate | |
| | Total cholesterol | |
| | Glucose (nonfasting) | |
| | Albumin | |

The following additional laboratory tests are also performed:

For women of childbearing potential, a serum sample for pregnancy test is collected at screening, and a urine sample on the day of surgery (with results available before study drug kit assignment) and on day 30.

All clinical laboratory test results outside the reference range are assessed by the investigator as belonging to one of the following categories: abnormal and not clinically significant; and abnormal and clinically significant.

A laboratory test result that is judged by the investigator as clinically significant will be recorded both on the source documentation and the CRF as an adverse event and will be monitored. An event may include a laboratory or diagnostic test abnormality that results in the withdrawal of the patient from the study, the temporary or permanent withdrawal of medical treatment, or further diagnostic work up. (NOTE: Abnormal laboratory or diagnostic test results at the screening visit that preclude a patient from entering the study or receiving study drug are not considered adverse events.)

Clinical laboratory tests (serum chemistry, hematology, and urinalysis) are performed at the time points detailed in Table 6. Blood samples (approximately 16 mL total per patient) are collected. Clinical laboratory tests will be performed using the central laboratory.

### Urine Drug Screen

A urine drug screen is performed at the screening visit and immediately before surgery (see Table 6). Urine screening is done for drugs of abuse/misuse, with testing during the screening period and on the day of surgery (with results available before study drug kit number assignment).

A positive result for any excluded drugs of misuse/abuse or their metabolites without medical explanation will preclude the patient from enrollment or continued participation in the study.

### Vital Signs

Vital signs, including body temperature, pulse, systolic and diastolic blood pressure, respiration rate, and oxygen saturation, are measured/recorded at screening, preoperatively, and at the following posttreatment time points: 0.5 hours (±5 minutes); 1, 2, 4 hours (±15 minutes); 8, 12 hours (±2 hours); 24, 48, 72 hours (±4 hours) prior to discharge; day 15 (±3 days); and day 30 (±3 days) (see Table 6).

Oxygen saturation is monitored by pulse oximetry during the inpatient study period. Oxygen saturation will be recorded at the individual time points listed above. Pulse oximeter alarms should be set according to clinic standards, with oxygen desaturation that occurs in concordance with the delay period and the specified limits recorded as an adverse event. Patients should be evaluated to ensure proper pulse oximeter placement and to ensure desaturation is not due to patient movement or device-related issues.

All vital sign results outside the reference ranges are judged by the investigator as belonging to one of the following categories: abnormal and not clinically significant; and abnormal and clinically significant.

### Physical Examinations

A complete physical examination is performed at screening and on day 30 (±3 days) after treatment (including screening body weight and height and posttreatment weight only) (see Table 6). A complete physical examination includes at a minimum skin, lungs, CV, respiratory, gastrointestinal, musculoskeletal, and neurological assessments. Any physical examination finding that is judged by the investigator as clinically significant (except at the screening visit) is considered an adverse event, recorded in the CRF, and monitored. Investigators should pay special attention to clinical signs related to previous serious diseases.

### Electrocardiography

A standard 12 lead ECG is performed locally and recorded (after the patient has been supine for at least 5 minutes) at screening and on day 30 (±3 days) (see Table 6). All ECG recordings are identified with the patient number, date, and time of the recording.

All ECG results outside of the reference ranges should be evaluated and are judged as belonging to one of the following categories: abnormal and not clinically significant; and abnormal and clinically significant.

Any ECG finding that is judged as clinically significant (except at the screening visit) will be considered an adverse event, recorded on the source documentation and on the CRF, and monitored.

### Example 14: A Randomized, Double-Blind, Placebo-Controlled Study to Evaluate the Efficacy and Safety of a 300-Mg Dose of the INL-001 (Bupivacaine Hydrochloride) Implant in Patients Undergoing Abdominoplasty

The primary objective of the study is to evaluate the analgesic effect of treatment (i.e., efficacy) with INL-001 implants compared with placebo implants after placement into the surgical site during abdominoplasty.

The secondary objective is to assess the safety and tolerability of INL-001 implants after placement in the surgical site during abdominoplasty.

### General Study Design

The study is a multicenter, randomized, double-blind, placebo-controlled efficacy and safety study of the INL-001 (bupivacaine HCl) implant, at 300 mg, in patients following abdominoplasty. On the day of surgery (study day 1), eligible patients will be randomly assigned to treatment in a 1:1 ratio to receive either INL-001 (three 100-mg implants containing a total dose of 300 mg of bupivacaine HCl) or 3 placebo collagen implants. Patients will then undergo abdominoplasty under general anesthesia and have INL-001 or placebo implanted intraoperatively.

The duration of study participation for each patient will be a maximum of 75 (±4) days, consisting of a screening period (up to 45 days before surgery), an inpatient period (preoperative, intraoperative, immediately postoperative) of approximately 4 days, and an outpatient follow-up period (up to 30 days [±3 days] after treatment) including an end-of-study visit. Efficacy assessments will be made through 72 hours after treatment (after implant placement). Posttreatment (time measured from Time 0 [placement of first implant]) safety assessments will be made throughout the study after the informed consent form (ICF) is signed, and as specifically scheduled through 72 hours posttreatment, on day 7 (±1 day) (telephone), on day 15 (±3 days) (clinic visit), and on day 30 (±3 days) (clinic visit). Unless the investigator determines further hospitalization is necessary, the patient will be discharged on the day occurring 72 hours (day 4) after surgery.

During the screening period, all patients will provide informed consent and undergo eligibility and other screening and safety assessments (medical history including review of prior medications, physical examination, urine drug screen, serum pregnancy test for women of childbearing potential, clinical laboratory tests [hematology, chemistry, urinalysis], vital signs measurement, and 12-lead electrocardiography [ECG]). Vital signs include body temperature, pulse, systolic and diastolic blood pressure, respiration rate, and oxygen saturation measured by pulse oximetry. The reason(s) a patient does not meet screening criteria will be recorded, if applicable. Recording of adverse events and concomitant medication use will commence once a patient signs the ICF.

On the day of surgery (day 1), patient eligibility will be reconfirmed before the start of surgery (including medical history, urine drug screen, urine pregnancy test for women of childbearing potential, vital signs), patients will be randomly assigned to treatment with study drug (INL-001 or placebo collagen implant), and adverse events and prior/concomitant medications will be reviewed.

Patients will undergo an abdominoplasty with rectus sheath plication using standard surgical procedures conducted under general anesthesia, with no other local anesthetic used at the surgical site. All patients should undergo an abdominoplasty procedure with an incision that does not extend above the umbilicus. The approach should be anterior. The incision should in general be from one anterior superior iliac spine (ASIS) to the other. The exact incision length may vary depending on the patient's anatomy and the desired cosmetic outcome. All packs/gauze should be removed and adequate hemostasis must be achieved prior to skin closure. Surgical drains should be placed at the discretion of the surgeon and their use recorded. Ancillary procedures (e.g., liposuction, breast augmentation/reduction) are prohibited.

The time of the first placement of study drug (placement of first implant) is considered Time 0 and will be recorded. Use of analgesic and all medications during surgery will be recorded. At the surgeon's discretion, if a significant surgical/medical complication is encountered during surgery, study drug will not be implanted and the patient will be considered enrolled but not treated.

After surgery, patients will be transferred to a postanesthesia care unit (PACU) or other postoperative recovery area for monitored observation. The times patients enter and are discharged from the PACU will be recorded to calculate time to discharge from the PACU. Patients will be monitored with pulse oximetry starting in the PACU through 24 hours posttreatment. After leaving the PACU (time in PACU to be at the discretion of the investigator), patients will be placed in the postoperative unit or clinical research unit for domiciled observation. Vital signs, including body temperature, pulse, systolic and diastolic blood pressure, respiration rate, and oxygen saturation measured by pulse oximetry, will be assessed at multiple time points through discharge, and in the clinic on days 15 and 30; 12-lead ECG will be done on day 30.

Adverse event and concomitant medication information, including use of rescue medication, will be collected throughout the study (inpatient and outpatient). Surgical wound healing assessments will be made at 24, 48, and 72 hours after Time 0 and on days 7, 15, and 30 using the specified list and assessed for and recorded as adverse events as appropriate. The Southampton Wound Grading System will also be completed 72 hours posttreatment/prior to discharge (±4 hours) and on days 15 and 30. Assessment for signs and symptoms potentially indicative of systemic bupivacaine toxicity will be made after Time 0 at the following time points: 0.5, 1, 2, 3, and 4 hours (each ±15 minutes), and 5, 7, 9, 12, 15, 18, 24, 48, and 72 hours (each ±1 hour), and days 7 (±1 day) and 15 (±3 days) using the specified list and assessment made and recorded as adverse events as appropriate.

At any time that a patient is determined to be exhibiting signs and/or symptoms suggestive of systemic bupivacaine toxicity, at the discretion of the investigator, a bupivacaine blood sample will be collected and 12-lead ECG will be performed. The patient may be treated at the discretion of the investigator, including obtaining repeat bupivacaine blood levels, 12-lead ECG, or removal of the implants.

After surgery, patient reports of pain intensity using an 11-point numeric pain rating scale (NPRS) will be recorded at multiple time points through 72 hours posttreatment. Scheduled pain intensity scores will be recorded after Time 0 at 0.5, 1, 2, 3, 4, 5, 6, 8, 10, 12, 18, 20, 24, 28, 32, 36, 48, and 72 hours. Each assessment prior to hour 10 has a ±15-minute window; each assessment after and including hour 10 has a ±30-minute window. The 0.5-hour and 1-hour NPRS assessments may be omitted if, on the basis of clinical judgement, the patient is not yet awake and alert enough to appropriately answer the NPRS after surgery. Pain intensity assessments scheduled between 2400 (midnight) and 0600 (6 am) may be limited to collection every 4 hours if the patient is sleeping. However, consecutive pain assessments may not be missed, and the hour 12, 24, 48, and 72 posttreatment pain assessments must be completed even if they fall between 2400 (midnight) and 0600 (6 am). A pain intensity score will also be collected before any rescue pain medication use.

Patients will be permitted rescue medication to manage breakthrough pain when it occurs. Oral acetaminophen at 1000 mg every 4-6 hours as needed for pain (maximum daily dosage 3000 mg) and/or oxycodone 5-mg tablet(s) may be given (not to exceed 10 mg in a 4-hour period during the inpatient stay). Immediately prior to receiving any rescue medication, a pain intensity score must be recorded. If the NPRS score is 4 or less, patients will be discouraged from taking opioid rescue medication; however, rescue medication may be requested and provided at any time. If patients require opioid rescue medication, but are unable to take oral medications, they will be permitted to receive intravenous (iv) morphine (2-3 mg) every 3 hours until they are able to take oral rescue medication. As assessed by the investigator, if a patient's pain is not relieved by oxycodone and/or acetaminophen, the patient is not yet eligible for further treatment with oxycodone and/or acetaminophen, and more than 3 hours have passed since the previous iv morphine dose, a patient may receive a dose of iv morphine (2-3 mg) for pain relief. If the pain remains unrelieved or increases in intensity before additional rescue medication is allowed, additional treatment options will be discussed with the medical monitor.

Following discharge, to report an adverse event, a patient will contact study staff by telephone and report adverse event information, including incidence, duration, and any associated treatment. Patients with pain intensity scores of 4 or more at discharge will be given a written prescription for immediate-release oxycodone at a dosage of 5-10 mg every 4-6 hours as needed as rescue medication for breakthrough pain. Patients prescribed opioid rescue medication will also be permitted to take oral acetaminophen at 1000 mg every 4-6 hours (maximum daily dosage 3000 mg) and/or ibuprofen at 400 mg every 4-6 hours as needed for pain, on an outpatient basis. Patients with pain intensity scores of less than 4 at discharge will be instructed to take oral acetaminophen at 1000 mg every 4-6 hours as needed for pain (maximum daily dosage 3000 mg) and/or ibuprofen at 400 mg every 4-6 hours as needed for pain, on an outpatient basis. Patients who do not receive a written prescription for oxycodone upon discharge will be permitted to request immediate-release oxycodone 5-10 mg if their pain is unrelieved by acetaminophen. Use of opioids, acetaminophen, nonsteroidal anti-inflammatory drugs (NSAIDs), or any other medications after discharge from the hospital will be recorded, with data reviewed by study staff at subsequent contacts.

Method of Randomization and Blinding: Eligible patients will be randomly assigned to treatment in a 1:1 ratio to receive either INL-001 or placebo collagen implants. Patients will not be aware of their treatment allocation and all study staff involved in efficacy and safety assessments will be blinded to treatment assignments until after database lock and release of unblinding randomization codes. Emergency unblinding is allowable if deemed necessary by the investigator, and discussed and agreed with the study medical monitor, for the safety of the patient and will be fully documented and included in protocol deviations. Patients will be stratified by study center and body mass index (BMI) (<30 kg/m² and ≥30 kg/m²).

Duration of Patient Participation and Maximal Exposure to Study Drug: The duration of study participation for each patient will be a maximum of 75 (±4) days, consisting of a screening period (up to 45 days before surgery), an inpatient period (preoperative, intraoperative, postoperative) lasting 4 days, and an outpatient follow-up period (up to 30 days after treatment [±3 days]) including an end-of-study visit.

### Planned Number of Patients and Countries

Up to approximately 432 patients will be screened in order to randomize 372 patients to achieve a minimum of 360 patients randomized and treated [180 evaluable patients per treatment group]). Details about the definition of evaluable patients and sample size are given herein.

The study is expected to start at a time to be determined, with an estimated enrollment period of approximately 6 months, including an interim analysis. Screening to the end of the study for each patient will be approximately 75 (±4) days.

### Justification for Study Design and Selection of Population

This is a randomized, double-blind, placebo-controlled study designed primarily to assess the efficacy and safety of INL-001 in adult patients scheduled for abdominoplasty. The study design employed in this study has previously been used successfully in 2 clinical Phase 3 studies con-ducted to demonstrate the efficacy and safety of INL 001 to manage acute postoperative pain in patients undergoing open inguinal hernia repair. The rationale for studying patients undergoing abdominoplasty was previously provided herein.

### Stopping Rules for the Study

Innocoll reserves the right to discontinue the study for safety or administrative reasons at any time.

The study will be stopped, until further benefit-risk evaluation is made, if 2 patients require removal of the INL-001 implants due to suspected systemic bupivacaine toxicity as outlined herein.

During the conduct of the study, serious adverse events will be reviewed, as they are reported from the investigational centers, to identify safety concerns.

The study may also be terminated by the sponsor for any reason at any time. For example, the sponsor could terminate the study in the event of:
new toxicologic or pharmacologic findings or safety issues from any source (e.g., other clinical studies, postmarketing experience) that invalidate the earlier positive benefit-risk assessment
discontinuation of the development of the investigational medical product

If the entire study is stopped or if elements of the study are stopped, the patients whose participation is terminated early will be monitored according to withdrawal criteria and procedures.

If the study is terminated prematurely, investigator(s) will inform their patients and arrange their appropriate follow-up.

### Schedule of Study Procedures and Assessments

Study procedures and assessments by visit with their respective time points are presented in Table 8. Detailed descriptions of each method of procedures and assessments are provided (efficacy assessments) and (safety assessments). The end of study is defined as the last visit of the last patient.

Table 9 provides a summary of demographic and other baseline characteristics of the patients.

TABLE 8
Schedule of Procedures/Assessments

| | | | | In-patient postoperative period ^{b} | | | | | |
|---|---|---|---|---|---|---|---|---|---|
| | Screening | | | (hours relative to Time 0 | | | Out-patient postoperative period | | |
| | period | Day of surgery | | implantation and day is relative to | | | (relative to day of surgery) | | |
| | (up to | (day 1)• | | day of surgery) | | | | Day | Day 30/ |
| | 45 days | Immediate | Intra- | Day 2 | Day 3 | Day 4 | Day | 15 ±3 d | EOS ±3 d |
| Procedure/ | before | preoperative | operative | (through | (through | (through | 7 ±1 d | (clinic | (clinic |
| Assessment ^{a} | surgery) | period | period | 24 h) | 48 h) | 72 h) | (telephone) | visit) | visit) |
| Written informed | X | | | | | | | | |
| consent | | | | | | | | | |
| Study-drug kit | | X | | | | | | | |
| number assignment | | (enrolled) | | | | | | | |
| (after preoperative | | | | | | | | | |
| assessments) | | | | | | | | | |
| Demographics/Medical | X | X | | | | | | | |
| history | | (update) | | | | | | | |
| Inclusion/Exclusion | X | X | | | | | | | |
| criteria | | | | | | | | | |
| Urine drug screen | X | X | | | | | | | |
| (results before study- | | | | | | | | | |
| drug kit assignment) | | | | | | | | | |
| Pregnancy test (for | X | X | | | | | | | X |
| women of childbearing | (serum) | (urine) | | | | | | | (urine) |
| potential) (results | | | | | | | | | |
| before study-drug | | | | | | | | | |
| kit assignment) | | | | | | | | | |
| Prior/Concomitant | X | [symbol] | | | | | | | |
| medications (including | | | | | | | | | |
| opioids and other | | | | | | | | | |
| analgesics) | | | | | | | | | |
| Clinical laboratory | X | | | | | | | | X |
| tests | (blood/ | | | | | | | | (blood |
| | urine) | | | | | | | | only) |
| Vital signs | X | X | | X | X | X | | X | X |
| (see details and time | | | | | | | | | |
| points below) | | | | | | | | | |
| Electrocardiography | X | | | | | | | | X |
| (12 lead) | | | | | | | | | |
| Physical examination | X | | | | | | | | X |
| (screening body | | | | | | | | | |
| weight and height/ | | | | | | | | | |
| posttreatment weight | | | | | | | | | |
| only) | | | | | | | | | |
| Adverse events | X | [symbol] | | | | | | | |
| Surgery/Implantation | | | X | | | | | | |
| | | | (Time 0 is | | | | | | |
| | | | recorded | | | | | | |
| | | | implantation | | | | | | |
| | | | time of first | | | | | | |
| | | | implant) | | | | | | |
| Assessment of signs | | | | X | X | X | X | X | |
| and symptoms poten- | | | | | | | | | |
| tially indicative of | | | | | | | | | |
| systemic bupivacaine | | | | | | | | | |
| toxicity (see time | | | | | | | | | |
| points below) | | | | | | | | | |
| Assessment of signs | | | | X | X | X | X | X | X |
| and symptoms related | | | | | | (prior to | | | |
| to wound healing | | | | | | discharge) | | | |
| Southampton Wound | | | | | | X | | X | X |
| Grading System | | | | | | (prior to | | | |
| | | | | | | discharge) | | | |
| Numeric pain rating | | | | X | X | X | | | |
| scale (NPRS) (see | | | | | | | | | |
| time points below) | | | | | | | | | |
| Discharge ^{c} | | | | | | X | | | |

Footnotes to table and detailed time points:
^{a} Procedures and assessments have timing windows that may go beyond those specified.
^{b} The times patients enter and are discharged from the postanesthesia care unit (PACU) will be recorded.
^{c} Patients will be discharged after all procedures/assessments have been completed. Whether a patient is prescribed opioid pain medication at hospital discharge will be recorded.
d = day(s); EOS = end of study; h = hour(s); m = minute(s).
Time points for record of vital signs measurements after Time 0: 0.5 hour (±5 m); 1, 2, 4 hours (±15 m); 8, 12 hours (±2 h); 24, 48 hours (±3 h); 72 hours (±4 h) (prior to discharge); days 15 (±3 d) and 30 (±3 d). (Vital signs include body temperature, pulse, systolic and diastolic blood pressure, respiration rate, and oxygen saturation measured by pulse oximetry.)
NOTE:
Measurements from 0.5 through 12 hours will/may occur on day 1.
Time points for assessment of signs and symptoms potentially indicative of systemic bupivacaine toxicity after Time 0: 0.5, 1, 2, 3, and 4 hours (each ±15 m); 5, 7, 9, 12, 15, 18, 24, 48, and 72 hours (each ±1 h); and days 7 (±1 day) and 15 (±3 days).
NOTE:
Assessments from 0.5 through 18 hours will/may occur on day 1.
Time points for NPRS for pain intensity after Time 0: 0.5, 1, 2, 3, 4, 5, 6, 8 hours (each ±15 m); 10, 12, 18, 20, 24, 28, 32, 36, 48, 72 hours (each ±30 m). (The 0.5-hour and 1-hour NPRS assessment may be omitted if, on the basis of clinical judgment, the patient is not yet awake and alert enough to appropriately answer the NPRS after surgery. In the case of use of a rescue pain medication, scores will also be obtained within 15 minutes before any rescue medication use.)
NOTE:
Assessments done 0.5 through 20 hours will/may occur on day 1. No NPRS scores will be collected after discharge.

TABLE 9
Demography of Patients

| Demographic | Statistic | INL-001 | | Placebo | | Total | |
|---|---|---|---|---|---|---|---|
| Characteristic | or category | (N = 181) | | (N = 184) | | (N = 365) | |
| Sex, n (%) | Male | 0 | | 3 | (1.6) | 3 | (0.8) |
| | Female | 181 | (100) | 181 | (98.4) | 362 | (99.2) |
| Age (years) | n | 181 | | 184 | | 365 | |
| | Mean (SD) | 43.3 | (8.95) | 43.1 | (8.98) | 43.2 | (8.95) |
| | Median | 43.0 | | 43.0 | | 43.0 | |
| | Minimum-Maximum | 20-65 | | 20-65 | | 20-65 | |
| Race, n (%) | American Indian or | 0 | | 0 | | 0 | |
| | Alaska Native | | | | | | |
| | Asian | 4 | (2.2) | 4 | (2.2) | 8 | (2.2) |
| | Black or African American | 44 | (24.3) | 49 | (26.6) | 93 | (25.5) |
| | Native Hawaiian or | 1 | (0.6) | 0 | | 1 | (0.3) |
| | Other Pacific Islander | | | | | | |
| | White | 130 | (71.8) | 128 | (69.6) | 258 | (70.7) |
| | Other | 2 | (1.1) | 3 | (1.6) | 5 | (1.4) |
| Ethnicity | Hispanic or Latino | 77 | (42.5) | 79 | (42.9) | 156 | (42.7) |
| group, n (%) | Not Hispanic or Latino | 101 | (55.8) | 102 | (55.4) | 203 | (55.6) |
| | Not Reported | 2 | (1.1) | 0 | | 2 | (0.5) |
| | Unknown | 1 | (0.6) | 3 | (1.6) | 4 | (1.1) |
| Height (cm) | n | 181 | | 184 | | 365 | |
| | Mean (SD) | 161.65 | (6.687) | 162.49 | (7.249) | 162.07 | (6.979) |
| | Median | 161.30 | | 161.30 | | 161.30 | |
| | Minimum-Maximum | 144.6-185.4 | | 142.0-185.4 | | 142.0-185.4 | |
| Weight (kg) | n | 181 | | 184 | | 365 | |
| | Mean (SD) | 71.89 | (10.604) | 72.90 | (9.752) | 72.40 | (10.182) |
| | Median | 72.20 | | 72.45 | | 72.20 | |
| | Minimum-Maximum | 45.8-101.7 | | 50.8-106.9 | | 45.8-106.9 | |
| Body Mass | n | 181 | | 184 | | 365 | |
| Index (kg/m²) | Mean (SD) | 27.442 | (3.1436) | 27.566 | (2.8295) | 27.504 | (2.9859) |
| | Median | 27.610 | | 27.915 | | 27.770 | |
| | Minimum-Maximum | 19.42-34.84 | | 19.88-33.62 | | 19.42-34.84 | |
| BMI Strata, | <30 kg/m2 | 144 | (79.6) | 146 | (79.3) | 290 | (79.5) |
| n (%) | >=30 kg/m2 | 37 | (20.4) | 38 | (20.7) | 75 | (20.5) |

% = 100 × n/N.
SD = Standard Deviation

### Statistical Considerations

Sample Size Rationale: The sample size was chosen primarily on the basis of previous clinical study data for INL 001 in inguinal hernia repair, but also with the consideration of the results of other bupivacaine containing products studied in abdominoplasty. Sample size is estimated at 360 patients, with 180 patients per treatment group. The effect size with INL-001 in the combined results of 2 clinical Phase 3 studies in postoperative analgesia after open inguinal hernia repair was 0.525 for sum of pain intensity (SPI) through 24 hours (SPI24). The effect size with INL-001 was 0.25. With the historical SPI through 48 hours (SPI48) effect size of 0.25, 360 evaluable patients will yield a power of at least 66%; however, it is believed that a greater separation between the INL-001 and placebo treatment groups will be observed for abdominoplasty given that postoperative pain is more severe and longer lasting than with inguinal hernia repair, yielding greater power. This is increased to 372 patients to allow for some attrition of patients randomized to those randomized and treated; all randomized and treated patients will be evaluable and included in the modified intent-to-treat (mITT) analyses.

Efficacy Analysis: The SPI (area under the concentration-time curve [AUC] of pain intensity) as measured by the NPRS through various time points up to 72 hours posttreatment will be calculated using the trapezoidal method with NPRS scores and the actual assessment times in hours. The primary efficacy variable will be SPI24, but the same general rules and calculations will apply for all SPI0-time. For SPI24 calculation, both scheduled and unscheduled values (if available) from Time 0 through 24 hours posttreatment will be used in the calculation. For patients who receive rescue medication, just prior to it being administered, a pain score will be obtained; this will be included in the calculation of the SPI. Pain score(s) for the duration of the rescue efficacy following treatment with an opioid rescue medication will be excluded from the calculation if they are lower than the pain score just prior to rescue medication administration; those that are equal to or higher will be included. This period will be 2 hours following iv morphine and 3 hours following oxycodone.

All efficacy comparisons will be based on the comparison of INL-001 vs placebo. The primary efficacy variable, SPI24, will be analyzed using an analysis of covariance (ANCOVA) model with treatment as the main effect and a covariate for BMI. Summary statistics (sample size, mean, standard deviation [SD], median, minimum, maximum, and 25th and 75th percentiles) will be presented along with the p values from the ANCOVA model. To account for the interim analysis and potential sample size increase, the method of Cui et al 1999 will be employed. For the final analysis and resulting p-value, the data will be split for those patients included in the interim analysis and those not included in the interim analysis; data from these groups will be analyzed completely independently, then combined using the inverse normal method to test the null hypotheses that there is no difference between the treatment groups. For maximum statistical efficiency, the weights are defined prospectively according to the square root of the planned proportion of participants in the 2 stages, relative to the preplanned total enrollment of 360 patients, as wi=√0.5. The calculation of these weights is fixed and will not be changed due to unblinded data; likewise, in the case of deviations from the planned proportions due to enrollment overrun, the weights will remain fixed. An approach identical to the primary efficacy analysis will be used for each of the continuous key secondary variables and the same statistics will be presented.

For key secondary outcomes of proportions, each proportion, the difference, and 95% confidence intervals (CIs) will be reported; difference will be tested with the 2-proportion Z test (and with the Cui Hung-Wang [CHW] method applied to account for the interim analysis). In the case of low counts (any expected cell count≤5), a Fisher's Exact test will be used. All continuous secondary efficacy variables will be summarized with appropriate descriptive statistics (sample size, mean, SD, coefficient of variation [CV], median, minimum, maximum, and 25th and 75th percentiles) and analyzed using ANCOVA models with treatment as the main effect and a covariate for BMI. Summary graphs of efficacy data including total use of opioid analgesia (TOpA) and NPRS scores by treatment group (arithmetic means and standard error [SE]) vs nominal time will be plotted). All categorical efficacy variables will be summarized with counts and proportions and compared by Cochran-Mantel-Haenszel tests (for ordered variables), Pearson chi-squared, or Fisher's exact tests as needed. The time to first use of opioid rescue medication, time to discharge from the PACU, and time to no longer using rescue medication during the study will be summarized using Kaplan-Meier methods. Log rank tests will be used to compare treatment groups. The median time to discharge will be estimated together with the associated 95% CI. Models containing additional blocking factors or covariates may be fit as secondary analyses.

Safety Analyses: Safety variables include assessment of adverse events (including assessment for signs and symptoms of systemic bupivacaine toxicity and assessment of wound healing), clinical laboratory test results, vital signs measurements (including body temperature, pulse, systolic and diastolic blood pressure, respiration rate, and oxygen saturation measured by pulse oximetry), ECG findings, surgical wound grading, and concomitant medications. These analyses will be conducted for the safety population. No formal statistical tests will be performed on safety evaluations.

Multiple Comparisons and Multiplicity: The key secondary efficacy variables will be tested sequentially each at the 0.05 level to control the overall Type-I error rate. Specifically, each key secondary variable will be tested in order. The next key secondary variable will be tested if the prior secondary variable comparison is statistically significant.

Planned Interim Analysis: An interim analysis will be performed when approximately 50% of the initially planned population is evaluable with respect to efficacy. This interim analysis will be performed by unblinded personnel separate from those responsible for the conduct and analysis of the study; all decisions will be made on the basis of SPI24, SPI48, and SPI72. An independent committee will review the data and recommend to the sponsor one of the following: increase the sample size by up to 180 patients or keep the current sample size and continue.

### Investigational Medicinal Product Used in the Study and Other Treatment Information

The study drugs used in this study are: INL-001 [XARACOLL (bupivacaine hydrochloride) im-plant] and placebo collagen implant.

INL-001 is a drug-device combination product containing 100 mg of bupivacaine HCl per im-plant, equivalent to 88.8 mg of bupivacaine, for placement in the surgical site. The dose to be evaluated is three 100-mg implants (300 mg bupivacaine HCl), equivalent to 266.4 mg of bupivacaine. Each implant is 5 cm×5 cm×0.5 cm in size and is white to off-white in color. Placebo implants contain collagen but no bupivacaine. Implants are terminally sterilized.

Additional details may be found in the IB for INL-001.

### Anesthetic Protocol

The standardized anesthetic regimen will include general anesthesia with fentanyl (maximum dose of 4 mcg/kg) and propofol (dose at discretion of the anesthesia provider), with or without volatile anesthetics or muscle relaxants. The standardized anesthetic regimen is a guide that should be followed to minimize interpatient variability to the greatest extent possible. However, it is understood that hemodynamic fluctuations and other intraoperative events may necessitate some deviation from this standard regimen. Neuraxial techniques, such as epidural and spinal an-esthesia, are not allowed. No epinephrine is permitted during the procedure. No local anesthetic other than INL-001 (study drug) in the surgical field or regional anesthesia is permitted. Lidocaine HCl 1% injection at a dose of no more than 20 mg may be administered once through iv access to decrease venous irritation (eg, as caused by propofol) at the time of surgical anesthesia. Intraoperatively, fentanyl (maximum dose of 4 mcg/kg) is permitted for analgesia. No other analgesic agents may be used during the procedure including, but not limited to, opioids (other than fentanyl), acetaminophen (oral or iv), NSAIDs (eg, ketorolac or COX-2 inhibitors), ketamine, pregabalin, and others. A preoperative dose of an antiemetic, ondansetron iv 4 mg, for nausea prophylaxis is allowed; however, postoperative antiemetic medications should be given to treat only patients who report nausea and/or vomiting. Administration of fentanyl should be avoided 30 minutes prior to the anticipated conclusion of the procedure if medically acceptable in the judgement of the anesthesiologist.

### Placement of Study Drug

Following tissue removal and repair of the abdominal musculature, 2 implants should be placed on the rectus diastasis at the site of rectus sheath plication and 1 implant should be placed below the abdominal incision between Scarpa's fascia and the subcutaneous fat. The implants should, to the greatest extent possible, be placed so they span the fascia that is exposed prior to surgical closure. Implants may be divided to accommodate placement, but an individual implant may not be cut into more than 2 halves.

### Efficacy Assessments

### Pain Intensity

Pain intensity will be assessed using NPRS at specified time points during the study (see Table 1). The NPRS is an 11-point scale on which 0 indicates “no pain” and 10 indicates the “worst possible pain.” All postsurgical medication use will be recorded. In the case of a rescue medication being used, an NPRS score will be obtained within 15 minutes before the patient is administered rescue medication.

The primary efficacy variable is the sum of time-weighted pain intensity (SPI) from Time 0 through 24 hours (SPI24) as assessed by the pain intensity score using an NPRS. SPI will also be calculated for other time points.

Key secondary pain intensity efficacy variables are as follows:
SPI from Time 0 through 48 hours (SPI148)
SPI from Time 0 through 72 hours (SPI172)
   NOTE: For order of statistical analysis for key secondary efficacy variables.
   Other secondary pain intensity efficacy variables are as follows:
SPI through the following posttreatment time points: 2, 3, 4, 5, 6, 8, 10, 12, 18, 20, 28, 32, 36 hours
pain intensity at each scheduled time point

Table 10 shows the SPI 24 data which shows that the primary endpoint is statistically significant compared to placebo implant.

Table 11 shows analysis of SPI through other time points, specifically SPI2 (sum of pain intensity through 2 hours), SPI3, SPI4, SPI5, SPI6, SPI8, SPI10, SPI12, SPI18, SPI20, SPI28, SPI32, and SPI36, indicating that the sum of pain intensity is statistically significantly different than placebo implant through 36 hours.

TABLE 10
Analysis of SPI24 using ANCOVA model

| Variable | | |
|---|---|---|
| Statistic | INL-001 | Placebo |
| SPI24 | (N = 181) | (N = 184) |
| n | 181 | 184 |
| Mean (SD) | 102.05 (42.888) | 116.95 (45.184) |
| Median | 104.63 | 118.09 |
| Q1-Q3 | 68.32-134.67 | 84.60-150.83 |
| Minimum-Maximum | 0.0-210.8 | 4.7-235.0 |
| LS Mean (SE) | 102.12 (3.271) | 116.88 (3.244) |
| 95% CI [a] | 95.69-108.55 | 110.50-123.26 |
| LS Mean Difference | −14.76 (−23.82- | |
| (95% CI) [b] | −5.70) | |
| p-value [c] | 0.002 | |

This analysis is based on the data with multiple imputation.
ANCOVA = Analysis of Covariance.
SE = Standard Error.
CI = Confidence Interval.
LS = Least Squares.
SD = Standard Deviation
Q1 = 25th Quantile, Q3 = 75th Quantile.
SPI24 = (time-weighted) Sum of pain intensity over 24 hours.
NPRS values collected 4 hours following oxycodone and acetaminophen rescue medication administration, and 2 hours following morphine will be censored in the calculation of SPIs; full details are given in the SAP.
[a] LS means, SE, and 95% CIs are based on ANCOVA model that includes Body Mass Index (BMI, continuous) as a covariate and treatment group as the main effect.
[b] Difference between Treatment Groups.
[c] Interim and post-interim p-values are combined using Cui, Hung, Wang methodology and inverse normal approach.

TABLE 11
Analysis of SPI through other time points using
ANCOVA model by treatment and time point

| | | INL-001 | | Placebo | |
|---|---|---|---|---|---|
| Variable | Statistic | (N = 181) | | (N = 184) | |
| SPI2 | n | 181 | | 184 | |
| | Mean (SD) | 9.68 | (3.233) | 10.51 | (2.836) |
| | Median | 10.00 | | 10.89 | |
| | Q1-Q3 | 7.50-12.20 | | 8.94-12.41 | |
| | Minimum-Maximum | 0.0-15.7 | | 2.5-15.0 | |
| | LS Mean (SE) | 9.69 | (0.225) | 10.50 | (0.224) |
| | 95% CI [a] | 9.24-10.13 | | 10.06-10.94 | |
| | LS Mean Difference | −0.82 | | | |
| | (95% CI) [b] | (−1.44-−0.19) | | | |
| | p-value | 0.010 | | | |
| SPI3 | n | 181 | | 184 | |
| | Mean (SD) | 16.23 | (5.322) | 17.85 | (4.584) |
| | Median | 16.68 | | 18.30 | |
| | Q1-Q3 | 12.50-20.25 | | 15.45-20.88 | |
| | Minimum-Maximum | 0.0-25.7 | | 3.5-25.0 | |
| | LS Mean (SE) [a] | 16.24 | (0.367) | 17.84 | (0.364) |
| | 95% CI [a] | 15.52-16.96 | | 17.12-18.55 | |
| | LS Mean Difference | −1.60 | | | |
| | (95% CI) [b] | (−2.61-−0.58) | | | |
| | p-value | 0.002 | | | |
| SPI4 | n | 181 | | 184 | |
| | Mean (SD) | 22.36 | (7.490) | 24.84 | (6.525) |
| | Median | 23.18 | | 25.59 | |
| | Q1-Q3 | 16.92-28.00 | | 21.76-29.07 | |
| | Minimum-Maximum | 0.0-35.7 | | 4.7-35.0 | |
| | LS Mean (SE) [a] | 22.37 | (0.519) | 24.82 | (0.515) |
| | 95% CI [a] | 21.35-23.39 | | 23.81-25.83 | |
| | LS Mean Difference | −2.45 | | | |
| | (95% CI) [b] | (−3.88-−1.01) | | | |
| | p-value | 0.001 | | | |
| SPI5 | n | 181 | | 184 | |
| | Mean (SD) | 28.14 | (9.629) | 31.55 | (8.550) |
| | Median | 29.18 | | 31.54 | |
| | Q1-Q3 | 21.00-36.00 | | 27.68-37.48 | |
| | Minimum-Maximum | 0.0-45.2 | | 4.7-45.0 | |
| | LS Mean (SE) [a] | 28.16 | (0.672) | 31.53 | (0.667) |
| | 95% CI [a] | 26.84-29.49 | | 30.21-32.84 | |
| | LS Mean Difference | −3.36 | | | |
| | (95% CI) [b] | (−5.22-−1.50) | | | |
| | p-value | <0.001 | | | |
| SPI6 | n | 181 | | 184 | |
| | Mean (SD) | 33.10 | (11.430) | 37.54 | (10.393) |
| | Median | 34.00 | | 38.19 | |
| | Q1-Q3 | 24.63-42.95 | | 32.20-44.58 | |
| | Minimum-Maximum | 0.0-54.7 | | 4.7-55.0 | |
| | LS Mean (SE) [a] | 33.13 | (0.806) | 37.51 | (0.799) |
| | 95% CI [a] | 31.55-34.72 | | 35.94-39.08 | |
| | LS Mean Difference | −4.38 | | | |
| | (95% CI) [b] | (−6.61-−2.15) | | | |
| | p-value | <0.001 | | | |
| SPI8 | n | 181 | | 184 | |
| | Mean (SD) | 41.34 | (14.676) | 47.81 | (13.977) |
| | Median | 42.92 | | 49.15 | |
| | Q1-Q3 | 30.44-52.17 | | 39.47-57.08 | |
| | Minimum-Maximum | 0.0-74.7 | | 4.7-75.0 | |
| | LS Mean (SE) [a] | 41.38 | (1.058) | 47.77 | (1.049) |
| | 95% CI [a] | 39.30-43.46 | | 45.71-49.84 | |
| | LS Mean Difference | −6.40 | | | |
| | (95% CI) [b] | (−9.33-−3.47) | | | |
| | p-value | <0.001 | | | |
| SPI10 | n | 181 | | 184 | |
| | Mean (SD) | 48.88 | (18.241) | 57.34 | (17.707) |
| | Median | 49.45 | | 57.48 | |
| | Q1-Q3 | 35.16-62.18 | | 46.63-68.80 | |
| | Minimum-Maximum | 0.0-94.7 | | 4.7-95.0 | |
| | LS Mean (SE) [a] | 48.92 | (1.329) | 57.29 | (1.318) |
| | 95% CI [a] | 46.31-51.53 | | 54.70-59.88 | |
| | LS Mean Difference | −8.37 | | | |
| | (95% CI) [b] | (−12.05-−4.69) | | | |
| | p-value | <0.001 | | | |
| SPI12 | n | 181 | | 184 | |
| | Mean (SD) | 56.35 | (21.825) | 66.54 | (21.500) |
| | Median | 56.73 | | 66.81 | |
| | Q1-Q3 | 39.26-72.50 | | 53.02-80.57 | |
| | Minimum-Maximum | 0.0-111.2 | | 4.7-115.0 | |
| | LS Mean (SE) [a] | 56.40 | (1.604) | 66.49 | (1.591) |
| | 95% CI [a] | 53.24-59.55 | | 63.36-69.62 | |
| | LS Mean Difference | −10.09 | | | |
| | (95% CI) [b] | (−14.54-−5.65) | | | |
| | p-value | <0.001 | | | |
| SPI18 | n | 181 | | 184 | |
| | Mean (SD) | 79.38 | (32.243) | 92.41 | (32.873) |
| | Median | 79.58 | | 91.63 | |
| | Q1-Q3 | 54.72-103.67 | | 69.93-115.60 | |
| | Minimum-Maximum | 0.0-157.5 | | 4.7-175.0 | |
| | LS Mean (SE) [a] | 79.44 | (2.416) | 92.35 | (2.396) |
| | 95% CI [a] | 74.69-84.19 | | 87.64-97.06 | |
| | LS Mean Difference | −12.91 | | | |
| | (95% CI) [b] | (−19.60-−6.22) | | | |
| | p-value | <0.001 | | | |
| SPI20 | n | 181 | | 184 | |
| | Mean (SD) | 86.91 | (35.740) | 100.41 | (36.870) |
| | Median | 88.32 | | 99.57 | |
| | Q1-Q3 | 59.02-115.45 | | 75.03-128.00 | |
| | Minimum-Maximum | 0.0-174.8 | | 4.7-195.0 | |
| | LS Mean (SE) [a] | 86.97 | (2.696) | 100.35 | (2.674) |
| | 95% CI [a] | 81.67-92.27 | | 95.09-105.61 | |
| | LS Mean Difference | −13.38 | | | |
| | (95% CI) [b] | (−20.85-−5.92) | | | |
| | p-value | <0.001 | | | |
| SPI28 | n | 181 | | 184 | |
| | Mean (SD) | 117.21 | (50.092) | 133.10 | (53.465) |
| | Median | 118.81 | | 133.52 | |
| | Q1-Q3 | 79.01-152.60 | | 93.06-173.47 | |
| | Minimum-Maximum | 0.0-244.2 | | 4.7-275.0 | |
| | LS Mean (SE) [a] | 117.28 | (3.849) | 133.02 | (3.817) |
| | 95% CI [a] | 109.71-124.85 | | 125.52-140.53 | |
| | LS Mean Difference | −15.74 | | | |
| | (95% CI) [b] | (−26.40-−5.08) | | | |
| | p-value | 0.004 | | | |
| SPI32 | n | 181 | | 184 | |
| | Mean (SD) | 132.49 | (57.540) | 148.82 | (61.761) |
| | Median | 137.00 | | 148.47 | |
| | Q1-Q3 | 92.65-172.72 | | 100.03-194.31 | |
| | Minimum-Maximum | 0.0-276.2 | | 4.8-315.0 | |
| | LS Mean (SE) [a] | 132.58 | (4.435) | 148.74 | (4.398) |
| | 95% CI [a] | 123.85-141.30 | | 140.09-157.39 | |
| | LS Mean Difference | −16.16 | | | |
| | (95% CI) [b] | (−28.45-−3.88) | | | |
| | p-value | 0.010 | | | |
| SPI36 | n | 181 | | 184 | |
| | Mean (SD) | 147.88 | (65.329) | 163.70 | (69.828) |
| | Median | 155.58 | | 162.00 | |
| | Q1-Q3 | 98.38-192.31 | | 108.02-216.30 | |
| | Minimum-Maximum | 0.0-310.8 | | 7.2-355.0 | |
| | LS Mean (SE) [a] | 147.98 | (5.025) | 163.61 | (4.983) |

| 95% CI [a] | 138.10-157.86 | 153.81-173.41 |
|---|---|---|
| LS Mean Difference | −15.63 | |
| (95% CI) [b] | (−29.54-−1.71) | |
| p-value | 0.028 | |

This analysis is based on the data with multiple imputation.
ANCOVA = Analysis of Covariance.
SE = Standard Error.
CI = Confidence Interval.
LS = Least Squares.
SD = Standard Deviation
Q1 = 25th Quantile, Q3 = 75th Quantile.
SPI = (time-weighted) Sum of pain intensity.
NPRS values collected 4 hours following oxycodone and acetaminophen rescue medication administration, and 2 hours following morphine will be censored in the calculation of SPIs; full details are given in the SAP.
[a] LS means, SE, 95% CIs, and p-values are based on ANCOVA model that includes Body Mass Index (BMI, continuous) as a covariate and treatment group as the main effect.
[b] Difference between Treatment Groups.

### Opioid Use

Opioid use will be captured throughout the study (see Table 8). Various parameters will be calculated relating to opioid use, posttreatment through discharge and after discharge.

Key secondary opioid-use efficacy variables are as follows:
proportion of patients who are opioid free through 24 hours
proportion of patients who are opioid free through 48 hours
proportion of patients that are opioid free through 72 hours
   NOTE: For order of statistical analysis for key secondary efficacy variables.

Other secondary opioid-use efficacy variables are as follows:
proportion of patients who are opioid free from 24 through 48 hours, from 48 through 72 hours, and through day 7
proportion of patients who do not receive opioid rescue medication at discharge
proportion of patients who do not use opioids following discharge
total use of opioid analgesia (TOpA) through the following posttreatment time points: 2, 4, 6, 8, 10, 12, 18, 20, 22, 24, 26, 28, 30, 32, 34, 36, 42, 44, 46, 48, and 72 hours, and prior to discharge
total use of parenteral opioid analgesia (POpA) from Time 0 through 24 hours
time to first use of opioid rescue medication
time to no longer using opioid rescue medication during the study
proportion of patients who used any oral opioid rescue medication through the following posttreatment time points: 2, 4, 6, 8, 10, 12, 18, 20, 22, 24, 26, 28, 30, 32, 34, 36, 42, 44, 46, 48, and 72 hours
total rescue medication use through the following posttreatment time points: 2, 4, 6, 8, 10, 12, 18, 20, 22, 24, 26, 28, 30, 32, 34, 36, 42, 44, 46, 48, and 72 hours
proportion of patients who receive any rescue medication through 24, 48, and 72 hours

Table 12 shows the analysis of patients who are opioid free through 24, 48 and 72 hours, all showing numeric difference between XARACOLL and placebo implant.

Table 13 shows analysis of total use of opioid analgesia (mg) in MEQ, indicating that all time points show a statistically significant difference between XARACOLL and placebo implant through 72 hours.

### Other Assessments: Time to Discharge From the Postanesthesia Care Unit (PACU)

The times a patient enters and is discharged from the PACU will be recorded to measure time to discharge from the PACU, another secondary efficacy variable.

### Clinical Laboratory Tests

### Hematology, Serum Chemistry, and Urinalysis

Blood and urine samples for clinical laboratory tests will be collected at the screening visit and at the day 30 follow-up visit (see Table 8).

The clinical laboratory tests performed are shown in Table 14:

TABLE 14

| Hematology (complete | Serum | |
|---|---|---|
| blood count [CBC]) | chemistry: | Urinalysis |
| Hematocrit | Blood urea nitrogen | Color and |
| Hemoglobin | Creatinine | appearance |
| Platelet count | Total bilirubin | Specific gravity |
| White blood cell | Alanine | pH |
| (WBC) count | aminotransferase (ALT) | Ketones |
| WBC differential | Aspartate | Protein |
| (percentage and absolute): | aminotransferase (AST) | Blood |
| basophils | Alkaline phosphatase | Glucose |
| eosinophils | Sodium | |
| lymphocytes | Potassium | |
| monocytes | Calcium | |
| neutrophils | Chloride | |
| | Phosphate | |
| | Serum bicarbonate | |
| | Total cholesterol | |
| | Glucose (nonfasting) | |
| | Albumin | |

The following additional laboratory tests will also be performed:

For women of childbearing potential, a serum sample for pregnancy test will be collected at screening, and a urine sample on the day of surgery (with results available before study drug kit assignment) and on day 30.

All clinical laboratory test results outside the reference range will be assessed by the investigator as belonging to one of the following categories:
abnormal and not clinically significant
abnormal and clinically significant

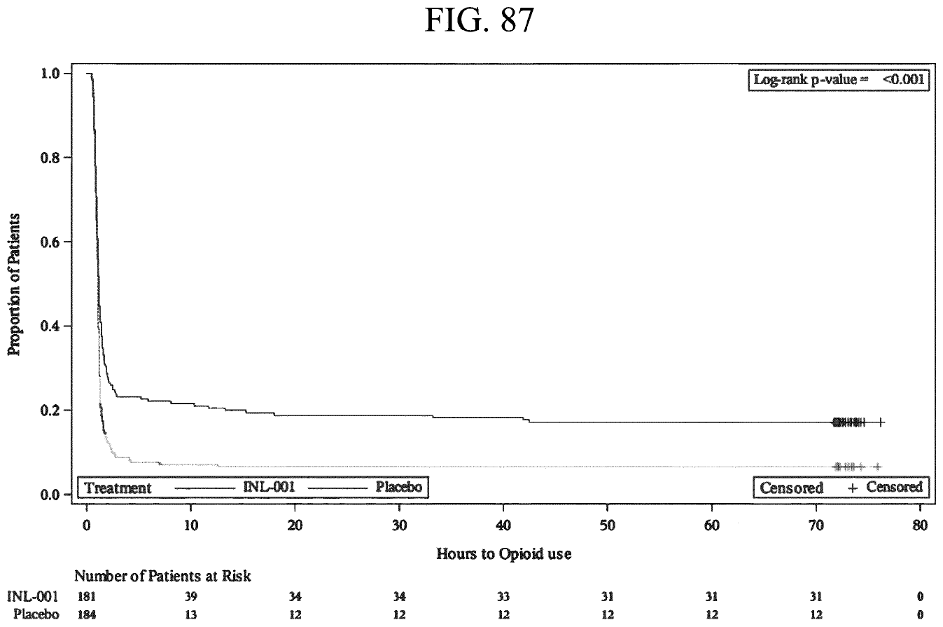

TABLE 12
Analysis of patients who are opioid free through 24, 48 and 72 hours
by treatment and time point (FIG. 87 also illustrates the comparison
of time to first opioid use between treatment groups, and the difference
in percentage of patients that stay opioid free on the drug delivery
device described herein, compared to placebo implant).

| | | INL-001 | Placebo |
|---|---|---|---|
| Time Point | Statistic | (N = 181) | (N = 184) |
| 0-24 | n (%) | 34 (18.8) | 12 (6.5) |
| Hours | 95% CI of % | 13.09-24.47 | 2.95-10.09 |
| | Difference (%) [a] | 12.26 | |
| | 95% CI of | 5.55-18.98 | |
| | Difference (%) [a] | | |
| | p-value [b] | <0.001 | |
| 0-48 | n (%) | 31 (17.1) | 12 (6.5) |
| Hours | 95% CI of % | 11.64-22.62 | 2.95-10.09 |
| | Difference (%) [a] | 10.61 | |
| | 95% CI of | 4.06-17.15 | |
| | Difference (%) [a] | | |
| | p-value [b] | 0.002 | |
| 0-72 | n (%) | 31 (17.1) | 12 (6.5) |
| Hours | 95% CI of % | 11.64-22.62 | 2.95-10.09 |
| | Difference (%) [a] | 10.61 | |
| | 95% CI of | 4.06-17.15 | |
| | Difference (%) [a] | | |
| | p-value [b] | 0.002 | |

% = 100 × n/N.
CI = Confidence Interval.
[a] Difference in the percentages along with the 95% CIs between treatment groups are based on 2-proportion Z test
[b] Interim and post-interim p-values are combined using Cui, Hung, Wang methodology and inverse normal approach.

TABLE 13
Analysis of total use of opioid analgesia (mg) in MEQ using ANCOVA model

| | | INL-001 | | Placebo | |
|---|---|---|---|---|---|
| Time Point | Statistic | (N = 181) | | (N = 184) | |
| Through | n | 181 | | 184 | |
| 2 Hour | Mean (SD) | 3.56 | (2.881) | 4.43 | (2.596) |
| | Median | 4.00 | | 5.00 | |
| | Q1-Q3 | 0.00-5.00 | | 3.00-7.00 | |
| | Minimum-Maximum | 0.0-14.0 | | 0.0-12.0 | |
| | LS Mean (SE) | 3.57 | (0.203) | 4.42 | (0.201) |
| | 95% CI [a] | 3.17-3.97 | | 4.02-4.81 | |
| | LS Mean Difference | −0.85 | | | |
| | (95% CI) [b] | (−1.41-−0.29) | | | |
| | p-value | 0.003 | | | |
| Through | n | 181 | | 184 | |
| 4 Hour | Mean (SD) | 3.97 | (2.996) | 5.17 | (2.907) |
| | Median | 4.00 | | 5.00 | |
| | Q1-Q3 | 2.00-7.00 | | 3.00-7.00 | |
| | Minimum-Maximum | 0.0-14.0 | | 0.0-13.3 | |
| | LS Mean (SE) | 3.97 | (0.218) | 5.16 | (0.217) |
| | 95% CI [a] | 3.54-4.40 | | 4.73-5.59 | |
| | LS Mean Difference | −1.19 | | | |
| | (95% CI) [b] | (−1.79-−0.58) | | | |
| | p-value | <0.001 | | | |
| Through | n | 181 | | 184 | |
| 6 Hour | Mean (SD) | 4.43 | (3.447) | 5.76 | (3.318) |
| | Median | 4.00 | | 5.00 | |
| | Q1-Q3 | 2.00-7.00 | | 3.00-7.00 | |
| | Minimum-Maximum | 0.0-18.0 | | 0.0-18.0 | |
| | LS Mean (SE) | 4.44 | (0.250) | 5.75 | (0.248) |
| | 95% CI [a] | 3.95-4.93 | | 5.27-6.24 | |
| | LS Mean Difference | −1.31 | | | |
| | (95% CI) [b] | (−2.00-−0.62) | | | |
| | p-value | <0.001 | | | |
| Through | n | 181 | | 184 | |
| 8 Hour | Mean (SD) | 4.71 | (3.591) | 6.25 | (3.710) |
| | Median | 5.00 | | 7.00 | |
| | Q1-Q3 | 2.00-7.00 | | 4.00-9.00 | |
| | Minimum-Maximum | 0.0-18.0 | | 0.0-20.0 | |
| | LS Mean (SE) | 4.72 | (0.270) | 6.24 | (0.268) |
| | 95% CI [a] | 4.18-5.25 | | 5.72-6.77 | |
| | LS Mean Difference | −1.53 | | | |
| | (95% CI) [b] | (−2.28-−0.78) | | | |
| | p-value | <0.001 | | | |
| Through | n | 181 | | 184 | |
| 10 Hour | Mean (SD) | 5.01 | (3.795) | 6.81 | (4.135) |
| | Median | 5.00 | | 7.00 | |
| | Q1-Q3 | 2.00-7.00 | | 4.00-9.00 | |
| | Minimum-Maximum | 0.0-19.0 | | 0.0-23.0 | |
| | LS Mean (SE) | 5.02 | (0.294) | 6.80 | (0.291) |
| | 95% CI [a] | 4.44-5.60 | | 6.23-7.37 | |
| | LS Mean Difference | −1.78 | | | |
| | (95% CI) [b] | (−2.59-−0.97) | | | |
| | p-value | <0.001 | | | |
| Through | n | 181 | | 184 | |
| 12 Hour | Mean (SD) | 5.39 | (4.063) | 7.20 | (4.491) |
| | Median | 5.00 | | 7.00 | |
| | Q1-Q3 | 2.00-8.00 | | 4.00-10.00 | |
| | Minimum-Maximum | 0.0-19.0 | | 0.0-26.3 | |
| | LS Mean (SE) | 5.40 | (0.317) | 7.19 | (0.315) |
| | 95% CI [a] | 4.78-6.03 | | 6.57-7.81 | |
| | LS Mean Difference | −1.79 | | | |
| | (95% CI) [b] | (−2.67-−0.91) | | | |
| | p-value | <0.001 | | | |
| Through | n | 181 | | 184 | |
| 18 Hour | Mean (SD) | 6.17 | (4.682) | 8.13 | (5.478) |
| | Median | 6.00 | | 7.00 | |
| | Q1-Q3 | 2.00-9.00 | | 4.00-11.00 | |
| | Minimum-Maximum | 0.0-24.0 | | 0.0-28.3 | |
| | LS Mean (SE) | 6.18 | (0.378) | 8.12 | (0.375) |
| | 95% CI [a] | 5.43-6.92 | | 7.39-8.86 | |
| | LS Mean Difference | −1.95 | | | |
| | (95% CI) [b] | (−3.00-−0.90) | | | |
| | p-value | <0.001 | | | |
| Through | n | 181 | | 184 | |
| 20 Hour | Mean (SD) | 6.41 | (4.910) | 8.55 | (5.840) |
| | Median | 6.00 | | 7.00 | |
| | Q1-Q3 | 2.00-9.00 | | 4.00-11.00 | |
| | Minimum-Maximum | 0.0-24.0 | | 0.0-28.3 | |
| | LS Mean (SE) | 6.42 | (0.401) | 8.54 | (0.397) |
| | 95% CI [a] | 5.63-7.21 | | 7.76-9.32 | |
| | LS Mean Difference | −2.12 | | | |
| | (95% CI) [b] | (−3.23-−1.01) | | | |
| | p-value | <0.001 | | | |
| Through | n | 181 | | 184 | |
| 22 Hour | Mean (SD) | 6.72 | (5.188) | 8.85 | (6.156) |
| | Median | 6.00 | | 7.00 | |
| | Q1-Q3 | 3.00-11.00 | | 4.00-11.50 | |
| | Minimum-Maximum | 0.0-28.0 | | 0.0-29.0 | |
| | LS Mean (SE) | 6.73 | (0.422) | 8.84 | (0.419) |
| | 95% CI [a] | 5.90-7.56 | | 8.02-9.66 | |
| | LS Mean Difference | −2.11 | | | |
| | (95% CI) [b] | (−3.28-−0.94) | | | |
| | p-value | <0.001 | | | |
| Through | n | 181 | | 184 | |
| 24 Hour | Mean (SD) | 6.94 | (5.394) | 9.20 | (6.505) |
| | Median | 7.00 | | 7.00 | |
| | Q1-Q3 | 3.00-11.00 | | 4.50-13.00 | |
| | Minimum-Maximum | 0.0-31.0 | | 0.0-31.0 | |
| | LS Mean (SE) | 6.96 | (0.444) | 9.19 | (0.440) |
| | 95% CI [a] | 6.08-7.83 | | 8.32-10.06 | |
| | LS Mean Difference | −2.23 | | | |
| | (95% CI) [b] | (−3.46-−1.01) | | | |
| | p-value | <0.001 | | | |
| Through | n | 181 | | 184 | |
| 26 Hour | Mean (SD) | 7.15 | (5.652) | 9.47 | (6.766) |
| | Median | 7.00 | | 8.00 | |
| | Q1-Q3 | 3.00-11.00 | | 4.50-13.00 | |
| | Minimum-Maximum | 0.0-35.0 | | 0.0-31.0 | |
| | LS Mean (SE) | 7.17 | (0.463) | 9.46 | (0.459) |
| | 95% CI [a] | 6.26-8.08 | | 8.56-10.36 | |
| | LS Mean Difference | −2.29 | | | |
| | (95% CI) [b] | (−3.58-−1.01) | | | |
| | p-value | <0.001 | | | |
| Through | n | 181 | | 184 | |
| 28 Hour | Mean (SD) | 7.40 | (5.924) | 9.72 | (7.034) |
| | Median | 7.00 | | 8.00 | |
| | Q1-Q3 | 3.00-11.00 | | 5.00-13.00 | |
| | Minimum-Maximum | 0.0-38.0 | | 0.0-33.0 | |
| | LS Mean (SE) | 7.42 | (0.483) | 9.71 | (0.479) |
| | 95% CI [a] | 6.47-8.36 | | 8.77-10.65 | |
| | LS Mean Difference | −2.30 | | | |
| | (95% CI) [b] | (−3.63-−0.96) | | | |
| | p-value | 0.001 | | | |
| Through | n | 181 | | 184 | |
| 30 Hour | Mean (SD) | 7.67 | (6.153) | 10.03 | (7.325) |
| | Median | 7.00 | | 8.00 | |
| | Q1-Q3 | 3.00-12.00 | | 5.00-14.00 | |
| | Minimum-Maximum | 0.0-40.0 | | 0.0-33.0 | |
| | LS Mean (SE) | 7.68 | (0.502) | 10.02 | (0.498) |
| | 95% CI [a] | 6.69-8.67 | | 9.04-11.00 | |
| | LS Mean Difference | −2.34 | | | |
| | (95% CI) [b] | (−3.73-−0.94) | | | |
| | p-value | 0.001 | | | |
| Through | n | 181 | | 184 | |
| 32 Hour | Mean (SD) | 7.89 | (6.298) | 10.40 | (7.683) |
| | Median | 7.00 | | 9.00 | |
| | Q1-Q3 | 3.00-12.00 | | 5.00-15.00 | |
| | Minimum-Maximum | 0.0-40.0 | | 0.0-35.0 | |
| | LS Mean (SE) | 7.90 | (0.522) | 10.38 | (0.517) |
| | 95% CI [a] | 6.88-8.93 | | 9.37-11.40 | |
| | LS Mean Difference | −2.48 | | | |
| | (95% CI) [b] | (−3.93-−1.04) | | | |
| | p-value | 0.001 | | | |
| Through | n | 181 | | 184 | |
| 34 Hour | Mean (SD) | 8.19 | (6.618) | 10.59 | (7.889) |
| | Median | 7.00 | | 9.00 | |
| | Q1-Q3 | 3.00-13.00 | | 5.00-15.00 | |
| | Minimum-Maximum | 0.0-44.0 | | 0.0-35.0 | |
| | LS Mean (SE) | 8.20 | (0.541) | 10.57 | (0.536) |
| | 95% CI [a] | 7.14-9.26 | | 9.52-11.63 | |
| | LS Mean Difference | −2.37 | | | |
| | (95% CI) [b] | (−3.87-−0.88) | | | |
| | p-value | 0.002 | | | |
| Through | n | 181 | | 184 | |
| 36 Hour | Mean (SD) | 8.51 | (6.864) | 10.93 | (8.301) |
| | Median | 7.00 | | 9.00 | |
| | Q1-Q3 | 3.00-13.00 | | 5.00-16.00 | |
| | Minimum-Maximum | 0.0-44.0 | | 0.0-38.0 | |
| | LS Mean (SB) | 8.53 | (0.566) | 10.92 | (0.561) |
| | 95% CI [a] | 7.41-9.64 | | 9.81-12.02 | |
| | LS Mean Difference | −2.39 | | | |
| | (95% CI) [b] | (−3.96-−0.82) | | | |
| | p-value | 0.003 | | | |
| Through | n | 181 | | 184 | |
| 42 Hour | Mean (SD) | 9.06 | (7.539) | 11.47 | (8.853) |
| | Median | 8.00 | | 9.00 | |
| | Q1-Q3 | 3.00-14.00 | | 5.00-17.00 | |
| | Minimum-Maximum | 0.0-55.0 | | 0.0-42.0 | |
| | LS Mean (SE) | 9.07 | (0.611) | 11.46 | (0.606) |
| | 95% CI [a] | 7.87-10.27 | | 10.27-12.65 | |
| | LS Mean Difference | −2.39 | | | |
| | (95% CI) [b] | (−4.08-−0.70) | | | |
| | p-value | 0.006 | | | |
| Through | n | 181 | | 184 | |
| 44 Hour | Mean (SD) | 9.18 | (7.641) | 11.64 | (9.087) |
| | Median | 8.00 | | 9.00 | |
| | Q1-Q3 | 3.00-15.00 | | 5.00-17.00 | |
| | Minimum-Maximum | 0.0-55.0 | | 0.0-46.0 | |
| | LS Mean (SE) | 9.19 | (0.624) | 11.62 | (0.619) |
| | 95% CI [a] | 7.96-10.42 | | 10.41-12.84 | |
| | LS Mean Difference | −2.44 | | | |
| | (95% CI) [b] | (−4.16-−0.71) | | | |
| | p-value | 0.006 | | | |
| Through | n | 181 | | 184 | |
| 46 Hour | Mean (SD) | 9.35 | (7.948) | 11.76 | (9.258) |
| | Median | 8.00 | | 9.00 | |
| | Q1-Q3 | 3.00-15.00 | | 5.00-18.50 | |
| | Minimum-Maximum | 0.0-59.0 | | 0.0-46.0 | |
| | LS Mean (SE) | 9.36 | (0.641) | 11.74 | (0.636) |
| | 95% CI [a] | 8.10-10.62 | | 10.49-12.99 | |
| | LS Mean Difference | −2.38 | | | |
| | (95% CI) [b] | (−4.16-−0.61) | | | |
| | p-value | 0.009 | | | |
| Through | n | 181 | | 184 | |
| 48 Hour | Mean (SD) | 9.40 | (8.011) | 11.92 | (9.468) |
| | Median | 8.00 | | 9.00 | |
| | Q1-Q3 | 3.00-15.00 | | 5.00-18.50 | |
| | Minimum-Maximum | 0.0-59.0 | | 0.0-46.0 | |
| | LS Mean (SE) | 9.42 | (0.652) | 11.91 | (0.646) |
| | 95% CI [a] | 8.13-10.70 | | 10.64-13.18 | |
| | LS Mean Difference | −2.49 | | | |
| | (95% CI) [b] | (−4.30-−0.69) | | | |
| | p-value | 0.007 | | | |
| Through | n | 181 | | 184 | |
| 72 Hour | Mean (SD) | 10.88 | (10.377) | 13.53 | (11.961) |
| | Median | 9.00 | | 9.00 | |
| | Q1-Q3 | 3.00-17.00 | | 5.00-20.00 | |
| | Minimum-Maximum | 0.0-87.0 | | 0.0-68.0 | |
| | LS Mean (SE) | 10.90 | (0.832) | 13.51 | (0.825) |
| | 95% CI [a] | 9.26-12.54 | | 11.89-15.14 | |
| | LS Mean Difference | −2.61 | | | |
| | (95% CI) [b] | (−4.92-−0.31) | | | |
| | p-value | 0.026 | | | |
| Prior to | n | 181 | | 184 | |
| discharge | Mean (SD) | 10.87 | (10.521) | 13.62 | (12.080) |

| Median | 9.00 | | 9.00 | |
|---|---|---|---|---|
| Q1-Q3 | 3.00-17.00 | | 5.00-20.50 | |
| Minimum-Maximum | 0.0-91.0 | | 0.0-68.0 | |
| LS Mean (SE) | 10.89 | (0.842) | 13.60 | (0.835) |

| 95% CI [a] | 9.23-12.54 | 11.96-15.24 |
|---|---|---|
| LS Mean Difference | −2.71 | |
| (95% CI) [b] | (−5.04-−0.38) | |
| p-value | 0.023 | |

MEQ = Morphine Equivalents.
ANCOVA = Analysis of Covariance.
Q1 = 25th Quantile, Q3 = 75th Quantile.
SE = Standard Error.
CI = Confidence Interval.
LS = Least Squares.
SD = Standard Deviation
Note:
Oxycodone mg will be converted to MEQ by multiplying by 1.5. Per the protocol, only morphine and oxycodone will be used as opioid rescue medications, but should another opioid moiety be used, the MEQ will be calculated using the CDC conversion guide: (https://www.cdc.gov/drugoverdose/pdf/calculating_total_daily_dose-a.pdf).
[a] LS means, SE, 95% CIs, and p-values are based on ANCOVA model that includes Body Mass Index (BMI, continuous) as a covariate and treatment group as the main effect.
[b] Difference between Treatment Groups.

A laboratory test result that is judged by the investigator as clinically significant will be recorded both on the source documentation and the CRF as an adverse event and will be monitored as described herein. An event may include a laboratory or diagnostic test abnormality that results in the withdrawal of the patient from the study, the temporary or permanent withdrawal of medical treatment, or further diagnostic work up. (NOTE: Abnormal laboratory or diagnostic test results at the screening visit that preclude a patient from entering the study or receiving study drug are not considered adverse events.)

Clinical laboratory tests (serum chemistry, hematology, and urinalysis) will be performed at the time points detailed in Table 8. Blood samples (approximately 16 mL total per patient) will be collected. Clinical laboratory tests will be performed using the central laboratory.

### Urine Drug Screen

A urine drug screen will be performed at the screening visit and immediately before surgery (see Table 8). Urine screening will be done for drugs of abuse/misuse, with testing during the screening period and on the day of surgery (with results available before study drug kit number assignment).

A positive result for any excluded drugs of misuse/abuse or their metabolites without medical explanation will preclude the patient from enrollment or continued participation in the study.

### Vital Signs

Vital signs, including body temperature, pulse, systolic and diastolic blood pressure, respiration rate, and oxygen saturation, will be measured/recorded at screening, preoperatively, and at the following posttreatment time points: 0.5 hours (±5 minutes); 1, 2, 4 hours (±15 minutes); 8, 12 hours (±2 hours); 24, 48, 72 hours (±4 hours) prior to discharge; day 15 (±3 days); and day 30 (±3 days) (see Table 8).

Oxygen saturation will be monitored by pulse oximetry during the inpatient study period. Oxy-gen saturation will be recorded at the individual time points listed above. Pulse oximeter alarms should be set according to clinic standards, with oxygen desaturation that occurs in concordance with the delay period and the specified limits recorded as an adverse event. Patients should be evaluated to ensure proper pulse oximeter placement and to ensure desaturation is not due to patient movement or device-related issues.

All vital sign results outside the reference ranges will be judged by the investigator as belonging to one 1 of the following categories:
abnormal and not clinically significant
abnormal and clinically significant

### Physical Examinations

A complete physical examination will be performed at screening and on day 30 (±3 days) after treatment (including screening body weight and height and posttreatment weight only) (see Table 1). A complete physical examination will include at a minimum skin, lungs, CV, respiratory, gastrointestinal, musculoskeletal, and neurological assessments. Any physical examination finding that is judged by the investigator as clinically significant (except at the screening visit) will be considered an adverse event, recorded in the CRF, and monitored as described herein. Investigators should pay special attention to clinical signs related to previous serious diseases.

### Electrocardiography

A standard 12 lead ECG will be performed locally and recorded (after the patient has been supine for at least 5 minutes) at screening and on day 30 (±3 days) (see Table 8). All ECG recordings will be identified with the patient number, date, and time of the recording.

All ECG results outside of the reference ranges should be evaluated and will be judged as belonging to one of the following categories:
abnormal and not clinically significant
abnormal and clinically significant

Any ECG finding that is judged as clinically significant (except at the screening visit) will be considered an adverse event, recorded on the source documentation and on the CRF, and monitored as described herein.

### Example 8: An Open-Label Study to Evaluate the Safety and Pharmacokinetics of the INL-001 (Bupivacaine Hydrochloride) Implant in Adults Following Various Open Soft-Tissue Surgeries: Open Ventral Hernia Repair, Abdominoplasty, Open Abdominal Hysterectomy, Laparoscopic-Assisted Colectomy. And Reduction Mammoplasty

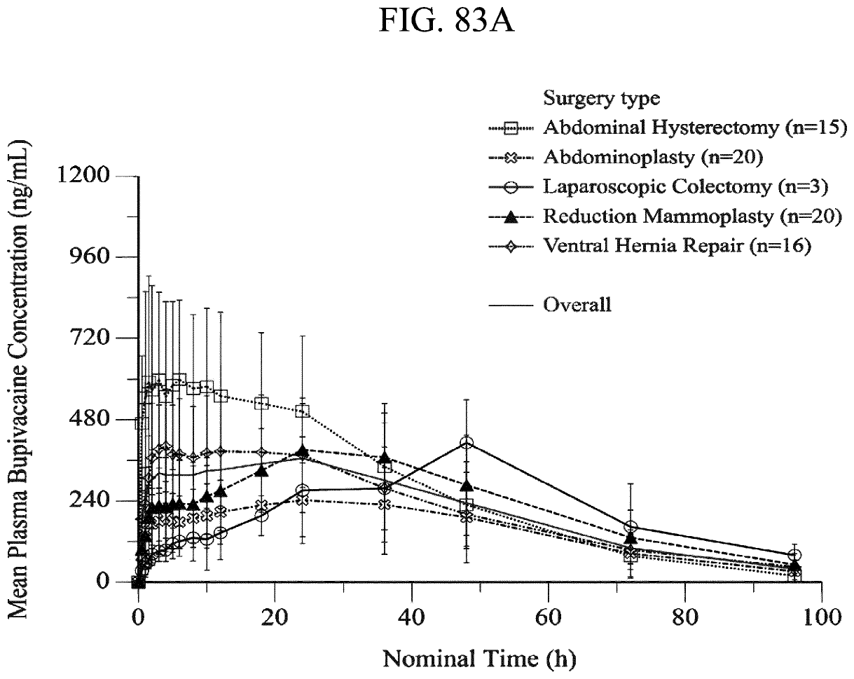

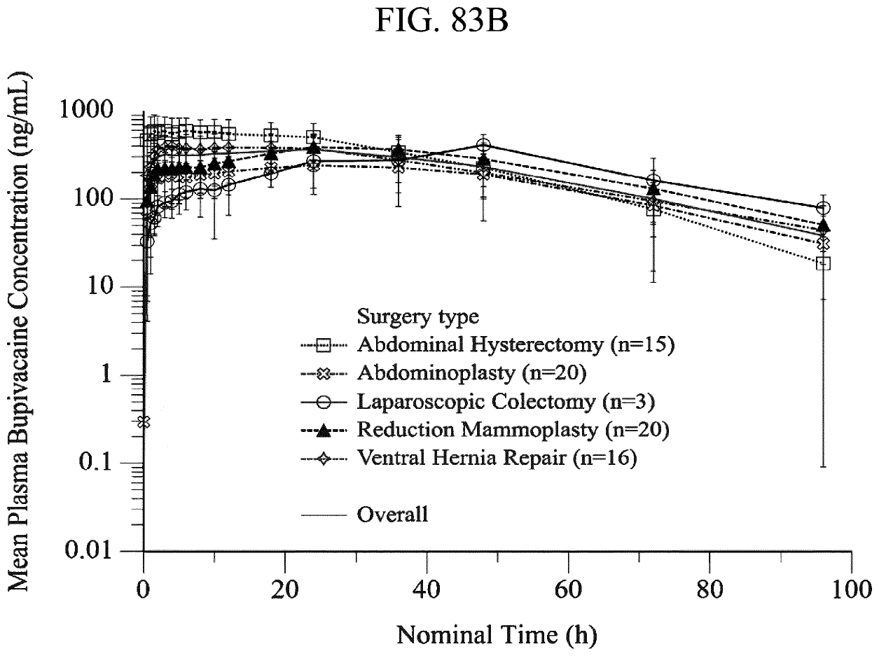

FIGS. 83A and 83B illustrate the Mean Plasma Bupivacaine Concentration-Time Profiles; Mean (±SD) Plasma Bupivacaine Concentrations by Surgery Type on Linear (FIG. 83A) and Semi-Logarithmic Scale (FIG. 83B)—PK Analysis Set.

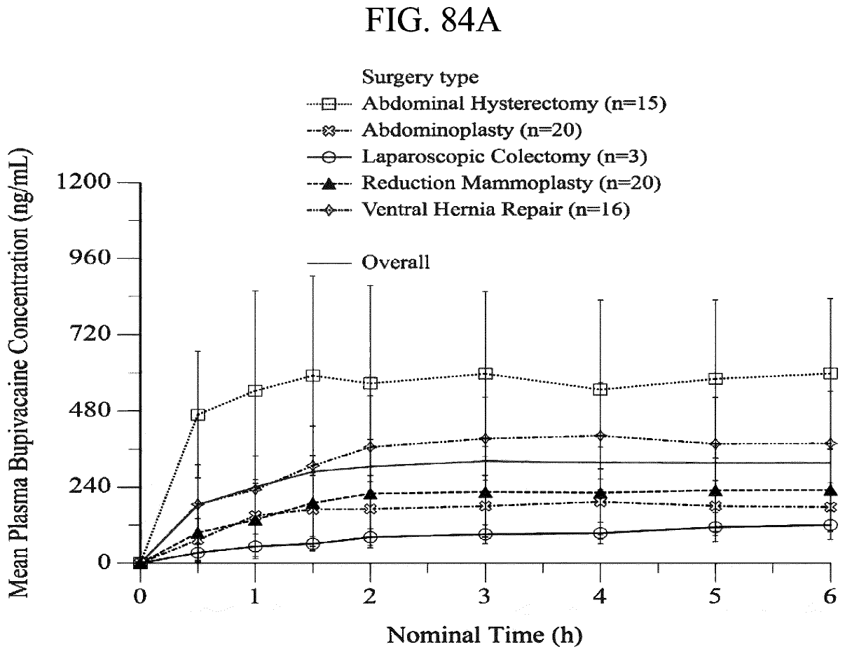

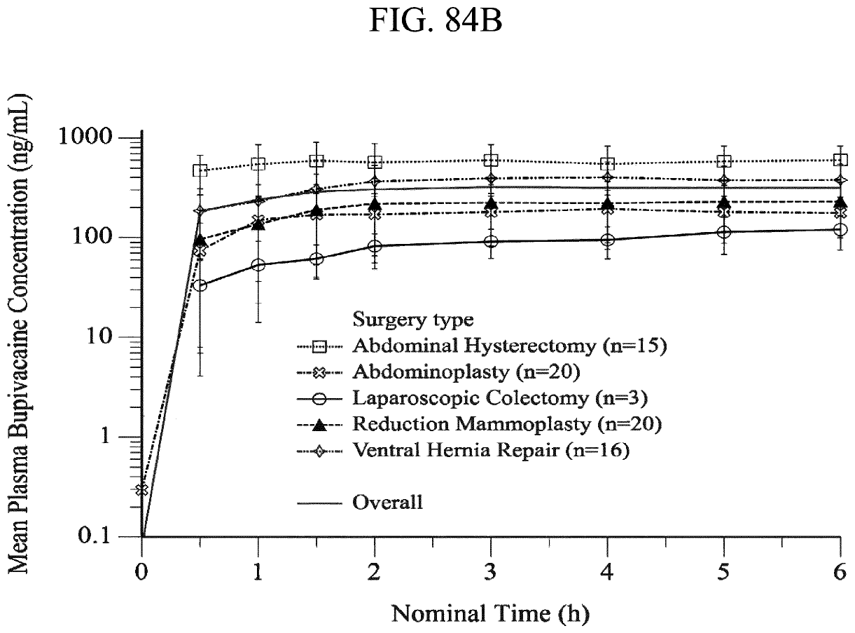

FIGS. 84A and 84B illustrate the Mean Plasma Bupivacaine Concentration-Time Profiles; Mean (±SD) Plasma Bupivacaine Concentrations by Surgery Type on Linear (FA) and Semi-Logarithmic Scale (FIG. 84B) (0-6 Hours)—PK Analysis Set.

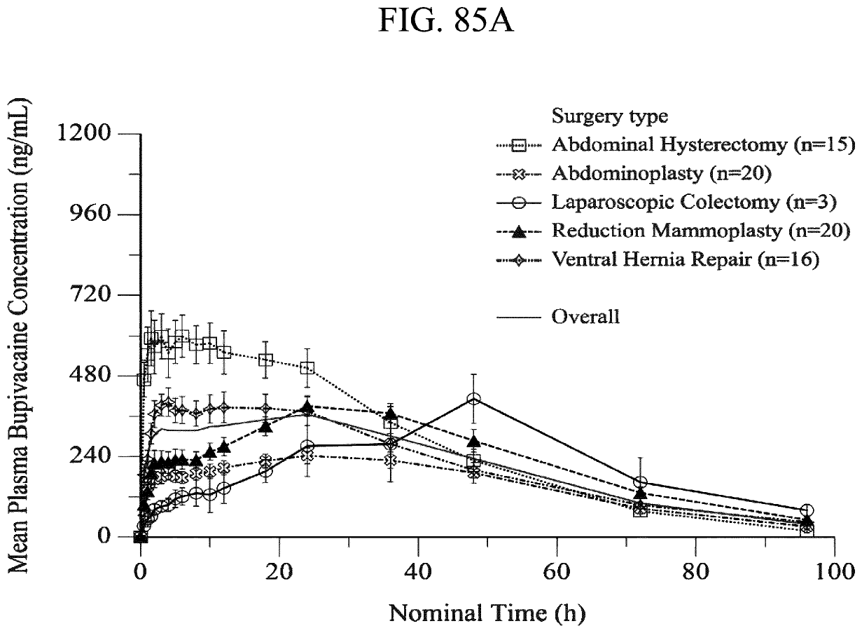

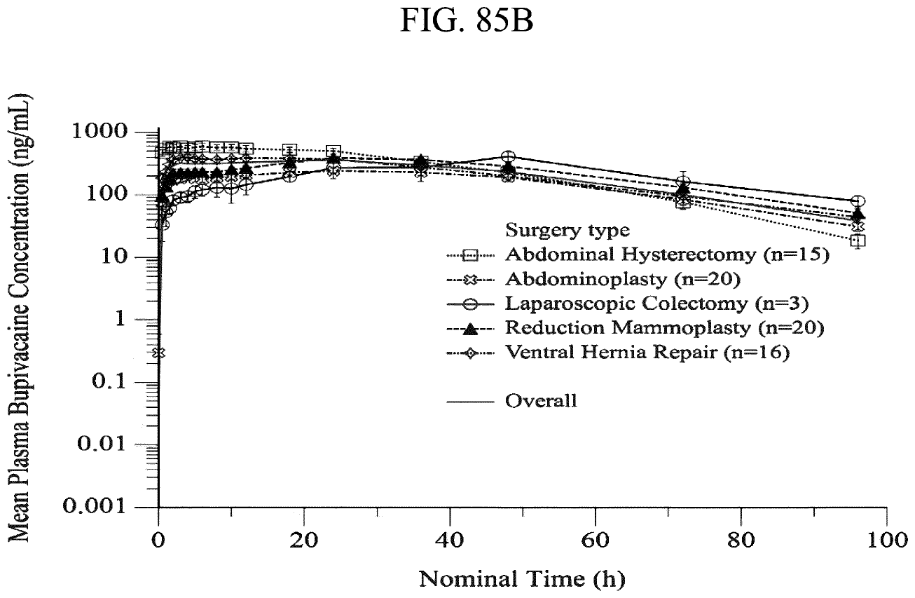

FIGS. 85A and 85B illustrate the Mean Plasma Bupivacaine Concentration-Time Profiles; Mean (±SE) Plasma Bupivacaine Concentrations by Surgery Type on Linear (FIG. 85A) and Semi-Logarithmic Scale (FIG. 85B)—PK Analysis Set.

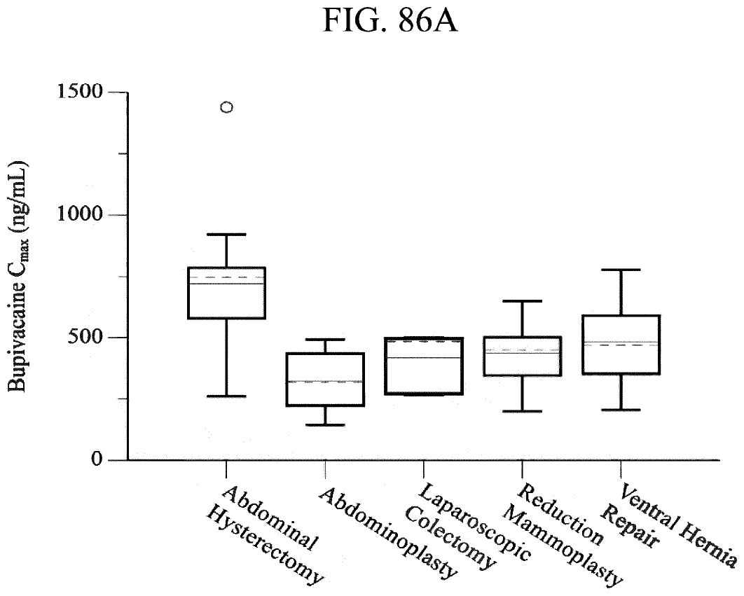

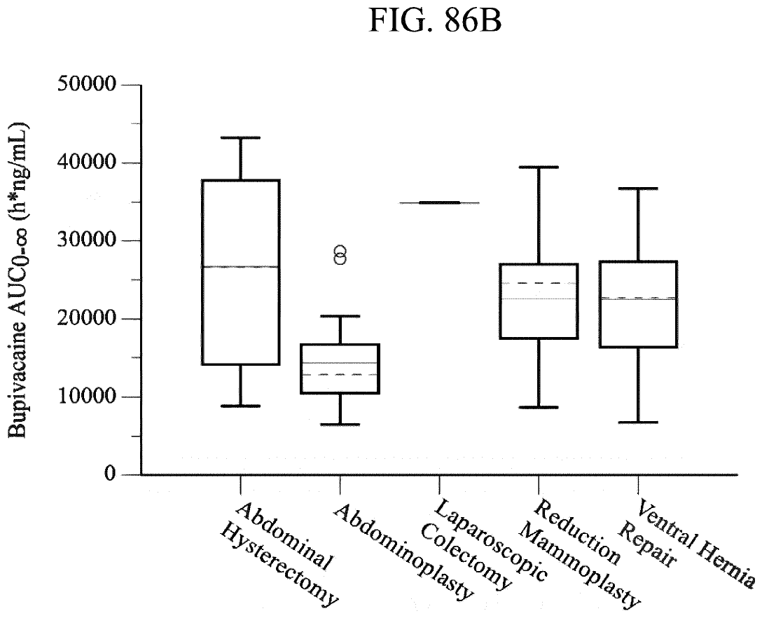

FIGS. 86A and 86B illustrate Box Plots of Plasma Bupivacaine Pharmacokinetic Parameters; FIG. 86A: Box Plots of Plasma Bupivacaine Cₘₐₓ by Surgery Type on Linear Scale—PK Analysis Set; the dashed line is the median; the solid line is the arithmetic mean. The ends of the “box” are the 25th and 75th percentiles. The whiskers show the lowest data value still within 1.5 IQR of the lower quartile, and the highest value still within 1.5 IQR of the upper quartile, where IQR is the interquartile range. Data values that do not fall between the whiskers are plotted as outliers; FIG. 86B: Box Plot of Plasma Bupivacaine AUC_{0-∞} by Surgery Type on Linear Scale—PK Analysis Set; The dashed line is the median; the solid line is the arithmetic mean. The ends of the “box” are the 25th and 75th percentiles. The whiskers show the lowest data value still within 1.5 IQR of the lower quartile, and the highest value still within 1.5 IQR of the upper quartile, where IQR is the interquartile range. Data values that do not fall between the whiskers are plotted as outliers.

TABLE 15
Individual and Summary Plasma Bupivacaine Concentrations Following Placement of INL-001 Bupivacaine
HCl Implants (300 mg) in Patients Following Open Ventral Hernia Repair (PK Analysis Set)

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | Subject | Bupivacaine Concentration (ng/mL) | | | | | | | | |
| Ventral | 102-002 | BLQ | 64.7 | 85.8 | 101 | 136 | 176 | 184 | 211 | 210 |
| Hernia | 102-003 | BLQ | 117 | 149 | 207 | 254 | 270 | 270 | 244 | 238 |
| Repair | 102-004 | BLQ | 93.4 | 206 | 330 | 348 | 392 | 291 | 291 | 283 |
| | 102-005 | BLQ | 142 | 201 | 305 | 361 | 435 | 474 | 379 | 376 |
| | 102-006 | BLQ | 107 | 216 | 305 | 372 | 496 | 518 | 491 | 514 |
| | 102-007 | BLQ | 345 | 367 | 503 | 659 | 470 | 472 | 462 | 339 |
| | 102-008 | BLQ | 144 | 249 | 411 | 392 | 364 | 325 | 347 | 366 |
| | 102-009 | BLQ | 213 | 213 | 213 | 200 | 258 | 240 | 224 | 225 |
| | 102-011 | BLQ | 98.7 | 200 | 273 | 323 | 418 | 357 | 370 | 359 |
| | 102-013 | BLQ | 156 | 283 | 376 | 543 | 593 | 682 | 605 | 691 |
| | 103-019 | BLQ | 44.4 | 53.3 | 144 | 197 | 337 | 366 | 344 | 416 |
| | 103-021 | BLQ | 420 | 425 | 406 | 431 | 525 | 535 | 485 | 500 |
| | 103-022 | BLQ | 234 | 368 | 535 | 425 | 458 | 405 | 391 | 372 |
| | 103-023 | BLQ | 102 | 115 | 179 | 205 | 179 | 174 | 142 | 122 |
| | 103-024 | BLQ | 457 | 349 | 387 | 703 | 589 | 777 | 693 | 719 |
| | 103-029 | BLQ | 229 | 235 | 247 | 316 | 325 | 359 | 348 | 320 |
| | N | 16 | 16 | 16 | 16 | 16 | 16 | 16 | 16 | 16 |
| | Mean | 0.000 | 185.5 | 232.2 | 307.6 | 366.6 | 392.8 | 401.8 | 376.7 | 378.1 |
| | SD | 0.0000 | 124.36 | 105.74 | 124.28 | 161.52 | 130.09 | 168.69 | 145.62 | 163.36 |
| | CV % | NC | 67.1 | 45.5 | 40.4 | 44.1 | 33.1 | 42.0 | 38.7 | 43.2 |
| | Min | 0.00 | 44.4 | 53.3 | 101 | 136 | 176 | 174 | 142 | 122 |
| | Median | 0.00 | 143 | 215 | 305 | 355 | 405 | 363 | 359 | 363 |
| | Max | 0.00 | 457 | 425 | 535 | 703 | 593 | 777 | 693 | 719 |
| | Geometric Mean | NC | 152.0 | 205.2 | 281.5 | 334.0 | 369.8 | 369.8 | 349.7 | 345.4 |
| | Geometric CV % | NC | 73.0 | 60.4 | 48.3 | 47.8 | 39.0 | 44.6 | 42.8 | 47.6 |
| | | Hour (h) | | | | | | | | |
| | | 8.0 | 10 | 12 | 18 | 24 | 36 | 48 | 72 | 96 |
| Surgery | Subject | Bupivacaine Concentration (ng/mL) | | | | | | | | |
| Ventral | 102-002 | 237 | 247 | 260 | 305 | 329 | 250 | 216 | 97.6 | 41.3 |
| Hernia | 102-003 | 284 | 291 | 283 | 321 | 346 | 269 | 197 | 112 | 43.8 |
| Repair | 102-004 | 253 | 263 | 294 | 392 | 464 | 321 | 248 | 113 | 40.6 |
| | 102-005 | 330 | 358 | 316 | 297 | 287 | 231 | 177 | 83.8 | 30.7 |
| | 102-006 | 504 | 577 | 608 | 546 | 471 | 498 | 373 | 217 | 86.5 |
| | 102-007 | 403 | 375 | 398 | 473 | . | 401 | 264 | 123 | 51.3 |
| | 102-008 | 294 | 296 | 312 | 353 | 376 | 266 | 174 | 94.1 | 61.2 |
| | 102-009 | 220 | 194 | 205 | 188 | 266 | 193 | 119 | 71.2 | 39.1 |
| | 102-011 | 323 | 288 | 257 | 242 | 188 | 125 | 93.0 | 56.2 | 41.0 |
| | 102-013 | 623 | 689 | 772 | 759 | 731 | 249 | 328 | 98.0 | 23.8 |
| | 103-019 | 453 | 532 | 554 | 509 | 557 | 323 | 215 | 88.3 | 33.6 |
| | 103-021 | 478 | 548 | 511 | 468 | 346 | 279 | 153 | 72.1 | 49.1 |
| | 103-022 | 356 | 403 | 385 | 388 | 484 | 465 | 342 | 92.9 | 22.2 |
| | 103-023 | 123 | 114 | 96.5 | 78.2 | 57.4 | 54.8 | 41.9 | 32.3 | 21.3 |
| | 103-024 | 691 | 644 | 638 | 581 | 517 | 392 | 154 | 95.0 | 84.7 |
| | 103-029 | 310 | 289 | 287 | 238 | 198 | 119 | 96.6 | 71.7 | 41.7 |
| | N | 16 | 16 | 16 | 16 | 15 | 16 | 16 | 16 | 16 |
| | Mean | 367.6 | 381.8 | 386.0 | 383.6 | 374.5 | 277.2 | 199.5 | 94.89 | 44.49 |
| | SD | 150.60 | 168.28 | 181.50 | 169.02 | 168.95 | 122.52 | 94.018 | 39.617 | 19.278 |
| | CV % | 41.0 | 44.1 | 47.0 | 44.1 | 45.1 | 44.2 | 47.1 | 41.8 | 43.3 |
| | Min | 123 | 114 | 96.5 | 78.2 | 57.4 | 54.8 | 41.9 | 32.3 | 21.3 |
| | Median | 327 | 327 | 314 | 371 | 346 | 268 | 187 | 93.5 | 41.2 |
| | Max | 691 | 689 | 772 | 759 | 731 | 498 | 373 | 217 | 86.5 |
| | Geometric Mean | 338.8 | 345.7 | 345.1 | 342.5 | 327.6 | 245.2 | 175.6 | 88.14 | 41.02 |
| | Geometric CV % | 44.9 | 50.7 | 54.8 | 58.0 | 67.2 | 61.7 | 61.3 | 41.8 | 43.1 |

BLQ = Below the limit of quantification (<1.00 ng/mL), treated as 0 for the calculation of PK parameters and descriptive statistics;
NC = Not calculated;
. = Value missing or not reportable

TABLE 16
Individual and Summary Plasma Bupivacaine Concentrations Following
Placement of INL-001 Bupivacaine HCl Implants (300 mg) in
Patients Following Abdominoplasty (PK Analysis Set)

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | Subject | Bupivacaine Concentration (ng/mL) | | | | | | | | |
| Abdomino- | 101-003 | BLQ | 30.6 | 50.0 | 64.9 | 95.0 | 85.3 | 104 | 96.2 | 104 |
| plasty | 101-005 | BLQ | 10.3 | 35.8 | 59.7 | 92.0 | 176 | 155 | 174 | 175 |
| | 101-008 | BLQ | 43.1 | 63.2 | 82.6 | 90.0 | 136 | 115 | 123 | 122 |
| | 101-009 | BLQ | 7.83 | 37.7 | 53.7 | 83.5 | 103 | 99.8 | 112 | 120 |
| | 101-040 | BLQ | 16.4 | 60.4 | 89.3 | 112 | 133 | 154 | 193 | 208 |
| | 105-001 | BLQ | 42.7 | 129 | 149 | 139 | 148 | 149 | 152 | 126 |
| | 105-002 | BLQ | 211 | 194 | 346 | 256 | 405 | 379 | 352 | 357 |
| | 105-004 | BLQ | 197 | 321 | 284 | 186 | 151 | 184 | 179 | 178 |
| | 105-005 | 5.91 | 37.4 | 71.1 | 45.0 | 79.3 | 83.4 | 95.5 | 97.0 | 101 |
| | 105-006 | BLQ | 39.2 | 69.1 | 112 | 105 | 117 | 112 | 114 | 150 |
| | 105-007 | BLQ | 107 | 411 | 331 | 378 | 290 | 294 | 246 | 245 |
| | 105-010 | BLQ | 26.7 | 55.0 | 60.9 | 41.0 | 62.1 | 63.8 | 53.1 | 55.6 |
| | 105-011 | BLQ | 50.8 | 177 | 205 | 182 | 219 | 207 | 232 | 210 |
| | 105-012 | BLQ | 27.0 | 125 | 130 | 134 | 123 | 450 | 134 | 120 |
| | 105-013 | BLQ | 145 | 196 | 209 | 224 | 234 | 228 | 259 | 266 |
| | 105-014 | BLQ | 194 | 261 | 363 | 359 | 328 | 291 | 266 | 252 |
| | 105-015 | BLQ | 58.9 | 155 | 160 | 190 | 154 | 162 | 173 | 173 |
| | 105-020 | BLQ | 93.0 | 132 | 130 | 134 | 157 | 165 | 181 | 183 |
| | 105-021 | BLQ | 22.5 | 80.7 | 105 | 131 | 129 | 132 | 128 | 92.2 |
| | 105-025 | BLQ | 124 | 373 | 411 | 410 | 367 | 331 | 340 | 296 |

| | | Hour (h) | | | | | | | | |
|---|---|---|---|---|---|---|---|---|---|---|
| | | 8.0 | 10 | 12 | 18 | 24 | 36 | 48 | 72 | 96 |
| Surgery | Subject | Bupivacaine Concentration (ng/mL) | | | | | | | | |
| Abdomino- | 101-003 | 110 | 111 | 123 | 223 | 295 | 363 | 387 | 203 | 69.5 |
| plasty | 101-005 | 173 | 203 | 195 | 157 | 219 | 180 | 166 | 89.6 | 27.1 |
| | 101-008 | 125 | 124 | 143 | 200 | 267 | 319 | 296 | 158 | 50.5 |
| | 101-009 | 136 | 137 | 155 | 174 | 121 | 117 | 66.3 | 39.8 | 20.0 |
| | 101-040 | 219 | 205 | 192 | 195 | 262 | 185 | 144 | 83.2 | 42.5 |
| | 105-001 | 169 | 150 | 144 | 148 | 158 | 147 | 127 | 63.8 | 31.8 |
| | 105-002 | 420 | 470 | 485 | 222 | 135 | 152 | 84.7 | 28.3 | 10.9 |
| | 105-004 | 153 | 123 | 158 | 154 | 100 | 80.9 | 47.8 | 8.95 | 3.35 |
| | 105-005 | 93.4 | 115 | 121 | 126 | 219 | 181 | 173 | 73.8 | 36.0 |
| | 105-006 | 128 | 143 | 144 | 202 | 209 | 212 | 128 | 29.7 | 5.59 |
| | 105-007 | 239 | 233 | 233 | 231 | 215 | 232 | 136 | 43.0 | 10.3 |
| | 105-010 | 50.9 | 76.3 | 91.9 | 143 | 123 | 127 | 103 | 48.9 | 33.3 |
| | 105-011 | 240 | 237 | 206 | 173 | 142 | 128 | 64.8 | 24.7 | 8.94 |
| | 105-012 | 117 | 108 | 110 | 138 | 190 | 211 | 165 | 66.9 | 22.3 |
| | 105-013 | 284 | 250 | 289 | 337 | 309 | 265 | 185 | 49.0 | 11.9 |
| | 105-014 | 276 | 304 | 317 | 479 | 481 | 327 | 352 | 170 | 49.6 |
| | 105-015 | 196 | 262 | 251 | 342 | 452 | 458 | 446 | 138 | 71.5 |
| | 105-020 | 180 | 173 | 218 | 281 | 285 | 267 | 207 | 101 | 35.2 |
| | 105-021 | 164 | 161 | 212 | 303 | 419 | 471 | 493 | 267 | 82.7 |
| | 105-025 | 293 | 317 | 335 | 308 | 244 | 149 | 43.6 | 6.88 | 1.08 |

BLQ = Below the limit of quantification (<1.00 ng/mL), treated as 0 for the calculation of PK parameters and descriptive statistics

TABLE 17
Individual and Summary Plasma Bupivacaine Concentrations Following Placement of INL-001 Bupivacaine
HCl Implants (300 mg) in Patients Following Abdominoplasty (PK Analysis Set) - Continued

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | | Bupivacaine Concentration (ng/mL) | | | | | | | | |
| Abdomino- | N | 20 | 20 | 20 | 20 | 20 | 20 | 20 | 20 | 20 |
| plasty | Mean | 0.2955 | 74.22 | 149.9 | 169.6 | 171.0 | 180.0 | 193.6 | 180.2 | 176.7 |
| | SD | 1.3215 | 66.204 | 113.19 | 116.46 | 105.33 | 97.120 | 104.33 | 80.780 | 77.025 |
| | CV % | 447.2 | 89.2 | 75.5 | 68.7 | 61.6 | 53.9 | 53.9 | 44.8 | 43.6 |
| | Min | 0.00 | 7.83 | 35.8 | 45.0 | 41.0 | 62.1 | 63.8 | 53.1 | 55.6 |
| | Median | 0.00 | 42.9 | 127 | 130 | 134 | 150 | 159 | 174 | 174 |
| | Max | 5.91 | 211 | 411 | 411 | 410 | 405 | 450 | 352 | 357 |
| | Geometric | NC | 49.45 | 114.9 | 135.2 | 145.0 | 158.9 | 170.5 | 163.2 | 160.8 |
| | Mean | | | | | | | | | |
| | Geometric | NC | 124.3 | 87.7 | 79.3 | 64.4 | 54.0 | 54.9 | 49.7 | 48.2 |
| | CV % | | | | | | | | | |

| | | Hour (h) | | | | | | | | |
|---|---|---|---|---|---|---|---|---|---|---|
| | | 8.0 | 10 | 12 | 18 | 24 | 36 | 48 | 72 | 96 |
| Surgery | | Bupivacaine Concentration (ng/mL) | | | | | | | | |
| Abdomino- | N | 20 | 20 | 20 | 20 | 20 | 20 | 20 | 20 | 20 |
| plasty | Mean | 188.3 | 195.1 | 206.1 | 226.8 | 242.3 | 228.6 | 190.8 | 84.68 | 31.20 |
| | SD | 85.757 | 94.130 | 94.550 | 89.997 | 109.01 | 110.22 | 133.68 | 69.507 | 23.955 |
| | CV % | 45.5 | 48.2 | 45.9 | 39.7 | 45.0 | 48.2 | 70.1 | 82.1 | 76.8 |
| | Min | 50.9 | 76.3 | 91.9 | 126 | 100 | 80.9 | 43.6 | 6.88 | 1.08 |
| | Median | 171 | 167 | 194 | 201 | 219 | 198 | 155 | 65.4 | 29.5 |
| | Max | 420 | 470 | 485 | 479 | 481 | 471 | 493 | 267 | 82.7 |
| | Geometric | 170.2 | 176.8 | 189.0 | 212.4 | 220.6 | 205.8 | 151.4 | 58.87 | 20.50 |
| | Mean | | | | | | | | | |
| | Geometric | 50.5 | 47.2 | 43.7 | 37.4 | 46.9 | 49.8 | 81.2 | 123.2 | 157.7 |
| | CV % | | | | | | | | | |

BLQ = Below the limit of quantification (<1.00 ng/mL), treated as 0 for the calculation of PK parameters and descriptive statistics;
NC = Not calculated

TABLE 18
Individual and Summary Plasma Bupivacaine Concentrations Following Placement of INL-001 Bupivacaine
HCl Implants (300 mg) in Patients Following Open Abdominal Hysterectomy (PK Analysis Set)

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | Subject | Bupivacaine Concentration (ng/mL) | | | | | | | | |
| Abdominal | 103-002 | BLQ | 356 | 467 | 575 | 545 | 558 | 547 | 550 | 503 |
| Hyster- | 103-005 | BLQ | 689 | 1410 | 1440 | 1400 | 1350 | 1280 | 1260 | 1100 |
| ectomy | 103-010 | BLQ | 236 | 481 | 511 | 532 | 631 | 550 | 567 | 551 |
| | 103-012 | BLQ | 513 | 727 | 751 | 742 | 672 | 692 | 661 | 606 |
| | 103-017 | BLQ | 553 | 621 | 547 | 356 | 612 | 702 | 636 | 839 |
| | 103-018 | BLQ | 254 | 340 | 360 | 370 | 578 | 702 | 724 | 809 |
| | 103-027 | BLQ | 552 | 502 | 746 | 703 | 668 | 680 | 595 | 605 |
| | 103-028 | BLQ | 512 | 600 | 566 | 619 | 563 | 581 | 479 | 458 |
| | 103-030 | BLQ | 159 | 261 | 250 | 224 | 231 | 205 | 200 | 122 |
| | 103-032 | BLQ | 288 | BLQ | BLQ | BLQ | 252 | 241 | 324 | 332 |
| | 103-033 | BLQ | 787 | 712 | 679 | 552 | 378 | 491 | 310 | 544 |
| | 103-036 | BLQ | 681 | 642 | 754 | 693 | 760 | 696 | 737 | 688 |
| | 103-039 | BLQ | 745 | 661 | 709 | 701 | 587 | 129 | 555 | 485 |
| | 103-040 | BLQ | 379 | 531 | 565 | 654 | 686 | 326 | 724 | 731 |
| | 103-044 | BLQ | 327 | 203 | 416 | 412 | 433 | 393 | 394 | . |
| | N | 15 | 15 | 15 | 15 | 15 | 15 | 15 | 15 | 14 |
| | Mean | 0.000 | 468.7 | 543.9 | 591.3 | 566.9 | 597.3 | 547.7 | 581.1 | 598.1 |
| | SD | 0.0000 | 199.78 | 315.07 | 313.74 | 308.86 | 259.74 | 281.38 | 248.96 | 236.05 |
| | CV % | NC | 42.6 | 57.9 | 53.1 | 54.5 | 43.5 | 51.4 | 42.8 | 39.5 |
| | Min | 0.00 | 159 | 0.00 | 0.00 | 0.00 | 231 | 129 | 200 | 122 |
| | Median | 0.00 | 512 | 531 | 566 | 552 | 587 | 550 | 567 | 578 |
| | Max | 0.00 | 787 | 1410 | 1440 | 1400 | 1350 | 1280 | 1260 | 1100 |
| | Geometric | NC | 425.1 | NC | NC | NC | 549.0 | 475.8 | 533.1 | 541.6 |
| | Mean | | | | | | | | | |
| | Geometric | NC | 50.7 | NC | NC | NC | 45.5 | 64.2 | 46.4 | 55.9 |
| | CV % | | | | | | | | | |
| | | Hour (h) | | | | | | | | |
| | | 8.0 | 10 | 12 | 18 | 24 | 36 | 48 | 72 | 96 |
| Surgery | Subject | Bupivacaine Concentration (ng/mL) | | | | | | | | |
| Abdominal | 103-002 | 575 | 545 | 547 | 551 | 570 | 239 | 195 | 73.6 | 21.8 |
| Hyster- | 103-005 | 1070 | 1090 | 1100 | 889 | 827 | 523 | 290 | 63.6 | 13.6 |
| ectomy | 103-010 | 587 | 625 | 663 | 731 | 724 | 638 | 473 | 167 | 29.2 |
| | 103-012 | 506 | 478 | 448 | 319 | 212 | 88.9 | 42.3 | 4.92 | 1.20 |
| | 103-017 | 593 | 586 | 531 | 453 | 323 | 212 | 186 | 94.7 | 29.1 |
| | 103-018 | 922 | 868 | 867 | 761 | 785 | 402 | 294 | 41.1 | 6.08 |
| | 103-027 | 592 | 545 | 501 | 530 | 473 | 363 | 234 | 69.7 | 13.7 |
| | 103-028 | 526 | 615 | 585 | 638 | 602 | 510 | 356 | 104 | 27.4 |
| | 103-030 | 173 | 176 | 80.7 | 173 | 193 | 141 | 77.7 | 21.7 | 3.64 |
| | 103-032 | 293 | 257 | 264 | 265 | 258 | 142 | 76.6 | 14.2 | 2.49 |
| | 103-033 | 489 | 698 | 653 | 632 | 689 | 603 | 368 | 176 | 40.1 |
| | 103-036 | 597 | 608 | 597 | 632 | 680 | 459 | 324 | 222 | 69.2 |
| | 103-039 | 448 | 397 | 289 | 268 | 196 | 113 | 72.2 | 11.5 | 2.06 |
| | 103-040 | 722 | 790 | 717 | 684 | 608 | 462 | 278 | 57.4 | 8.89 |
| | 103-044 | 492 | 382 | 400 | 389 | 414 | 218 | 150 | 33.2 | 9.79 |
| | N | 15 | 15 | 15 | 15 | 15 | 15 | 15 | 15 | 15 |
| | Mean | 572.3 | 577.3 | 549.5 | 527.7 | 503.6 | 340.9 | 227.8 | 76.97 | 18.55 |
| | SD | 218.25 | 232.71 | 248.35 | 209.73 | 223.74 | 186.82 | 127.94 | 65.624 | 18.459 |
| | CV % | 38.1 | 40.3 | 45.2 | 39.7 | 44.4 | 54.8 | 56.2 | 85.3 | 99.5 |
| | Min | 173 | 176 | 80.7 | 173 | 193 | 88.9 | 42.3 | 4.92 | 1.20 |
| | Median | 575 | 586 | 547 | 551 | 570 | 363 | 234 | 63.6 | 13.6 |
| | Max | 1070 | 1090 | 1100 | 889 | 827 | 638 | 473 | 222 | 69.2 |
| | Geometric | 530.1 | 528.2 | 480.1 | 481.6 | 448.7 | 286.1 | 186.0 | 49.79 | 10.79 |
| | Mean | | | | | | | | | |
| | Geometric | 45.2 | 49.1 | 68.2 | 49.8 | 56.6 | 72.7 | 83.3 | 150.5 | 177.3 |
| | CV % | | | | | | | | | |

BLQ = Below the limit of quantification (<1.00 ng/mL), treated as 0 for the calculation of PK parameters and descriptive statistics;
NC = Not calculated

TABLE 19
Individual and Summary Plasma Bupivacaine Concentrations Following Placement of INL-001 Bupivacaine
HCl Implants (300 mg) in Patients Following Laparoscopic-Assisted Colectomy (PK Analysis Set)

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | Subject | Bupivacaine Concentration (ng/mL) | | | | | | | | |
| Laparos- | 103-013 | BLQ | 16.2 | 44.1 | 39.6 | 74.9 | 99.0 | 99.8 | 142 | 151 |
| copic | 103-034 | BLQ | 63.4 | 95.9 | 85.4 | 112 | 117 | 126 | 138 | 142 |
| Colectomy | 103-035 | BLQ | 19.9 | 19.5 | 59.1 | 60.1 | 58.5 | 59.0 | 61.0 | 68.7 |
| | N | 3 | 3 | 3 | 3 | 3 | 3 | 3 | 3 | 3 |
| | Mean | 0.000 | 33.17 | 53.17 | 61.37 | 82.33 | 91.50 | 94.93 | 113.7 | 120.6 |
| | SD | 0.0000 | 26.248 | 38.999 | 22.984 | 26.737 | 29.962 | 33.764 | 45.654 | 45.143 |
| | CV % | NC | 79.1 | 73.4 | 37.5 | 32.5 | 32.7 | 35.6 | 40.2 | 37.4 |
| | Min | 0.00 | 16.2 | 19.5 | 39.6 | 60.1 | 58.5 | 59.0 | 61.0 | 68.7 |
| | Median | 0.00 | 19.9 | 44.1 | 59.1 | 74.9 | 99.0 | 99.8 | 138 | 142 |
| | Max | 0.00 | 63.4 | 95.9 | 85.4 | 112 | 117 | 126 | 142 | 151 |
| | Geometric | NC | 27.34 | 43.53 | 58.47 | 79.59 | 87.83 | 90.53 | 106.1 | 113.8 |
| | Mean | | | | | | | | | |
| | Geometric | NC | 84.7 | 94.1 | 39.9 | 32.4 | 37.4 | 40.4 | 50.9 | 46.0 |
| | CV % | | | | | | | | | |
| | | Hour (h) | | | | | | | | |
| | | 8.0 | 10 | 12 | 18 | 24 | 36 | 48 | 72 | 96 |
| Surgery | Subject | Bupivacaine Concentration (ng/mL) | | | | | | | | |
| Laparos- | 103-013 | 205 | 226 | 235 | 264 | 446 | 486 | 468 | 301 | 116 |
| copic | 103-034 | 116 | 105 | 116 | 162 | 140 | 104 | 501 | 53.4 | 67.0 |
| Colectomy | 103-035 | 70.8 | 47.6 | 85.0 | 161 | 227 | 239 | 267 | 135 | 55.4 |
| | N | 3 | 3 | 3 | 3 | 3 | 3 | 3 | 3 | 3 |
| | Mean | 130.6 | 126.2 | 145.3 | 195.7 | 271.0 | 276.3 | 412.0 | 163.1 | 79.47 |
| | SD | 68.281 | 91.070 | 79.185 | 59.181 | 157.67 | 193.72 | 126.65 | 126.17 | 32.166 |
| | CV % | 52.3 | 72.2 | 54.5 | 30.2 | 58.2 | 70.1 | 30.7 | 77.3 | 40.5 |
| | Min | 70.8 | 47.6 | 85.0 | 161 | 140 | 104 | 267 | 53.4 | 55.4 |
| | Median | 116 | 105 | 116 | 162 | 227 | 239 | 468 | 135 | 67.0 |
| | Max | 205 | 226 | 235 | 264 | 446 | 486 | 501 | 301 | 116 |
| | Geometric | 119.0 | 104.1 | 132.3 | 190.2 | 242.0 | 229.5 | 397.1 | 129.5 | 75.51 |
| | Mean | | | | | | | | | |
| | Geometric | 57.2 | 91.3 | 55.9 | 29.0 | 63.5 | 90.2 | 35.6 | 105.6 | 39.8 |
| | CV % | | | | | | | | | |

BLQ = Below the limit of quantification (<1.00 ng/mL), treated as 0 for the calculation of PK parameters and descriptive statistics

TABLE 20
Individual and Summary Plasma Bupivacaine Concentrations Following
Placement of INL-001 Bupivacaine HCl Implants (300 mg) in Patients
Following Reduction Mammoplasty (PK Analysis Set)

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | Subject | Bupivacaine Concentration (ng/mL) | | | | | | | | |
| Reduction | 101-013 | BLQ | 274 | 323 | 438 | 431 | 350 | 370 | 386 | 403 |
| Mammo- | 101-016 | BLQ | 166 | 230 | 336 | 336 | 263 | 202 | 191 | 156 |
| plasty | 101-020 | BLQ | 104 | 231 | 300 | 343 | 356 | 316 | 321 | 284 |
| | 101-021 | BLQ | 14.4 | 24.9 | . | 37.1 | 51.5 | 78.4 | 108 | 117 |
| | 101-022 | BLQ | 182 | 292 | 351 | 342 | 323 | 306 | 357 | 351 |
| | 101-023 | BLQ | 19.3 | 39.1 | 73.0 | 38.0 | 80.9 | 95.3 | 142 | 156 |
| | 101-024 | BLQ | 20.2 | 43.9 | 52.6 | 63.2 | 94.9 | 98.1 | 93.0 | 100 |
| | 101-029 | BLQ | 328 | 375 | 538 | 520 | 483 | 557 | 593 | 500 |
| | 101-030 | BLQ | 33.0 | 42.6 | 51.6 | 41.5 | 67.3 | 61.9 | 71.7 | 78.2 |
| | 101-034 | BLQ | 29.5 | 40.8 | 86.3 | 64.6 | 163 | 169 | 177 | 225 |
| | 101-036 | BLQ | 21.5 | 25.2 | 43.3 | 41.6 | 102 | 83.8 | 119 | 155 |
| | 101-038 | BLQ | 23.0 | 28.8 | 31.6 | 35.1 | 48.0 | 68.2 | 92.6 | 92.0 |
| | 101-042 | BLQ | 212 | 248 | 284 | 290 | 260 | 210 | 209 | 209 |
| | 101-043 | BLQ | 99.7 | 152 | 188 | 225 | 217 | 239 | 271 | 238 |
| | 101-044 | BLQ | 95.6 | 91.9 | 136 | 89.7 | 87.0 | 69.6 | 63.5 | 68.2 |
| | 103-001 | BLQ | 54.6 | 91.8 | 177 | 352 | 420 | 387 | 319 | 360 |
| | 103-007 | BLQ | 87.0 | 87.1 | 87.2 | 221 | 319 | 337 | 340 | 327 |
| | 103-009 | BLQ | 54.4 | 246 | 249 | 489 | 396 | 382 | 384 | 379 |
| | 103-014 | BLQ | 2.51 | 22.6 | 33.3 | 53.6 | 60.8 | 69.3 | 82.6 | 98.4 |
| | 103-015 | BLQ | 104 | 102 | 152 | 374 | 348 | 340 | 277 | 324 |

| | | Hour (h) | | | | | | | | |
|---|---|---|---|---|---|---|---|---|---|---|
| | | 8.0 | 10 | 12 | 18 | 24 | 36 | 48 | 72 | 96 |
| Surgery | Subject | Bupivacaine Concentration (ng/mL) | | | | | | | | |
| Reduction | 101-013 | 408 | 403 | 496 | 508 | 508 | 389 | 315 | 94.3 | 30.7 |
| Mammo- | 101-016 | 132 | 142 | 94.4 | 188 | 341 | 338 | 252 | 115 | 34.3 |
| plasty | 101-020 | 273 | 243 | 254 | 355 | 462 | 342 | 154 | 30.1 | 9.91 |
| | 101-021 | 130 | 95.7 | 107 | 168 | 227 | 309 | 147 | 89.0 | 65.2 |
| | 101-022 | 363 | 381 | 312 | 240 | 276 | 493 | 327 | 71.7 | 25.4 |
| | 101-023 | 196 | 220 | 266 | 355 | 504 | 445 | 428 | 180 | 63.2 |
| | 101-024 | 113 | 132 | 146 | 222 | 332 | 437 | 372 | 219 | 80.5 |
| | 101-029 | 465 | 471 | 470 | 546 | 510 | 410 | 193 | 97.5 | 31.0 |
| | 101-030 | 82.1 | 121 | 132 | 210 | 264 | 241 | 190 | 111 | 55.2 |
| | 101-034 | 254 | 289 | 317 | 480 | 639 | 637 | 641 | 285 | 123 |
| | 101-036 | 187 | 242 | 271 | 390 | 366 | 473 | 473 | 191 | 67.5 |
| | 101-038 | 102 | 132 | 176 | 258 | 330 | 355 | 273 | 238 | 106 |
| | 101-042 | 243 | 251 | 291 | 457 | 528 | 429 | 325 | 110 | 32.0 |
| | 101-043 | 208 | 197 | 195 | 234 | 274 | 153 | 88.0 | 21.2 | 9.55 |
| | 101-044 | 76.1 | 108 | 138 | 199 | 190 | 145 | 94.9 | 26.1 | 7.16 |
| | 103-001 | 279 | 345 | 414 | 539 | 649 | 581 | 514 | 253 | 81.2 |
| | 103-007 | 368 | 354 | 351 | 324 | 434 | 372 | 257 | 154 | 75.9 |
| | 103-009 | 237 | 382 | 309 | 279 | 211 | 154 | 84.2 | 25.5 | 6.75 |
| | 103-014 | 150 | 213 | 257 | 250 | 341 | 344 | 310 | 186 | 66.5 |
| | 103-015 | 319 | 354 | 389 | 399 | 423 | 324 | 293 | 131 | 51.8 |

BLQ = Below the limit of quantification (<1.00 ng/mL), treated as 0 for the calculation of PK parameters and descriptive statistics
. = Value missing or not reportable

TABLE 21
Individual and Summary Plasma Bupivacaine Concentrations Following Placement of INL-001 Bupivacaine
HCl Implants (300 mg) in Patients Following Reduction Mammoplasty (PK Analysis Set) - Continued

| | | Hour (h) | | | | | | | | |
|---|---|---|---|---|---|---|---|---|---|---|
| | | 0.0 | 0.50 | 1.0 | 1.5 | 2.0 | 3.0 | 4.0 | 5.0 | 6.0 |
| Surgery | | Bupivacaine Concentration (ng/mL) | | | | | | | | |
| Reduction | N | 20 | 20 | 20 | 19 | 20 | 20 | 20 | 20 | 20 |
| Mammo- | Mean | 0.000 | 96.24 | 136.9 | 189.9 | 219.4 | 224.5 | 222.0 | 229.9 | 231.0 |
| plasty | SD | 0.0000 | 92.117 | 114.99 | 149.63 | 170.80 | 143.62 | 145.20 | 141.42 | 127.75 |
| | CV % | NC | 95.7 | 84.0 | 78.8 | 77.9 | 64.0 | 65.4 | 61.5 | 55.3 |
| | Min | 0.00 | 2.51 | 22.6 | 31.6 | 35.1 | 48.0 | 61.9 | 63.5 | 68.2 |
| | Median | 0.00 | 70.8 | 91.9 | 152 | 223 | 239 | 206 | 200 | 217 |
| | Max | 0.00 | 328 | 375 | 538 | 520 | 483 | 557 | 593 | 500 |
| | Geometric | NC | 56.35 | 91.22 | 134.4 | 143.5 | 173.3 | 175.0 | 189.1 | 195.9 |
| | Mean | | | | | | | | | |
| | Geometric | NC | 181.1 | 125.8 | 112.6 | 138.6 | 93.7 | 85.3 | 74.3 | 67.5 |
| | CV % | | | | | | | | | |
| | | Hour (h) | | | | | | | | |
| | | 8.0 | 10 | 12 | 18 | 24 | 36 | 48 | 72 | 96 |
| Surgery | | Bupivacaine Concentration (ng/mL) | | | | | | | | |
| Reduction | N | 20 | 20 | 20 | 20 | 20 | 20 | 20 | 20 | 20 |
| Mammo- | Mean | 229.3 | 253.8 | 269.3 | 330.1 | 390.5 | 368.6 | 286.6 | 131.4 | 51.14 |
| plasty | SD | 112.79 | 113.33 | 117.57 | 123.85 | 135.90 | 131.11 | 148.40 | 79.658 | 33.155 |
| | CV % | 49.2 | 44.7 | 43.7 | 37.5 | 34.8 | 35.6 | 51.8 | 60.6 | 64.8 |
| | Min | 76.1 | 95.7 | 94.4 | 168 | 190 | 145 | 84.2 | 21.2 | 6.75 |
| | Median | 223 | 243 | 269 | 302 | 354 | 364 | 283 | 113 | 53.5 |
| | Max | 465 | 471 | 496 | 546 | 649 | 637 | 641 | 285 | 123 |
| | Geometric | 201.9 | 228.1 | 242.8 | 308.7 | 367.8 | 342.6 | 248.1 | 102.7 | 38.10 |
| | Mean | | | | | | | | | |
| | Geometric | 57.6 | 52.2 | 51.7 | 39.0 | 37.2 | 43.6 | 63.3 | 96.0 | 111.0 |
| | CV % | | | | | | | | | |

NC = Not calculated

TABLE 22
Individual Plasma Bupivacaine Concentrations Following Placement of INL-001 Bupivacaine HCl Implants
(300 mg) in Patients Following Unspecified Surgery (Subject 103-043) (PK Analysis Set)

| | | Hour (h) | | | | | | | | | | | | | | | | | |
|---|---|---|---|---|---|---|---|---|---|---|---|---|---|---|---|---|---|---|---|
| | | 0.0 | 0.50 | 1.0 | 1.5 | 2.0 | 3.0 | 4.0 | 5.0 | 6.0 | 8.0 | 10 | 12 | 18 | 24 | 36 | 48 | 72 | 96 |
| Surgery | Subject | Bupivacaine Concentration (ng/mL) | | | | | | | | | | | | | | | | | |
| . | 103-043 | BLQ | 220 | 290 | 397 | 438 | 521 | 543 | 458 | 475 | 524 | 504 | 504 | 449 | 400 | 204 | 115 | 54.2 | 17.8 |

BLQ = Below the limit of quantification (<1.00 ng/mL), treated as 0 for the calculation of PK parameters and descriptive statistics
. = Value missing or not reportable

TABLE 23
Summary Plasma Bupivacaine Concentrations Following Placement
of INL-001 Bupivacaine HCl Implants (300 mg) in Patients Following
Various Soft Tissue Surgeries (Overall) (PK Analysis Set)

| | Hour (h) | | | | | | | | |
|---|---|---|---|---|---|---|---|---|---|
| | 0.0 | 0.50 | 1.0 | 1.5 | 2.0 | 3.0 | 4.0 | 5.0 | 6.0 |
| | Bupivacaine Concentration (ng/mL) | | | | | | | | |
| N | 75 | 75 | 75 | 74 | 75 | 75 | 75 | 75 | 74 |
| Mean | 0.07880 | 183.0 | 240.8 | 288.8 | 304.8 | 321.7 | 317.1 | 316.6 | 316.4 |
| SD | 0.68243 | 191.63 | 230.26 | 242.33 | 239.78 | 226.02 | 224.89 | 216.45 | 215.80 |
| CV % | 866.0 | 104.7 | 95.6 | 83.9 | 78.7 | 70.2 | 70.9 | 68.4 | 68.2 |
| Min | 0.00 | 2.51 | 0.00 | 0.00 | 0.00 | 48.0 | 59.0 | 53.1 | 55.6 |
| Median | 0.00 | 107 | 200 | 248 | 256 | 290 | 291 | 271 | 259 |
| Max | 5.91 | 787 | 1410 | 1440 | 1400 | 1350 | 1280 | 1260 | 1100 |
| Geometric | NC | 99.67 | NC | NC | NC | 247.6 | 246.2 | 252.2 | 251.9 |
| Mean | | | | | | | | | |
| Geometric | NC | 189.0 | NC | NC | NC | 90.4 | 86.1 | 79.7 | 79.1 |
| CV % | | | | | | | | | |

Hour (h)

| | | | | | | | | |
|---|---|---|---|---|---|---|---|---|
| 8.0 | 10 | 12 | 18 | 24 | 36 | 48 | 72 | 96 |

Bupivacaine Concentration (ng/mL)

| | | | | | | | | | |
|---|---|---|---|---|---|---|---|---|---|
| N | 75 | 75 | 75 | 75 | 74 | 75 | 75 | 75 | 75 |
| Mean | 316.5 | 328.4 | 331.6 | 349.7 | 365.4 | 300.3 | 233.4 | 100.5 | 38.58 |
| SD | 202.99 | 208.18 | 204.30 | 179.38 | 179.74 | 146.42 | 136.23 | 71.175 | 28.586 |
| CV % | 64.1 | 63.4 | 61.6 | 51.3 | 49.2 | 48.8 | 58.4 | 70.8 | 74.1 |
| Min | 50.9 | 47.6 | 80.7 | 78.2 | 57.4 | 54.8 | 41.9 | 4.92 | 1.08 |
| Median | 273 | 263 | 287 | 305 | 331 | 269 | 197 | 89.0 | 33.3 |
| Max | 1070 | 1090 | 1100 | 889 | 827 | 638 | 641 | 301 | 123 |
| Geometric | 259.1 | 269.8 | 276.5 | 307.8 | 320.7 | 262.5 | 192.3 | 74.19 | 25.93 |
| Mean | | | | | | | | | |
| Geometric | 73.0 | 72.2 | 68.1 | 55.0 | 58.0 | 59.4 | 74.8 | 108.4 | 145.7 |
| CV % | | | | | | | | | |

NC = Not calculated

TABLE 24
Individual and Summary Plasma Bupivacaine Pharmacokinetic Parameters
Following Placement of INL-001 Bupivacaine HCl Implants (300 mg) in
Patients Following Open Ventral Hernia Repair (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| Ventral | 102-002 | 17640 | 18840 | 329 | 24.00 | 0.50 | 96.00 | 20.11 |
| Hernia | 102-003 | 18880 | 20350 | 346 | 24.00 | 0.50 | 96.00 | 23.33 |
| Repair | 102-004 | 22230 | 23300 | 464 | 24.00 | 0.50 | 96.00 | 18.39 |
| | 102-005 | 17790 | 18630 | 474 | 4.00 | 0.50 | 96.00 | 18.99 |
| | 102-006 | 33910 | 36760 | 608 | 12.00 | 0.50 | 96.00 | 22.77 |
| | 102-007 | 26170 | 27680 | 659 | 2.00 | 0.50 | 96.00 | 20.38 |
| | 102-008 | 19530 | 22050 | 411 | 1.50 | 0.50 | 96.00 | 28.60 |
| | 102-009 | 13340 | 15020 | 266 | 24.00 | 0.50 | 96.00 | 29.89 |
| | 102-011 | 12620 | 14820 | 418 | 3.00 | 0.50 | 96.00 | 37.23 |
| | 102-013 | 32110 | 32540 | 772 | 12.00 | 0.50 | 96.00 | 12.68 |
| | 103-019 | 24460 | 25330 | 557 | 24.00 | 0.50 | 96.00 | 17.93 |
| | 103-021 | 21680 | 23760 | 548 | 10.00 | 0.50 | 96.00 | 29.27 |
| | 103-022 | 26670 | 27060 | 535 | 1.50 | 0.50 | 96.00 | 12.17 |
| | 103-023 | 5318 | 6736 | 205 | 2.00 | 0.50 | 96.00 | 46.13 |
| | 103-024 | 28390 | 31600 | 777 | 4.00 | 0.50 | 96.00 | 26.25 |
| | 103-029 | 13050 | 15510 | 359 | 4.00 | 0.50 | 96.00 | 40.85 |
| | N | 16 | 16 | 16 | 16 | 16 | 16 | 16 |
| | Mean | 20862 | 22499 | 483.0 | 11.000 | 0.500 | 96.000 | 25.310 |
| | SD | 7675.81 | 7704.04 | 167.54 | 9.6661 | 0.0000 | 0.0000 | 9.6673 |
| | CV % | 36.8 | 34.2 | 34.7 | 87.9 | 0.0 | 0.0 | 38.2 |
| | Min | 5318 | 6736 | 205 | 1.50 | 0.50 | 96.00 | 12.17 |
| | Median | 20610 | 22680 | 469 | 7.00 | 0.50 | 96.00 | 23.05 |
| | Max | 33910 | 36760 | 777 | 24.00 | 0.50 | 96.00 | 46.13 |
| | Geometric | 19209 | 21022 | 454.4 | 6.777 | 0.500 | 96.000 | 23.667 |
| | Mean | | | | | | | |
| | Geometric | 48.4 | 42.8 | 38.5 | 151.0 | 0.0 | 0.0 | 39.4 |
| | CV % | | | | | | | |

TABLE 25
Individual and Summary Plasma Bupivacaine Pharmacokinetic Parameters Following Placement of INL-
001 Bupivacaine HCl Implants (300 mg) in Patients Following Abdominoplasty (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| Abdominoplasty | 101-003 | 22540 | NC | 387 | 48.00 | 0.50 | 96.00 | NC |
| | 101-005 | 12960 | 13680 | 219 | 24.00 | 0.50 | 96.00 | 18.36 |
| | 101-008 | 18950 | 20320 | 319 | 36.00 | 0.50 | 96.00 | 18.81 |
| | 101-009 | 7699 | 8500 | 174 | 18.00 | 0.50 | 96.00 | 27.76 |
| | 101-040 | 13420 | 15160 | 262 | 24.00 | 0.50 | 96.00 | 28.33 |
| | 105-001 | 10390 | 11490 | 169 | 8.00 | 0.50 | 96.00 | 24.03 |
| | 105-002 | 12660 | 12910 | 485 | 12.00 | 0.50 | 96.00 | 15.78 |
| | 105-004 | 6416 | 6477 | 321 | 1.00 | 0.50 | 96.00 | 12.58 |
| | 105-005 | 11680 | 12780 | 219 | 24.00 | NC | 96.00 | 21.19 |
| | 105-006 | 10600 | 10680 | 212 | 36.00 | 0.50 | 96.00 | 10.63 |
| | 105-007 | 13490 | 13690 | 411 | 1.00 | 0.50 | 96.00 | 13.39 |
| | 105-010 | 7903 | 9328 | 143 | 18.00 | 0.50 | 96.00 | 29.67 |
| | 105-011 | 8822 | 9039 | 240 | 8.00 | 0.50 | 96.00 | 16.80 |
| | 105-012 | 11930 | 12470 | 450 | 4.00 | 0.50 | 96.00 | 16.62 |
| | 105-013 | 16420 | 16630 | 337 | 18.00 | 0.50 | 96.00 | 12.13 |
| | 105-014 | 26510 | 27720 | 481 | 24.00 | 0.50 | 96.00 | 16.98 |
| | 105-015 | 26810 | 28690 | 458 | 36.00 | 0.50 | 96.00 | 18.17 |
| | 105-020 | 16660 | 17610 | 285 | 24.00 | 0.50 | 96.00 | 18.78 |
| | 105-021 | 29740 | NC | 493 | 48.00 | 0.50 | 96.00 | NC |
| | 105-025 | 11600 | 11610 | 411 | 1.50 | 0.50 | 96.00 | 9.00 |
| | N | 20 | 18 | 20 | 20 | 19 | 20 | 18 |
| | Mean | 14860 | 14377 | 323.8 | 20.675 | 0.500 | 96.000 | 18.278 |
| | SD | 6761.69 | 6059.39 | 116.66 | 14.6739 | 0.0000 | 0.0000 | 6.0124 |
| | CV % | 45.5 | 42.1 | 36.0 | 71.0 | 0.0 | 0.0 | 32.9 |
| | Min | 6416 | 6477 | 143 | 1.00 | 0.50 | 96.00 | 9.00 |
| | Median | 12810 | 12850 | 320 | 21.00 | 0.50 | 96.00 | 17.58 |
| | Max | 29740 | 28690 | 493 | 48.00 | 0.50 | 96.00 | 29.67 |
| | Geometric | 13572 | 13356 | 302.4 | 13.125 | 0.500 | 96.000 | 17.358 |
| | Mean | | | | | | | |
| | Geometric | 45.2 | 40.2 | 40.5 | 186.0 | 0.0 | 0.0 | 34.3 |
| | CV % | | | | | | | |

NC = Not calculated

TABLE 26
Individual and Summary Plasma Bupivacaine Pharmacokinetic Parameters
Following Placement of INL-001 Bupivacaine HCl Implants (300 mg)
in Patients Following Open Abdominal Hysterectomy (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| Abdominal | 103-002 | 24790 | 25260 | 575 | 1.50 | 0.50 | 96.00 | 15.18 |
| Hysterectomy | 103-005 | 43040 | 43260 | 1440 | 1.50 | 0.50 | 96.00 | 10.87 |
| | 103-010 | 40090 | 40600 | 731 | 18.00 | 0.50 | 96.00 | 11.95 |
| | 103-012 | 14030 | 14050 | 751 | 1.50 | 0.50 | 96.00 | 9.45 |
| | 103-017 | 22960 | 23710 | 839 | 6.00 | 0.50 | 96.00 | 17.94 |
| | 103-018 | 33830 | 33900 | 922 | 8.00 | 0.50 | 96.00 | 8.58 |
| | 103-027 | 26400 | 26630 | 746 | 1.50 | 0.50 | 96.00 | 11.72 |
| | 103-028 | 32830 | 33350 | 638 | 18.00 | 0.50 | 96.00 | 12.97 |
| | 103-030 | 8773 | 8833 | 261 | 1.00 | 0.50 | 96.00 | 11.45 |
| | 103-032 | 10980 | 11020 | 332 | 6.00 | 0.50 | 96.00 | 9.71 |
| | 103-033 | 37040 | 37960 | 787 | 0.50 | 0.50 | 96.00 | 15.81 |
| | 103-036 | 36990 | 39300 | 760 | 3.00 | 0.50 | 96.00 | 23.09 |
| | 103-039 | 12760 | 12780 | 745 | 0.50 | 0.50 | 96.00 | 9.35 |
| | 103-040 | 31560 | 31680 | 790 | 10.00 | 0.50 | 96.00 | 9.66 |
| | 103-044 | 18280 | 18450 | 492 | 8.00 | 0.50 | 96.00 | 12.19 |
| | N | 15 | 15 | 15 | 15 | 15 | 15 | 15 |
| | Mean | 26290 | 26718 | 720.6 | 5.667 | 0.500 | 96.000 | 12.663 |
| | SD | 11278.9 | 11562.2 | 270.94 | 5.8909 | 0.0000 | 0.0000 | 3.9179 |
| | CV % | 42.9 | 43.3 | 37.6 | 104.0 | 0.0 | 0.0 | 30.9 |
| | Min | 8773 | 8833 | 261 | 0.50 | 0.50 | 96.00 | 8.58 |
| | Median | 26400 | 26630 | 746 | 3.00 | 0.50 | 96.00 | 11.72 |
| | Max | 43040 | 43260 | 1440 | 18.00 | 0.50 | 96.00 | 23.09 |
| | Geometric | 23600 | 23928 | 670.9 | 3.139 | 0.500 | 96.000 | 12.191 |
| | Mean | | | | | | | |
| | Geometric | 54.7 | 55.4 | 42.8 | 180.4 | 0.0 | 0.0 | 28.0 |
| | CV % | | | | | | | |

NC = Not calculated

TABLE 27
Individual and Summary Plasma Bupivacaine Pharmacokinetic Parameters
Following Placement of INL-001 Bupivacaine HCl Implants (300 mg) in
Patients Following Laparoscopic-Assisted Colectomy (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| Laparoscopic | 103-013 | 30950 | 34940 | 486 | 36.00 | 0.50 | 96.00 | 23.85 |
| Colectomy | 103-034 | 16290 | NC | 501 | 48.00 | 0.50 | 96.00 | NC |
| | 103-035 | 15540 | NC | 267 | 48.00 | 0.50 | 96.00 | NC |
| | N | 3 | 1 | 3 | 3 | 3 | 3 | 1 |
| | Mean | 20925 | 34937 | 418.0 | 44.000 | 0.500 | 96.000 | 23.852 |
| | SD | 8685.78 | NC | 130.98 | 6.9282 | 0.0000 | 0.0000 | NC |
| | CV % | 41.5 | NC | 31.3 | 15.7 | 0.0 | 0.0 | NC |
| | Min | 15540 | 34940 | 267 | 36.00 | 0.50 | 96.00 | 23.85 |
| | Median | 16290 | 34940 | 486 | 48.00 | 0.50 | 96.00 | 23.85 |
| | Max | 30950 | 34940 | 501 | 48.00 | 0.50 | 96.00 | 23.85 |
| | Geometric | 19861 | 34937 | 402.1 | 43.611 | 0.500 | 96.000 | 23.852 |
| | Mean | | | | | | | |
| | Geometric | 39.9 | NC | 36.6 | 16.7 | 0.0 | 0.0 | NC |
| | CV % | | | | | | | |

NC = Not calculated

TABLE 28
Individual and Summary Plasma Bupivacaine Pharmacokinetic Parameters
Following Placement of INL-001 Bupivacaine HCl Implants (300 mg)
in Patients Following Reduction Mammoplasty (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| Reduction | 101-013 | 26750 | 27380 | 508 | 18.00 | 0.50 | 96.00 | 14.29 |
| Mammoplasty | 101-016 | 18390 | 19220 | 341 | 24.00 | 0.50 | 96.00 | 16.68 |
| | 101-020 | 18050 | 18210 | 462 | 24.00 | 0.50 | 96.00 | 11.61 |
| | 101-021 | 13680 | 17530 | 309 | 36.00 | 0.50 | 96.00 | 40.93 |
| | 101-022 | 22670 | 23150 | 493 | 36.00 | 0.50 | 96.00 | 13.02 |
| | 101-023 | 27330 | 28920 | 504 | 24.00 | 0.50 | 96.00 | 17.39 |
| | 101-024 | 24100 | 26620 | 437 | 36.00 | 0.50 | 96.00 | 21.74 |
| | 101-029 | 26120 | 26880 | 593 | 5.00 | 0.50 | 96.00 | 16.95 |
| | 101-030 | 14620 | 16870 | 264 | 24.00 | 0.50 | 96.00 | 28.29 |
| | 101-034 | 39460 | NC | 641 | 48.00 | 0.50 | 96.00 | NC |
| | 101-036 | 27770 | NC | 473 | 36.00 | 0.50 | 96.00 | NC |
| | 101-038 | 22260 | 27640 | 355 | 36.00 | 0.50 | 96.00 | 35.17 |
| | 101-042 | 25250 | 25910 | 528 | 24.00 | 0.50 | 96.00 | 14.35 |
| | 101-043 | 10980 | 11180 | 274 | 24.00 | 0.50 | 96.00 | 14.57 |
| | 101-044 | 8536 | 8669 | 199 | 18.00 | 0.50 | 96.00 | 12.87 |
| | 103-001 | 37340 | 39450 | 649 | 24.00 | 0.50 | 96.00 | 18.03 |
| | 103-007 | 24180 | 27120 | 434 | 24.00 | 0.50 | 96.00 | 26.84 |
| | 103-009 | 12480 | 12610 | 489 | 2.00 | 0.50 | 96.00 | 13.34 |
| | 103-014 | 21720 | 23800 | 344 | 36.00 | 0.50 | 96.00 | 21.61 |
| | 103-015 | 23940 | 25380 | 423 | 24.00 | 0.50 | 96.00 | 19.20 |
| | N | 20 | 18 | 20 | 20 | 20 | 20 | 18 |
| | Mean | 22281 | 22585 | 436.0 | 26.150 | 0.500 | 96.000 | 19.828 |
| | SD | 7978.99 | 7518.94 | 123.71 | 10.9173 | 0.0000 | 0.0000 | 8.1466 |
| | CV % | 35.8 | 33.3 | 28.4 | 41.7 | 0.0 | 0.0 | 41.1 |
| | Min | 8536 | 8669 | 199 | 2.00 | 0.50 | 96.00 | 11.61 |
| | Median | 23310 | 24590 | 450 | 24.00 | 0.50 | 96.00 | 17.17 |
| | Max | 39460 | 39450 | 649 | 48.00 | 0.50 | 96.00 | 40.93 |
| | Geometric | 20820 | 21230 | 417.7 | 22.263 | 0.500 | 96.000 | 18.554 |
| | Mean | | | | | | | |
| | Geometric | 40.9 | 39.6 | 31.9 | 84.1 | 0.0 | 0.0 | 37.2 |
| | CV % | | | | | | | |

NC = Not calculated

TABLE 29
Individual Plasma Bupivacaine Pharmacokinetic Parameters Following
Placement of INL-001 Bupivacaine HCl Implants (300 mg) in Patients
Following Unspecified Surgery (Subject 103-043) (PK Analysis Set)

| | | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|---|
| Surgery | Subject | (h*ng/mL) | (h*ng/mL) | (ng/mL) | (h) | (h) | (h) | (h) |
| . | 103-043 | 19410 | 19870 | 543 | 4.00 | 0.50 | 96.00 | 17.57 |

. = Value missing or not reportable

TABLE 30
Summary Plasma Bupivacaine Pharmacokinetic Parameters Following Placement
of INL-001 Bupivacaine HCl Implants (300 mg) in Patients Following
Various Soft Tissue Surgeries (Overall) (PK Analysis Set)

| | AUC₀₋ₗₐₛₜ | AUC_{0-∞} | Cₘₐₓ | Tₘₐₓ | T_{lag} | Tₗₐₛₜ | t_{1/2} |
|---|---|---|---|---|---|---|---|
| | (h*ng/mL) | (h*ng/mL) | (ng/mL) | (h) | (h) | (h) | (h) |
| N | 75 | 69 | 75 | 75 | 74 | 75 | 69 |
| Mean | 20709 | 21462 | 473.7 | 17.780 | 0.500 | 96.000 | 19.163 |
| SD | 9077.00 | 9306.22 | 215.61 | 14.2832 | 0.0000 | 0.0000 | 8.2645 |
| CV % | 43.8 | 43.4 | 45.5 | 80.3 | 0.0 | 0.0 | 43.1 |
| Min | 5318 | 6477 | 143 | 0.50 | 0.50 | 96.00 | 8.58 |
| Median | 19410 | 20320 | 462 | 18.00 | 0.50 | 96.00 | 17.57 |
| Max | 43040 | 43260 | 1440 | 48.00 | 0.50 | 96.00 | 46.13 |
| Geometric | 18669 | 19385 | 429.8 | 10.181 | 0.500 | 96.000 | 17.660 |
| Mean | | | | | | | |
| Geometric | 50.3 | 49.8 | 47.4 | 203.0 | 0.0 | 0.0 | 41.7 |
| CV % | | | | | | | |

TABLE 31
Summary Bupivacaine Pharmacokinetic Parameters Following
Administration of a Single Dose of POSIMIR 5 mL (660 mg)

| | | Cₘₐₓ | AUC₀₋ₜ^{#} | Tₘₐₓ | T_{1/2} |
|---|---|---|---|---|---|
| Surgical Procedure | | (ng/mL) | (h · ng/mL) | (h)^{\hat{ }} | (h) |
| Arthroscopic | N | 36 | 36 | 36 | 36 |
| subacromial | Mean | 593 | 19395 | 5.9^{\hat{ }} | 16.4 |
| decompression | SD | 299 | 12056 | 1.0-24.0^{\hat{ }} | 5.1 |
| Study 1* | | | | | |
| Arthroscopic | N | 18 | 18 | 18 | 18 |
| subacromial | Mean | 1006 | 47015 | 8.0^{\hat{ }} | 26.1 |
| decompression | SD | 454 | 20040 | 2.1-26.9^{\hat{ }} | 8.2 |
| Study 3 | | | | | |

^{#}t = last sampling time
^{\hat{ }}Median/Range (min-max)
*Drug leakage from the surgical site was suspected.

TABLE 32
Individual Plasma Bupivacaine Terminal Pharmacokinetic Parameters Following Placement of INL-001 Bupivacaine
HCl Implants (300 mg) in Patients Following Open Ventral Hernia Repair (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| Ventral | 102-002 | 0.9990 | 3 | 0.0345 | 48.00 | 96.00 | 6.36 |
| Hernia | 102-003 | 0.9765 | 4 | 0.0297 | 36.00 | 96.00 | 7.24 |
| Repair | 102-004 | 0.9886 | 3 | 0.0377 | 48.00 | 96.00 | 4.62 |
| | 102-005 | 0.9858 | 3 | 0.0365 | 48.00 | 96.00 | 4.51 |
| | 102-006 | 0.9564 | 3 | 0.0304 | 48.00 | 96.00 | 7.73 |
| | 102-007 | 0.9987 | 4 | 0.0340 | 36.00 | 96.00 | 5.45 |
| | 102-008 | 0.9752 | 4 | 0.0242 | 36.00 | 96.00 | 11.45 |
| | 102-009 | 0.9961 | 3 | 0.0232 | 48.00 | 96.00 | 11.23 |
| | 102-011 | 0.9759 | 4 | 0.0186 | 36.00 | 96.00 | 14.86 |
| | 102-013 | 0.9958 | 3 | 0.0547 | 48.00 | 96.00 | 1.34 |
| | 103-019 | 0.9989 | 3 | 0.0387 | 48.00 | 96.00 | 3.43 |
| | 103-021 | 0.9324 | 3 | 0.0237 | 48.00 | 96.00 | 8.73 |
| | 103-022 | 0.9985 | 3 | 0.0570 | 48.00 | 96.00 | 1.44 |
| | 103-023 | 0.9807 | 4 | 0.0150 | 36.00 | 96.00 | 21.04 |
| | 103-024 | 0.8158 | 5 | 0.0264 | 24.00 | 96.00 | 10.15 |
| | 103-029 | 0.9744 | 4 | 0.0170 | 36.00 | 96.00 | 15.85 |

TABLE 33
Individual Plasma Bupivacaine Terminal Pharmacokinetic Parameters Following Placement of INL-
001 Bupivacaine HCl Implants (300 mg) in Patients Following Abdominoplasty (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| Abdominoplasty | 101-003 | NC | 0 | NC | NC | NC | NC |
| | 101-005 | 0.9342 | 3 | 0.0378 | 48.00 | 96.00 | 5.25 |
| | 101-008 | 0.9455 | 3 | 0.0368 | 48.00 | 96.00 | 6.75 |
| | 101-009 | 0.9854 | 3 | 0.0250 | 48.00 | 96.00 | 9.42 |
| | 101-040 | 0.9943 | 4 | 0.0245 | 36.00 | 96.00 | 11.46 |
| | 105-001 | 1.0000 | 3 | 0.0288 | 48.00 | 96.00 | 9.59 |
| | 105-002 | 0.9974 | 4 | 0.0439 | 36.00 | 96.00 | 1.92 |
| | 105-004 | 0.9816 | 4 | 0.0551 | 36.00 | 96.00 | 0.94 |
| | 105-005 | 0.9951 | 3 | 0.0327 | 48.00 | 96.00 | 8.61 |
| | 105-006 | 0.9970 | 3 | 0.0652 | 48.00 | 96.00 | 0.80 |
| | 105-007 | 0.9940 | 4 | 0.0518 | 36.00 | 96.00 | 1.45 |
| | 105-010 | 0.9724 | 4 | 0.0234 | 36.00 | 96.00 | 15.28 |
| | 105-011 | 0.9995 | 3 | 0.0413 | 48.00 | 96.00 | 2.40 |
| | 105-012 | 0.9936 | 3 | 0.0417 | 48.00 | 96.00 | 4.29 |
| | 105-013 | 0.9993 | 3 | 0.0572 | 48.00 | 96.00 | 1.25 |
| | 105-014 | 0.9569 | 3 | 0.0408 | 48.00 | 96.00 | 4.38 |
| | 105-015 | 0.9485 | 3 | 0.0381 | 48.00 | 96.00 | 6.54 |
| | 105-020 | 0.9762 | 3 | 0.0369 | 48.00 | 96.00 | 5.41 |
| | 105-021 | NC | 0 | NC | NC | NC | NC |
| | 105-025 | 1.0000 | 3 | 0.0770 | 48.00 | 96.00 | 0.12 |

NC = Not calculated

TABLE 34
Individual Plasma Bupivacaine Terminal Pharmacokinetic Parameters Following Placement of INL-001 Bupivacaine
HCl Implants (300 mg) in Patients Following Open Abdominal Hysterectomy (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| Abdominal | 103-002 | 0.9919 | 3 | 0.0456 | 48.00 | 96.00 | 1.89 |
| Hysterectomy | 103-005 | 1.0000 | 3 | 0.0637 | 48.00 | 96.00 | 0.49 |
| | 103-010 | 0.9584 | 3 | 0.0580 | 48.00 | 96.00 | 1.24 |
| | 103-012 | 0.9952 | 6 | 0.0733 | 18.00 | 96.00 | 0.12 |
| | 103-017 | 0.9518 | 3 | 0.0386 | 48.00 | 96.00 | 3.18 |
| | 103-018 | 0.9999 | 3 | 0.0808 | 48.00 | 96.00 | 0.22 |
| | 103-027 | 0.9858 | 3 | 0.0591 | 48.00 | 96.00 | 0.87 |
| | 103-028 | 0.9989 | 3 | 0.0534 | 48.00 | 96.00 | 1.54 |
| | 103-030 | 0.9867 | 4 | 0.0605 | 36.00 | 96.00 | 0.68 |
| | 103-032 | 0.9998 | 3 | 0.0714 | 48.00 | 96.00 | 0.32 |
| | 103-033 | 0.9623 | 4 | 0.0438 | 36.00 | 96.00 | 2.41 |
| | 103-036 | 0.9150 | 4 | 0.0300 | 36.00 | 96.00 | 5.87 |
| | 103-039 | 0.9993 | 3 | 0.0741 | 48.00 | 96.00 | 0.22 |
| | 103-040 | 0.9954 | 3 | 0.0717 | 48.00 | 96.00 | 0.39 |
| | 103-044 | 0.9927 | 3 | 0.0569 | 48.00 | 96.00 | 0.93 |

TABLE 35
Individual Plasma Bupivacaine Terminal Pharmacokinetic Parameters Following Placement of INL-001 Bupivacaine
HCl Implants (300 mg) in Patients Following Laparoscopic-Assisted Colectomy (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| Laparoscopic | 103-013 | 0.9140 | 3 | 0.0291 | 48.00 | 96.00 | 11.43 |
| Colectomy | 103-034 | NC | 0 | NC | NC | NC | NC |
| | 103-035 | NC | 0 | NC | NC | NC | NC |

NC = Not calculated

TABLE 36
Individual Plasma Bupivacaine Terminal Pharmacokinetic Parameters Following Placement of INL-001
Bupivacaine HCl Implants (300 mg) in Patients Following Reduction Mammoplasty (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| Reduction | 101-013 | 0.9991 | 3 | 0.0485 | 48.00 | 96.00 | 2.31 |
| Mammoplasty | 101-016 | 0.9701 | 3 | 0.0415 | 48.00 | 96.00 | 4.30 |
| | 101-020 | 0.9879 | 4 | 0.0597 | 36.00 | 96.00 | 0.91 |
| | 101-021 | 0.9640 | 3 | 0.0169 | 48.00 | 96.00 | 21.96 |
| | 101-022 | 0.9768 | 3 | 0.0532 | 48.00 | 96.00 | 2.06 |
| | 101-023 | 0.9941 | 3 | 0.0399 | 48.00 | 96.00 | 5.48 |
| | 101-024 | 0.9388 | 3 | 0.0319 | 48.00 | 96.00 | 9.48 |
| | 101-029 | 0.9770 | 4 | 0.0409 | 36.00 | 96.00 | 2.82 |
| | 101-030 | 0.9904 | 4 | 0.0245 | 36.00 | 96.00 | 13.35 |
| | 101-034 | NC | 0 | NC | NC | NC | NC |
| | 101-036 | NC | 0 | NC | NC | NC | NC |
| | 101-038 | 0.7123 | 3 | 0.0197 | 48.00 | 96.00 | 19.46 |
| | 101-042 | 0.9972 | 3 | 0.0483 | 48.00 | 96.00 | 2.56 |
| | 101-043 | 0.9786 | 4 | 0.0476 | 36.00 | 96.00 | 1.80 |
| | 101-044 | 1.0000 | 3 | 0.0538 | 48.00 | 96.00 | 1.53 |
| | 103-001 | 0.9648 | 3 | 0.0384 | 48.00 | 96.00 | 5.35 |
| | 103-007 | 0.9928 | 4 | 0.0258 | 36.00 | 96.00 | 10.84 |
| | 103-009 | 0.9989 | 4 | 0.0519 | 36.00 | 96.00 | 1.03 |
| | 103-014 | 0.9273 | 3 | 0.0321 | 48.00 | 96.00 | 8.71 |
| | 103-015 | 0.9967 | 3 | 0.0361 | 48.00 | 96.00 | 5.65 |

NC = Not calculated

TABLE 37
Individual Plasma Bupivacaine Terminal Pharmacokinetic Parameters Following Placement of INL-001
Bupivacaine HCl Implants (300 mg) in Patients Following Unspecified Surgery (PK Analysis Set)

| | | | | | | | AUC_% |
|---|---|---|---|---|---|---|---|
| | | R² | | Lambda_z | Lambda_z_lower | Lambda_z_upper | Extrap_obs |
| Surgery | Subject | Adjusted | No_points_lambda_z | (1/h) | (h) | (h) | (%) |
| . | 103-043 | 0.9898 | 4 | 0.0395 | 36.00 | 96.00 | 2.27 |

. = Value missing or not reportable

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

1. A drug delivery device comprising a collagen matrix and bupivacaine or a salt thereof, the drug delivery device comprising a level of impurities selected from less than about 175 ppm ethylene chlorohydrin, less than about 0.9 ppm ethylene oxide, less than about 1,000 ppm ethylene glycol, and less than about 5 ppm elemental impurities.

2. The drug delivery device of claim 1, wherein the drug delivery device has a tensile strength selected from a dry tensile strength of about 1.6 N to about 2.4 N, and a wet tensile strength of about 0.6 N to about 1.2 N.

3. The drug delivery device of claim 1 or 2, wherein the device comprises about 4 mg/cm³ to about 8 mg/cm³ collagen and about 6 mg/cm³ to about 10 mg/cm³ bupivacaine or a salt thereof.

4. The drug delivery device of any one of claims 1 to 3, wherein the collagen matrix comprises about 6 mg/cm³ collagen and about 8 mg/cm³ bupivacaine, or a salt thereof.

5. The drug delivery device of any one of claims 1 to 4, comprising bupivacaine hydrochloride.

6. The drug delivery device of any one of claims 1 to 5, the device having dimensions of about 5 cm×about 5 cm×about 0.5 cm.

7. The drug delivery device of any one of claims 1 to 6, wherein the bupivacaine, or a salt thereof, is released from the device with an in vitro release profile of 50%±10% in the first 30 minutes, 75%±10% within 2 hours, and greater than 80% within 6 hours.

8. A method of making a mature lyophilized milled collagen (mLMC), the method comprising: providing isolated collagen, optionally an isolated collagen dispersion; freezing the isolated collagen; dehydrating the frozen collagen; and maturing the dehydrated collagen.

9. The method of claim 8, further comprising placing the dehydrated collagen in a permeable pouch before the maturing.

10. The method of claim 8 or 9, wherein the maturing comprises heating the dehydrated collagen in an environment with controlled temperature and controlled humidity.

11. The method of claim 10, wherein the heating is between about 35° C. and about 45° C., at between about 60% to about 70% relative humidity.

12. The method of claim 11, wherein the heating is at about 40° C. in an environment of about 65% relative humidity.

13. The method of any one of claims 10 to 12, further comprising maintaining the dehydrated collagen in the environment with controlled temperature and controlled humidity until the dehydrated collagen reaches a LOD (loss on drying) of between about 17% and about 22%.

14. The method of claim 13, wherein the dehydrated collagen reaches a LOD of about 18%.

15. The method of claim 14, wherein an aqueous dispersion comprising 0.9 wt % of the dehydrated collagen matured to an LOD of about 18% has a viscosity of between about 110 cP and about 250 cP.

16. The method of any of claims 8 to 15, further comprising dehumidifying the mature LMC to form dehumidified mature LMC.

17. The method of claim 16, further comprising placing the mature LMC in a permeable pouch before dehumidifying.

18. The method of claim 17, wherein the dehumidifying comprises heating the mature LMC in an environment with controlled temperature and controlled humidity.

19. The method of claim 18, comprising dehumidifying the mature LMC at a temperature of between about 20° C. and about 30° C. in an environment of between about 10% and about 20% relative humidity.

20. The method of claim 19, comprising dehumidifying the mature LMC at a temperature of about 25° C. in an environment of about 15% relative humidity.

21. The method of any one of claims 18 to 20, wherein the mature LMC is dehumidified until a loss on drying of between about 8% and about 12% is reached.

22. The method of claim 21, wherein the mature LMC is dehumidified until a loss on drying of about 10% is reached.

23. A method of making a drug delivery device, the method comprising: forming a dispersion of dehumidified mature lyophilized milled collagen (mLMC); adding a solution of bupivacaine, or a salt thereof, to the dispersion to form a bupivacaine-collagen mixture; filling a container with the bupivacaine-collagen mixture; and freeze drying the contents of the container to form a drug delivery device comprising a collagen matrix comprising bupivacaine, or a salt thereof.

24. The method of claim 23, wherein the dehumidified mature LMC comprises dehydrated LMC that has a loss on drying (LOD) of between about 17% and about 22% and which has been dehumidified to an LOD of between about 8% and about 12%.

25. The method of claim 24, wherein the dehydrated LMC has an LOD of about 18% and has been dehumidified to an LOD of about 10%.

26. The method of any one of claims 23 to 25, wherein the method further comprises: placing the containers into a secondary packaging; sterilizing the drug delivery device in the secondary packaging; and aerating the drug delivery device in secondary packaging.

27. The method of claim 26, wherein the sterilizing comprises sterilizing the drug delivery device with a mixture of about 6% ethylene oxide and about 94% CO₂.

28. The method of claim 26 or 27, wherein the aerating comprises aerating the drug delivery device until it comprises less than about 175 ppm ethylene chlorohydrin.

29. The method of any one of claims 26 to 28, wherein the aerating comprises aerating the drug delivery device until it comprises less than about 0.9 ppm ethylene oxide, less than about 1,000 ppm ethylene glycol, or both.

30. The method of any one of claims 23 to 29, wherein the drug delivery device comprises about 4 mg/cm³ to about 8 mg/cm³ collagen and about 6 mg/cm³ to about 10 mg/cm³ bupivacaine, or a salt thereof.

31. The method of any one of claims 23 to 30, wherein the drug delivery device comprises about 6 mg/cm³ collagen and about 8 mg/cm³ bupivacaine, or a salt thereof.

32. A drug delivery device made by the method of any one of claims 23 to 31.

33. A method of performing a soft tissue surgery procedure in a subject in need thereof, comprising placing the drug delivery device of any one of claims 1 to 7, or 32, at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia, or nerve blockade which lasts for at least about one day.

34. The method of claim 33, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

35. The method of claim 33, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

36. The method of claim 33, wherein the surgery procedure is open ventral hernia repair comprising mesh placement at different subcutaneous layers, including intraperitoneal placement.

37. The method of claim 33, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

38. The method of claim 33, wherein the surgery procedure is open ventral hernia repair comprising onlay mesh repair, wherein the drug delivery device is placed in part in the preperitoneal space between the closed peritoneum and posterior rectus sheath, and in part laid in direct contact with the closed anterior rectus sheath.

39. The method of claim 33, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

40. The method of claim 33, wherein the surgery procedure is laparoscopic-assisted colectomy comprising placing the drug delivery device in part at the site of fascial repair, and in part superficially above the abdominal wall musculature and beneath extraction site incision.

41. The method of claim 33, wherein the surgery procedure is reduction mammoplasty comprising placing the drug delivery device in part in one or both breast pockets.

42. The method of any one of claims 33 to 41, wherein more than one drug delivery device is placed at the surgical site.

43. The method of claim 42, wherein three drug delivery devices are placed at multiple layers in the soft tissue at the surgical site.

44. The method of any one of claims 33 to 43, wherein bupivacaine, or a salt thereof, is present in the drug delivery device in an amount sufficient to provide a duration of local analgesia, local anesthesia, or nerve blockade which lasts for at least about one day after administration.

45. The method of claim 44, wherein bupivacaine, or a salt thereof, is present in the drug delivery device in an amount sufficient to provide a duration of local analgesia, local anesthesia, or nerve blockade which lasts for at least about 72 hours after administration.

46. The method of any one of claims 33 to 45, further comprising partitioning the drug delivery device into segments with a predetermined size, wherein each segment is placed at the surgical site independently.

47. The method of claim 46, wherein the release dissolution profile of the sum of drug delivery device segments is substantially similar to the release dissolution profile of the unpartitioned drug delivery device.

48. The method of claim 47, wherein the release dissolution profile of the sum of drug delivery device segments is within about 1% and about 15% at any point in time substantially similar to the release dissolution profile of the unpartitioned drug delivery device.

49. A kit for performing a soft tissue surgery procedure in a subject in need thereof, the kit comprising the drug delivery device of any one of claims 1-7, or 32, and instructions for placement of the drug delivery device at a surgical site for controlled and/or sustained release of bupivacaine hydrochloride, wherein the bupivacaine hydrochloride is substantially homogeneously dispersed in the collagen matrix and is present in the delivery device in an amount sufficient to provide a duration of local analgesia, local anesthesia or nerve blockade which lasts for at least about one day.

50. The kit of claim 49, wherein the surgery procedure is an abdominoplasty comprising one or more of rectus sheath plication, general anesthesia, making an incision that does not extend above the umbilicus, an anterior approach, making an incision from one anterior superior iliac spine (ASIS) to the other, placing one or more delivery devices on the rectus diastasis at the site of rectus sheath plication, or placing a delivery device below the abdominal incision between Scarpa's fascia and the subcutaneous fat.

51. The kit of claim 49, wherein the surgery procedure is unilateral inguinal hernia repair comprising placing a drug delivery device into the hernia repair site below the site of mesh placement, closing the muscle/fascial layer, placing a drug delivery device between the fascia/muscle closure and the skin closure.

52. The kit of claim 49, wherein the surgery procedure is open ventral hernia repair comprising mesh placement at different subcutaneous layers, including intraperitoneal placement.

53. The kit of claim 49, wherein the surgery procedure is open ventral hernia repair comprising underlay or inlay mesh repair, wherein the drug delivery device is placed in part in the preperitoneal space between the closed peritoneum and posterior rectus sheath, and in part subcutaneously lying in direct contact with the closed anterior rectus sheath with the subcutaneous tissue closed over the matrices.

54. The kit of claim 49, wherein the surgery procedure is open ventral hernia repair comprising mesh repair, wherein the drug delivery device is placed in part in the preperitoneal space between the closed peritoneum and posterior rectus sheath, and in part laid in direct contact with the closed anterior rectus sheath.

55. The kit of claim 49, wherein the surgery procedure is open abdominal hysterectomy comprising placing the drug delivery device in part at the vaginal vault, in part at the site of the peritoneal incision between the visceral peritoneum and muscle, and in part on the fascia immediately below the subcutaneous fat under the site of the incision.

56. The kit of claim 49, wherein the surgery procedure is laparoscopic-assisted colectomy comprising placing the drug delivery device in part at the site of fascial repair, and in part superficially above the abdominal wall musculature and beneath extraction site incision.

57. The kit of claim 49, wherein the surgery procedure is reduction mammoplasty comprising placing the drug delivery device in part in one or both breast pockets.

58. The kit of any one of claims 49-57, further comprising instructions for partitioning the drug delivery device into segments with a predetermined size, wherein each segment is placed at the surgical site independently.

59. The kit of claim 58, wherein the release dissolution profile of the sum of drug delivery device segments is substantially similar to the release dissolution profile of the unpartitioned drug delivery device.

60. The kit of claim 59, wherein the release dissolution profile of the sum of drug delivery device segments is within about 1% and about 15% at any point in time substantially similar to the release dissolution profile of the unpartitioned drug delivery device.
